(19) United States
(12) Patent Application Publication
(10) Pub. No.: US 2008/0096861 A1
(43) Pub. Date: Apr. 24, 2008

(54) Chemical Compounds

(75) Inventors: Kristjan Gudmundsson, Durham, NC (US); John Franklin Miller, Durham, NC (US); Elizabeth Madalena Turner, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 11/573,116

(22) PCT Filed: Jul. 29, 2005
(86) PCT No.: PCT/US05/26797

§ 371 Date: Feb. 2, 2007

Related U.S. Application Data
(60) Provisional application No. 60/598,030, filed on Aug. 2, 2004.

(51) Int. Cl. A61K 31/4709; A61K 31/496; A61K 31/5377; A61P 17/02; A61P 29/00; A61P 31/18; A61P 35/00; A61P 37/00; C07D 401/12; C07D 401/14

(52) U.S. Cl. 514/210.21; 514/235.2; 514/253.06; 514/314; 544/128; 544/363; 546/166

(57) ABSTRACT

The present invention provides compounds that demonstrate protective effects on target cells from HIV infection in a manner as to bind to chemokine receptor, and which affect the binding of the natural ligand or chemokine to a receptor such as CXCR4 of a target cell.

### FIELD OF THE INVENTION

The present invention provides novel compounds that demonstrate protective effects on target cells from HIV infection in a manner as to bind specifically to the chemokine receptor, and which affect the binding of the natural ligand or chemokine to a receptor such as CXCR4 and/or CCR5 of a target cell.

### BACKGROUND OF THE INVENTION

HIV gains entry into host cells by means of the CD4 receptor and at least one co-receptor expressed on the surface of the cell membrane. M-tropic strains of HIV utilize the chemokine receptor CCR5, whereas T-tropic strains of HIV mainly use CXCR4 as the co-receptor. HIV co-receptor usage largely depends on hyper-variable regions of the V3 loop located on the viral envelope protein gp120. Binding of gp120 with CD4 and the appropriate co-receptor results in a conformational change and unmasking of a second viral envelope protein called gp41. The protein gp41 subsequently interacts with the host cell membrane resulting in fusion of the viral envelop with the cell. Subsequent transfer of viral genetic information into the host cell allows for the continuation of viral replication. Thus infection of host cells with HIV is usually associated with the virus gaining entry into the cell via the formation of the ternary complex of CCR5 or CXCR4, CD4, and gp120.

A pharmacological agent that would inhibit the interaction of gp120 with either CCR5/CD4 or CXCR4/CD4 would be a useful therapeutic in the treatment of a disease, disorder, or condition characterized by infection with M-tropic or T-tropic strains, respectively, either alone or in combination therapy.

Evidence that administration of a selective CXCR4 antagonist could result in an effective therapy comes from in vitro studies that have demonstrated that addition of ligands selective for CXCR4 as well as CXCR4-neutralizing antibodies to cells can block HIV viral/host cell fusion. In addition, human studies with the selective CXCR4 antagonist AMD-3100, have demonstrated that such compounds can significantly reduce T-tropic HIV viral load in those patients that are either dual tropic or those where only the T-tropic form of the virus is present.

In addition to serving as a co-factor for HIV entry, it has been recently suggested that the direct interaction of the HIV viral protein gp120 with CXCR4 could be a possible cause of CD8⁺ T-cell apoptosis and AIDS-related dementia via induction of neuronal cell apoptosis.

The signal provided by SDF-1 on binding to CXCR4 may also play an important role in tumor cell proliferation and regulation of angiogenesis associated with tumor growth; the known angiogenic growth factors VEG-F and bFGF up-regulate levels of CXCR4 in endothelial cells and SDF-1 can induce neovascularization in vivo. In addition, leukemia cells that express CXCR4 migrate and adhere to lymph nodes and bone marrow stromal cells that express SDF-1.

The binding of SDF-1 to CXCR4 has also been implicated in the pathogenesis of atherosclerosis, renal allograft rejection, asthma, and allergic airway inflammation, Alzheimer's disease, and arthritis.

The present invention is directed to compounds that can act as agents that modulate chemokine receptor activity. Such chemokine receptors include, but are not limited to, CCR1, CCR2, CCR3, CCR4, CCR5, CCR6, CCR7, CCR8, CXCR1, CXCR2, CXCR3, CXCR4, and CXCR5.

The present invention provides novel compounds that demonstrate protective effects on target cells from HIV infection in a manner as to bind specifically to the chemokine receptor, and which affect the binding of the natural ligand or chemokine to a receptor, such as CXCR4 and/or CCR5 of a target cell.

### SUMMARY OF THE INVENTION

The present invention includes compounds of formula (I):

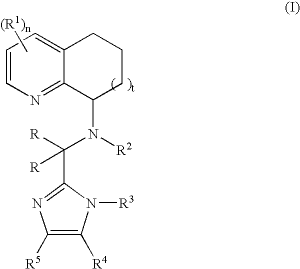

including salts, solvates, and physiologically functional derivatives thereof wherein:
t is 0, 1, or 2;
each R independently is H, alkyl, alkenyl, alkynyl, haloalkyl, cycloalkyl, —R^{a}Ay, —R^{a}OR¹⁰, or —R^{a}S(O)ₘR¹⁰;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)ₘR¹⁰, —S(O)ₘAy, cyano, nitro, or azido;
n is 0, 1, or 2, such that R¹ may be substituted throughout the depicted tetrahydroquinoline;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₘR¹⁰ wherein R² is not amine or alkylamine, or substituted with amine or aklyamine;

### R³ is -Het where Het is optionally substituted, —R^{a}Het where Het is optionally substituted, R^{a}NR⁶R⁷, -Ay[NR⁶R⁷]ₚ, —R^{a}Ay[NR⁶R⁷]ₚ, -Ay[R^{a}NR⁶R⁷]ₚ, —R^{a}Ay[R^{a}NR⁶R⁷]ₚ, -Het[NR⁶R⁷]ₚ, —R^{a}Het[NR⁶R⁷]ₚ, -Het[R^{a}NR⁶R⁷]ₚ, —R^{a}Het[R^{a}NR⁶R⁷]ₚ, —R^{a}Het[R^{a}Het]ₚ, or —R^{a}Het[R^{a}Ay]ₚ;

each p independently is 1 or 2;
each of R⁴ and R⁵ independently are selected from H, alkyl, alkenyl, alkynyl, cycloalkyl, -Ay, -Het, —R^{a}Ay —R^{a}Het, —OR¹⁰, —NR⁶R⁷, —R^{a}NR⁶R⁷, —C(O)R¹⁰, —CO₂R¹⁰, —C(O)NR⁶R⁷, —S(O)₂NR⁶R⁷, —S(O)ₘR¹⁰, cyano, nitro, or azido; or

### R⁴ and R⁵ may combine to form a ring containing one or more degrees of unsaturation that is fused with the depicted imidazole ring optionally substituted with (R¹)ₙ;

each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het, —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group.

In one embodiment, preferably R⁴ and R⁵ combine to form a benzene ring so as to form a benzimidazole.

In one embodiment R⁴ and R⁵ are independently H, alkyl, Ay, Het, —NR⁶R⁷, —R^{a}NR⁶R⁷, —C(O)R¹⁰, —C(O)NR⁶R⁷, —C(O)Ay, —C(O)Het, or —SO₂NR⁶R⁷. Preferably R⁴ and R⁵ are independently H, alkyl, Ay, or —R^{a}NR⁶R⁷. Preferably alkyl is C₁-C₆ alkyl and Ay is phenyl.

In one embodiment n is 1 or 2 and each R¹ independently is selected from halogen, C₁-C₆ alkyl, —OR¹⁰, —NR⁶R⁷, —C(O)R¹⁰, —CO₂R¹⁰, —C(O)NR⁶R⁷, or —S(O)₂NR⁶R⁷.

In one embodiment n is 0.

In one embodiment R² is H, C₁-C₆ alkyl, C₁-C₆ haloalkyl, or C₃-C₆ cycloalkyl. Preferably R² is C₁-C₆ alkyl, C₁-C₆ haloalkyl, or C₃-C₆ cycloalkyl. More preferably R² is C₁-C₆ alkyl or C₃-C₆ cycloalkyl. Most preferably R² is C₁-C₆ alkyl.

In one embodiment each of R⁶ and R⁷ independently are selected from H, C₁-C₆ alkyl, C₃-C₆ cycloalkyl, —R¹⁰H, —R^{a}OR¹⁰.

In one embodiment R¹⁰ is H, C₁-C₆ alkyl or C₃-C₆ cycloalkyl.

In one embodiment R^{a} is C₁-C₆ alkylene or C₃-C₆ cycloalkylene.

In one embodiment R is H, alkyl, or cycloalkyl. More preferably R is H.

In one embodiment R³ is -Het where Het is optionally substituted, —R^{a}Het where Het is optionally substituted, —R^{a}NR⁶R⁷, -Het[NR⁶R⁷]ₚ, —R^{a}Het[NR⁶R⁷]ₚ, -Het[R^{a}NR⁶R⁷]ₚ or —R^{a}Het[R^{a}NR⁶R⁷]ₚ. Most preferably R³ is -Het where Het is optionally substituted, —R^{a}Het where Het is optionally substituted, -Het[NR⁶R⁷]ₚ or —R^{a}Het[NR⁶R⁷]ₚ. In one embodiment, R³ is R^{a}Het where -Het is a nitrogen-containing heterocyclyl or heteroaryl ring, optionally substituted with one or more of C₁-C₆ alkyl, C₃-C₆ cycloalkyl, amino, C₁-C₆alkylamino, hydroxyl, C₁-C₆ alkylhydroxyl, C₁-C₆ alkoxy, C₁-C₆ cycloalkoxy, imidamide, and halogen. In one embodiment R³ is -Het, —R^{a}NR⁶R⁷, -Het[NR⁶R⁷]ₚ, —R^{a}Het[NR⁶R⁷]ₚ, -Het[R^{a}NR⁶R⁷]ₚ, —R^{a}Het[R^{a}NR⁶R⁷]ₚ, or —R^{a}Het, and -Het is a nitrogen-containing heterocyclyl or heteroaryl ring optionally substituted with one or more C₁-C₆ alkyl, C₃-C₆ cycloalkyl, amino, C₁-C₆ alkylamino, hydroxyl, C₁-C₆ alkoxy, C₁-C₆ cycloalkoxy, and halogen.

In one embodiment R³ is -Het, -Het[NR⁶R⁷]ₚ, —R^{a}Het[NR⁶R⁷]ₚ; or —R^{a}Het, and -Het is a nitrogen-containing heterocyclyl or heteroaryl ring optionally substituted with one or more C₁-C₆ alkyl, C₃-C₆ cycloalkyl, amino, C₁-C₆ alkylamino, hydroxyl, C₁-C₆ alkoxy, C₁-C₆ cycloalkoxy, and halogen.

In one embodiment -Het is a nitrogen-containing heterocyclyl or heteroaryl ring.

Preferably, in the present invention, -Het is optionally substituted pyridinyl, piperidinyl, piperazinyl, morpholinyl, pyrrolidinyl, imidazolyl, or azetedinyl. Further, -Het is optionally substituted with one or more C₁-C₆ alkyl, C₃-C₆ cycloalkyl, amino, C₁-C₆ alkylamino, hydroxyl, C₁-C₆ alkylhydroxyl, C₁-C₆ alkoxy, C₁-C₆ cycloalkoxy, imidamide (that is —C(NH)NH₂ and substituted versions thereof) and halogen.

In one embodiment Het is piperidine substituted with H or C₁-C₈ alkyl.

In another embodiment Het is pyrrolidine substituted with H or C₁-C₆ alkyl.

Preferably, in the present invention -Ay is optionally substituted phenyl.

Further, -Ay is optionally substituted with one or more C₁-C₆ alkyl, C₃-C₆ cycloalkyl, amino, C₁-C₆alkylamino, hydroxyl, C₁-C₆ alkoxy, C₁-C₆ cycloalkoxy, and halogen.

In one embodiment t is 1 or 2. In yet another embodiment t is 1.

In one embodiment R³ is —R^{a}Het, and -Het is a nitrogen-containing heterocyclyl or heteroaryl ring optionally substituted with one or more C₁-C₆ alkyl, C₃-C₆ cycloalkyl, amino, C₁-C₆alkylamino, hydroxyl, C₁-C₆ alkoxy, C₁-C₆ cycloalkoxy, and halogen.

Particularly preferred compounds of the present invention include:
N-Methyl-N-{[1-(3-pyridinylmethyl)-1H-benzimidazol-2-yl]methyl}-5,6,7,8-tetrahydro-8-quinolinamine;
N-Methyl-N-({-[2-(1-piperidinyl)ethyl]-1H-benzimidazol-2-yl}methyl)-5,6,7,8-tetrahydro-8-quinolinamine;
N-Methyl-N-({1-[2-(4-morpholinyl)ethyl]-1H-benzimidazol-2-yl}methyl)-5,6,7,8-tetrahydro-8-quinolinamine;
N-Methyl-N-{[1-(4-piperidinylmethyl)-1H-benzimidazol-2-yl]methyl}-5,6,7,8-tetrahydro-8-quinolinamine;
N-Methyl-N-({1-[(1-methyl-3-piperidinyl)methyl]-1H-benzimidazol-2-yl}methyl)-5,6,7,8-tetrahydro-8-quinolinamine;
N-Methyl-N-({1-[(1-methyl-3-pyrrolidinyl)methyl]-1H-benzimidazol-2-yl}methyl)-5,6,7,8-tetrahydro-8-quinolinamine;
N-Methyl-N-({[1-(3-(4-methyl-1-piperazinyl)propyl]-1H-benzimidazol-2-yl}methyl)-5,6,7,8-tetrahydro-8-quinolinamine;
N-Methyl-N-({1-[(1-methyl-3-azetidinyl)methyl]-1H-benzimidazol-2-yl}methyl)-5,6,7,8-tetrahydro-8-quinolinamine;
N-Methyl-N-{[1-(1-methyl-4-piperidinyl)-1H-benzimidazol-2-yl]methyl}-5,6,7,8-tetrahydro-8-quinolinamine;
N-Methyl-N-({1-[(4-{[(2-pyridinylmethyl)amino]methyl}phenyl)methyl]-1H-benzimidazol-2-yl}methyl)-5,6,7,8-tetrahydro-8-quinolinamine;
N-{[1-(4-Aminobutyl)-1H-benzimidazol-2-yl]methyl}-N-methyl-5,6,7,8-tetrahydro-8-quinolinamine;
N-[(1-{[4-(Aminomethyl)phenyl]methyl}-1H-benzimidazol-2-yl)methyl]-N-methyl-5,6,7,8-tetrahydro-8-quinolinamine;
N-({1-[3-(Dimethylamino)propyl]-1H-benzimidazol-2-yl}methyl)-N-methyl-5,6,7,8-tetrahydro-8-quinolinamine;
N-Methyl-N-[(1-{3-[(2-pyridinylmethyl)amino]propyl}-1H-benzimidazol-2-yl)methyl]-5,6,7,8-tetrahydro-8-quinolinamine;
N-({1-[5-(Dimethylamino)pentyl]-1H-benzimidazol-2-yl}methyl)-N-methyl-5,6,7,8-tetrahydro-8-quinolinamine;
N-{[1-(2-Aminoethyl)-1H-benzimidazol-2-yl]methyl}-N-methyl-5,6,7,8-tetrahydro-8-quinolinamine;
N-{[1-(3-Aminopropyl)-1H-benzimidazol-2-yl]methyl}-5,6,7,8-tetrahydro-8-quinolinamine;
N-{[1-(3-Aminopropyl)-1H-benzimidazol-2-yl]methyl}-N-ethyl-5,6,7,8-tetrahydro-8-quinolinamine;
N-{[1-(3-Aminopropyl)-1H-benzimidazol-2-yl]methyl}-N-(phenylmethyl)-5,6,7,8-tetrahydro-8-quinolinamine;
N-{[1-(3-Aminopropyl)-1H-benzimidazol-2-yl]methyl}-N-(3-methylbutyl)-5,6,7,8-tetrahydro-8-quinolinamine;
N-Methyl-N-({1-[2-(1-methyl-2-piperidinyl)ethyl]-1H-benzimidazol-2-yl}methyl)-5,6,7,8-tetrahydro-8-quinolinamine;
N-({1-[(2Z)-4-(Dimethylamino)-2-buten-1-yl]-1H-benzimidazol-2-yl}methyl)-N-methyl-5,6,7,8-tetrahydro-8-quinolinamine;
N-Methyl-N-({1-[(4-methyl-2-morpholinyl)methyl]-1H-benzimidazol-2-yl}methyl)-5,6,7,8-tetrahydro-8-quinolinamine;
N-Methyl-N-{[1-(2-pyridinylmethyl)-1H-benzimidazol-2-yl]methyl}-5,6,7,8-tetrahydro-8-quinolinamine;
N-Methyl-N-{[1-(4-pyridinylmethyl)-1H-benzimidazol-2-yl]methyl}-5,6,7,8-tetrahydro-8-quinolinamine;
2-(2-{[Methyl(5,6,7,8-tetrahydro-8-quinolinyl)amino]methyl}-1H-benzimidazol-1-yl)-N-(4-pyridinylmethyl)acetamide;
2-(2-{[Methyl(5,6,7,8-tetrahydro-8-quinolinyl)amino]methyl}-1H-benzimidazol-1-yl)-N-(3-pyridinylmethyl)acetamide;
N-(3-Aminopropyl)-2-(2-{[methyl(5,6,7,8-tetrahydro-8-quinolinyl)amino]methyl}-1H-benzimidazol-1-yl)acetamide;
N-(2-Aminoethyl)-2-(2-{[methyl(5,6,7,8-tetrahydro-8-quinolinyl)amino]methyl}-1H-benzimidazol-1-yl)acetamide;
N-Methyl-N-({1-[2-oxo-2-(1-piperazinyl)ethyl]-1H-benzimidazol-2-yl}methyl)-5,6,7,8-tetrahydro-8-quinolinamine;
2-(2-{[Methyl(5,6,7,8-tetrahydro-8-quinolinyl)amino]methyl}-1H-benzimidazol-1-yl)-N-(2-pyridinylmethyl)acetamide;
N-Methyl-N-({1-[(1-methyl-3-pyrrolidinyl)methyl]-1H-benzimidazol-2-yl}methyl)-5,6,7,8-tetrahydro-8-quinolinamine HCl salt;
N-({1-[trans-4-(Dimethylamino)cyclohexyl]-1H-benzimidazol-2-yl}methyl)-N-methyl-5,6,7,8-tetrahydro-8-quinolinamine;
N-Methyl-N-({1-[2-(3-pyridinyl)ethyl]-1H-benzimidazol-2-yl}methyl)-5,6,7,8-tetrahydro-8-quinolinamine;
N-methyl-N-({1-[2-(2-pyridinyl)ethyl]-1H-benzimidazol-2-yl}methyl)-5,6,7,8-tetrahydro-8-quinolinamine;
N-({1-[3-(Dimethylamino)propyl]-1H-imidazol-2-yl}methyl)-N-methyl-5,6,7,8-tetrahydro-8-quinolinamine;
(8S)—N-Methyl-N-[(1-{[(3S)-1-methyl-3-piperidinyl]methyl}-1H-benzimidazol-2-yl)methyl]-5,6,7,8-tetrahydro-8-quinolinamine;
(8S)—N-Methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(3-pyridinylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(4-pyridinylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(phenylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(2-methylpropyl)-3-piperidinyl]methyl}-1H-benzimidazol-2-yl)methyl]-5,6,7,8-tetrahydro-8-quinolinamine;
(8S)—N-[(1-{[(3S)-1-(1H-Imidazol-2-ylmethyl)-3-piperidinyl]methyl}-1H-benzimidazol-2-yl)methyl]-N-methyl-5,6,7,8-tetrahydro-8-quinolinamine;
2-((3S)-3-{[2-({Methyl[(8S)-5,6,7,8-tetrahydro-8-quinolinyl]amino}methyl)-1H-benzimidazol-1-yl]methyl}-1-piperidinyl)ethanol;
3-((3S)-3-{[2-({Methyl[(8S)-5,6,7,8-tetrahydro-8-quinolinyl]amino}methyl)-1H-benzimidazol-1-yl]methyl}-1-piperidinyl)-1-propanol;
N-{[1-({3-[(Dimethylamino)methyl]phenyl}methyl)-1H-benzimidazol-2-yl]methyl}-N-methyl-5,6,7,8-tetrahydro-8-quinolinamine;
N-({1-[6-(Dimethylamino)hexyl]-1H-benzimidazol-2-yl}methyl)-N-methyl-5,6,7,8-tetrahydro-8-quinolinamine;
N-{[1-({2-[(Dimethylamino)methyl]phenyl}methyl)-1H-benzimidazol-2-yl]methyl}-N-methyl-5,6,7,8-tetrahydro-8-quinolinamine;
N-[4-(2-{[Methyl(5,6,7,8-tetrahydro-8-quinolinyl)amino]methyl}-1H-benzimidazol-1-yl)butyl]methanesulfonamide;
N-{[1-(3-Aminopropyl)-1H-benzimidazol-2-yl]methyl}-N-(2-phenylethyl)-5,6,7,8-tetrahydro-8-quinolinamine;
N-{[1-(4-Aminobutyl)-1H-benzimidazol-2-yl]methyl}-N-(2-phenylethyl)-5,6,7,8-tetrahydro-8-quinolinamine;
N-{[1-(4-Aminobutyl)-1H-benzimidazol-2-yl]methyl}-N-(3-methylbutyl)-5,6,7,8-tetrahydro-8-quinolinamine;
N-{[1-(4-Aminobutyl)-1H-benzimidazol-2-yl]methyl}-N-(phenylmethyl)-5,6,7,8-tetrahydro-8-quinolinamine;
N-({1-[4-(Dimethylamino)-2-butyn-1-yl]-1H-benzimidazol-2-yl}methyl)-N-methyl-5,6,7,8-tetrahydro-8-quinolinamine;
N-Methyl-N-({1-[3-(4-morpholinyl)propyl]-1H-benzimidazol-2-yl}methyl)-5,6,7,8-tetrahydro-8-quinolinamine;
N-({1-[(2E)-4-Amino-2-buten-1-yl]-1H-benzimidazol-2-yl}methyl)-N-methyl-5,6,7,8-tetrahydro-8-quinolinamine;
N-Methyl-N-({1-[3-(1-methyl-2-piperidinyl)propyl]-1H-benzimidazol-2-yl}methyl)-5,6,7,8-tetrahydro-8-quinolinamine;
N-({1-[3-(Dimethylamino)propyl]-1H-benzimidazol-2-yl}methyl)-N-(1-methylethyl)-5,6,7,8-tetrahydro-8-quinolinamine;
N-[3-(2-{[Methyl(5,6,7,8-tetrahydro-8-quinolinyl)amino]methyl}-1H-benzimidazol-1-yl)propyl]guanidine;
N-[3-(2-{[Methyl(5,6,7,8-tetrahydro-8-quinolinyl)amino]methyl}-1H-benzimidazol-1-yl)propyl]benzenesulfonamide;
N-[3-(2-{[Methyl(5,6,7,8-tetrahydro-8-quinolinyl)amino]methyl}-1H-benzimidazol-1-yl)propyl]methanesulfonamide;
N-Methyl-N-[(1-{3-[(3-methylbutyl)amino]propyl}-1H-benzimidazol-2-yl)methyl]-5,6,7,8-tetrahydro-8-quinolinamine;
N-[(1-{3-[Bis(3-methylbutyl)amino]propyl}-1H-benzimidazol-2-yl)methyl]-N-methyl-5,6,7,8-tetrahydro-8-quinolinamine;
N-({1-[3-(Dimethylamino)-2,2-dimethylpropyl]-1H-benzimidazol-2-yl}methyl)-N-(3-methylbutyl)-5,6,7,8-tetrahydro-8-quinolinamine;
N-({1-[3-(Dimethylamino)-2,2-dimethylpropyl]-1H-benzimidazol-2-yl}methyl)-N-methyl-5,6,7,8-tetrahydro-8-quinolinamine;
N-[2,2-Dimethyl-3-(2-{[methyl(5,6,7,8-tetrahydro-8-quinolinyl)amino]methyl}-1H-benzimidazol-1-yl)propyl]guanidine;
N-[(1-{2,2-Dimethyl-3-[(3-methylbutyl)amino]propyl}-1H-benzimidazol-2-yl)methyl]-N-methyl-5,6,7,8-tetrahydro-8-quinolinamine;
N-({1-[2-(1H-Imidazol-1-yl)ethyl]-1H-benzimidazol-2-yl}methyl)-N-methyl-5,6,7,8-tetrahydro-8-quinolinamine;
N-Methyl-N-({1-[2-(1-methyl-1H-imidazol-5-yl)ethyl]-1H-benzimidazol-2-yl}methyl)-5,6,7,8-tetrahydro-8-quinolinamine;
N-Methyl-N-({1-[2-(1-methyl-1H-imidazol-4-yl)ethyl]-1H-benzimidazol-2-yl}methyl)-5,6,7,8-tetrahydro-8-quinolinamine;
N-[(1-{4-[(Dimethylamino)methyl]phenyl}-1H-benzimidazol-2-yl)methyl]-N-methyl-5,6,7,8-tetrahydro-8-quinolinamine;
N-[(1-{2-[(Dimethylamino)methyl]phenyl}-1H-benzimidazol-2-yl)methyl]-N-methyl-5,6,7,8-tetrahydro-8-quinolinamine;
(8S)—N-Methyl-N-[(1-{[(3R)-1-methyl-3-piperidinyl]methyl}-1H-benzimidazol-2-yl)methyl]-5,6,7,8-tetrahydro-8-quinolinamine;
(8R)—N-Methyl-N-[(1-{[(3S)-1-methyl-3-piperidinyl]methyl}-1H-benzimidazol-2-yl)methyl]-5,6,7,8-tetrahydro-8-quinolinamine;
(8R)—N-Methyl-N-[(1-{[(3R)-1-methyl-3-piperidinyl]methyl}-1H-benzimidazol-2-yl)methyl]-5,6,7,8-tetrahydro-8-quinolinamine;
(8R)—N-Methyl-N-[(1-{[(3R)-1-(3-methylbutyl)-3-piperidinyl]methyl}-1H-benzimidazol-2-yl)methyl]-5,6,7,8-tetrahydro-8-quinolinamine;
(8R)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine;
(8S)—N-Methyl-N-[(1-{[(3R)-1-(3-methylbutyl)-3-piperidinyl]methyl}-1H-benzimidazol-2-yl)methyl]-5,6,7,8-tetrahydro-8-quinolinamine;
(8S)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine;
(8R)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine;
(8R)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine;
(3S)-3-{[2-({Methyl[(8R)-5,6,7,8-tetrahydro-8-quinolinyl]amino}methyl)-1H-benzimidazol-1-yl]methyl}-1-piperidinecarboximidamide;
(3S)-3-{[2-({Methyl[(8S)-5,6,7,8-tetrahydro-8-quinolinyl]amino}methyl)-1H-benzimidazol-1-yl]methyl}-1-piperidinecarboximidamide;
(3R)-3-{[2-({Methyl[(8R)-5,6,7,8-tetrahydro-8-quinolinyl]amino}methyl)-1H-benzimidazol-1-yl]methyl}-1-piperidinecarboximidamide;
(3R)-3-{[2-({Methyl[(8S)-5,6,7,8-tetrahydro-8-quinolinyl]amino}methyl)-1H-benzimidazol-1-yl]methyl}-1-piperidinecarboximidamide;
(3R)—N-Cyano-3-{[2-({methyl[(8S)-5,6,7,8-tetrahydro-8-quinolinyl]amino}methyl)-1H-benzimidazol-1-yl]methyl}-N-propyl-1-piperidinecarboximidamide;
(3R)—N-Cyano-3-{[2-({methyl[(8S)-5,6,7,8-tetrahydro-8-quinolinyl]amino}methyl)-1H-benzimidazol-1-yl]methyl}-1-piperidinecarboximidamide;
(3R)—N′-Cyano-N,N-dimethyl-3-{[2-({methyl[(8S)-5,6,7,8-tetrahydro-8-quinolinyl]amino}methyl)-1H-benzimidazol-1-yl]methyl}-1-piperidinecarboximidamide;
(8S)—N-[(1-{[(3S)-1-Methyl-3-piperidinyl]methyl}-1H-benzimidazol-2-yl)methyl]-N-propyl-5,6,7,8-tetrahydro-8-quinolinamine;
(8S)—N-[(1-{[(3S)-1-Methyl-3-piperidinyl]methyl}-1H-benzimidazol-2-yl)methyl]-N-(2-methylpropyl)-5,6,7,8-tetrahydro-8-quinolinamine;
2-{[(1-{[(3S)-1-Methyl-3-piperidinyl]methyl}-1H-benzimidazol-2-yl)methyl][(8S)-5,6,7,8-tetrahydro-8-quinolinyl]amino}ethanol;
3-{[(1-{[(3S)-1-Methyl-3-piperidinyl]methyl}-1H-benzimidazol-2-yl)methyl][(8S)-5,6,7,8-tetrahydro-8-quinolinyl]amino}-1-propanol;
(8S)—N-[(1-{[(3S)-1-(1-Methylethyl)-3-piperidinyl]methyl}-1H-benzimidazol-2-yl)methyl]-N-propyl-5,6,7,8-tetrahydro-8-quinolinamine;
(8S)—N-[(1-{[(3S)-1-(1-Methylethyl)-3-piperidinyl]methyl}-1H-benzimidazol-2-yl)methyl]-N-(2-methylpropyl)-5,6,7,8-tetrahydro-8-quinolinamine;
2-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}ethanol;
3-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}-1-propanol;
(8S)—N-{[1-({(3S)-1-[3-(Dimethylamino)-2,2-dimethylpropyl]-3-piperidinyl}methyl)-1H-benzimidazol-2-yl]methyl}-N-methyl-5,6,7,8-tetrahydro-8-quinolinamine;
(8S)—N-Methyl-N-[(1-{[(3S)-1-(2-thienylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(1,3-thiazol-2-ylmethyl)-3-piperidinyl]methyl}-1H-benzimidazol-2-yl)methyl]-5,6,7,8-tetrahydro-8-quinolinamine;
(8S)—N-Methyl-N-{[1-({(3S)-1-[(1-methyl-1H-pyrrol-2-yl)methyl]-3-piperidinyl}methyl)-1H-benzimidazol-2-yl]methyl}-5,6,7,8-tetrahydro-8-quinolinamine;
(8S)—N-{[1-({(3S)-1-[2-(Dimethylamino)ethyl]-3-piperidinyl}methyl)-1H-benzimidazol-2-yl]methyl}-N-methyl-5,6,7,8-tetrahydro-8-quinolinamine;
(8S)—N-Methyl-N-({1-[((3S)-1-{[(2S)-1-methyl-2-pyrrolidinyl]methyl}-3-piperidinyl)methyl]-1H-benzimidazol-2-yl}methyl)-5,6,7,8-tetrahydro-8-quinolinamine;
(8S)—N-Methyl-N-({1-[((3S)-1-{[(2S)-1-(1-methylethyl)-2-pyrrolidinyl]methyl}-3-piperidinyl)methyl]-1H-benzimidazol-2-yl}methyl)-5,6,7,8-tetrahydro-8-quinolinamine
(8S)—N-Methyl-N-({1-[((3S)-1-{[(2R)-1-methyl-2-pyrrolidinyl]methyl}-3-piperidinyl)methyl]-1H-benzimidazol-2-yl}methyl)-5,6,7,8-tetrahydro-8-quinolinamine;
(8S)—N-Methyl-N-({1-[((3S)-1-{[(2R)-1-(1-methylethyl)-2-pyrrolidinyl]methyl}-3-piperidinyl)methyl]-1H-benzimidazol-2-yl}methyl)-5,6,7,8-tetrahydro-8-quinolinamine;
(8S)—N-{[1-(3-Aminopropyl)-1H-benzimidazol-2-yl]methyl}-N-methyl-5,6,7,8-tetrahydro-8-quinolinamine;
(8S)—N-({1-[3-(Dimethylamino)propyl]-1H-benzimidazol-2-yl}methyl)-N-methyl-5,6,7,8-tetrahydro-8-quinolinamine;
(8S)—N-Methyl-N-[(1-{3-[(2-methylpropyl)amino]propyl}-1H-benzimidazol-2-yl)methyl]-5,6,7,8-tetrahydro-8-quinolinamine;
(8S)—N-Methyl-N-[(1-{3-[(1-methylethyl)amino]propyl}-1H-benzimidazol-2-yl)methyl]-5,6,7,8-tetrahydro-8-quinolinamine; and
(8S)—N-[(1-{3-[(1H-Imidazol-2-ylmethyl)amino]propyl}-1H-benzimidazol-2-yl)methyl]-N-methyl-5,6,7,8-tetrahydro-8-quinolinamine;
   including salts, solvates, and physiologically functional derivatives thereof.

More particularly, the present invention includes the following compounds, which demonstrate enhanced potency:
N-Methyl-N-{[1-(4-piperidinylmethyl)-1H-benzimidazol-2-yl]methyl}-5,6,7,8-tetrahydro-8-quinolinamine;
N-Methyl-N-({1-[(1-methyl-3-piperidinyl)methyl]-1H-benzimidazol-2-yl}methyl)-5,6,7,8-tetrahydro-8-quinolinamine;
N-Methyl-N-({1-[(1-methyl-3-pyrrolidinyl)methyl]-1H-benzimidazol-2-yl}methyl)-5,6,7,8-tetrahydro-8-quinolinamine;
N-Methyl-N-({1-[3-(4-methyl-1-piperazinyl)propyl]-1H-benzimidazol-2-yl}methyl)-5,6,7,8-tetrahydro-8-quinolinamine;
N-Methyl-N-({1-[(1-methyl-3-azetidinyl)methyl]-1H-benzimidazol-2-yl}methyl)-5,6,7,8-tetrahydro-8-quinolinamine;
N-Methyl-N-{[1-(1-methyl-4-piperidinyl)-1H-benzimidazol-2-yl]methyl}-5,6,7,8-tetrahydro-8-quinolinamine;
N-({1-[3-(Dimethylamino)propyl]-1H-benzimidazol-2-yl}methyl)-N-methyl-5,6,7,8-tetrahydro-8-quinolinamine;
N-{[1-(3-Aminopropyl)-1H-benzimidazol-2-yl]methyl}-5,6,7,8-tetrahydro-8-quinolinamine;
N-{[1-(3-Aminopropyl)-1H-benzimidazol-2-yl]methyl}-N-ethyl-5,6,7,8-tetrahydro-8-quinolinamine;
N-Methyl-N-({1-[2-(1-methyl-2-piperidinyl)ethyl]-1H-benzimidazol-2-yl}methyl)-5,6,7,8-tetrahydro-8-quinolinamine; and
N-({1-[(2Z)-4-(Dimethylamino)-2-buten-1-yl]-1H-benzimidazol-2-yl}methyl)-N-methyl-5,6,7,8-tetrahydro-8-quinolinamine, including salts, solvates, and physiologically functional derivatives thereof.

More particularly, the present invention includes the following compounds, which demonstrate enhanced potency:
N-Methyl-N-({1-[(1-methyl-3-piperidinyl)methyl]-1H-benzimidazol-2-yl}methyl)-5,6,7,8-tetrahydro-8-quinolinamine;
N-({1-[3-(Dimethylamino)propyl]-1H-benzimidazol-2-yl}methyl)-N-methyl-5,6,7,8-tetrahydro-8-quinolinamine;
N-{[1-(3-Aminopropyl)-1H-benzimidazol-2-yl]methyl}-N-ethyl-5,6,7,8-tetrahydro-8-quinolinamine;
N-Methyl-N-({1-[2-(1-methyl-2-piperidinyl)ethyl]-1H-benzimidazol-2-yl}methyl)-5,6,7,8-tetrahydro-8-quinolinamine; and
N-({1-[(2Z)-4-(Dimethylamino)-2-buten-1-yl]-1H-benzimidazol-2-yl}methyl)-N-methyl-5,6,7,8-tetrahydro-8-quinolinamine, including salts, solvates, and physiologically functional derivatives thereof.

Another aspect of the present invention includes compounds selected from the group consisting of:
N-Methyl-N-({1-[(1-methyl-3-azetidinyl)methyl]-1H-benzimidazol-2-yl}methyl)-5,6,7,8-tetrahydro-8-quinolinamine;
N-Methyl-N-({1-[2-(1-methyl-2-piperidinyl)ethyl]-1H-benzimidazol-2-yl}methyl)-5,6,7,8-tetrahydro-8-quinolinamine;
(8S)—N-Methyl-N-({1-[(3R)-3-piperidinylmethyl]-1H-benzimidazol-2-yl}methyl)-5,6,7,8-tetrahydro-8-quinolinamine;
N-Methyl-N-{[1-(4-piperidinylmethyl)-1H-benzimidazol-2-yl]methyl}-5,6,7,8-tetrahydro-8-quinolinamine;
N-Methyl-N-({1-[(1-methyl-3-piperidinyl)methyl]-1H-benzimidazol-2-yl}methyl)-5,6,7,8-tetrahydro-8-quinolinamine;
N-Methyl-N-{[1-(3-pyrrolidinylmethyl)-1H-benzimidazol-2-yl]methyl}-5,6,7,8-tetrahydro-8-quinolinamine;
N-Methyl-N-({1-[(1-methyl-3-pyrrolidinyl)methyl]-1H-benzimidazol-2-yl}methyl)-5,6,7,8-tetrahydro-8-quinolinamine;
N-Methyl-N-({1-[3-(4-methyl-1-piperazinyl)propyl]-1H-benzimidazol-2-yl}methyl)-5,6,7,8-tetrahydro-8-quinolinamine;
N-Methyl-N-({1-[(1-methyl-3-azetidinyl)methyl]-1H-benzimidazol-2-yl}methyl)-5,6,7,8-tetrahydro-8-quinolinamine;
N-Methyl-N-{[1-(4-piperidinyl)-1H-benzimidazol-2-yl]methyl}-5,6,7,8-tetrahydro-8-quinolinamine;
N-Methyl-N-{[1-(1-methyl-4-piperidinyl)-1H-benzimidazol-2-yl]methyl}-5,6,7,8-tetrahydro-8-quinolinamine;
N-{[1-(3-Aminopropyl)-1H-benzimidazol-2-yl]methyl}-N-methyl-5,6,7,8-tetrahydro-8-quinolinamine;
N-({1-[3-(Dimethylamino)propyl]-1H-benzimidazol-2-yl}methyl)-N-methyl-5,6,7,8-tetrahydro-8-quinolinamine;
N-{[1-(3-Aminopropyl)-1H-benzimidazol-2-yl]methyl}-5,6,7,8-tetrahydro-8-quinolinamine;
N-{[1-(3-Aminopropyl)-1H-benzimidazol-2-yl]methyl}-N-ethyl-5,6,7,8-tetrahydro-8-quinolinamine;
N-Methyl-N-({1-[2-(2-piperidinyl)ethyl]-1H-benzimidazol-2-yl}methyl)-5,6,7,8-tetrahydro-8-quinolinamine;
N-({1-[(2Z)-4-(Dimethylamino)-2-buten-1-yl]-1H-benzimidazol-2-yl}methyl)-N-methyl-5,6,7,8-tetrahydro-8-quinolinamine;
2-(2-{[Methyl(5,6,7,8-tetrahydro-8-quinolinyl)amino]methyl}-1H-benzimidazol-1-yl)-N-(3-pyridinylmethyl)acetamide;
N-Methyl-N-({1-[(1-methyl-3-pyrrolidinyl)methyl]-1H-benzimidazol-2-yl}methyl)-5,6,7,8-tetrahydro-8-quinolinamine HCl salt;
(8S)—N-Methyl-N-[(1-{[(3S)-1-methyl-3-piperidinyl]methyl}-1H-benzimidazol-2-yl)methyl]-5,6,7,8-tetrahydro-8-quinolinamine;
(8S)—N-Methyl-N-({1-[(3S)-3-piperidinylmethyl]-1H-benzimidazol-2-yl}methyl)-5,6,7,8-tetrahydro-8-quinolinamine;
(8S)—N-Methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine;
(8S)—N-Methyl-N-[1-{[(3S)-1-(3-pyridinylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(4-pyridinylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(phenylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(2-methylpropyl)-3-piperidinyl]methyl}-1H-benzimidazol-2-yl)methyl]-5,6,7,8-tetrahydro-8-quinolinamine;
(8S)—N-[(1-{[(3S)-1-(1H-Imidazol-2-ylmethyl)-3-piperidinyl]methyl}-1H-benzimidazol-2-yl)methyl]-N-methyl-5,6,7,8-tetrahydro-8-quinolinamine;
2-((3S)-3-{[2-({Methyl[(8S)-5,6,7,8-tetrahydro-8-quinolinyl]amino}methyl)-1H-benzimidazol-1-yl]methyl}-1-piperidinyl)ethanol;
3-((3S)-3-{[2-({Methyl[(8S)-5,6,7,8-tetrahydro-8-quinolinyl]amino}methyl)-1H-benzimidazol-1-yl]methyl}-1-piperidinyl)-1-propanol;
N-({1-[3-(Dimethylamino)propyl]-1H-benzimidazol-2-yl}methyl)-N-(1-methylethyl)-5,6,7,8-tetrahydro-8-quinolinamine;
N-[3-(2-{[Methyl(5,6,7,8-tetrahydro-8-quinolinyl)amino]methyl}-1H-benzimidazol-1-yl)propyl]guanidine;
N-Methyl-N-[(1-{3-[(3-methylbutyl)amino]propyl}-1H-benzimidazol-2-yl)methyl]-5,6,7,8-tetrahydro-8-quinolinamine;
N-({1-[3-(Dimethylamino)-2,2-dimethylpropyl]-1H-benzimidazol-2-yl}methyl)-N-methyl-5,6,7,8-tetrahydro-8-quinolinamine;
N-[2,2-Dimethyl-3-(2-{[methyl(5,6,7,8-tetrahydro-8-quinolinyl)amino]methyl}-1H-benzimidazol-1-yl)propyl]guanidine;
(8S)—N-Methyl-N-[(1-{[(3R)-1-methyl-3-piperidinyl]methyl}-1H-benzimidazol-2-yl)methyl]-5,6,7,8-tetrahydro-8-quinolinamine;
(8R)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine;
(8S)—N-Methyl-N-[(1-{[(3R)-1-(3-methylbutyl)-3-piperidinyl]methyl}-1H-benzimidazol-2-yl)methyl]-5,6,7,8-tetrahydro-8-quinolinamine;
(8S)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine;
(8R)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine;
(8R)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine;
(3S)-3-{[2-({Methyl[(8S)-5,6,7,8-tetrahydro-8-quinolinyl]amino}methyl)-1H-benzimidazol-1-yl]methyl}-1-piperidinecarboximidamide;
(3R)-3-{[2-({Methyl[(8S)-5,6,7,8-tetrahydro-8-quinolinyl]amino}methyl)-1H-benzimidazol-1-yl]methyl}-1-piperidinecarboximidamide;
(3R)—N-Cyano-3-{[2-({methyl[(8S)-5,6,7,8-tetrahydro-8-quinolinyl]amino}methyl)-1H-benzimidazol-1-yl]methyl}-1-piperidinecarboximidamide;
(8S)—N-[(1-{[(3S)-1-Methyl-3-piperidinyl]methyl}-1H-benzimidazol-2-yl)methyl]-N-propyl-5,6,7,8-tetrahydro-8-quinolinamine;
2-{[(1-{[(3S)-1-Methyl-3-piperidinyl]methyl}-1H-benzimidazol-2-yl)methyl][(8S)-5,6,7,8-tetrahydro-8-quinolinyl]amino}ethanol;
3-{[(1-{[(3S)-1-Methyl-3-piperidinyl]methyl}-1H-benzimidazol-2-yl)methyl][(8S)-5,6,7,8-tetrahydro-8-quinolinyl]amino}-1-propanol;
(8S)—N-[(1-{[(3S)-1-(1-Methylethyl)-3-piperidinyl]methyl}-1H-benzimidazol-2-yl)methyl]-N-propyl-5,6,7,8-tetrahydro-8-quinolinamine;
2-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}ethanol;
3-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}-1-propanol;
(8S)—N-{[1-({(3S)-1-[3-(Dimethylamino)-2,2-dimethylpropyl]-3-piperidinyl}methyl)-1H-benzimidazol-2-yl]methyl}-N-methyl-5,6,7,8-tetrahydro-8-quinolinamine;
(8S)—N-Methyl-N-[(1-{[(3S)-1-(2-thienylmethyl)-3-piperidinyl]methyl}-1H-benzimidazol-2-yl)methyl]-5,6,7,8-tetrahydro-8-quinolinamine;
(8S)—N-Methyl-N-{[1-({(3S)-1-[(1-methyl-1H-pyrrol-2-yl)methyl]-3-piperidinyl}methyl)-1H-benzimidazol-2-yl]methyl}-5,6,7,8-tetrahydro-8-quinolinamine;
(8S)—N-{[1-({(3S)-1-[2-(Dimethylamino)ethyl]-3-piperidinyl}methyl)-1H-benzimidazol-2-yl]methyl}-N-methyl-5,6,7,8-tetrahydro-8-quinolinamine;
(8S)—N-Methyl-N-({1-[((3S)-1-{[(2S)-1-methyl-2-pyrrolidinyl]methyl}-3-piperidinyl)methyl]-1H-benzimidazol-2-yl}methyl)-5,6,7,8-tetrahydro-8-quinolinamine;
(8S)—N-Methyl-N-({1-[((3S)-1-{[(2S)-1-(1-methylethyl)-2-pyrrolidinyl]methyl}-3-piperidinyl)methyl]-1H-benzimidazol-2-yl}methyl)-5,6,7,8-tetrahydro-8-quinolinamine;
(8S)—N-Methyl-N-({1-[((3S)-1-{[(2R)-1-methyl-2-pyrrolidinyl]methyl}-3-piperidinyl)methyl]-1H-benzimidazol-2-yl}methyl)-5,6,7,8-tetrahydro-8-quinolinamine; and
(8S)—N-{[1-(3-Aminopropyl)-1H-benzimidazol-2-yl]methyl}-N-methyl-5,6,7,8-tetrahydro-8-quinolinamine; and salts, solvates, and physiological functional derivatives thereof.

Another aspect of the present invention includes compounds selected from the group consisting of
N-Methyl-N-({1-[(1-methyl-3-piperidinyl)methyl]-1H-benzimidazol-2-yl}methyl)-5,6,7,8-tetrahydro-8-quinolinamine;
N-Methyl-N-{[1-(3-pyrrolidinylmethyl)-1H-benzimidazol-2-yl]methyl}-5,6,7,8-tetrahydro-8-quinolinamine;
N-Methyl-N-({1-[(1-methyl-3-pyrrolidinyl)methyl]-1H-benzimidazol-2-yl}methyl)-5,6,7,8-tetrahydro-8-quinolinamine;
N-Methyl-N-({1-[2-(2-piperidinyl)ethyl]-1H-benzimidazol-2-yl}methyl)-5,6,7,8-tetrahydro-8-quinolinamine;
N-Methyl-N-({1-[2-(1-methyl-2-piperidinyl)ethyl]-1H-benzimidazol-2-yl}methyl)-5,6,7,8-tetrahydro-8-quinolinamine;
N-Methyl-N-({1-[(1-methyl-3-pyrrolidinyl)methyl]-1H-benzimidazol-2-yl}methyl)-5,6,7,8-tetrahydro-8-quinolinamine HCl salt;
(8S)—N-Methyl-N-({1-[(3S)-3-piperidinylmethyl]-1H-benzimidazol-2-yl}methyl)-5,6,7,8-tetrahydro-8-quinolinamine;
(8S)—N-Methyl-N-[(1-{[(3S)-1-methyl-3-piperidinyl]methyl}-1H-benzimidazol-2-yl)methyl]-5,6,7,8-tetrahydro-8-quinolinamine;
(8S)—N-Methyl-N-[(1-{[(3S)-1-(2-methylpropyl)-3-piperidinyl]methyl}-1H-benzimidazol-2-yl)methyl]-5,6,7,8-tetrahydro-8-quinolinamine;
2-((3S)-3-{[2-({Methyl[(8S)-5,6,7,8-tetrahydro-8-quinolinyl]amino}methyl)-1H-benzimidazol-1-yl]methyl}-1-piperidinyl)ethanol;
3-((3S)-3-{[2-({Methyl[(8S)-5,6,7,8-tetrahydro-8-quinolinyl]amino}methyl)-1H-benzimidazol-1-yl]methyl}-1-piperidinyl)-1-propanol;
(8S)—N-Methyl-N-({1-[(3R)-3-piperidinylmethyl]-1H-benzimidazol-2-yl}methyl)-5,6,7,8-tetrahydro-8-quinolinamine;
(8S)—N-Methyl-N-[(1-{[(3R)-1-methyl-3-piperidinyl]methyl}-1H-benzimidazol-2-yl)methyl]-5,6,7,8-tetrahydro-8-quinolinamine;
(8S)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine;
(8S)—N-[(1-{[(3S)-1-Methyl-3-piperidinyl]methyl}-1H-benzimidazol-2-yl)methyl]-N-propyl-5,6,7,8-tetrahydro-8-quinolinamine;
2-{[(1-{[(3S)-1-Methyl-3-piperidinyl]methyl}-1H-benzimidazol-2-yl)methyl][(8S)-5,6,7,8-tetrahydro-8-quinolinyl]amino}ethanol;
3-{[(1-{[(3S)-1-Methyl-3-piperidinyl]methyl}-1H-benzimidazol-2-yl)methyl][(8S)-5,6,7,8-tetrahydro-8-quinolinyl]amino}-1-propanol;
(8S)—N-[(1-{[(3S)-1-(1-Methylethyl)-3-piperidinyl]methyl}-1H-benzimidazol-2-yl)methyl]-N-propyl-5,6,7,8-tetrahydro-8-quinolinamine;
(8S)—N-[(1-{[(3S)-1-(1-Methylethyl)-3-piperidinyl]methyl}-1H-benzimidazol-2-yl)methyl]-N-(2-methylpropyl)-5,6,7,8-tetrahydro-8-quinolinamine;
2-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}ethanol; and
3-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}-1-propanol; and salts, solvates and physiological functional derivatives thereof.

Another aspect of the present invention includes compounds selected from the group consisting of
(8S)—N-Methyl-N-({1-[(3S)-3-piperidinylmethyl]-1H-benzimidazol-2-yl}methyl)-5,6,7,8-tetrahydro-8-quinolinamine;
5 (8S)—N-Methyl-N-[(1-{[(3S)-1-methyl-3-piperidinyl]methyl}-1H-benzimidazol-2-yl)methyl]-5,6,7,8-tetrahydro-8-quinolinamine;
(8S)—N-Methyl-N-[(1-{[(3S)-1-(2-methylpropyl)-3-piperidinyl]methyl}-1H-benzimidazol-2-yl)methyl]-5,6,7,8-tetrahydro-8-quinolinamine;

(8S)—N-Methyl-N-({1-[(3R)-3-piperidinylmethyl]-1H-benzimidazol-2-yl}methyl)-5,6,7,8-tetrahydro-8-quinolinamine
(8S)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine; and
(8S)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine; and salts, solvates and physiological functional derivatives thereof.

Another aspect of the present invention includes the compounds of the present invention substantially as hereinbefore defined with reference to any one of the Examples.

Another aspect of the present invention includes a pharmaceutical composition comprising one or more compounds of the present invention and a pharmaceutically acceptable carrier.

Another aspect of the present invention includes compounds of the present invention for use as an active therapeutic substance.

Another aspect of the present invention includes compounds of the present invention for use in the treatment or prophylaxis of diseases and conditions caused by inappropriate activity of CXCR4.

Another aspect of the present invention includes compounds of the present invention for use in the treatment or prophylaxis of diseases and conditions caused by inappropriate activity of CCR5.

Another aspect of the present invention includes compounds of the present invention for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers.

Another aspect of the present invention includes compounds of the present invention for use where the condition or disease is HIV infection, rheumatoid arthritis, inflammation, or cancer.

Another aspect of the present invention includes use of the compounds of the present invention in the manufacture of a medicament for use in the treatment or prophylaxis of a condition or disease modulated by a chemokine receptor.

Another aspect of the present invention includes use of the compounds of the present invention wherein the chemokine receptor is CXCR4 or CCR5.

Another aspect of the present invention includes use of the compounds of the present invention in the manufacture of a medicament for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers.

Another aspect of the present invention includes use of the compounds of the present invention wherein the condition or disorder is HIV infection, rheumatoid arthritis, inflammation, or cancer.

Another aspect of the present invention includes a method for the treatment or prophylaxis of a condition or disease modulated by a chemokine receptor through the administration of one or more of the compounds of the present invention. Preferably the chemokine receptor is CXCR4 or CCR5.

Another aspect of the present invention includes a method for the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of one or more of the compounds of the present invention.

Another aspect of the present invention includes a method for the treatment or prophylaxis of HIV infection, rheumatoid arthritis, inflammation, or cancer comprising the administration of one or more of the compounds of the present invention.

Another aspect of the invention includes a process for the preparation of a compound of formula (I)

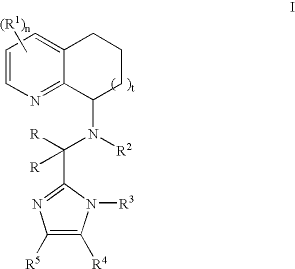

wherein t is 1, each R is H
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, C(O)Het, —S(O)₂NR⁶R⁷, —S(O)ₘR¹⁰, —S(O)ₘAy, cyano, nitro, or azido;
n is 0, 1, or 2;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₘR¹⁰, wherein R² is not amine or alkylamine, or substituted with amine or aklyamine;

### R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}NR⁶R⁷]ₚ, —R^{a}Ay[R^{a}NR⁶R⁷]ₚ, -Het[NR⁶R⁷]ₚ, —R^{a}Het[NR⁶R⁷]ₚ, -Het[R^{a}NR⁶R⁷]ₚ, or —R^{a}Het[R^{a}NR⁶R⁷]ₚ;

each p independently is 1 or 2;
each of R⁴ and R⁵ independently are selected from H, alkyl, alkenyl, alkynyl, cycloalkyl, -Ay, -Het, R^{a}Ay, —R^{a}Het, —OR¹⁰, —NR⁶R⁷, —R^{a}NR⁶R⁷, —C(O)R¹⁰, —CO₂R¹, —C(O)NR⁶R⁷, —S(O)₂NR⁶R⁷, —S(O)ₘR¹⁰, cyano, nitro, or azido; or

### R⁴ and R⁵ may combine to form a ring containing one or more degrees of unsaturation that is fused with the depicted imidazole ring;

each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het, —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group; by reacting a compound of formula (VII)

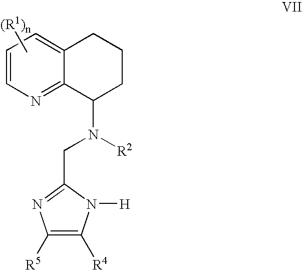

wherein all variables are as defined above, with a compound of formula Lg-R³ wherein Lg is a leaving group and R³ is as defined above, to form a compound of formula (I).

Another aspect of the invention includes a process for the preparation of a compound of formula (I)

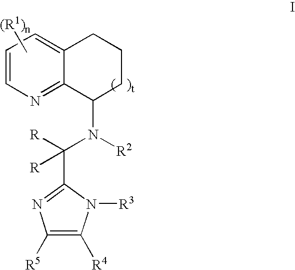

wherein t is 1, each R is H, and all other variables are as defined above, by reacting a compound of formula (II)

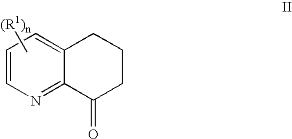

wherein R¹ and n are as defined above; with a compound of formula (VIII)

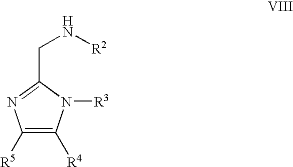

wherein R², R³, R⁴ and R⁵ are as defined above; under reductive amination conditions to form a compound of formula (I).

Another aspect of the invention includes a process for the preparation of a compound of formula (I)

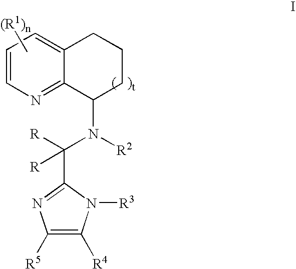

wherein t is 1, each R is H, and all other variables are as defined above, by reacting a compound of formula (IV)

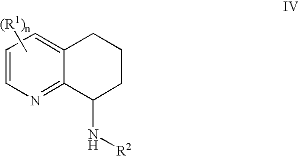

wherein R¹, R² and n are as defined above; with a compound of formula (IX)

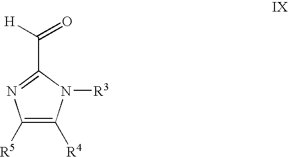

wherein R³, R⁴ and R⁵ are as defined above; under reductive amination conditions to form a compound of formula (I).

Another aspect of the invention includes a process for the preparation of a compound of formula (I)

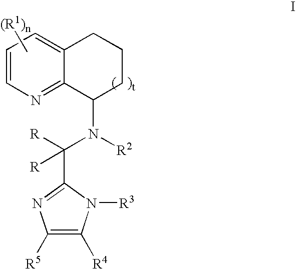

wherein t is 1, each R is H, each of R⁴ and R⁵ combine to form a ring containing one or more degrees of unsaturation that is fused with the depicted imidazole ring and substituted with (R¹)ₙ; and all other variables are as defined above, by reacting a compound of formula (XV)

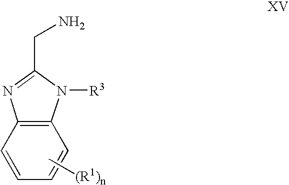

wherein R¹, R³ and n are as defined above; with a compound of formula (II)

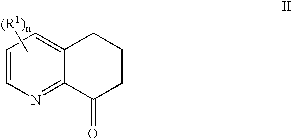

wherein R¹ and n are as defined above; to form a compound of formula (I-A);

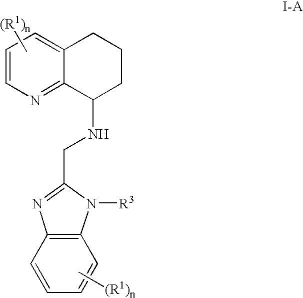

wherein R¹, R³ and n are as defined above; and subsequent reductive amination of formula (I-A) with an aldehyde to form a compound of formula (I).

Another aspect of the invention includes a process for the preparation of a compound of formula (I)

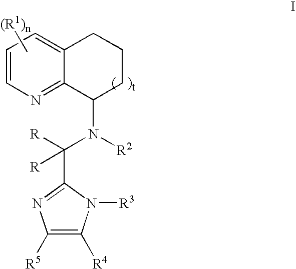

wherein t is 1, each R is H, each of R⁴ and R⁵ combine to form a ring containing one or more degrees of unsaturation that is fused with the depicted imidazole ring and substituted with (R¹)ₙ; and all other variables are as defined above, by treating a compound of formula (XVII)

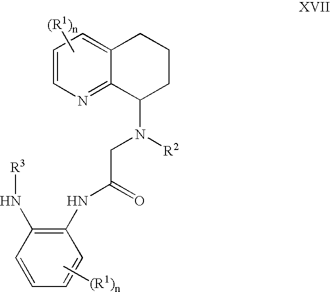

wherein R¹, R², R³ and n are as defined above; with an acid to form a compound of formula (I).

Another aspect of the invention includes the process of the preparation of compounds of formula (I)

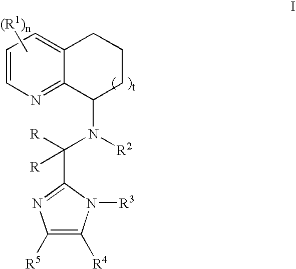

wherein t is 1, each R is H, each of R⁴ and R⁵ combine to form a ring containing one or more degrees of unsaturation that is fused with the depicted imidazole ring and substituted with (R¹)ₙ, and all other variables are as defined above by reacting a compound of formula (XVIII)

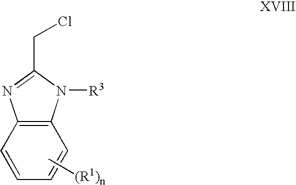

wherein R¹, R³ and n are as defined above; with an amine of formula (IV)

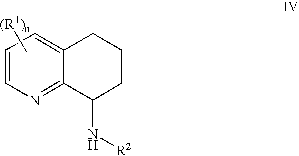

wherein R¹, R² and n are as defined above; to form a compound of formula (I).

Another aspect of the invention includes the preparation of a compound of formula (I)

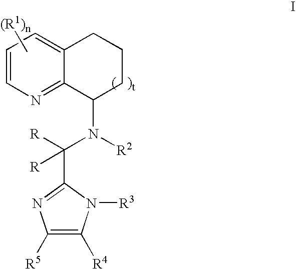

wherein t is 1, each R is H, each of R⁴ and R⁵ combine to form a ring containing one or more degrees of unsaturation that is fused with the depicted imidazole ring and substituted with (R¹)ₙ, and all other variables are as defined above, by reacting a compound of formula (XX)

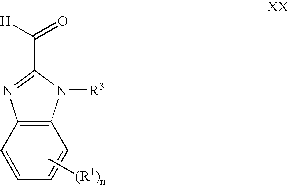

wherein R¹, R³ and n are as defined above; with a compound of formula (IV)

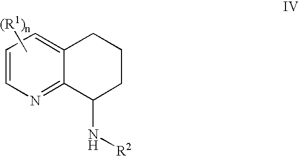

wherein R¹, R² and n are as defined above; to form a compound of formula (I).

### DETAILED DESCRIPTION OF THE INVENTION

Terms are used within their accepted meanings. The following definitions are meant to clarify, but not limit, the terms defined.

As used herein the term “alkyl” refers to a straight or branched chain hydrocarbon, preferably having from one to twelve carbon atoms. Examples of “alkyl” as used herein include, but are not limited to, methyl, ethyl, propyl, isopropyl, isobutyl, n-butyl, tert-butyl, isopentyl, n-pentyl.

As used throughout this specification, the preferred number of atoms, such as carbon atoms, will be represented by, for example, the phrase “Cₓ—C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds. Examples include, but are not limited to, ethynyl and the like.

As used herein, the term “alkylene” refers to a straight or branched chain divalent hydrocarbon radical, preferably having from one to ten carbon atoms. Examples of “alkylene” as used herein include, but are not limited to, methylene, ethylene, n-propylene, n-butylene, and the like.

As used herein, the term “alkenylene” refers to a straight or branched chain divalent hydrocarbon radical, preferably having from one to ten carbon atoms, containing one or more carbon-to-carbon double bonds. Examples include, but are not limited to, vinylene, allylene or 2-propenylene, and the like.

As used herein, the term “alkynylene” refers to a straight or branched chain divalent hydrocarbon radical, preferably having from one to ten carbon atoms, containing one or more carbon-to-carbon triple bonds. Examples include, but are not limited to, ethynylene and the like.

As used herein, the term “cycloalkyl” refers to an optionally substituted non-aromatic cyclic hydrocarbon ring. Exemplary “cycloalkyl” groups include, but are not limited to, cyclopropyl, cyclobutyl, cyclopentyl, cyclohexyl, and cycloheptyl. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane. Preferred substituent groups include alkyl, alkenyl, alkynyl, alkoxy, hydroxyl, oxo, halogen, haloalkyl, cycloalkyl, cycloalkoxy, cyano, amide, amino, and alkylamino.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, and cycloheptenyl. Preferred substituent groups include alkyl, alkenyl, alkynyl, alkoxy, hydroxyl, oxo, halogen, haloalkyl, cycloalkyl, cycloalkoxy, cyano, amide, amino, and alkylamino.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, and cycloheptylene.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, and cycloheptenylene.

As used herein, the term “heterocycle” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. More preferably, the heteroatom is N.

Preferably the heterocyclyl ring is three to twelve-membered and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, piperazine, pyrrolidine, morpholine, azetidine tetrahydrothiopyran, and tetrahydrothiophene. Preferred substituent groups include alkyl, alkenyl, alkynyl, alkoxy, hydroxyl, alkylhydroxy, halogen, haloalkyl, cycloalkyl, cycloalkoxy, cyano, amide, amino, alkylamino and imidamide (that is —C(NH)NH₂ and substituted versions thereof).

As used herein, the term “aryl” refers to an optionally substituted benzene ring or to an optionally substituted fused benzene ring system, for example anthracene, phenanthrene, or naphthalene ring systems. Examples of “aryl” groups include, but are not limited to, phenyl, 2-naphthyl, and 1-naphthyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “heteroaryl” refers to an optionally substituted monocyclic five to seven membered aromatic ring, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Preferably, the heteroatom is N.

Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl, and pyrazolopyrimidinyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to a group —OR′, where R′ is alkyl as defined.

As used herein the term “cycloalkoxy” refers to a group —OR′, where R′ is cycloalkyl as defined.

As used herein the term “alkoxycarbonyl” refers to groups such as:

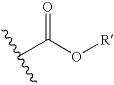

where the R′ represents an alkyl group as herein defined.

As used herein the term “aryloxycarbonyl” refers to groups such as:

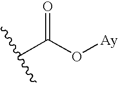

where the Ay represents an aryl group as herein defined.

As used herein imidamide refers to —C(NH)NH₂ and substituted versions thereof for example, C(N(CN)N(alkyl)₂ and analogs.

As used herein the term “nitro” refers to a group —NO₂.

As used herein the term “cyano” refers to a group —CN.

As used herein the term “azido” refers to a group —N₃.

As used herein the term amino refers to a group —NR′R″, where R′ and R″ independently represent H, alkyl, alkenyl, alkynyl, cycloalkyl, heterocyclyl, aryl, or heteroaryl. Similarly, the term “alkylamino” includes an alkylene linker through which the amino group is attached. Examples of “alkylamino” as used herein include groups such as —(CH₂)ₙNH₂, where x is preferably 1 to 6.

As used herein the term “amide” refers to a group —C(O)NR′R″, where R′ and R″ independently represent H, alkyl, alkenyl, alkynyl, cycloalkyl, heterocyclyl, aryl, or heteroaryl. Examples of “amide” as used herein include groups such as —C(O)NH₂, —C(O)NH(CH₃), —C(O)N(CH₃)₂, and the like.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, carbonate, sulfate, tartrate, borate, calcium edetate, camsylate, carbonate, clavulanate, citrate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water or a pharmaceutically acceptable alcohol.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “modulators” as used herein is intended to encompass antagonist, agonist, inverse agonist, partial agonist or partial antagonist, inhibitors and activators. In one preferred embodiment of the present invention, the compounds demonstrate protective effects against HIV infection by inhibiting binding of HIV to a chemokine receptor such as CXCR4 and/or CCR5 of a target cell. The invention includes a method that comprises contacting the target cell with an amount of the compound that is effective at inhibiting the binding of the virus to the chemokine receptor.

In addition to the role chemokine receptors play in HIV infection this receptor class has also been implicated in a wide variety of diseases. Thus CXCR4 modulators may also have a therapeutic role in the treatment of diseases associated with hematopoiesis, including but not limited to, controlling the side effects of chemotherapy, enhancing the success of bone marrow transplantation, enhancing wound healing and burn treatment, as well as combating bacterial infections in leukemia. In addition, compounds may also have a therapeutic role in diseases associated with inflammation, including but not limited to inflammatory or allergic diseases such as asthma, allergic rhinitis, hypersensitivity lung diseases, hypersensitivity pneumonitis, eosinophilic pneumonitis, delayed-type hypersensitivity, interstitial lung disease (ILD) (e.g. idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases such as rheumatoid arthritis, psoriatic arthritis, systemic lupus erythematosus, myastenia gravis, juvenile onset diabetes; glomerulonephritis, autoimmune throiditis, graft rejection, including allograft rejection or graft-versus-host disease; inflammatory bowel diseases, such as Crohn's disease and ulcerative colitus; spondyloarthropathies; scleroderma; psoriasis (including T-cell-mediated psoriasis) and inflammatory dermatoses such as dermatitis, eczema, atopic dermatitis, allergic contact dermatitis, urticaria, vasculitis (e.g. necrotizing, cutaneous, and hypersensitivity vasculitis); eoosinophilic myotis, eosinophilic fasciitis; and cancers.

For use in therapy, therapeutically effective amounts of a compound of formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of compounds of the formula (I) and salts, solvates, and physiological functional derivatives thereof, and one or more pharmaceutically acceptable carriers, diluents, or excipients. The compounds of formula (I) and salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formula (I) or salts, solvates, and physiological functional derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of formula (I) for the treatment of humans suffering from frailty, generally, should be in the range of 0.1 to 100 mg/kg body weight of recipient (mammal) per day. More usually the effective amount should be in the range of 0.1 to 10 mg/kg body weight per day. Thus, for a 70 kg adult mammal the actual amount per day would usually be from 7 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. The compounds may be used in combination with any other pharmaceutical composition where such combined therapy may be useful to modulate chemokine receptor activity and thereby prevent and treat inflammatory and/or immunoregulatory diseases.

The present invention may be used in combination with one or more agents useful in the prevention or treatment of HIV. Examples of such agents include:

Nucleotide reverse transcriptase inhibitors such as zidovudine, didanosine, lamivudine, zalcitabine, abacavir, stavidine, adefovir, adefovir dipivoxil, fozivudine, todoxil, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, and similar agents;

Protease inhibitors such as saquinavir, ritonavir, indinavir, nelfinavir, aprenavir, palinavir, lasinavir, and similar agents;

Entry inhibitors such as T-20, T-1249, PRO-542, PRO-140, TNX-355, BMS-806, 5-Helix and similar agents;

Integrase inhibitors such as L-870,180 and similar agents;

Budding inhibitors such as PA-344 and PA-457, and similar agents; and

Other CXCR4 and/or CCR5 inhibitors such as Sch-C, Sch-D, TAK779, UK 427,857, TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be administered prior to, concurrent to, or subsequent to the administration of other agent(s).

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the scope of the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### Experimental Section

Abbreviations

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

g (grams); mg (milligrams);

### L (liters); mL (milliliters);

### μL (microliters); psi (pounds per square inch);

### M (molar); mM (millimolar);

### Hz (Hertz); MHz (megahertz);

mol (moles); mmol (millimoles);

### RT (room temperature); h (hours);

min (minutes); TLC (thin layer chromatography);
mp (melting point); RP (reverse phase);

### Tr (retention time); TFA (trifluoroacetic acid);

### TEA (triethylamine); THF (tetrahydrofuran);

### TFAA (trifluoroacetic anhydride); CD₃OD (deuterated methanol);

### CDCl₃ (deuterated chloroform); DMSO (dimethylsulfoxide);

### SiO₂ (silica); atm (atmosphere);

### EtOAc (ethyl acetate); CHCl₃ (chloroform);

### HCl (hydrochloric acid); Ac (acetyl);

### DMF (N,N-dimethylformamide); Me (methyl);

### Cs₂CO₃ (cesium carbonate); EtOH (ethanol);

### Et (ethyl); tBu (tert-butyl);

### MeOH (methanol).

Unless otherwise indicated, all temperatures are expressed in ° C. (degrees Centigrade). All reactions conducted at room temperature unless otherwise noted.

¹H-NMR spectra were recorded on a Varian VXR-300, a Varian Unity-300, a Varian Unity-400 instrument, or a General Electric QE-300. Chemical shifts are expressed in parts per million (ppm, 8 units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds was assigned by Ab Initio Vibrational Circular Dichroism (VCD) Spectroscopy. The experimental VCD spectrum were acquired in CDCl₃ using a Bomem Chirall® VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure with (R)- or (S)-configuration. Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality 12 (2000) 172; and Gaussian 98, Revision A. 11.4, M. J. Frisch et al., Gaussian, Inc., Pittsburgh Pa., 2002.

Compounds of formula (I) where t is 1 and R is H and all other variables are as defined in connection with formula (I) and Lg is a suitable leaving group and P is a suitable protecting group can be prepared according to Scheme 1.

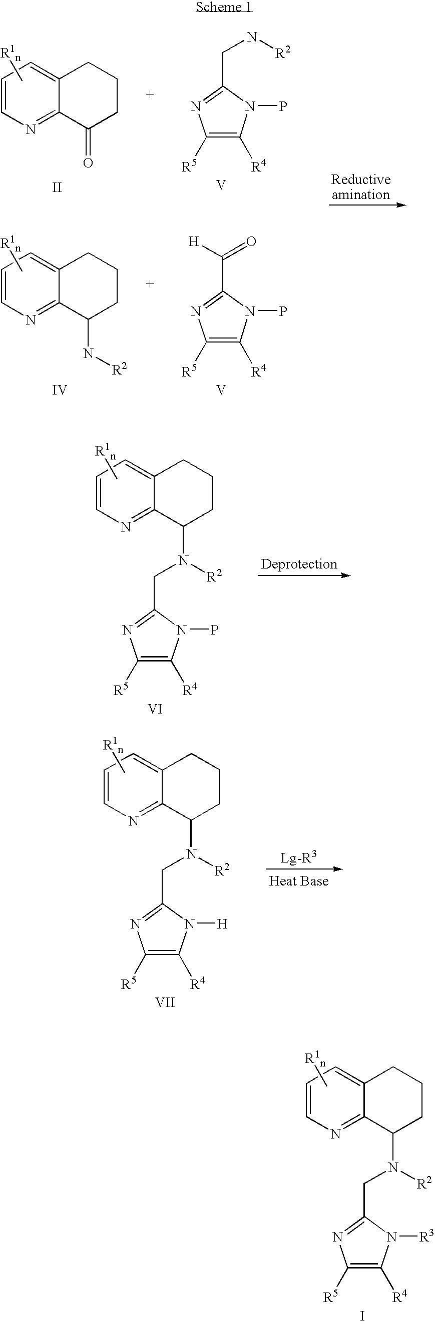

Generally, the process for preparing compounds of formula (I) wherein t is 1 and R is H and Lg is a leaving group and P is a protecting group and all other variables are as defined in connection with formula (I) comprises the following steps:

(a) reacting a compound of formula (II) or a compound of formula (IV) with a compound of formula (III) or a compound of formula (V), respectively, to prepare a compound of formula (VI);

(b) deprotecting a compound of formula (VI) to prepare a compound of formula (VII); and

(c) reacting a compound of formula (VII) with a compound (Lg-R³) to prepare the compound of formula (I).

Compounds of formula (I) where t is 0 or t is 2 can be prepared in a similar fashion.

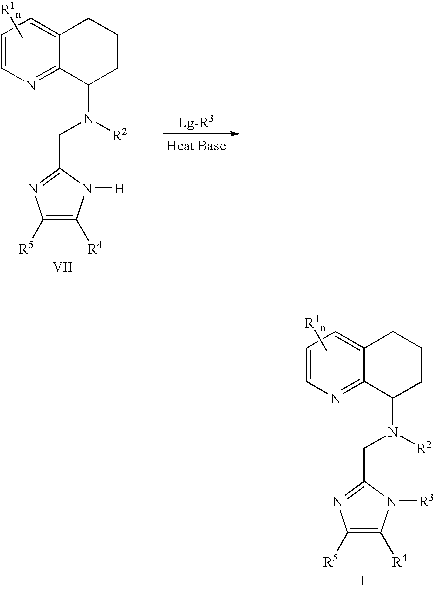

More specifically, compounds of formula (I) can be prepared by reacting a compound of formula (VII) with a compound Lg-R³, wherein all variables are as defined in connection with Scheme 1.

The condensation is conveniently carried out by treating the compound of formula (VII) with a compound Lg-R³ in an inert solvent, optionally in the presence of a base. The reaction may be heated to 50-150° C., optionally in a microwave, or performed at ambient temperature. Suitable inert solvents include N,N-dimethylformamide, dimethylsulfoxide, N-methylpyrrolidone, acetonitrile, nitromethane and the like. The base is typically sodium hydride, sodium alkoxide, potassium carbonate, cesium carbonate, or an amine base such as triethylamine, diisopropylethylamine and the like.

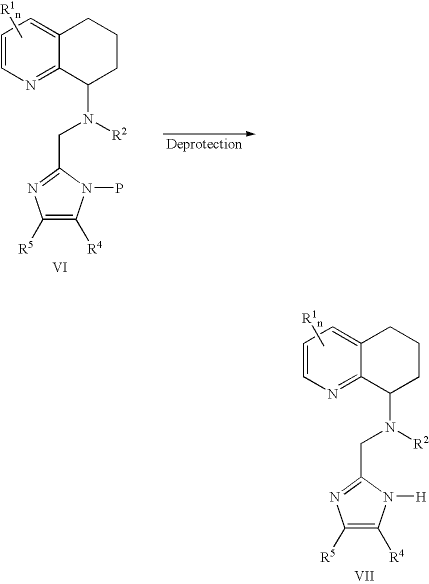

Compounds of formula (VII) may be conveniently prepared by deprotection of compounds of formula (VI), wherein all variables are as defined in connection with Scheme 1.

Deprotection methods depend on the choice of protecting group and are well known to those skilled in the art of synthetic organic chemistry. In one example the protecting group would be a t-butoxycarbonyl (BOC) and would be deprotected using acidic conditions, such as hydrochloric acid or trifluoroacetic acid, in a suitable solvent.

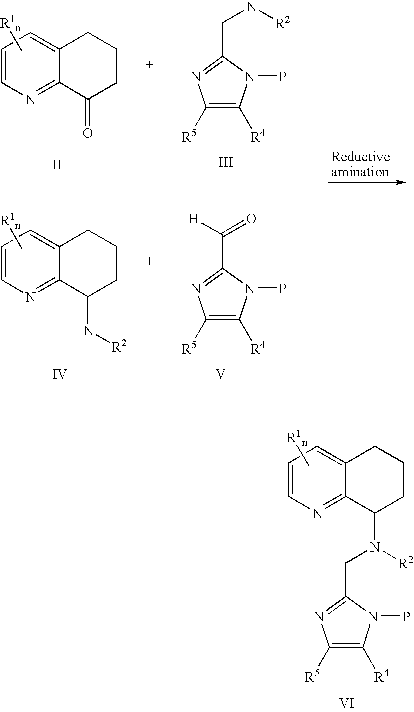

More specifically, compounds of formula (VI) can be prepared by reacting a compound of formula (II) with a compound (III) or alternatively reacting a compound of formula (IV) with a compound of formula (V) under reductive conditions.

Compounds of formula II, III, IV and V can be purchased or prepared using methods that are known in the literature. The reductive amination can be carried out by treating the compound of formula (II) or (IV) with a compound of formula (III) or (V) in an inert solvent in the presence of a reducing agent. The reaction may be heated to 50-150° C. or performed at ambient temperature. Suitable solvents include dichloromethane, dichloroethane, tetrahydrofuran, acetonitrile, toluene and the like. The reducing agent is typically sodium borohydride, sodium cyanoborohydride, sodium triacetoxyborohydride and the like. Optionally the reaction can be run in presence of acid, such as acetic acid, para-toluene sulfonic acid, and the like. A compound of formula (IV) can be prepared from compound of formula (II) via reductive amination. Compounds of formula (III) and of formula (V) can be prepared as described in the literature (e.g. Tet. Lett. 1998, 39, 7467; Science of Synthesis 2002, 12, 529 for compounds where R⁴ and R⁵ are linked to form a benzimidazole).

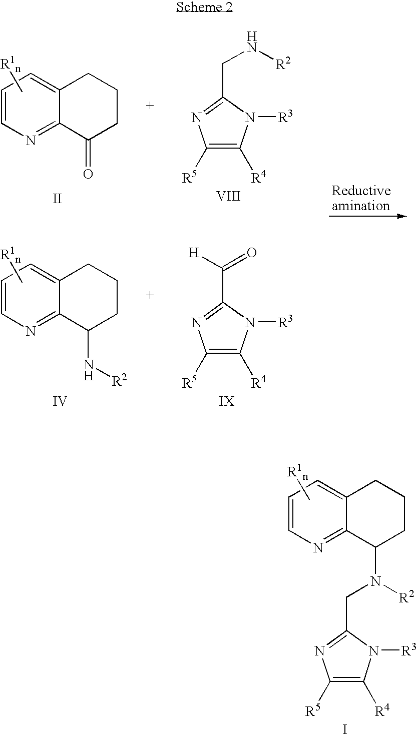

Compounds of formula (I) where t is 1 and R is H and all other variables are as defined as in formula (I) herein, can be prepared directly by reaction of compound of formula (II) or (IV) with a compound of formula (VIII) or (IX) under reductive amination conditions, similar to those described above or well known to those skilled in the art. Compound of formula (II) can be prepared as described in the literature (J. Org. Chem. 2002, 67, 2197-2205) cited herein and incorporated by reference with regard to such synthesis.

Compounds of formula (I-B), where t is 1, R is H and R⁴ and R⁵ are combined together and unsaturated to form a benzene ring fused with the depicted imidazole ring (namely, to form benzimidazoles) could be prepared as outlined in Scheme 3, where Pr is any suitable protecting group and all other variables are as defined in connection with formula (I).

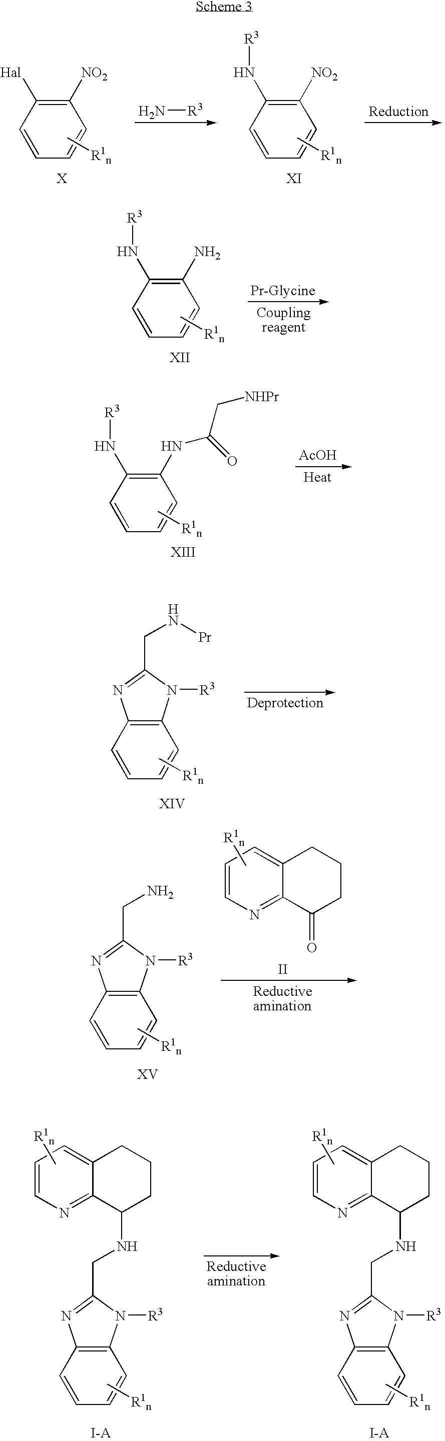

More specifically, compounds of formula (I-B) can be prepared by reductive amination of a compound of formula (I-A) with an aldehyde or a ketone. The reductive amination can be carried out by treating the compound of formula (I-A) with an aldehyde or a ketone in an inert solvent in the presence of a reducing agent. The reaction may be heated to 50-150° C. or performed at ambient temperature. Suitable solvents include dichloromethane, dichloroethane, tetrahydrofuran, acetonitrile, toluene and the like. The reducing agent is typically sodium borohydride, sodium cyanoborohydride, sodium triacetoxyborohydride and the like.

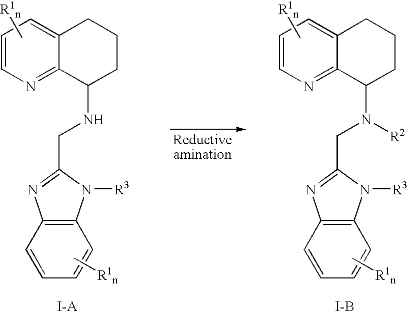

Compounds of formula (I-A) can be prepared from compounds of formula (XV) and compound of formula (II) via reductive amination. The reaction may be heated to 50-150° C. or performed at ambient temperature. Suitable solvents include dichloromethane, dichloroethane, tetrahydrofuran, acetonitrile, toluene and the like. The reducing agent is typically sodium borohydride, sodium cyanoborohydride, sodium triacetoxyborohydride and the like. Compounds of formula (XV) can be prepared in a similar manner as described in the literature (e.g. Tet. Lett. 1998, 39, 7467; Tet. Lett. 2002, 43, 3003; J. Med. Chem. 2002, 45, 713) or as outlined in Scheme 3 by methods well known to those skilled in the art of organic chemistry.

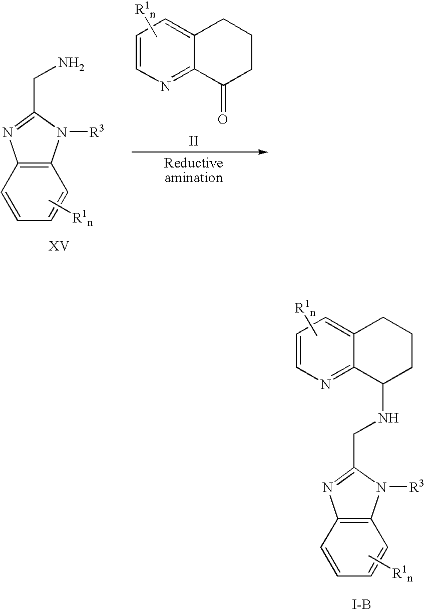

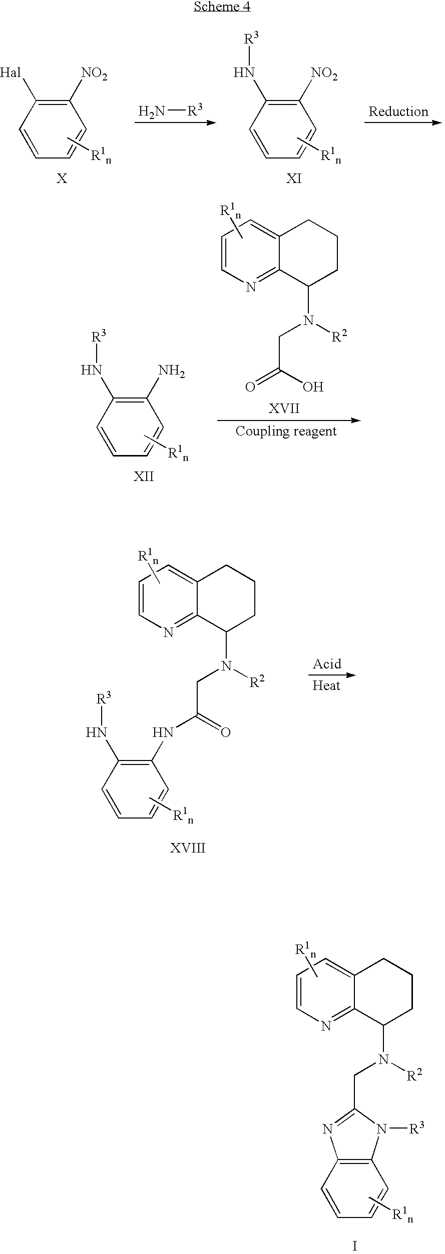

More specifically, compounds of formula (I) can be prepared by treatment of compound of formula (XVIII) under acidic conditions optionally with heating. The reaction can be carried out by treating the compound of formula (XVIII) with a suitable acid optionally in the presence of an inert solvent. The reaction may be heated to 50-200° C. or performed at ambient temperature. Suitable acids include acetic acid, trifluoroacetic acid, hydrochloric acid and the like. The reaction can be carried out using the acid as a solvent. Other suitable solvents include tetrahydrofuran, acetonitrile, toluene and the like.

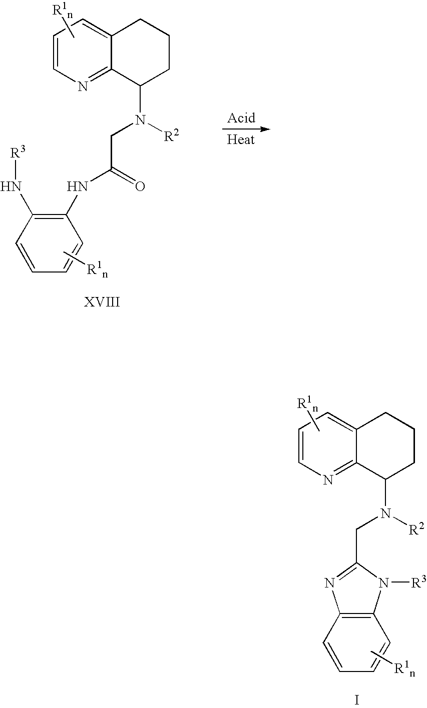

More specifically compounds of formula (XVIII) can be prepared by coupling of a compound of formula (XII) with a compound of formula (XVII). This coupling can be carried out using a variety of coupling reagent well know to those skilled in the art of organic synthesis (e.g. EDC, HOBt/HBTu; BOPCI). The reaction can be carried out with heating or at ambient temperature. Suitable solvents for this reaction include acetonitrile, tetrahydrofuran and the like. Compounds of formula (XII) are commercially available or can be prepared by methods known in the literature and outlined in Scheme 4.

Compounds of formula (XVII) can be prepared from tetrahydroquinoline-8-one and a protected glycine derivative by reductive amination, followed by deprotection.

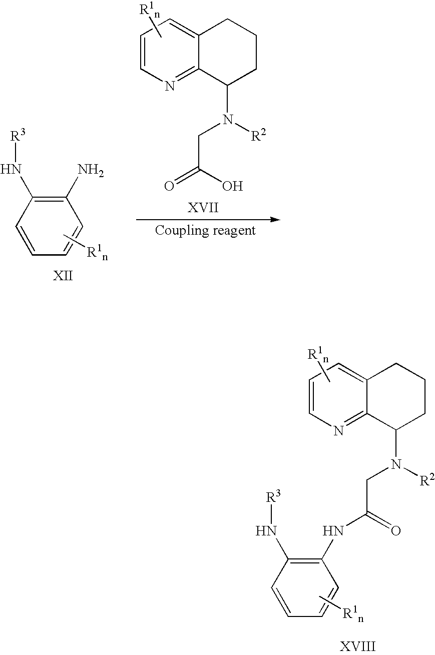

A compound of formula (I) where t is 1, R is H and R⁴ and R⁵ are combined together to form a ring that is fused with the imidazole ring and substituted with (R¹)ₙ and all other variables are as defined in connection with compound of formula (I) can be prepared as outlined in Scheme 5.

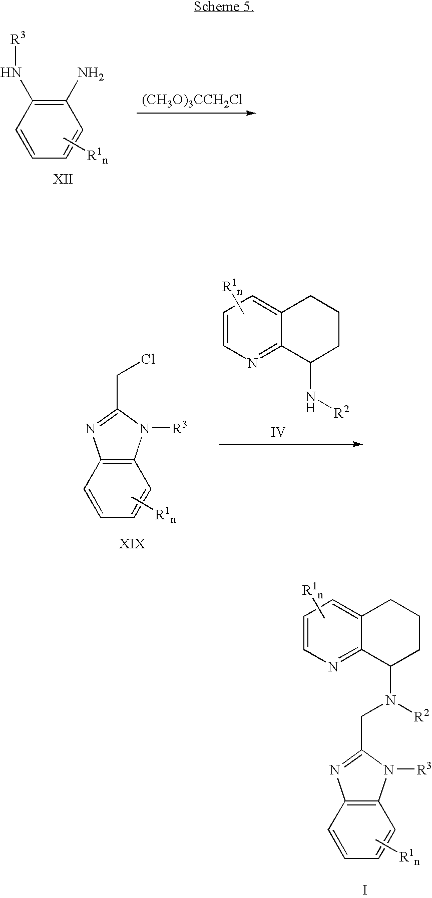

More specifically a compound of formula (I) can be prepared by condensation of a compound of formula (XIX) with a compound of formula (IV) in a suitable solvent, optionally with heating, optionally in the presence of a catalyst and a base. Suitable solvents for this reaction include acetonitrile, N,N-dimethylformamide, nitromethane, dimethylsulfoxide, lower alcohols and the like. The reaction can be carried out at room temperature or optionally with heating between 40-150° C. Optionally the reaction can be carried out in a microwave. Suitable catalyst for this reaction include sodium iodide, potassium iodide, tertbutylammonium iodide and the like. Suitable bases include sodium carbonate, potassium carbonate, cesium carbonate, pyridine, dimethylaminopyridine, triethylamine, diisopropylethylamine and the like. Compounds of formula (IV) can be prepared as described in connection with previous Schemes.

Compounds of formula (XIX) can be prepared from compound of formula (XII) by treatment with 2-chloro-1,1,1-trimethoxyethane optionally in the presence of an acid in a suitable solvent. Suitable acids include p-toluenesulfonic acid and the like. Suitable solvents include dichloromethane, toluene, tetrahydrofuran and the like.

A compound of formula (I) where t is 1, R is H, R⁴ and R⁵ are combined together to form a ring that is fused with the imidazole ring and substituted with (R¹)ₙ and all other variables are as defined in connection with compound of formula (I) can be prepared as outlined in Scheme 6.

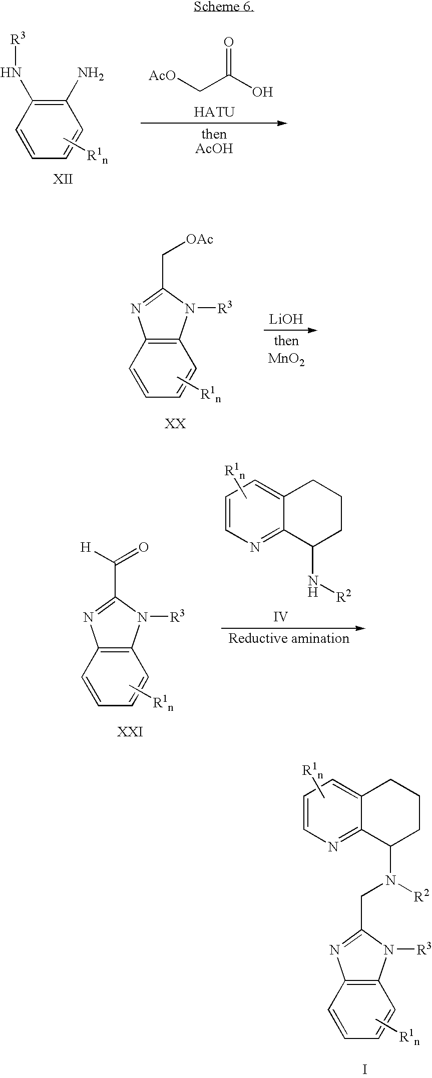

Compound of formula (I) can be prepared by reductive amination of a compound of formula (XXI) with a compound of formula (IV). The reductive amination can be carried out by treating the compound of formula (IV) with a compound of formula (XXI) in an inert solvent in the presence of a reducing agent. The reaction may be heated to 50-150° C. or performed at ambient temperature. Suitable solvents include dichloromethane, dichloroethane, tetrahydrofuran, acetonitrile, toluene and the like.

The reducing agent is typically sodium borohydride, sodium cyanoborohydride, sodium triacetoxyborohydride and the like. Optionally the reaction can be run in presence of acid, such as acetic acid, para-toluene sulfonic acid, and the like. Compound of formula (XXI) can be prepared from a compound of formula (XX) by removal of the acetoxy groups by methods well known to those skilled in the art followed by oxidation to an aldehyde. Suitable oxidizing agents include manganese dioxide and the like and suitable solvents include dichloromethane and the like. A compound of formula (XX) can be prepared from a compound of formula (XII) by treatment with acetoxyacetic acid and a suitable coupling reagent (e.g. HATU: O-(7-Azabenzotriazol-1-yl)-N,N,N,N-tetramethyluronium hexafluorophosphate) followed by ring closing of the resulting amide to a benzimidazole by treatment with acid optionally with heating as outlined in Scheme 6. Suitable acids include acetic acid, trifluoroacetic acid, hydrochloric acid and the like. The reaction can be heated between 20-200° C.

### EXAMPLES

### Example 1

### N-(1H-Benzimidazol-2-ylmethyl)-5,6,7,8-tetrahydro-8-quinolinamine

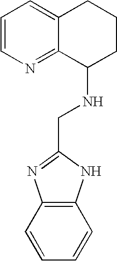

To a stirred solution of 6,7-dihydro-8(5H)-quinolinone (2.00 g, 13.6 mmol, J. Org. Chem., 2002, 67, 2197-2205) in 30 mL of anhydrous MeOH was added 2-(aminomethyl)benzimidazole dihydrochloride (2.99 g, 13.6 mmol, Lancaster). The resulting solution quickly changed color from clear to blue and a solid precipitated shortly thereafter. After 18 hours the suspension was treated with NaBH₄ (1.03 g, 27.2 mmol) over a 5 minute period. Two hours following the NaBH₄ addition an additional portion of NaBH₄ (200 mg, 5.29 mmol) was added and stirring at RT continued. After an additional 2 hours the suspension was concentrated to a volume of approximately 10 mL by rotary evaporation. The mixture was partitioned between dichloromethane and 10% aqueous Na₂CO₃. The aqueous phase was extracted twice with dichloromethane. The two extracts were combined with the original dichloromethane solution, washed twice with water, dried over Na₂SO₄, and concentrated to dryness at reduced pressure. The crude residue was purified by flash chromatography (silica gel, gradient elution of dichloromethane to 9:1 dichloromethane/2M NH₃ in MeOH) to afford 2.77 g (74%) of N-(1H-benzimidazol-2-ylmethyl)-5,6,7,8-tetrahydro-8-quinolinamine as a yellow foam. ¹H NMR (DMSO-d₆): δ 12.39 (brs, 1H), 8.43 (d, 1H), 7.65-7.40 (m, 3H), 7.32-7.09 (m, 3H), 4.12 (s, 2H), 3.79 (t, 2H), 3.39 (s, 1H), 2.79 (m, 2H), 2.10 (m, 1H), 1.99 (m, 1H), 1.69 (m, 2H). MS m/z 279 (M+1).

### Example 2

### N-(1H-Benzimidazol-2-ylmethyl)-N-methyl-5,6,7,8-tetrahydro-8-quinolinamine

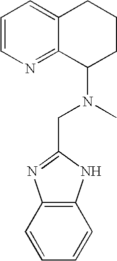

A mixture of N-(1H-benzimidazol-2-ylmethyl)-5,6,7,8-tetrahydro-8-quinolinamine (3.20 g, 11.5 mmol), 37% aqueous formaldehyde (1.40 mL, 17.3 mmol), glacial acetic acid (3.30 mL, 57.5 mmol), and NaBH(OAc)₃ (9.70 g, 46.0 mmol) in 150 mL of 1,2-dichloroethane was stirred at rt for 18 hours. The solution was then diluted with 100 mL of dichoromethane followed by 150 mL of 10% aqueous Na₂CO₃ and the resulting mixture stirred vigorously for 30 minutes. The mixture was transferred to a separatory funnel and the phases separated. The organic solution was washed once with 10% aqueous Na₂CO₃, once with aqueous brine, dried over Na₂SO₄, and concentrated to dryness at reduced pressure. The yellow oil was dissolved in 50 mL of MeOH and stirred with addition of 50 mL of 6N aqueous HCl. After 2 h the solution was cooled in an ice water bath and treated with 70 mL of 5N aqueous NaOH. The resulting mixture was extracted with EtOAc (3×). The combined EtOAc extracts were washed once with aqueous brine, dried over Na₂SO₄, and concentrated to dryness at reduced pressure. The crude product was purified by flash chromatography (silica gel, gradient elution of dichloromethane to 9:1 dichloromethane/2M NH₃ in EtOH) followed by recrystallization from EtOAc/hexane to afford 2.72 g (82%) of N-(1H-benzimidazol-2-ylmethyl)-N-methyl-5,6,7,8-tetrahydro-8-quinolinamine as an off-white powder. ¹H NMR (DMSO-d₆): δ 12.34 (br s, 1H), 8.52 (d, 1H), 7.60-7.45 (m, 3H), 7.30-7.08 (m, 3H), 4.09 (s, 2H), 3.98 (dd, 1H), 2.87 (m, 1H), 2.72 (m, 1H), 2.31 (s, 3H), 2.18-1.90 (m, 3H), 1.71 (m, 1H). MS m/z 293 (M+1).

### Example 3

### N-Methyl-N-{[1-(3-pyridinylmethyl)-1H-benzimidazol-2-yl]methyl}-5,6,7,8-tetrahydro-8-quinolinamine

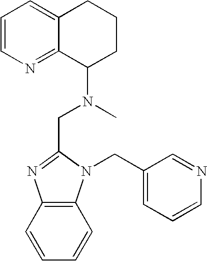

A mixture of N-(1H-benzimidazol-2-ylmethyl)-N-methyl-5,6,7,8-tetrahydro-8-quinolinamine (0.100 g, 0.342 mmol), 3-(chloromethyl)pyridine hydrochloride (0.112 g, 0.684 mmol), and K₂CO₃ (0.236 g, 1.71 mmol) in 5 mL of anhydrous DMF was heated to 70° C. with stirring. After 4 hours the mixture was cooled to RT, diluted with EtOAc, washed with aqueous brine (3×), dried over Na₂SO₄, and concentrated to dryness at reduced pressure. The crude product was purified by flash chromatography (silica gel, gradient elution of dichloromethane to 85:15 dichloromethane/2M NH₃ in MeOH) to afford 87 mg (66%) of N-methyl-N-{[1-(3-pyridinylmethyl)-1H-benzimidazol-2-yl]methyl}-5,6,7,8-tetrahydro-8-quinolinamine as a tacky, yellow foam. ¹H NMR (DMSO-d₆): δ 8.50 (m, 2H), 8.34 (d, 1H), 7.67 (m, 1H), 7.51 (d, 2H), 7.42-7.29 (m, 2H), 7.27-7.12 (m, 3H), 5.79 (dd, 2H), 4.26 (d, 1H), 4.11 (d, 1H), 4.01 (t, 1H), 2.72 (m, 2H), 2.19 (s, 3H), 1.98-1.80 (m, 3H), 1.63 (m, 1H). MS m/z 384 (M+1).

### Example 4

### N-Methyl-N-({1-[2-(1-piperidinyl)ethyl]-1H-benzimidazol-2-yl}methyl)-5,6,7,8-tetrahydro-8-quinolinamine

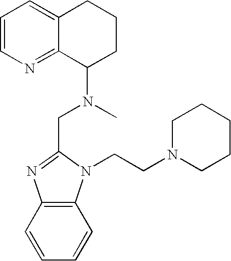

A mixture of N-(1H-benzimidazol-2-ylmethyl)-N-methyl-5,6,7,8-tetrahydro-8-quinolinamine (50.0 mg, 0.171 mmol), N-(2-chloroethyl)piperidine hydrochloride (63.0 mg, 0.342 mmol, Lancaster), potassium iodide (57.0 mg, 0.342 mmol), and K₂CO₃ (0.118 g, 0.855 mmol) in 4 mL of anhydrous DMF was heated to 80° C. with stirring. After 3.5 hours the mixture was cooled to RT, diluted with EtOAc, washed with aqueous brine (3×), dried over Na₂SO₄, and concentrated to dryness at reduced pressure. The crude product was subjected to reverse phase HPLC(C8, gradient elution of H₂O/0.1% TFA to MeCN over 40 minutes). Fractions containing pure product were combined and concentrated to dryness at reduced pressure. The residue was dissolved in EtOAc. The solution was washed once with 10% aqueous Na₂CO₃ followed by aqueous brine. The solution was dried over Na₂SO₄ and concentrated to dryness at reduced pressure to afford 12 mg (17%) of N-methyl-N-({1-[2-(1-piperidinyl)ethyl]-1H-benzimidazol-2-yl}methyl)-5,6,7,8-tetrahydro-8-quinolinamine as a thick, transparent oil. ¹H NMR (CDCl₃): δ 8.51 (d, 1H), 7.71 (d, 1H), 7.43-7.32 (m, 2H), 7.27-7.18 (m, 2H), 7.06 (dd, 1H), 4.67-4.45 (m, 2H), 4.17 (d, 1H), 4.11-3.99 (m, 2H), 2.83 (m, 1H), 2.78-2.21 (m, 9H), 2.19-1.98 (m, 4H), 1.72 (m, 1H), 1.64-1.33 (m, 6H). MS m/z 404 (M+1).

### Example 5

### N-Methyl-N-({1-[2-(4-morpholinyl)ethyl]-1H-benzimidazol-2-yl}methyl)-5,6,7,8-tetrahydro-8-quinolinamine

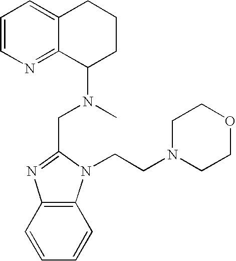

Reaction of N-(1H-benzimidazol-2-ylmethyl)-N-methyl-5,6,7,8-tetrahydro-8-quinolinamine (50.0 mg, 0.171 mmol) with N-(2-chloroethyl)morpholine hydrochloride (95.0 mg, 0.513 mmol, Acros) as described herein for the preparation of N-methyl-N-({1-[2-(1-piperidinyl)ethyl]-1H-benzimidazol-2-yl}methyl)-5,6,7,8-tetrahydro-8-quinolinamine, followed by reverse phase HPLC purification as described in the same procedure, afforded 29 mg (42%) of N-methyl-N-({1-[2-(4-morpholinyl)ethyl]-1H-benzimidazol-2-yl}methyl)-5,6,7,8-tetrahydro-8-quinolinamine as a thick, transparent oil. ¹H NMR (CDCl₃): δ 8.50 (d, 1H), 7.72 (d, 1H), 7.39-7.32 (m, 2H), 7.29-7.18 (m, 2H), 7.04 (m, 1H), 4.62 (m, 1H), 4.53 (m, 1H), 4.15 (d, 1H), 4.10-3.97 (m, 2H), 3.61 (t, 4H), 2.82 (m, 1H), 2.78-2.60 (m, 3H), 2.39 (m, 4H), 2.30 (s, 3H), 2.19-1.97 (m, 3H), 1.72 (m, 1H). MS m/z 406 (M+1).

### Example 6

### N-Methyl-N-{[1-(4-piperidinylmethyl)-1H-benzimidazol-2-yl]methyl}-5,6,7,8-tetrahydro-8-quinolinamine

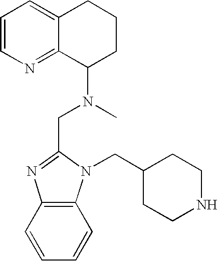

### a) t-Butyl 4-(chloromethyl)-1-piperidinecarboxylate

A suspension of PS-triphenylphosphine resin (3.20 g, 6.69 mmol at 2.20 mmol/g, Argonaut Technologies) in 25 mL of anhydrous dichloromethane was treated with CCl₄ (0.670 mL, 6.96 mmol). After stirring at RT for 30 minutes, a solution of t-butyl 4-(hydroxymethyl)-1-piperidinecarboxylate (0.500 g, 2.32 mmol, Aldrich) in 5 mL of dichloromethane was added. After stirring the mixture at RT overnight the resin was removed by vacuum filtration. The resin was washed with dichloromethane (2×) followed by MeOH (2×). The filtrate was concentrated to dryness at reduced pressure to afford 0.54 g (100%) of t-butyl 4-(chloromethyl)-1-piperidinecarboxylate as a white, crystalline solid. ¹H NMR (DMSO-d₆): δ 3.91 (d, 2H), 3.51 (d, 2H), 2.77-2.55 (br s, 2H), 1.80-1.62 (m, 3H), 1.36 (s, 9H), 1.06 (m, 1H).

### b) t-Butyl 4-[(2-{[methyl(5,6,7,8-tetrahydro-8-quinolinyl)amino]methyl}-1H-benzimidazol-1-yl)methyl]-1-piperidinecarboxylate

Reaction of N-(1H-benzimidazol-2-ylmethyl)-N-methyl-5,6,7,8-tetrahydro-8-quinolinamine (50 mg, 0.171 mmol) with t-butyl 4-(chloromethyl)-1-piperidinecarboxylate (0.160 g, 0.684 mmol) as described herein for the preparation of N-methyl-N-({1-[2-(1-piperidinyl)ethyl]-1H-benzimidazol-2-yl}methyl)-5,6,7,8-tetrahydro-8-quinolinamine, followed by reverse phase HPLC purification as described in the same procedure, afforded 60 mg (71%) of t-butyl 4-[(2-{[methyl(5,6,7,8-tetrahydro-8-quinolinyl)amino]methyl}-1H-benzimidazol-1-yl)methyl]-1-piperidinecarboxylate as an off-white solid. ¹H NMR (CDCl₃): δ 8.56 (d, 1H), 7.79 (m, 1H), 7.42 (d, 1H), 7.38-7.22 (m, 3H), 7.12 (m, 1H), 4.48 (dd, 1H), 4.38-3.88 (m, 6H), 2.92 (m, 1H), 2.80 (m, 1H), 2.67-2.42 (m, 2H), 2.37 (s, 3H), 2.23-1.69 (m, 5H), 1.60-1.07 (m, 1H), 1.00 (m, 2H). MS m/z 490 (M+1).

### c) N-Methyl-N-{[1-(4-piperidinylmethyl)-1H-benzimidazol-2-yl]methyl}-5,6,7,8-tetrahydro-8-quinolinamine

A solution of t-butyl 4-[(2-{[methyl(5,6,7,8-tetrahydro-8-quinolinyl)amino]methyl}-1H-benzimidazol-1-yl)methyl]-1-piperidinecarboxylate (50.0 mg, 0.102 mmol) in 2 mL of anhydrous MeOH was treated with 2 mL of 4N HCl/dioxane and the resulting solution was stirred at RT. After 1 hour the solution was concentrated to dryness at reduced pressure. The solid residue was partitioned between EtOAc and 10% aqueous Na₂CO₃. The EtOAc solution was washed once with aqueous brine, dried over Na₂SO₄, and concentrated to dryness at reduced pressure to afford 35 mg (88%) of N-methyl-N-{[1-(4-piperidinylmethyl)-1H-benzimidazol-2-yl]methyl}-5,6,7,8-tetrahydro-8-quinolinamine as a tacky, white foam. ¹H NMR (CDCl₃): δ 8.52 (d, 1H), 7.71 (d, 1H), 7.38 (d, 1H), 7.30 (m, 1H), 7.26-7.18 (m, 2H), 7.09 (m, 1H), 4.38 (dd, 1H), 4.22 (dd, 1H), 4.15 (d, 1H), 4.03-3.94 (m, 2H), 3.09-2.91 (m, 2H), 2.85 (m, 1H), 2.73 (m, 1H), 2.52-2.38 (m, 2H), 2.30 (s, 3H), 2.15-2.00 (m, 3H), 1.99-1.66 (m, 3H), 1.50 (d, 1H), 1.40 (d, 1H), 1.18 (m, 1H), 1.03 (m, 1H). MS m/z 390 (M+1).

### Example 7

### N-Methyl-N-({1-[(1-methyl-3-piperidinyl)methyl]-1H-benzimidazol-2-yl}methyl)-5,6,7,8-tetrahydro-8-quinolinamine

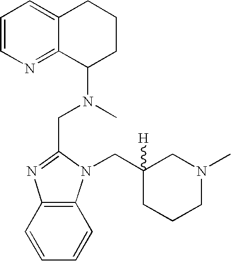

### a) t-Butyl 3-(hydroxymethyl)-1-piperidinecarboxylate

A stirred solution of 3-(hydroxymethyl)-1-piperidine (0.500 g, 4.34 mmol, Aldrich) in 20 mL of anhydrous dichloromethane was treated with di-t-butyl dicarbonate (1.04 g, 4.77 mmol) dissolved in 10 mL of anhydrous dichloromethane. After 18 hours the solution was diluted with dichloromethane, washed with 10% aqueous citric acid (2×), saturated aqueous NaHCO₃ (2×), dried over Na₂SO₄, and concentrated at reduced pressure to give a transparent, viscous oil. The crude material was subjected to flash chromatography (silica gel, hexane/EtOAc) to afford 0.88 g (95%) of t-butyl 3-(hydroxymethyl)-1-piperidinecarboxylate as a white, crystalline solid. ¹H NMR (DMSO-d₆): δ 4.48 (t, 1H), 3.96 (br s, 1H), 3.77 (d, 1H), 3.26 (m, 1H), 3.17 (m, 1H), 2.67 (t, 1H), 2.43 (br s, 1H), 1.65 (m, 1H), 1.56 (m, 1H), 1.50-1.33 (m, 10H), 1.26 (m, 1H), 1.06 (m, 1H).

### b) t-Butyl 3-(chloromethyl)-1-piperidinecarboxylate

Reaction of t-butyl 3-(hydroxymethyl)-1-piperidinecarboxylate (0.850 g, 3.95 mmol) with PS-triphenylphosphine and CCl₄ as described herein for the preparation of t-butyl 4-(chloromethyl)-1-piperidinecarboxylate, afforded 0.86 g (93%) of t-butyl 3-(chloromethyl)-1-piperidinecarboxylate as an oil. ¹H NMR (DMSO-d₆): δ 3.92 (br s, 1H), 3.70 (m, 1H), 3.52 (dd, 1H), 3.47 (dd, 1H), 2.74 (t, 1H), 2.68-2.41 (br, 1H), 1.81-1.61 (m, 2H), 1.58 (m, 1H), 1.36 (s, 9H), 1.33-1.12 (m, 2H).

### c) t-Butyl 3-[(2-{[methyl(5,6,7,8-tetrahydro-8-quinolinyl)amino]methyl}-1H-benzimidazol-1-yl)methyl]-1-piperidinecarboxylate

Reaction of N-(1H-benzimidazol-2-ylmethyl)-N-methyl-5,6,7,8-tetrahydro-8-quinolinamine (50.0 mg, 0.171 mmol) with t-butyl 3-(chloromethyl)-1-piperidinecarboxylate (0.160 g, 0.684 mmol) as described herein for the preparation of N-methyl-N-({1-[2-(1-piperidinyl)ethyl]-1H-benzimidazol-2-yl}methyl)-5,6,7,8-tetrahydro-8-quinolinamine, followed by reverse phase HPLC purification as described in the same procedure, afforded 66 mg (79%) of t-butyl 3-[(2-{[methyl(5,6,7,8-tetrahydro-8-quinolinyl)amino]methyl}-1H-benzimidazol-1-yl)methyl]-1-piperidinecarboxylate as a light yellow foam. The diastereomers were indistinguishable by analytical RP-HPLC, however, ¹H NMR analysis is consistent with a 1:1 diastereomer mixture. ¹H NMR (CDCl₃): δ 8.51 (d, 1H), 7.70 (d, 1H), 7.39 (d, 1H), 7.32-7.13 (m, 3H), 7.06 (m, 1H), 4.49-3.42 (m, 5H), 2.93-2.47 (m, 4H), 2.40-1.94 (m, 7H), 1.87-1.05 (m, 16H). MS m/z 490 (M+1).

### d) N-Methyl-N-{[1-(3-piperidinylmethyl)-1H-benzimidazol-2-yl]methyl}-5,6,7,8-tetrahydro-8-quinolinamine

Deprotection of t-butyl 3-[(2-{[methyl(5,6,7,8-tetrahydro-8-quinolinyl)amino]methyl}-1H-benzimidazol-1-yl)methyl]-1-piperidinecarboxylate (64.0 mg, 0.130 mmol) as described herein for the preparation of N-methyl-N-{[1-(4-piperidinylmethyl)-1H-benzimidazol-2-yl]methyl}-5,6,7,8-tetrahydro-8-quinolinamine afforded 48 mg (94%) of N-methyl-N-{[1-(3-piperidinylmethyl)-1H-benzimidazol-2-yl]methyl}-5,6,7,8-tetrahydro-8-quinolinamine as a light yellow, tacky foam. The diastereomers were indistinguishable by analytical RP-HPLC, however, ¹H NMR analysis is consistent with a 1:1 diastereomer mixture. ¹H NMR (CDCl₃): δ 8.51 (m, 1H), 7.70 (m, 1H), 7.52-7.00 (m, 5H), 4.75-3.60 (m, 5H), 3.10-2.62 (m, 5H), 2.60-1.00 (m, 14H). MS m/z 390 (M+1).

### e) N-Methyl-N-({1-[(1-methyl-3-piperidinyl)methyl]-1H-benzimidazol-2-yl}methyl)-5,6,7,8-tetrahydro-8-quinolinamine

A mixture of N-methyl-N-{[1-(3-piperidinylmethyl)-1H-benzimidazol-2-yl]methyl}-5,6,7,8-tetrahydro-8-quinolinamine (40.0 mg, 0.100 mmol), 37% aqueous formaldehyde (25.0 μL, 0.310 mmol) and NaBH(OAc)₃ (65.0 mg, 0.310 mmol) in 5 mL of anhydrous 1,2-dichloroethane was stirred at RT. After 18 hours the solution was diluted with an equal volume of 10% aqueous Na₂CO₃ and stirred vigorously for 25 minutes. The mixture was diluted with dichloromethane and the phases separated. The organic solution was washed once with aqueous brine, dried over Na₂SO₄, and concentrated to dryness at reduced pressure to afford 33 mg (79%) of N-methyl-N-({1-[(1-methyl-3-piperidinyl)methyl]-1H-benzimidazol-2-yl}methyl)-5,6,7,8-tetrahydro-8-quinolinamine as a viscous yellow oil. The diastereomers were indistinguishable by analytical RP-HPLC, however, ¹H NMR analysis is consistent with a 1:1 diastereomer mixture. ¹H NMR (CDCl₃): δ 8.52 (m, 1H), 7.70 (m, 1H), 7.38 (m, 1H), 7.35-7.14 (m, 3H), 7.08 (m, 1H), 4.41 (m, 1H), 4.22 (m, 1H), 4.10 (m, 1H), 4.04-3.88 (m, 2H), 2.83 (m, 1H), 2.80-1.98 (m, 13H), 1.93-1.20 (m, 6H), 1.02-0.70 (m, 1H). MS m/z 404 (M+1).

### Example 8

### N-Methyl-N-({1-[(1′-methyl-3-pyrrolidinyl)methyl]-1H-benzimidazol-2-yl}methyl)-5,6,7,8-tetrahydro-8-quinolinamine

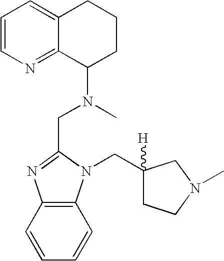

### a) t-Butyl 3-(chloromethyl)-1-pyrrolidinecarboxylate

Reaction of t-butyl 3-(hydroxymethyl)-1-pyrrolidinecarboxylate (0.500 g, 2.48 mmol) with PS-triphenylphosphine and CCl₄ as described herein for the preparation of t-butyl 4-(chloromethyl)-1-piperidinecarboxylate, afforded 0.34 g (62%) of t-butyl 3-(chloromethyl)-1-pyrrolidinecarboxylate as a light yellow oil. ¹H NMR (DMSO-d₆): δ 3.70 (d, 2H), 3.53-3.32 (m, 2H), 3.22 (m, 1H), 3.02 (dd, 1H), 2.58 (m, 1H), 2.00 (m, 1H), 1.69 (m, 1H), 1.44 (s, 9H).

### b) t-Butyl 3-[(2-{[methyl(5,6,7,8-tetrahydro-8-quinolinyl)amino]methyl}-1H-benzimidazol-1-yl)methyl]-1-pyrrolidinecarboxylate

A mixture of N-(1H-benzimidazol-2-ylmethyl)-N-methyl-5,6,7,8-tetrahydro-8-quinolinamine (50.0 mg, 0.171 mmol), t-butyl 3-(chloromethyl)-1-pyrrolidinecarboxylate (75.0 mg, 0.342 mmol), potassium iodide (57.0 mg, 0.342 mmol), and K₂CO₃ (0.142 g, 1.03 mmol) in 5 mL of anhydrous DMF in a sealed tube was heated to 120° C. with stirring. After 3 hours, the mixture was treated with an additional 75 mg of t-butyl 3-(chloromethyl)-1-pyrrolidinecarboxylate and 60 mg of potassium iodide and stirring at 120° C. was continued. After 18 hours the mixture was cooled to RT, diluted with EtOAc, washed with saturated aqueous NaHCO₃ (3×), dried over Na₂SO₄, and concentrated to dryness at reduced pressure. The crude product was purified by reverse phase HPLC as described herein for the preparation of N-methyl-N-({1-[2-(1-piperidinyl)ethyl]-1H-benzimidazol-2-yl}methyl)-5,6,7,8-tetrahydro-8-quinolinamine to afford 44 mg (54%) of t-butyl 3-[(2-{[methyl(5,6,7,8-tetrahydro-8-quinolinyl)amino]methyl}-1H-benzimidazol-1-yl)methyl]-1-pyrrolidinecarboxylate as a viscous yellow oil. The diastereomers were indistinguishable by analytical RP-HPLC, however, ¹H NMR analysis is consistent with a 1:1 diastereomer mixture. ¹H NMR (CDCl₃): δ 8.48 (m, 1H), 7.72 (m, 1H), 7.47-7.18 (m, 4H), 7.07 (m, 1H), 4.63-3.90 (m, 5H), 3.20-2.63 (m, 4H), 2.51-1.92 (m, 7H), 1.83-1.25 (m, 14H). MS m/z 476 (M+1).

### c) N-Methyl-N-{[1-(3-pyrrolidinylmethyl)-1H-benzimidazol-2-yl]methyl}-5,6,7,8-tetrahydro-8-quinolinamine

Deprotection of t-butyl 3-[(2-{[methyl(5,6,7,8-tetrahydro-8-quinolinyl)amino]methyl}-1H-benzimidazol-1-yl)methyl]-1-pyrrolidinecarboxylate (40 mg, 0.084 mmol) as described herein for the preparation of N-methyl-N-{[1-(4-piperidinylmethyl)-1H-benzimidazol-2-yl]methyl}-5,6,7,8-tetrahydro-8-quinolinamine afforded 15 mg (47%) of N-methyl-N-{[1-(3-pyrrolidinylmethyl)-1H-benzimidazol-2-yl]methyl}-5,6,7,8-tetrahydro-8-quinolinamine as a light yellow foam. The diastereomers were indistinguishable by analytical RP-HPLC, however, ¹H NMR analysis is consistent with a 1:1 diastereomer mixture. ¹H NMR (CDCl₃): δ 8.57, 8.48 (d, 1H total, 2 diastereomers), 7.72 (m, 1H), 7.49-7.31 (m, 2H), 7.28-7.19 (m, 2H), 7.13 (m, 1H), 4.61-3.48 (m, 5H), 3.35-2.50 (m, 5H), 2.40, 2.22 (s, 3H total, 2 diastereomers), 2.16-0.80 (m, 9H). MS m/z 376 (M+1).

### d) N-Methyl-N-({1-[(1-methyl-3-pyrrolidinyl)methyl]-1H-benzimidazol-2-yl}methyl)-5,6,7,8-tetrahydro-8-quinolinamine

Reductive methylation of N-methyl-N-{[1-(3-pyrrolidinylmethyl)-1H-benzimidazol-2-yl]methyl}-5,6,7,8-tetrahydro-8-quinolinamine (12 mg, 0.032 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine afforded 9.8 mg (78%) of N-methyl-N-({1-[(1-methyl-3-pyrrolidinyl)methyl]-1H-benzimidazol-2-yl}methyl)-5,6,7,8-tetrahydro-8-quinolinamine as a viscous yellow oil. The diastereomers were indistinguishable by analytical RP-HPLC, however, ¹H NMR analysis is consistent with a 1:1 diastereomer mixture. ¹H NMR (CDCl₃): δ 8.50 (d, 1H), 7.70 (d, 1H), 7.39 (m, 2H), 7.28-7.17 (m, 2H), 7.08 (m, 1H), 4.50-3.87 (m, 5H), 2.95-2.23 (m, 10H), 2.17-0.80 (m, 9H). MS m/z 390 (M+1).

### Example 9

### N-Methyl-N-({1-[3-(4-methyl-1-piperazinyl)propyl]-1H-benzimidazol-2-yl}methyl)-5,6,7,8-tetrahydro-8-quinolinamine

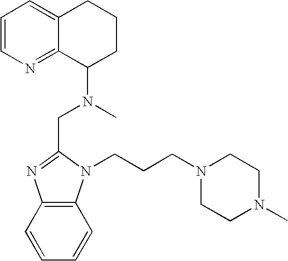

### a) t-Butyl 4-(3-chloropropyl)-1-piperazinecarboxylate

A mixture of t-butyl 1-piperazinecarboxylate (0.500 g, 2.68 mmol, Lancaster), 1-chloro-3-iodopropane (1.10 g, 5.36 mmol, Aldrich) and K₂CO₃ (1.85 g, 13.4 mmol) in 20 mL of anhydrous DMF was stirred at RT. After 18 hours the solution was diluted with water and the resulting mixture extracted with EtOAc (3×). The combined extracts were washed with saturated aqueous brine (2×), dried over Na₂SO₄, and concentrated at reduced pressure. The crude product was purified by flash chromatography (silica gel, hexane/EtOAc) to afford 0.339 g (48%) of t-butyl 4-(3-chloropropyl)-1-piperazinecarboxylate as a yellow oil. ¹H NMR (DMSO-d₆): δ 3.70 (t, 2H), 3.32 (m, 4H), 2.43 (t, 2H), 2.34 (m, 4H), 1.90 (m, 2H), 1.42 (s, 9H).

### b) t-Butyl 4-[3-(2-{[methyl(5,6,7,8-tetrahydro-8-quinolinyl)amino]methyl}-1H-benzimidazol-1-yl)propyl]-1-piperazinecarboxylate

Reaction of N-(1H-benzimidazol-2-ylmethyl)-N-methyl-5,6,7,8-tetrahydro-8-quinolinamine (50 mg, 0.17 mmol) with t-butyl 4-(3-chloropropyl)-1-piperazinecarboxylate (90 mg, 0.34 mmol) as described herein for the preparation of N-methyl-N-({1-[2-(1-piperidinyl)ethyl]-1H-benzimidazol-2-yl}methyl)-5,6,7,8-tetrahydro-8-quinolinamine, followed by reverse phase HPLC purification as described in the same procedure, afforded 84 mg (94%) of t-butyl 4-[3-(2-{[methyl(5,6,7,8-tetrahydro-8-quinolinyl)amino]methyl}-1H-benzimidazol-1-yl)propyl]-1-piperazinecarboxylate as a tacky, white foam. ¹H NMR (CDCl₃): δ 8.50 (d, 1H), 7.70 (d, 1H), 7.37 (m, 2H), 7.28-7.19 (m, 2H), 7.06 (m, 1H), 4.58 (m, 1H), 4.41 (m, 1H), 4.14 (d, 1H), 4.07-3.98 (m, 2H), 3.50-3.32 (m, 4H), 2.82 (m, 1H), 2.71 (m, 1H), 2.52-1.81 (m, 14H), 1.73 (m, 1H), 1.46 (s, 9H). MS m/z 519 (M+1).

### c) N-Methyl-N-({1-[3-(1-piperazinyl)propyl]-1H-benzimidazol-2-yl}methyl)-5,6,7,8-tetrahydro-8-quinolinamine

Deprotection of t-butyl 4-[3-(2-{[methyl(5,6,7,8-tetrahydro-8-quinolinyl)amino]methyl}-1H-benzimidazol-1-yl)propyl]-1-piperazinecarboxylate (80 mg, 0.15 mmol) as described herein for the preparation of N-methyl-N-{[1-(4-piperidinylmethyl)-1H-benzimidazol-2-yl]methyl}-5,6,7,8-tetrahydro-8-quinolinamine afforded 52 mg (80%) of N-methyl-N-({1-[3-(1-piperazinyl)propyl]-1H-benzimidazol-2-yl}methyl)-5,6,7,8-tetrahydro-8-quinolinamine as a viscous yellow oil. ¹H NMR (CDCl₃): δ 8.49 (d, 1H), 7.68 (d, 1H), 7.35 (d, 2H), 7.23-7.14 (m, 2H), 7.04 (m, 1H), 4.56 (m, 1H), 4.38 (m, 1H), 4.13 (d, 1H), 4.04-3.93 (m, 2H), 2.90-2.87 (m, 5H), 2.71 (m, 1H), 2.42-1.97 (m, 13H), 1.88 (m, 2H), 1.72 (m, 1H). MS m/z 419 (M+1).

### d) N-Methyl-N-({1-[3-(4-methyl-1-piperazinyl)propyl]-1H-benzimidazol-2-yl}methyl)-5,6,7,8-tetrahydro-8-quinolinamine

Reductive methylation of N-methyl-N-({1-[3-(1-piperazinyl)propyl]-1H-benzimidazol-2-yl}methyl)-5,6,7,8-tetrahydro-8-quinolinamine (43.0 mg, 0.103 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine afforded 36 mg (80%) of N-methyl-N-({1-[3-(4-methyl-1-piperazinyl)propyl]-1H-benzimidazol-2-yl}methyl)-5,6,7,8-tetrahydro-8-quinolinamine as a thick, yellow oil. ¹H NMR (CDCl₃): δ 8.49 (d, 1H), 7.69 (d, 1H), 7.36 (d, 2H), 7.23-7.13 (m, 2H), 7.05 (m, 1H), 4.58 (m, 1H), 4.37 (m, 1H), 4.13 (d, 1H), 4.03-3.92 (m, 2H), 2.83 (m, 1H), 2.72 (m, 1H), 2.59-1.93 (m, 19H), 1.88 (m, 2H), 1.72 (m, 1H). MS m/z 433 (M+1).

### Example 10

### N-Methyl-N-({1-[(1-methyl-3-azetidinyl)methyl]-1H-benzimidazol-2-yl}methyl)-5,6,7,8-tetrahydro-8-quinolinamine

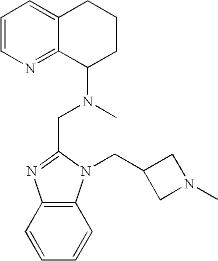

### a) t-Butyl 3-(hydroxymethyl)-1-azetidinecarboxylate

A stirred solution of 1-(t-butoxycarbonyl)-3-azetidinecarboxylic acid (0.730 g, 3.63 mmol, Fluka) and 4-methylmorpholine (0.440 mL, 3.99 mmol) in 7 mL of anhydrous THF was cooled in a NaCl/ice water bath. The solution was treated with isobutyl chloroformate (0.520 mL, 3.99 mmol) by drop-wise addition. A white solid rapidly precipitated from the solution. After stirring the cold suspension for 10 minutes, the solid was removed by vacuum filtration and the filter cake washed with three 2 mL portions of THF. The filtrate was cooled in the NaCl/ice water bath and was then treated with NaBH₄ (0.210 g, 5.45 mmol) dissolved in 3 mL of water. Vigorous gas evolution occurred during the addition. The solution was allowed to slowly warm to RT with melting of the ice bath. After 1.5 hours the solution was concentrated to dryness at reduced pressure. The residue was suspended in EtOAc. The resulting mixture was washed with 10% aqueous citric acid (2×), saturated aqueous NaHCO₃ (2×), aqueous brine (1×), dried over Na₂SO₄, and concentrated to dryness at reduced pressure. The crude product was purified by flash chromatography (silica gel, EtOAc/hexane) to afford 0.33 g (49%) of t-butyl 3-(hydroxymethyl)-1-azetidinecarboxylate as a transparent viscous oil. ¹H NMR (DMSO-d₆): δ 4.74 (t, 1H), 3.79 (br s, 2H), 3.53 (br s, 2H), 3.46 (t, 2H), 2.56 (m, 1H), 1.35 (s, 9H).

### b) t-Butyl 3-(chloromethyl)-1-azetidinecarboxylate

Reaction of 3-(hydroxymethyl)-1-azetidinecarboxylate (0.471 g, 2.52 mmol) with PS-triphenylphosphine and CCl₄ as described herein for the preparation of t-butyl 4-(chloromethyl)-1-piperidinecarboxylate, followed by purification by flash chromatography (silica gel, EtOAc/hexane), afforded 0.327 g (63%) of t-butyl 3-(chloromethyl)-1-azetidinecarboxylate as a clear oil. ¹H NMR (DMSO-d₆): δ 3.88 (br s, 2H), 3.80 (d, 2H), 3.56 (br s, 2H), 2.88 (m, 1H), 1.38 (s, 9H).

### c) t-Butyl 3-[(2-{[methyl(5,6,7,8-tetrahydro-8-quinolinyl)amino]methyl}-1H-benzimidazol-1-yl)methyl]-1-azetidinecarboxylate

A mixture of N-(1H-benzimidazol-2-ylmethyl)-N-methyl-5,6,7,8-tetrahydro-8-quinolinamine (0.155 g, 0.530 mmol), t-butyl 3-(chloromethyl)-1-azetidinecarboxylate (0.218 g, 1.06 mmol), potassium iodide (0.176 g, 1.06 mmol) and K₂CO₃ (0.440 g, 3.18 mmol) in 8 mL of anhydrous DMF was heated to 100° C. with stirring. After 7 h the mixture was cooled to RT and stirred overnight. The mixture was then diluted with EtOAc, washed with water (1×), aqueous NaHCO₃ (3×), dried over Na₂SO₄, and concentrated to dryness at reduced pressure. The crude product was purified by flash chromatography (silica gel, gradient elution of dichloromethane to 9:1 dichloromethane/2M NH₃ in MeOH) to afford 0.104 g (42%) of t-butyl 3-[(2-{[methyl(5,6,7,8-tetrahydro-8-quinolinyl)amino]methyl}-1H-benzimidazol-1-yl)methyl]-1-azetidinecarboxylate as a white foam. ¹H NMR (CDCl₃): δ 8.51 (d, 1H), 7.72 (d, 1H), 7.40 (d, 1H), 7.36 (d, 1H), 7.28-7.20 (m, 2H), 7.10 (dd, 1H), 4.83 (dd, 1H), 4.53 (dd, 1H), 4.11 (d, 1H), 4.03 (m, 1H), 3.92-3.72 (m, 4H), 3.58 (dd, 1H), 3.02 (m, 1H), 2.85 (m, 1H), 2.74 (m, 1H), 2.31 (s, 3H), 2.16-2.00 (m, 3H), 1.78 (m, 1H), 1.43 (s, 9H). MS m/z 462 (M+1).

### d) N-{[1-(3-Azetidinylmethyl)-1H-benzimidazol-2-yl]methyl}-N-methyl-5,6,7,8-tetrahydro-8-quinolinamine

A solution of t-butyl 3-[(2-{[methyl(5,6,7,8-tetrahydro-8-quinolinyl)amino]methyl}-1H-benzimidazol-1-yl)methyl]-1-azetidinecarboxylate (50 mg, 0.11 mmol) in 8 mL of 1:1 TFA/dichoromethane was stirred at RT for 10 minutes and was then concentrated to dryness by rotary evaporation. The residue was dissolved in EtOAc. The solution was washed once with 10% aqueous Na₂CO₃, twice with aqueous brine, dried over Na₂SO₂ and concentrated at reduced pressure to afford 26 mg (67%) of N-{[1-(3-azetidinylmethyl)-1H-benzimidazol-2-yl]methyl}-N-methyl-5,6,7,8-tetrahydro-8-quinolinamine as a transparent, viscous oil. ¹H NMR (CDCl₃): δ 8.53 (d, 1H), 7.70 (d, 1H), 7.42 (d, 1H), 7.33 (d, 1H), 7.31-7.19 (m, 2H), 7.12 (dd, 1H), 4.74 (dd, 1H), 4.57 (dd, 1H), 4.12-4.01 (m, 2H), 3.88 (d, 1H), 3.60 (t, 2H), 3.46 (t, 1H), 3.35 (t, 1H), 3.16 (m, 1H), 2.93-2.65 (m, 3H), 2.30 (s, 3H), 2.19-1.99 (m, 3H), 1.75 (m, 1H). MS m/z 362 (M+1).

### e) N-Methyl-N-({1-[(1-methyl-3-azetidinyl)methyl]-1H-benzimidazol-2-yl}methyl)-5,6,7,8-tetrahydro-8-quinolinamine

Reductive methylation of N-{[1-(3-azetidinylmethyl)-1H-benzimidazol-2-yl]methyl}-N-methyl-5,6,7,8-tetrahydro-8-quinolinamine (21 mg, 0.058 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine afforded 16 mg (73%) of N-methyl-N-({1-[(1-methyl-3-azetidinyl)methyl]-1H-benzimidazol-2-yl}methyl)-5,6,7,8-tetrahydro-8-quinolinamine as a viscous, yellow oil. ¹H NMR (CDCl₃): δ 8.51 (d, 1H), 7.71 (d, 1H), 7.39 (d, 2H), 7.28-7.16 (m, 2H), 7.08 (dd, 1H), 4.71 (dd, 1H), 4.49 (dd, 1H), 4.07 (d, 1H), 3.98 (t, 1H), 3.87 (d, 1H), 3.25-3.10 (m, 2H), 2.94-2.80 (m, 3H), 2.79-2.64 (m, 2H), 2.30 (s, 3H), 2.21 (s, 3H), 2.14-1.97 (m, 3H), 1.72 (m, 1H). MS m/z 376 (M+1).

### Example 11

### N-Methyl-N-{[1-(1-methyl-4-piperidinyl)-1H-benzimidazol-2-yl]methyl}-5,6,7,8-tetrahydro-8-quinolinamine

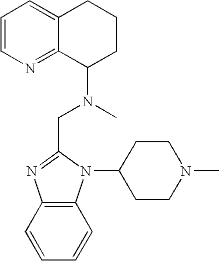

### a) t-Butyl 4-[(2-nitrophenyl)amino]-1-piperidinecarboxylate

A mixture of 1-chloro-2-nitrobenzene (0.394 g, 2.50 mmol, Aldrich), t-butyl 4-amino-1-piperidinecarboxylate (0.500 g, 2.50 mmol, EMKA-Chemie) and K₂CO₃ (1.04 g, 7.50 mmol) in 5 mL of anhydrous DMF in a sealed tube was heated to 120° C. with stirring. After 18 hours, the mixture was cooled to RT and filtered through a medium fritted funnel to remove solids. The filter cake was rinsed with EtOAc (3×) and the filtrate diluted with an additional 50 mL of EtOAc. The EtOAc solution was washed with aqueous brine (4×), dried over Na₂SO₄, and concentrated to dryness at reduced pressure. The crude product was purified by flash chromatography (silica gel, hexane/EtOAc) to afford 0.257 g (32%) of t-butyl 4-[(2-nitrophenyl)amino]-1-piperidinecarboxylate as a yellow glass. ¹H NMR (DMSO-d₆): δ 8.05 (d, 1H), 7.90 (d, 1H), 7.52 (t, 1H), 7.17 (d, 1H), 6.69 (t, 1H), 3.94-3.74 (m, 3H), 2.97 (br s, 2H), 1.92 (d, 2H), 1.50-1.32 (m, 1H). MS m/z 344 (M+Na).

### b) t-Butyl 4-[(2-aminophenyl)amino]-1-piperidinecarboxylate

A solution of t-butyl 4-[(2-nitrophenyl)amino]-1-piperidinecarboxylate (0.250 g, 0.778 mmol) in 20 mL of MeOH was subjected to catalytic hydrogenation at 50 psi in the presence of 25 mg of 10% Pd on charcoal. After 2.5 hours the reaction vessel was purged with nitrogen, catalyst removed by filtration through celite, and the filtrate concentrated to dryness at reduced pressure to afford 0.214 g (94%) of t-butyl 4-[(2-aminophenyl)amino]-1-piperidinecarboxylate as a brown foam. NMR (DMSO-d₆): δ 6.57-6.43 (m, 3H), 6.40 (t, 1H), 4.48 (s, 2H), 4.13 (d, 1H), 3.88 (d, 2H), 3.37 (m, 1H), 3.00-2.72 (br s, 2H), 1.89 (d, 2H), 1.40 (s, 9H), 1.24 (m, 2H).

### c) t-Butyl 4-({2-[(N-{[(phenylmethyl)oxy]carbonyl}glycyl)amino]phenyl}amino)-1-piperidinecarboxylate

A solution of t-butyl 4-[(2-aminophenyl)amino]-1-piperidinecarboxylate (0.214 g, 0.734 mmol), Cbz-glycine (0.184 g, 0.881 mmol), N,N-diisopropylethylamine (0.153 mL, 0.881 mmol) and bis(2-oxo-3-oxazolidinyl)phosphinic chloride (0.224 g, 0.881 mmol, Aldrich) in 8 mL of anhydrous MeCN was stirred at RT. After 18 hours the solution was evaporated to dryness and the residue dissolved in EtOAc. The solution was washed with aqueous brine (3×), dried over Na₂SO₄, and concentrated to dryness to afford 0.300 g (85%) of t-butyl 4-({2-[(N-{[(phenylmethyl)oxy]carbonyl}glycyl)amino]phenyl}amino)-1-piperidinecarboxylate which was determined by ¹H-NMR to be sufficiently pure for use in the next synthetic step. NMR (DMSO-d₆): δ 9.14 (s, 1H), 7.60 (t, 1H), 7.40-7.22 (m, 5H), 7.11-6.97 (m, 2H), 6.70 (d, 1H), 6.53 (t, 1H), 5.02 (s, 2H), 4.60 (d, 1H), 3.92-3.71 (m, 4H), 3.43 (m, 1H), 2.91 (br s, 2H), 1.83 (d, 2H), 1.38 (s, 9H), 1.27 (m, 2H).

### d) t-Butyl 4-{2-[({[(phenylmethyl)oxy]carbonyl}amino)methyl]-1H-benzimidazol-1-yl}-1-piperidinecarboxylate

A solution of t-butyl 4-({2-[(N-{[(phenylmethyl)oxy]carbonyl}glycyl)amino]phenyl}amino)-1-piperidinecarboxylate (0.300 g, 0.622 mmol) in 6 mL of glacial acetic acid was heated to 60° C. with stirring. After 5.5 hours the solution was cooled to RT and concentrated to dryness at reduced pressure. The residue was dissolved in EtOAc. The solution was washed with 10% aqueous Na₂CO₃ (2×), saturated aqueous brine (1×), dried over Na₂SO₄, and concentrated to dryness. The crude product was purified by flash chromatography (silica gel, gradient elution of EtOAc to 95:5 EtOAc/MeOH) to afford 0.250 g (87%) of t-butyl 4-{2-[({[(phenylmethyl)oxy]carbonyl}amino)methyl]-1H-benzimidazol-1-yl}-1-piperidinecarboxylate as an off-white foam. NMR (DMSO-d₆): δ 7.92 (m, 1H), 7.60-7.45 (m, 2H), 7.38-7.23 (m, 5H), 7.20-7.07 (m, 2H), 5.04 (s, 2H), 4.66-4.47 (m, 3H), 4.13-3.92 (m, 2H), 2.77 (br s, 2H), 2.15 (m, 2H), 1.75 (d, 2H), 1.41 (s, 9H). MS m/z 465 (M+1).

### e) t-Butyl 4-[2-(aminomethyl)-1H-benzimidazol-1-yl]-1-piperidinecarboxylate

A solution of t-butyl 4-{2-[({[(phenylmethyl)oxy]carbonyl}amino)methyl]-1H-benzimidazol-1-yl}-1-piperidinecarboxylate (0.250 g, 0.538 mmol) in 20 mL of MeOH was subjected to catalytic hydrogenation at 50 psi in the presence of 50 mg of 10% Pd on charcoal. After 4 hours the reaction vessel was purged with nitrogen, catalyst removed by filtration through celite, and the filtrate concentrated to dryness at reduced pressure to afford 0.163 g (92%) of t-butyl 4-[2-(aminomethyl)-1H-benzimidazol-1-yl]-1-piperidinecarboxylate as a white foam. ¹H NMR (CDCl₃): δ 7.72 (d, 1H), 7.46 (d, 1H), 7.21 (m, 2H), 4.52 (m, 1H), 4.36 (br s, 2H), 4.16 (s, 2H), 3.97-2.79 (m, 2H), 2.52-2.31 (m, 2H), 1.91 (d, 2H), 1.62 (m, 2H), 1.51 (s, 9H). MS m/z 331 (M+1).

### f) t-Butyl 4-{2-[(5,6,7,8-tetrahydro-8-quinolinylamino)methyl]-1H-benzimidazol-1-yl}-1-piperidinecarboxylate

A solution of t-butyl 4-[2-(aminomethyl)-1H-benzimidazol-1-yl]-1-piperidinecarboxylate (0.142 g, 0.429 mmol), 6,7-dihydro-8(5H)-quinolinone (76.0 mg, 0.515 mmol, J. Org. Chem., 2002, 67, 2197-2205) and glacial acetic acid (37.0 μL, 0.644 mmol) in 7 mL of 1,2-dichloroethane was stirred at RT for 15 minutes. The solution was then treated with NaBH(OAc)₃ (0.136 g, 0.644 mmol) by portion-wise addition over a one hour period. After 3 hours the solution was diluted with dichloromethane followed by 10% aqueous Na₂CO₃ and the mixture was stirred vigorously for 25 minutes. The mixture was transferred to a separatory funnel and the phases separated. The organic solution was washed with saturated aqueous brine (2×), dried over Na₂SO₄, and concentrated to dryness at reduced pressure. The crude product was purified by flash chromatography (silica gel, gradient elution of dichloromethane to 95:5 dichloromethane/2M NH₃ in MeOH) to afford 0.150 g (76%) of t-butyl 4-{2-[(5,6,7,8-tetrahydro-8-quinolinylamino)methyl]-1H-benzimidazol-1-yl}-1-piperidinecarboxylate as a light yellow foam. ¹H NMR (CDCl₃): δ 8.39 (d, 1H), 7.72 (d, 1H), 7.48 (d, 1H), 7.39 (d, 1H), 7.24-7.16 (m, 2H), 7.08 (dd, 1H), 4.70 (m, 1H), 4.42 (m, 4H), 3.86 (m, 1H), 2.90-2.68 (m, 5H), 2.51-2.30 (m, 2H), 2.14 (m, 1H), 2.03-1.85 (m, 3H), 1.82-1.71 (m, 2H), 1.51 (s, 9H). MS m/z 462 (M+1).

### g) t-Butyl 4-(2-{[methyl(5,6,7,8-tetrahydro-8-quinolinyl)amino]methyl}-1H-benzimidazol-1-yl)-1-piperidinecarboxylate

Reductive methylation of t-butyl 4-{2-[(5,6,7,8-tetrahydro-8-quinolinylamino)methyl]-1H-benzimidazol-1-yl}-1-piperidinecarboxylate (70.0 mg, 0.152 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine afforded 55 mg (76%) of t-butyl 4-(2-{[methyl(5,6,7,8-tetrahydro-8-quinolinyl)amino]methyl}-1H-benzimidazol-1-yl)-1-piperidinecarboxylate as a white foam. ¹H NMR (CDCl₃): δ 8.47 (d, 1H), 7.71 (d, 1H), 7.46 (d, 1H), 7.40 (d, 1H), 7.22-7.13 (m, 2H), 7.10 (dd, 1H), 5.04 (m, 1H), 4.45-4.13 (m, 3H), 4.04-3.90 (m, 2H), 3.00-2.65 (m, 4H), 2.47-2.28 (m, 2H), 2.19 (s, 3H), 2.11-1.94 (m, 3H), 1.92-1.63 (m, 3H), 1.51 (s, 9H). MS m/z 476 (M+1).

### h) N-Methyl-N-{[1-(4-piperidinyl)-1H-benzimidazol-2-yl]methyl}-5,6,7,8-tetrahydro-8-quinolinamine

A solution of t-butyl 4-(2-{[methyl(5,6,7,8-tetrahydro-8-quinolinyl)amino]methyl}-1H-benzimidazol-1-yl)-1-piperidinecarboxylate (50.0 mg, 0.105 mmol) in 5 mL of 1:1 TFA/dichloromethane was stirred at RT for 2 hours and then concentrated to dryness by rotary evaporation. The residue was dissolved in EtOAc. The solution was washed once with 10% aqueous Na₂CO₃, once with aqueous brine, dried over Na₂SO₄, and concentrated to dryness at reduced pressure to afford 29 mg (74%) of N-methyl-N-{[1-(4-piperidinyl)-1H-benzimidazol-2-yl]methyl}-5,6,7,8-tetrahydro-8-quinolinamine as a white foam. ¹H NMR (CDCl₃): δ 8.49 (d, 1H), 7.71 (m, 1H), 7.62 (m, 1H), 7.40 (d, 1H), 7.21-7.12 (m, 2H), 7.09 (dd, 1H), 4.85 (m, 1H), 4.25 (d, 1H), 4.02-3.89 (m, 2H), 3.28 (t, 2H), 2.92-2.67 (m, 4H), 2.50-2.33 (m, 2H), 2.19 (s, 3H), 2.14-1.67 (m, 7H). MS m/z 376 (M+1).

### i) N-Methyl-N-{[1-(1-methyl-4-piperidinyl)-1H-benzimidazol-2-yl]methyl}-5,6,7,8-tetrahydro-8-quinolinamine

Reductive methylation of N-methyl-N-{[1-(4-piperidinyl)-1H-benzimidazol-2-yl]methyl}-5,6,7,8-tetrahydro-8-quinolinamine (25 mg, 0.067 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine afforded 23 mg (88%) of N-methyl-N-{[1-(1-methyl-4-piperidinyl)-1H-benzimidazol-2-yl]methyl}-5,6,7,8-tetrahydro-8-quinolinamine as a white foam. ¹H NMR (CDCl₃): δ 8.49 (d, 1H), 7.72-7.62 (m, 2H), 7.40 (d, 1H), 7.20-7.05 (m, 3H), 4.76 (m, 1H), 4.25 (d, 1H), 4.00-3.88 (m, 2H), 3.00 (t, 2H), 2.84 (m, 1H), 2.73 (m, 1H), 2.58 (m, 2H), 2.39 (s, 3H), 2.22-1.97 (m, 7H), 1.90-1.65 (m, 4H). MS m/z 390 (M+1).

### Example 12

### N-Methyl-N-({1-[(4-{[(2-pyridinylmethyl)amino]methyl}phenyl)methyl]-1H-benzimidazol-2-yl}methyl)-5,6,7,8-tetrahydro-8-quinolinamine

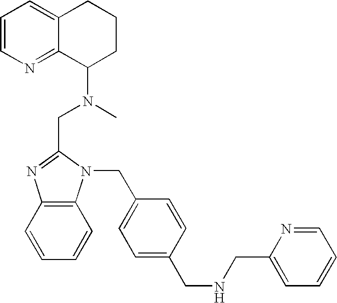

### a) t-Butyl (2-pyridinylmethyl)carbamate

A solution of (2-pyridinylmethyl)amine (5 mL, 48.5 mmol) and 4-dimethylaminopyridine (0.30 g, 2.42 mmol) in anhydrous dichloromethane (50 mL) was treated with di-t-butyl dicarbonate (12.7 g, 58.2 mmol) and the reaction was allowed to stir at room temperature. After 18 h, the reaction was washed with saturated aqueous NaHCO₃. The aqueous layer was washed with dichloromethane. The organic layers were combined and washed with saturated aqueous NaHCO₃, brine, dried (MgSO₄) and concentrated under reduced pressure. The crude product was purified by flash chromatography (silica gel, 0 to 50% EtOAc in hexanes) to afford 8.04 g (80%) of t-butyl (2-pyridinylmethyl)carbamate as a yellow oil. ¹H NMR (CDCl₃): δ 8.53 (d, 1H), 7.65 (td, 1H), 7.27 (m, 1H), 7.18 (m, 1H), 5.56 (brs, 1H), 4.44 (d, 2H), 1.46 (s, 9H). MS m/z 209 (M+1).

### b) Methyl 4-{[{[t-butyloxy]carbonyl}(2-pyridinylmethyl)amino]methyl}benzoate

A solution of t-butyl (2-pyridinylmethyl)carbamate (8.04 g, 38.6 mmol) in anhydrous DMF was treated with 60% sodium hydride (1.85 g, 46.3 mmol) in portions over 30 min. Once the addition was complete, let stir 1 h, then added methyl 4-(bromomethyl)benzoate (9.73 g, 42.5 mmol). After 2 h, quenched with water, then partitioned the reaction between water and EtOAc. The aqueous layer was extracted again with EtOAc (2×). The combined organic layers were washed with water, brine, dried (MgSO₄) and concentrated under reduced pressure. The crude product was purified by flash chromatography (silica gel, 0 to 50% EtOAc in hexanes) to afford 8.91 g (65%) of methyl 4-{[{[t-butyloxy]carbonyl}(2-pyridinylmethyl)amino]methyl}benzoate as a gold oil. ¹H NMR (CDCl₃): δ 8.53 (d, 1H), 7.97 (d, 2H), 7.65 (t, 1H), 7.34-7.16 (m, 4H), 4.60-4.47 (m, 4H), 3.91 (s, 3H), 1.44 (s, 9H). MS m/z 379 (M+Na).

### c) t-Butyl {[4-(hydroxymethyl)phenyl]methyl}(2-pyridinylmethyl)carbamate

A solution of lithium aluminum hydride (27.5 mL of a 1.0 M solution in THF, 27.5 mmol) in THF (20 mL) was cooled to 0° C. To this was added a solution of 4-{[{[t-butyloxy]carbonyl}(2-pyridinylmethyl)amino]methyl}benzoate (9.79 g, 27.5 mmol) in THF (50 mL). The reaction was stirred for 15 min, then treated with water (1 mL), 3 N NaOH (3 mL) and water (3 mL). Each of these additions was done slowly and the mixture was stirred thoroughly in between. The reaction was filtered and the filtrate was partitioned between Et₂O and water. The aqueous layer was washed with additional Et₂O, then the organic layers were combined, washed with brine, dried (MgSO₄) and concentrated under reduced pressure. The crude product was purified by flash chromatography (silica gel, 0 to 100% EtOAc in hexanes) to afford 7.47 g (83%) of t-butyl {[4-(hydroxymethyl)phenyl]methyl}(2-pyridinylmethyl)carbamate as a pale yellow oil. ¹H NMR (DMSO-d₆): δ 8.51 (d, 1H), 7.77 (td, 1H), 7.28 (m, 3H), 7.19 (br m, 3H), 5.15 (br s, 1H), 4.47-4.35 (m, 6H), 1.35 (m, 9H). MS m/z 351 (M+Na).

### d) t-Butyl {[4-(chloromethyl)phenyl]methyl}(2-pyridinylmethyl)carbamate

Reaction of t-butyl {[4-(hydroxymethyl)phenyl]methyl}(2-pyridinylmethyl)carbamate (145 mg, 0.44 mmol) with PS-triphenylphosphine and CCl₄ as described herein for the preparation of t-butyl 4-(chloromethyl)-1-piperidinecarboxylate, afforded 129 mg (84%) of t-butyl {[4-(chloromethyl)phenyl]methyl}(2-pyridinylmethyl)carbamate as an oil. ¹H NMR (CDCl₃): δ 8.52 (s, 1H), 8.01 (m, 1H), 7.64-7.26 (m, 6H), 4.87 (m, 2H), 4.61 (m, 2H), 4.53 (s, 2H), 1.46 (m, 9H).

### e) t-Butyl ({4-[(2-{[methyl(5,6,7,8-tetrahydro-8-quinolinyl)amino]methyl}-1H benzimidazol-1-yl)methyl]phenyl}methyl)(2-pyridinylmethyl)carbamate

Reaction of t-butyl {[4-(chloromethyl)phenyl]methyl}(2-pyridinylmethyl)carbamate (129 mg, 0.37 mmol) and N-(1H-benzimidazol-2-ylmethyl)-N-methyl-5,6,7,8-tetrahydro-8-quinolinamine (57 mg, 0.19 mmol) as described herein for the preparation of N-methyl-N-{[1-(3-pyridinylmethyl)-1H-benzimidazol-2-yl]methyl}-5,6,7,8-tetrahydro-8-quinolinamine, afforded 57 mg (48%) of t-butyl ({4-[(2-{[methyl(5,6,7,8-tetrahydro-8-quinolinyl)amino]methyl}-1H-benzimidazol-1-yl)methyl]phenyl}methyl)(2-pyridinylmethyl)carbamate as a yellow oil. ¹H NMR (DMSO-d₆): δ 8.48 (d, 1H), 8.33 (d, 1H), 7.72 (m, 1H), 7.59 (m, 1H), 7.47 (d, 1H), 7.38 (brs, 1H), 7.24 (m, 1H), 7.15 (m, 6H), 7.05 (m, 2H), 5.67 (m, 2H), 4.44-4.34 (m, 4H), 4.20-4.02 (m, 2H), 3.93 (t, 1H), 2.67 (m, 2H), 2.13 (s, 3H), 1.89 (m, 3H), 1.60 (m, 1H), 1.32 (m, 9H). MS m/z 603 (M+1).

### f) N-Methyl-N-({1-[(4-{[(2-pyridinylmethyl)amino]methyl}phenyl)methyl]-1H-benzimidazol-2-yl}methyl)-5,6,7,8-tetrahydro-8-quinolinamine

Deprotection of t-butyl ({4-[(2-{[methyl(5,6,7,8-tetrahydro-8-quinolinyl)amino]methyl}-1H-benzimidazol-1-yl)methyl]phenyl}methyl)(2-pyridinylmethyl)carbamate (56 mg, 0.093 mmol) as described herein for the preparation of N-{[1-(3-azetidinylmethyl)-1H-benzimidazol-2-yl]methyl}-N-methyl-5,6,7,8-tetrahydro-8-quinolinamine, afforded 45 mg (96%) of N-methyl-N-({1-[(4-{[(2-pyridinylmethyl)amino]methyl}phenyl)methyl]-1H-benzimidazol-2-yl}methyl)-5,6,7,8-tetrahydro-8-quinolinamine as a sticky solid. ¹H NMR (DMSO-d₆): δ 8.47 (d, 1H), 8.34 (d, 1H), 7.73 (td, 1H), 7.59 (m, 1H), 7.44 (m, 2H), 7.35 (m, 1H), 7.25 (m, 3H), 7.13 (m, 3H), 7.04 (m, 2H), 5.67 (m, 2H), 4.20-4.03 (m, 2H), 3.94 (t, 1H), 3.75 (s, 2H), 3.68 (s, 2H), 2.67 (m, 2H), 2.14 (s, 3H), 1.89 (m, 3H), 1.59 (m, 1H). MS m/z 503 (M+1).

### Example 13

### N-{[1-(4-Aminobutyl)-1H-benzimidazol-2-yl]methyl}-N-methyl-5,6,718-tetrahydro-8-quinolinamine

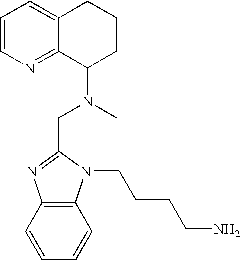

### a) t-Butyl (4-chlorobutyl)carbamate

Reaction of t-butyl (4-hydroxybutyl)carbamate (175 mg, 0.92 mmol) with PS-triphenylphosphine and CCl₄ as described herein for the preparation of t-butyl 4-(chloromethyl)-1-piperidinecarboxylate, afforded 184 mg (96%) of t-butyl (4-chlorobutyl)carbamate as a colorless oil. ¹H NMR (CDCl₃): δ 4.53 (br s, 1H), 3.56 (t, 2H), 3.16 (m, 2H), 1.81 (m, 2H), 1.64 (m, 2H), 1.44 (s, 9H).

### b) t-Butyl [4-(2-{[methyl(5,6,7,8-tetrahydro-8-quinolinyl)amino]methyl}-1H-benzimidazol-1-yl)butyl]carbamate

Reaction of t-butyl (4-chlorobutyl)carbamate (184 mg, 0.88 mmol) and N-(1H-benzimidazol-2-ylmethyl)-N-methyl-5,6,7,8-tetrahydro-8-quinolinamine (120 mg, 0.41 mmol) as described herein for the preparation of N-methyl-N-{[1-(3-pyridinylmethyl)-1H-benzimidazol-2-yl]methyl}-5,6,7,8-tetrahydro-8-quinolinamine, afforded 61 mg (32%) of t-butyl [4-(2-{[methyl(5,6,7,8-tetrahydro-8-quinolinyl)amino]methyl}-1H-benzimidazol-1-yl)butyl]carbamate as a yellow semisolid. ¹H NMR (DMSO-d₆): δ 8.45 (d, 1H), 7.53 (m, 3H), 7.16 (m, 3H), 6.84 (m, 1H), 4.32-3.96 (m, 5H), 2.96-2.67 (m, 5H), 2.09 (s, 3H), 1.99 (m, 3H), 1.69 (m, 2H), 1.43-1.30 (m, 11H). MS m/z 464 (M+1).

### c) N-{[1-(4-Aminobutyl)-1H-benzimidazol-2-yl]methyl}-N-methyl-5,6,7,8-tetrahydro-8-quinolinamine

Deprotection of t-butyl [4-(2-{[methyl(5,6,7,8-tetrahydro-8-quinolinyl)amino]methyl}-1H-benzimidazol-1-yl)butyl]carbamate (61 mg, 0.13 mmol) as described herein for the preparation of N-{[1-(3-azetidinylmethyl)-1H-benzimidazol-2-yl]methyl}-N-methyl-5,6,7,8-tetrahydro-8-quinolinamine, afforded 38 mg (81%) of N-{[1-(4-aminobutyl)-1H-benzimidazol-2-yl]methyl}-N-methyl-5,6,7,8-tetrahydro-8-quinolinamine as a tan oil. ¹H NMR (DMSO-d₆): δ 8.44 (d, 1H), 7.51 (m, 3H), 7.15 (m, 3H), 4.31 (m, 2H), 4.22-4.02 (m, 2H), 3.95 (m, 1H), 2.79-2.65 (m, 2H), 2.56 (t, 1H), 2.08 (s, 3H), 1.96 (m, 3H), 1.72 (m, 3H), 1.35 (m, 2H), 1.22 (m, 1H). MS m/z 364 (M+1).

### Example 14

### N-[(1-{[4-(Aminomethyl)phenyl]methyl}-1H-benzimidazol-2-yl)methyl]-N-methyl-5,6,7,8-tetrahydro-8-quinolinamine

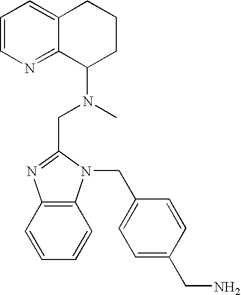

### a) 4-[(2-{[Methyl(5,6,7,8-tetrahydro-8-quinolinyl)amino]methyl}-1H-benzimidazol-1-yl)methyl]benzonitrile

Reaction of N-(1H-benzimidazol-2-ylmethyl)-N-methyl-5,6,7,8-tetrahydro-8-quinolinamine (106 mg, 0.36 mmol) and 4-cyanobenzyl bromide (78 mg, 0.40 mmol) as described herein for the preparation of N-methyl-N-{[1-(3-pyridinylmethyl)-1H-benzimidazol-2-yl]methyl}-5,6,7,8-tetrahydro-8-quinolinamine, afforded 84 mg (57%) of 4-[(2-{[methyl(5,6,7,8-tetrahydro-8-quinolinyl)amino]methyl}-1H-benzimidazol-1-yl)methyl]benzonitrile as a yellow oil. ¹H NMR (DMSO-d₆): δ 8.29 (d, 1H), 7.76 (d, 2H), 7.62 (d, 1H), 7.46 (d, 1H), 7.29 (m, 1H), 7.23 (d, 2H), 7.19-7.10 (m, 3H), 5.85 (m, 2H), 4.19-3.99 (m, 2H), 3.93 (m, 1H), 2.67 (m, 3H), 2.10 (s, 3H), 1.83 (m, 3H). MS m/z 408 (M+1).

### b) N-[(1-{[4-(Aminomethyl)phenyl]methyl}-1H-benzimidazol-2-yl)methyl]-N-methyl-5,6,7,8-tetrahydro-8-quinolinamine

A solution of 4-[(2-{[methyl(5,6,7,8-tetrahydro-8-quinolinyl)amino]methyl}-1H-benzimidazol-1-yl)methyl]benzonitrile (84 mg, 0.21 mmol) in 75 mL of 7N NH₃ in MeOH was subjected to catalytic hydrogenation at 50 psi in the presence of Raney nickel. After 16 h, the reaction vessel was purged with nitrogen, catalyst removed by filtration through celite and the filtrate concentrated under reduced pressure to afford 71 mg (85%) of N-[(1-{[4-(aminomethyl)phenyl]methyl}-1H-benzimidazol-2-yl)methyl]-N-methyl-5,6,7,8-tetrahydro-8-quinolinamine as a tan foam. ¹H NMR (DMSO-d₆): δ 8.34 (d, 1H), 7.56 (m, 1H), 7.45 (d, 1H), 7.32 (m, 1H), 7.20 (m, 1H), 7.10 (m, 4H), 7.02-6.95 (m, 2H), 5.62 (m, 2H), 4.18-3.91 (m, 5H), 2.74-2.62 (m, 2H), 2.12 (s, 3H), 1.89 (m, 3H), 1.61 (m, 1H). MS m/z 412 (M+1).

### Example 15

### N-({1-[3-(Dimethylamino)propyl]-1H-benzimidazol-2-yl}methyl)-N-methyl-5,6,7,8-tetrahydro-8-quinolinamine

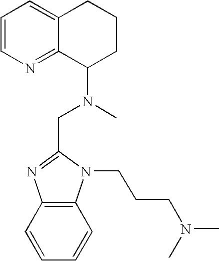

### a) 3-(2-{[Methyl(5,6,7,8-tetrahydro-8-quinolinyl)amino]methyl}-1H-benzimidazol-1-yl)propanenitrile

Reaction of N-(1H-benzimidazol-2-ylmethyl)-N-methyl-5,6,7,8-tetrahydro-8-quinolinamine (197 mg, 0.67 mmol) and 3-bromopropionitrile (84 μL, 1.00 mmol) as described herein for the preparation of N-methyl-N-{[1-(3-pyridinylmethyl)-1H-benzimidazol-2-yl]methyl}-5,6,7,8-tetrahydro-8-quinolinamine, afforded 192 mg (82%) of 3-(2-{[methyl(5,6,7,8-tetrahydro-8-quinolinyl)amino]methyl}-1H-benzimidazol-1-yl)propanenitrile as a yellow oil. ¹H NMR (DMSO-d₆): δ 8.46 (d, 1H), 7.65 (m, 1H), 7.55 (m, 1H), 7.48 (m, 1H), 7.24-7.11 (m, 3H), 4.81 (m, 1H), 4.64 (m, 1H), 4.18-4.01 (m, 3H), 3.27-3.15 (m, 2H), 2.81-2.64 (m, 3H), 2.03 (s, 3H), 1.93 (m, 2H), 1.65 (m, 1H). MS m/z 346 (M+1).

### b) N-{[1-(3-Aminopropyl)-1H-benzimidazol-2-yl]methyl}-N-methyl-5,6,7,8-tetrahydro-8-quinolinamine

Reduction of 3-(2-{[methyl(5,6,7,8-tetrahydro-8-quinolinyl)amino]methyl}-1H-benzimidazol-1-yl)propanenitrile (192 mg, 0.56 mmol) as herein described for the preparation of N-[(1-{[4-(aminomethyl)phenyl]methyl}-1H-benzimidazol-2-yl)methyl]-N-methyl-5,6,7,8-tetrahydro-8-quinolinamine, afforded 150 mg (77%) of N-{[1-(3-aminopropyl)-1H-benzimidazol-2-yl]methyl}-N-methyl-5,6,7,8-tetrahydro-8-quinolinamine as a yellow oil, after flash chromatography (silica gel, 0 to 10% NH₄OH in acetonitrile). ¹H NMR (DMSO-d₆): δ 8.43 (d, 1H), 7.53-7.48 (m, 3H), 7.19-7.09 (m, 3H), 4.36 (t, 2H), 4.19-3.96 (m, 3H), 2.81-2.64 (m, 3H), 2.52 (m, 1H), 2.07 (s, 3H), 1.95 (m, 3H), 1.79 (m, 2H), 1.62 (m, 1H). MS m/z 350 (M+1).

### c) N-({1-[3-(Dimethylamino)propyl]-1H-benzimidazol-2-yl}methyl)-N-methyl-5,6,7,8-tetrahydro-8-quinolinamine

Reductive methylation of N-{[1-(3-aminopropyl)-1H-benzimidazol-2-yl]methyl}-N-methyl-5,6,7,8-tetrahydro-8-quinolinamine (200 mg, 0.57 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 162 mg (75%) of N-({1-[3-(dimethylamino)propyl]-1H-benzimidazol-2-yl}methyl)-N-methyl-5,6,7,8-tetrahydro-8-quinolinamine as a colorless oil. ¹H NMR (DMSO-d₆): δ 8.42 (d, 1H), 7.53-7.46 (m, 3H), 7.19-7.09 (m, 3H), 4.31 (m, 2H), 4.20-4.05 (m, 2H), 3.94 (t, 1H), 2.81-2.64 (m, 2H), 2.12 (m, 2H), 2.08 (s, 9H), 1.94 (m, 3H), 1.83 (m, 2H), 1.64 (m, 1H). MS m/z 378 (M+1).

### Example 16

### N-Methyl-N-[(1-{3-[(2-pyridinylmethyl)amino]propyl}-1H-benzimidazol-2-yl)methyl]-5,6,7,8-tetrahydro-8-quinolinamine

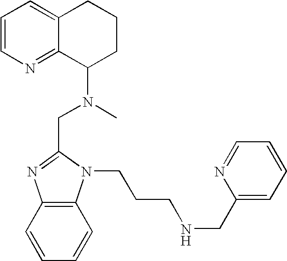

A solution of N-{[1-(3-aminopropyl)-1H-benzimidazol-2-yl]methyl}-N-methyl-5,6,7,8-tetrahydro-8-quinolinamine (66 mg, 0.19 mmol) in anhydrous DMF (5 mL) was treated with 2-picolyl chloride hydrochloride (28 mg, 0.17 mmol) and K₂CO₃ (78 mg, 0.57 mmol). The reaction was stirred at RT for 66 h, then partitioned between EtOAc and water. The aqueous layer was washed with EtOAc. The combined organic layers were washed with brine, dried (Na₂SO₄), and concentrated under reduced pressure. The crude product was purified by reverse phase HPLC as described herein for the preparation of N-methyl-N-({1-[2-(1-piperidinyl)ethyl]-1H-benzimidazol-2-yl}methyl)-5,6,7,8-tetrahydro-8-quinolinamine to afford 24 mg (29%) of N-methyl-N-[(1-{3-[(2-pyridinylmethyl)amino]propyl}-1H-benzimidazol-2-yl)methyl]-5,6,7,8-tetrahydro-8-quinolinamine as a yellow oil. ¹H NMR (DMSO-d₆): δ 8.44 (d, 1H), 8.39 (d, 1H), 7.67 (td, 1H), 7.53-7.44 (m, 3H), 7.36 (m, 1H), 7.21-7.06 (m, 4H), 4.38 (m, 2H), 4.20-4.02 (m, 2H), 3.94 (m, 1H), 3.75 (s, 2H), 2.77-2.61 (m, 2H), 2.51 (m, 1H), 2.04 (s, 3H), 1.90 (m, 6H), 1.56 (m, 1H). MS m/z 441 (M+1).

### Example 17

### N-([1-[5-(Dimethylamino)pentyl]-1H-benzimidazol-2-yl]methyl)-N-methyl-5,6,7,8-tetrahydro-8-quinolinamine

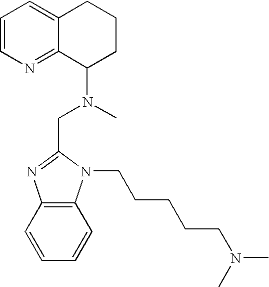

### a) 5-(2-{[Methyl(5,6,7,8-tetrahydro-8-quinolinyl)amino]methyl}-1H-benzimidazol-1-yl)pentanenitrile

Reaction of N-(1H-benzimidazol-2-ylmethyl)-N-methyl-5,6,7,8-tetrahydro-8-quinolinamine (199 mg, 0.68 mmol) and 5-bromovaleronitrile (87 μL, 0.75 mmol) as described herein for the preparation of N-methyl-N-{[1-(3-pyridinylmethyl)-1H-benzimidazol-2-yl]methyl}-5,6,7,8-tetrahydro-8-quinolinamine, afforded 141 mg (56%) of 5-(2-{[methyl(5,6,7,8-tetrahydro-8-quinolinyl)amino]methyl}-1H-benzimidazol-1-yl)pentanenitrile as a gold oil. ¹H NMR (DMSO-d₆): δ 8.44 (d, 1H), 7.55-7.50 (m, 3H), 7.23-7.12 (m, 3H), 4.37 (m, 2H), 4.22-3.96 (m, 3H), 2.81-2.65 (m, 2H), 2.53 (m, 2H), 2.07 (s, 3H), 1.97 (m, 3H), 1.83 (m, 2H), 1.65 (m, 1H), 1.56 (m, 2H). MS m/z 374 (M+1).

### b) N-{[1-(5-Aminopentyl)-1H-benzimidazol-2-yl]methyl}-N-methyl-5,6,7,8-tetrahydro-8-quinolinamine

Reduction of 5-(2-{[methyl(5,6,7,8-tetrahydro-8-quinolinyl)amino]methyl}-1H-benzimidazol-1-yl)pentanenitrile (133 mg, 0.36 mmol) as herein described for the preparation of N-{[1-(3-aminopropyl)-1H-benzimidazol-2-yl]methyl}-N-methyl-5,6,7,8-tetrahydro-8-quinolinamine, afforded 108 mg (81%) of N-{[1-(5-aminopentyl)-1H-benzimidazol-2-yl]methyl}-N-methyl-5,6,7,8-tetrahydro-8-quinolinamine as a brown oil. ¹H NMR (DMSO-d₆): δ 8.42 (d, 1H), 7.53-7.45 (m, 3H), 7.19-7.09 (m, 3H), 4.27-3.91 (m, 5H), 2.78-2.65 (m, 2H), 2.44 (m, 1H), 2.07 (s, 3H), 2.04 (m, 1H), 1.94 (m, 3H), 1.65 (m, 3H), 1.34-1.20 (m, 4H). MS m/z 378 (M+1).

### c) N-({1-[5-(Dimethylamino)pentyl]-1H-benzimidazol-2-yl}methyl)-N-methyl-5,6,7,8-tetrahydro-8-quinolinamine

Reductive methylation of N-{[1-(5-aminopentyl)-1H-benzimidazol-2-yl]methyl}-N-methyl-5,6,7,8-tetrahydro-8-quinolinamine (40 mg, 0.11 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 29 mg (67%) of N-({1-[5-(dimethylamino)pentyl]-1H-benzimidazol-2-yl}methyl)-N-methyl-5,6,7,8-tetrahydro-8-quinolinamine as a yellow oil. ¹H NMR (DMSO-d₆): δ 8.42 (d, 1H), 7.53-7.45 (m, 3H), 7.18-7.09 (m, 3H), 4.30-3.91 (m, 5H), 2.81-2.64 (m, 2H), 2.10 (m, 1H), 2.06 (s, 3H), 2.04 (s, 6H), 1.93 (m, 3H), 1.67 (m, 3H), 1.35 (m, 2H), 1.21 (m, 3H). MS m/z 406 (M+1).

### Example 18

### N-{[1-(2-Aminoethyl)-1H-benzimidazol-2-yl]methyl}-N-methyl-5,6,7,8-tetrahydro-8-quinolinamine

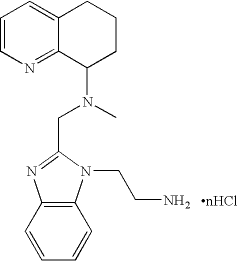

### a) t-Butyl (2-chloroethyl)carbamate

Reaction of t-butyl (2-hydroxyethyl)carbamate (0.20 mL, 1.29 mmol) with PS-triphenylphosphine and CCl₄ as described herein for the preparation of t-butyl 4-(chloromethyl)-1-piperidinecarboxylate, afforded 0.23 g (100%) of t-butyl (2-chloroethyl)carbamate as a colorless oil. ¹H NMR (CDCl₃): δ 5.30 (br s, 1H), 3.60 (m, 2H), 3.47 (d, 2H), 1.45 (s, 9H).

### b) t-Butyl [2-(2-{[methyl(5,6,7,8-tetrahydro-8-quinolinyl)amino]methyl}-1H-benzimidazol-1-yl)ethyl]carbamate

Reaction of t-butyl (2-chloroethyl)carbamate (230 mg, 1.28 mmol) and N-(1H-benzimidazol-2-ylmethyl)-N-methyl-5,6,7,8-tetrahydro-8-quinolinamine (100 mg, 0.34 mmol) as described herein for the preparation of N-methyl-N-{[1-(3-pyridinylmethyl)-1H-benzimidazol-2-yl]methyl}-5,6,7,8-tetrahydro-8-quinolinamine, afforded 55 mg (37%) of t-butyl [2-(2-{[methyl(5,6,7,8-tetrahydro-8-quinolinyl)amino]methyl}-1H-benzimidazol-1-yl)ethyl]carbamate as a yellow solid. ¹H NMR (DMSO-d₆): δ 8.42 (d, 1H), 7.53-7.46 (m, 3H), 7.29 (m, 1H), 7.19-7.09 (m, 3H), 4.38 (m, 2H), 4.20-4.02 (m, 3H), 3.32 (m, 2H), 2.80-2.64 (m, 2H), 2.04 (m, 4H), 1.92 (m, 2H), 1.63 (m, 1H), 1.22 (s, 9H). MS m/z 436 (M+1).

### c) N-{[1-(2-Aminoethyl)-1H-benzimidazol-2-yl]methyl}-N-methyl-5,6,7,8-tetrahydro-8-quinolinamine

A solution of t-butyl [2-(2-{[methyl(5,6,7,8-tetrahydro-8-quinolinyl)amino]methyl}-1H-benzimidazol-1-yl)ethyl]carbamate (55 mg, 0.13 mmol) in anhydrous MeOH (2 mL) was treated with 2 mL of 4N HCl/dioxane. After 2 h, the solution was concentrated under reduced pressure. Ethanol was added and concentrated (3×). The residue was dissolved in EtOH, and Et₂O was added. The resulting gold solid was filtered under a N₂ blanket and dried under vacuum. This afforded 31 mg (55%) of N-{[1-(2-aminoethyl)-1H-benzimidazol-2-yl]methyl}-N-methyl-5,6,7,8-tetrahydro-8-quinolinamine as the hydrochloride salt. ¹H NMR (D₂O): δ 8.41 (d, 1H), 8.09 (d, 1H), 7.71-7.59 (m, 3H), 7.49 (m, 2H), 4.72 (m, 2H), 4.42-4.31 (m, 3H), 3.43 (m, 2H), 2.84 (m, 2H), 2.28-2.20 (m, 4H), 2.09-1.96 (m, 2H), 1.72 (m, 1H). MS m/z 336 (M+1).

### Example 19

### N-{[1-(3-Aminopropyl)-1H-benzimidazol-2-yl]methyl}-5,6,7,8-tetrahydro-8-quinolinamine

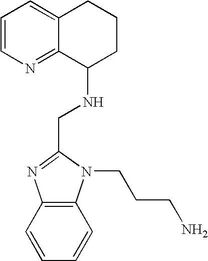

### a) 3-{2-[(5,6,7,8-Tetrahydro-8-quinolinylamino)methyl]-1H-benzimidazol-1-yl}propanenitrile

Reaction of N-(1H-benzimidazol-2-ylmethyl)-5,6,7,8-tetrahydro-8-quinolinamine (215 mg, 0.77 mg) and 3-bromopropionitrile (0.19 mL, 2.32 mmol) as described herein for the preparation of N-methyl-N-{[1-(3-pyridinylmethyl)-1H-benzimidazol-2-yl]methyl}-5,6,7,8-tetrahydro-8-quinolinamine, afforded 225 mg (88%) of 3-{2-[(5,6,7,8-tetrahydro-8-quinolinylamino)methyl]-1H-benzimidazol-1-yl}propanenitrile as a light brown oil. ¹H NMR (DMSO-d₆): δ 8.36 (d, 1H), 7.63 (d, 1H), 7.57 (d, 1H), 7.48 (d, 1H), 7.22-7.14 (m, 3H), 4.63 (m, 2H), 4.19 (m, 2H), 3.76 (m, 1H), 3.11 (m, 3H), 2.72 (m, 2H), 2.04 (m, 1H), 1.87 (m, 1H), 1.67 (m, 2H). MS m/z 332 (M+1).

### b) N-{[1-(3-Aminopropyl)-1H-benzimidazol-2-yl]methyl}-5,6,7,8-tetrahydro-8-quinolinamine

Reduction of 3-{2-[(5,6,7,8-tetrahydro-8-quinolinylamino)methyl]-1H-benzimidazol-1-yl}propanenitrile (44 mg, 0.13 mmol) as herein described for the preparation of N-{[1-(3-aminopropyl)-1H-benzimidazol-2-yl]methyl}-N-methyl-5,6,7,8-tetrahydro-8-quinolinamine, afforded 22 mg (50%) of N-{[1-(3-aminopropyl)-1H-benzimidazol-2-yl]methyl}-5,6,7,8-tetrahydro-8-quinolinamine as a light brown oil. ¹H NMR (DMSO-d₆): δ 8.37 (d, 1H), 7.57-7.49 (m, 3H), 7.20-7.12 (m, 3H), 4.32 (m, 2H), 4.21-4.06 (m, 2H), 3.78 (m, 1H), 2.74 (m, 2H), 2.53 (m, 5H), 2.10 (m, 1H), 1.93-1.68 (m, 5H). MS m/z 336 (M+1).

### Example 20

### N-{[1-(3-Aminopropyl)-1H-benzimidazol-2-yl]methyl}-N-ethyl-5,6,7,8-tetrahydro-8-quinolinamine

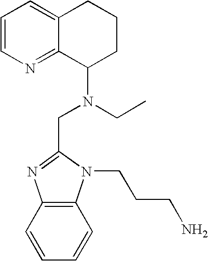

### a) 3-(2-{[Ethyl(5,6,7,8-tetrahydro-8-quinolinyl)amino]methyl}-1H-benzimidazol-1-yl)propanenitrile

A mixture of 3-{2-[(5,6,7,8-tetrahydro-8-quinolinylamino)methyl]-1H-benzimidazol-1-yl}propanenitrile (129 mg, 0.39 mmol), acetaldehyde (26 μL, 0.47 mmol), NaBH(OAc)₃ (165 mg, 0.78 mmol) and AcOH (67 μL, 1.17 mmol) in anhydrous 1,2-dichloroethane (5 mL) was allowed to stir at RT for 18 h. The reaction was partitioned between CH₂Cl₂ and saturated aqueous NaHCO₃. The aqueous layer was extracted again with CH₂Cl₂. The combined organic layers were washed with brine, dried (Na₂SO₄) and concentrated under reduced pressure. The crude product was purified by flash chromatography (silica gel, gradient elution of dichloromethane to 9:1 dichloromethane/2M NH₃ in MeOH) to afford 91 mg (65%) of 3-(2-{[ethyl(5,6,7,8-tetrahydro-8-quinolinyl)amino]methyl}-1H-benzimidazol-1-yl)propanenitrile as a yellow solid. ¹H NMR (DMSO-d₆): δ 8.42 (d, 1H), 7.64 (d, 1H), 7.54 (d, 1H), 7.44 (d, 1H), 7.22-7.11 (m, 3H), 4.89 (m, 1H), 4.66 (m, 1H), 4.18-3.95 (m, 3H), 3.36 (m, 2H), 3.29 (m, 2H), 2.78-2.62 (m, 2H), 2.05 (m, 1H), 1.92-1.82 (m, 2H), 1.58 (m, 1H), 0.85 (t, 3H). MS m/z 360 (M+1).

### b) N-{[1-(3-Aminopropyl)-1H-benzimidazol-2-yl]methyl}-N-ethyl-5,6,7,8-tetrahydro-8-quinolinamine

Reduction of 3-(2-{[ethyl(5,6,7,8-tetrahydro-8-quinolinyl)amino]methyl}-1H-benzimidazol-1-yl)propanenitrile (91 mg, 0.25 mmol) as herein described for the preparation of N-{[1-(3-aminopropyl)-1H-benzimidazol-2-yl]methyl}-N-methyl-5,6,7,8-tetrahydro-8-quinolinamine, afforded 68 mg (74%) of N-{[1-(3-aminopropyl)-1H-benzimidazol-2-yl]methyl}-N-ethyl-5,6,7,8-tetrahydro-8-quinolinamine as a pale yellow oil. ¹H NMR (DMSO-d₆): δ 8.42 (d, 1H), 7.53-7.45 (m, 3H), 7.19-7.10 (m, 3H), 4.45 (m, 2H), 4.21-4.00 (m, 3H), 2.77-2.52 (m, 6H), 1.96-1.80 (m, 5H), 1.60 (m, 1H), 0.84 (t, 3H). MS m/z 364 (M+1).

### Example 21

### N-{[1-(3-Aminopropyl)-1H-benzimidazol-2-yl]methyl}-N-(phenylmethyl)-5,6,7,8-tetrahydro-8-quinolinamine

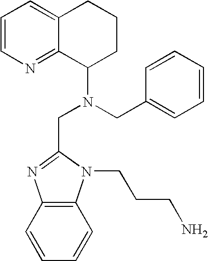

### a) N-(1H-Benzimidazol-2-ylmethyl)-N-(phenylmethyl)-5,6,7,8-tetrahydro-8-quinolinamine

Reaction of N-(1H-benzimidazol-2-ylmethyl)-5,6,7,8-tetrahydro-8-quinolinamine (238 mg, 0.86 mmol) and benzaldehyde (0.10 mL, 1.03 mmol) as herein described for the preparation of 3-(2-{[ethyl(5,6,7,8-tetrahydro-8-quinolinyl)amino]methyl}-1H-benzimidazol-1-yl)propanenitrile, afforded 229 mg (73%) of N-(1H-benzimidazol-2-ylmethyl)-N-(phenylmethyl)-5,6,7,8-tetrahydro-8-quinolinamine as a colorless oil. ¹H NMR (DMSO-d₆): δ 12.53 (s, 1H), 8.58 (d, 1H), 7.53-7.40 (m, 5H), 7.29-7.03 (m, 6H), 4.06-3.69 (m, 5H), 2.81-2.60 (m, 2H), 2.13 (m, 1H), 1.92 (m, 2H), 1.57 (m, 1H). MS m/z 369 (M+1).

### b) 3-(2-{[(Phenylmethyl)(5,6,7,8-tetrahydro-8-quinolinyl)amino]methyl}-1H-benzimidazol-1-yl)propanenitrile

Reaction of N-(1H-benzimidazol-2-ylmethyl)-N-(phenylmethyl)-5,6,7,8-tetrahydro-8-quinolinamine (123 mg, 0.33 mmol) and 3-bromopropionitrile (83 μL, 1.00 mmol) as described herein for the preparation of N-methyl-N-{[1-(3-pyridinylmethyl)-1H-benzimidazol-2-yl]methyl}-5,6,7,8-tetrahydro-8-quinolinamine, afforded 110 mg (78%) of 3-(2-{[(phenylmethyl)(5,6,7,8-tetrahydro-8-quinolinyl)amino]methyl}-1H-benzimidazol-1-yl)propanenitrile as a brown foam. ¹H NMR (DMSO-d₆): δ 8.52 (d, 1H), 7.55 (m, 2H), 7.46 (d, 1H), 7.34 (m, 2H), 7.30-7.22 (m, 2H), 7.19-7.11 (m, 4H), 4.94 (m, 1H), 4.33 (m, 1H), 4.02 (m, 2H), 3.83 (m, 1H), 3.57 (m, 2H), 3.21 (m, 1H), 3.08 (m, 1H), 2.81-2.61 (m, 2H), 2.13 (m, 1H), 1.92 (m, 2H), 1.51 (m, 1H). MS m/z 422 (M+1).

### c) N-{[1-(3-Aminopropyl)-1H-benzimidazol-2-yl]methyl}-N-(phenylmethyl)-5,6,7,8-tetrahydro-8-quinolinamine

Reduction of 3-(2-{[(phenylmethyl)(5,6,7,8-tetrahydro-8-quinolinyl)amino]methyl}-1H-benzimidazol-1-yl)propanenitrile (110 mg, 0.26 mmol) as described herein for the preparation of N-{[1-(3-aminopropyl)-1H-benzimidazol-2-yl]methyl}-N-methyl-5,6,7,8-tetrahydro-8-quinolinamine, afforded 82 mg (74%) of N-{[1-(3-aminopropyl)-1H-benzimidazol-2-yl]methyl}-N-(phenylmethyl)-5,6,7,8-tetrahydro-8-quinolinamine as a white foam. ¹H NMR (DMSO-d₆): δ 8.49 (d, 1H), 7.50 (d, 1H), 7.45 (m, 2H), 7.33 (m, 2H), 7.22 (t, 2H), 7.16-7.07 (m, 4H), 4.41 (m, 1H), 4.27-3.90 (m, 4H), 3.75-3.57 (m, 2H), 2.80-2.59 (m, 2H), 2.34 (m, 2H), 2.02-1.86 (m, 3H), 1.62-1.48 (m, 3H). MS m/z 426 (M+1).

### Example 22

### N-{[1-(3-Aminopropyl)-1H-benzimidazol-2-yl]methyl}-N-(3-methylbutyl)-5,6,7,8-tetrahydro-8-quinolinamine

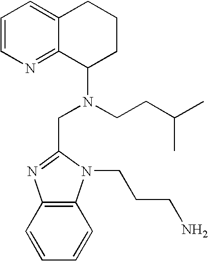

### a) N-(1H-Benzimidazol-2-ylmethyl)-N-(3-methylbutyl)-5,6,7,8-tetrahydro-8-quinolinamine

Reaction of N-(1H-benzimidazol-2-ylmethyl)-5,6,7,8-tetrahydro-8-quinolinamine (318 mg, 1.14 mmol) and isovaleraldehyde (0.15 mL, 1.37 mmol) as herein described for the preparation of 3-(2-{[ethyl(5,6,7,8-tetrahydro-8-quinolinyl)amino]methyl}-1H-benzimidazol-1-yl)propanenitrile, afforded 338 mg (85%) of N-(1H-benzimidazol-2-ylmethyl)-N-(3-methylbutyl)-5,6,7,8-tetrahydro-8-quinolinamine as an off-white foam. ¹H NMR (DMSO-d₆): δ 12.44 (s, 1H), 8.51 (d, 1H), 7.49 (m, 3H), 7.17 (m, 1H), 7.07 (m, 2H), 4.05-3.87 (m, 3H), 2.81-2.55 (m, 4H), 2.08 (m, 1H), 1.87 (m, 2H), 1.66-1.47 (m, 2H), 1.18 (m, 2H), 0.67 (d, 3H), 0.62 (d, 3H). MS m/z 349 (M+1).

### b) 3-(2-{[(3-Methylbutyl)(5,6,7,8-tetrahydro-8-quinolinyl)amino]methyl}-1H-benzimidazol-1-yl)propanenitrile

Reaction of N-(1H-benzimidazol-2-ylmethyl)-N-(3-methylbutyl)-5,6,7,8-tetrahydro-8-quinolinamine (164 mg, 0.47 mmol) and 3-bromopropionitrile (0.12 mL, 1.41 mmol) as described herein for the preparation of N-methyl-N-{[1-(3-pyridinylmethyl)-1H-benzimidazol-2-yl]methyl}-5,6,7,8-tetrahydro-8-quinolinamine, afforded 157 mg (83%) of 3-(2-{[(3-methylbutyl)(5,6,7,8-tetrahydro-8-quinolinyl)amino]methyl}-1H-benzimidazol-1-yl)propanenitrile as a brown oil. ¹H NMR (DMSO-d₆): δ 8.43 (d, 1H), 7.63 (d, 1H), 7.54 (d, 1H), 7.45 (d, 1H), 7.22-7.11 (m, 3H), 5.00 (m, 1H), 4.63 (m, 1H), 4.15-3.93 (m, 3H), 3.35-3.19 (m, 3H), 2.79-2.62 (m, 3H), 2.05 (m, 1H), 1.86 (m, 2H), 1.58 (m, 1H), 1.44 (m, 1H), 1.17 (m, 2H), 0.64 (d, 3H), 0.57 (d, 3H). MS m/z 402 (M+1).

### c) N-{[1-(3-Aminopropyl)-1H-benzimidazol-2-yl]methyl}-N-(3-methylbutyl)-5,6,7,8-tetrahydro-8-quinolinamine

Reduction of 3-(2-{[(3-methylbutyl)(5,6,7,8-tetrahydro-8-quinolinyl)amino]methyl}-1H-benzimidazol-1-yl)propanenitrile (157 mg, 0.39 mmol) as described herein for the preparation of N-{[1-(3-aminopropyl)-1H-benzimidazol-2-yl]methyl}-N-methyl-5,6,7,8-tetrahydro-8-quinolinamine, afforded 102 mg (65%) of N-{[1-(3-aminopropyl)-1H-benzimidazol-2-yl]methyl}-N-(3-methylbutyl)-5,6,7,8-tetrahydro-8-quinolinamine as a gold oil. ¹H NMR (DMSO-d₆): δ 8.41 (d, 1H), 7.51 (m, 2H), 7.44 (d, 1H), 7.18-7.08 (m, 3H), 4.46 (m, 2H), 4.17-3.96 (m, 3H), 2.78-2.54 (m, 3H), 2.52-2.41 (m, 2H), 1.96-1.82 (m, 4H), 1.79 (m, 2H), 1.55 (m, 1H), 1.43 (m, 1H), 1.11 (m, 2H), 0.60 (d, 3H), 0.57 (d, 3H). MS m/z 402 (M+1).

### Example 23

### N-Methyl-N-({1-[2-(1-methyl-2-piperidinyl)ethyl]-1H-benzimidazol-2-yl}methyl)-5,6,7,8-tetrahydro-8-quinolinamine

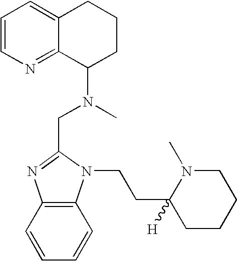

### a) t-Butyl 2-(2-hydroxyethyl)-1-piperidinecarboxylate

Reaction of 2-(2-piperidinyl)ethanol (1.00 g, 7.74 mmol) with di-t-butyl dicarbonate (1.86 g, 8.51 mmol) as described herein for the preparation of t-butyl 3-(hydroxymethyl)-1-piperidinecarboxylate, afforded 1.81 g (100%) of t-butyl 2-(2-hydroxyethyl)-1-piperidinecarboxylate as a colorless oil. ¹H NMR (DMSO-d₆): δ 4.29 (t, 1H), 4.16 (brs, 1H), 3.77 (brd, 1H), 3.29 (m, 1H), 2.69 (brt, 1H), 1.73 (m, 1H), 1.58-1.37 (m, 6H), 1.35 (s, 9H), 1.21 (m, 1H).

### b) t-Butyl 2-(2-chloroethyl)-1-piperidinecarboxylate

Reaction of t-butyl 2-(2-hydroxyethyl)-1-piperidinecarboxylate (0.88 g, 3.84 mmol) with PS-triphenylphosphine and CCl₄ as described herein for the preparation of t-butyl 4-(chloromethyl)-1-piperidinecarboxylate, afforded 0.86 g (90%) of t-butyl 2-(2-chloroethyl)-1-piperidinecarboxylate as a cloudy oil. ¹H NMR (CDCl₃): δ 4.40 (m, 1H), 4.00 (br d, 1H), 3.48 (m, 2H), 2.72 (t, 1H), 2.23 (m, 1H), 1.80 (m, 1H), 1.66-1.54 (m, 4H), 1.51 (s, 1H), 1.44 (s, 9H), 1.36 (m, 1H).

### c) t-Butyl 2-[2-(2-{[methyl(5,6,7,8-tetrahydro-8-quinolinyl)amino]methyl}-1H-benzimidazol-1-yl)ethyl]-1-piperidinecarboxylate

Reaction of t-butyl 2-(2-chloroethyl)-1-piperidinecarboxylate (0.86 g, 3.47 mmol) and N-(1H-benzimidazol-2-ylmethyl)-N-methyl-5,6,7,8-tetrahydro-8-quinolinamine (250 mg, 0.86 mmol) as described herein for the preparation of N-methyl-N-{[1-(3-pyridinylmethyl)-1H-benzimidazol-2-yl]methyl}-5,6,7,8-tetrahydro-8-quinolinamine, afforded 98 mg (23%) of t-butyl 2-[2-(2-{[methyl(5,6,7,8-tetrahydro-8-quinolinyl)amino]methyl}-1H-benzimidazol-1-yl)ethyl]-1-piperidinecarboxylate as a brown oil. The diastereomers were indistinguishable by analytical RP-HPLC, however, ¹H NMR analysis is consistent with a 1:1 diasteromer mixture. ¹H NMR (DMSO-d₆): δ 8.41 (m, 1H), 7.54 (m, 1H), 7.47 (m, 1H), 7.41 (m, 1H), 7.21-7.11 (m, 3H), 4.34-4.12 (m, 5H), 3.99-3.86 (m, 2H), 2.86-2.63 (m, 3H), 2.08 (m, 4H), 1.92 (m, 4H), 1.51 (m, 6H), 1.33 (s, 9H), 1.25 (m, 1H). MS m/z 504 (M+1).

### d) N-Methyl-N-({1-[2-(2-piperidinyl)ethyl]-1H-benzimidazol-2-yl}methyl)-5,6,7,8-tetrahydro-8-quinolinamine

A solution of t-butyl 2-[2-(2-{[methyl(5,6,7,8-tetrahydro-8-quinolinyl)amino]methyl}-1H-benzimidazol-1-yl)ethyl]-1-piperidinecarboxylate (98 mg, 0.19 mmol) in 4 mL of anhydrous MeOH was treated with 2 mL of 4N HCl/dioxane. After 2 h, the reaction was concentrated under reduced pressure. Ethanol was added and concentrated (4×), then hexane was added and concentrated (4×). The resulting gold foam was dried under vacuum. This afforded 96 mg (96%) of N-methyl-N-({1-[2-(2-piperidinyl)ethyl]-1H-benzimidazol-2-yl}methyl)-5,6,7,8-tetrahydro-8-quinolinamine as the hydrochloride salt. The diastereomers were indistinguishable by analytical RP-HPLC, however, ¹H NMR analysis is consistent with a 1:1 diasteromer mixture. ¹H NMR (D₂O): δ 8.49 (d, 1H), 8.21 (d, 1H), 7.74-7.67 (m, 3H), 7.53 (m, 2H), 4.46-4.29 (m, 5H), 3.31-3.21 (m, 2H), 2.87 (m, 3H), 2.19 (m, 4H), 2.15-1.92 (m, 5H), 1.76 (m, 3H), 1.55-1.40 (m, 3H). MS m/z 404 (M+1).

### e) N-Methyl-N-({1-[2-(1-methyl-2-piperidinyl)ethyl]-1H-benzimidazol-2-yl}methyl)-5,6,7,8-tetrahydro-8-quinolinamine

Reductive methylation of the hydrochloride salt of N-methyl-N-({1-[2-(2-piperidinyl)ethyl]-1H-benzimidazol-2-yl}methyl)-5,6,7,8-tetrahydro-8-quinolinamine (50 mg, 0.097 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 26 mg (64%) of N-methyl-N-({1-[2-(1-methyl-2-piperidinyl)ethyl]-H-benzimidazol-2-yl}methyl)-5,6,7,8-tetrahydro-8-quinolinamine as a yellow oil. The diastereomers were indistinguishable by analytical RP-HPLC, however, ¹H NMR analysis is consistent with a 1:1 diasteromer mixture. ¹H NMR (CD₃OD): δ 8.40 (m, 1H), 7.57 (m, 1H), 7.49-7.43 (m, 2H), 7.28-7.13 (m, 3H), 4.51-4.30 (m, 2H), 4.14-3.94 (m, 3H), 2.91-2.72 (m, 3H), 2.25, 2.24 (s, 3H total, 2 diastereomers), 2.17, 2.15 (s, 3H total, 2 diastereomers), 2.14-2.06 (m, 6H), 1.74 (m, 4H), 1.59 (m, 2H), 1.32 (m, 2H). MS m/z 418 (M+1).

### Example 24

### N-({1-[(2Z)-4-(Dimethylamino)-2-buten-1-yl]-1H-benzimidazol-2-yl}methyl)-N-methyl-5,6,7,8-tetrahydro-8-quinolinamine

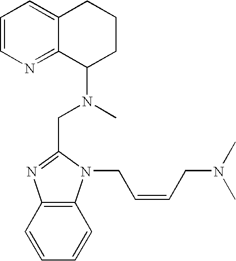

### a) t-Butyl [(2Z)-4-chloro-2-buten-1-yl]carbamate

A suspension of (2Z)-4-chloro-2-buten-1-amine (1.00 g, 7.00 mmol) in 35 mL THF and 0.2 mL water was treated with N,N-diisopropylethylamine (2.7 mL, 15.5 mmol) and di-t-butyl dicarbonate (1.80 g, 8.20 mmol). After 2.5 h, the reaction was partitioned between Et₂O and saturated aqueous NaHCO₃. The aqueous layer was extracted again with Et₂O. The combined organic layers were washed with brine, dried (MgSO₄) and concentrated under reduced pressure. The crude product was purified by flash chromatography (silica gel, 0 to 50% EtOAc in hexanes) to afford 1.15 g (80%) of t-butyl [(2Z)-4-chloro-2-buten-1-yl]carbamate as a white solid. ¹H NMR (CDCl₃): δ 5.74 (m, 1H), 5.62 (m, 1H), 4.57 (br s, 1H), 4.11 (m, 2H), 3.82 (m, 2H), 1.43 (s, 9H).

### b) t-Butyl [(2Z)-4-(2-{[methyl(5,6,7,8-tetrahydro-8-quinolinyl)amino]methyl}-1H-benzimidazol-1-yl)-2-buten-1-yl]carbamate

A solution of N-(1H-benzimidazol-2-ylmethyl)-N-methyl-5,6,7,8-tetrahydro-8-quinolinamine (150 mg, 0.51 mmol), t-butyl [(2Z)-4-chloro-2-buten-1-yl]carbamate (0.42 g, 2.05 mmol) and K₂CO₃ (0.35 g, 2.60 mmol) in 5 mL of DMF was stirred at RT. After 18 h, the reaction was partitioned between EtOAc and water. The aqueous layer was extracted again with EtOAc. The combined organic layers were washed with brine, dried (Na₂SO₄) and concentrated under reduced pressure. The crude product was purified by flash chromatography (silica gel, gradient elution of dichloromethane to 9:1 dichloromethane/2M NH₃ in MeOH) to afford 198 mg (84%) of t-butyl [(2Z)-4-(2-{[methyl(5,6,7,8-tetrahydro-8-quinolinyl)amino]methyl}-1H-benzimidazol-1-yl)-2-buten-1-yl]carbamate as a white foam. ¹H NMR (DMSO-d₆): δ 8.41 (d, 1H), 7.52 (m, 1H), 7.45 (m, 2H), 7.13 (m, 4H), 5.46-5.36 (m, 2H), 5.13 (m, 2H), 4.20-4.07 (m, 2H), 3.95 (m, 1H), 3.79 (t, 2H), 2.79-2.63 (m, 2H), 2.06 (s, 3H), 1.96-1.86 (m, 3H), 1.62 (m, 1H), 1.38 (s, 9H). MS m/z 462 (M+1).

### c) N-({1-[(2Z)-4-Amino-2-buten-1-yl]-1H-benzimidazol-2-yl}methyl)-N-methyl-5,6,7,8-tetrahydro-8-quinolinamine

Reaction of t-butyl [(2Z)-4-(2-{[methyl(5,6,7,8-tetrahydro-8-quinolinyl)amino]methyl}-1H-benzimidazol-1-yl)-2-buten-1-yl]carbamate (93 mg, 0.20 mmol) as described herein for the preparation of N-methyl-N-({1-[2-(2-piperidinyl)ethyl]-1H-benzimidazol-2-yl}methyl)-5,6,7,8-tetrahydro-8-quinolinamine, afforded 100 mg of the hydrochloride salt of N-({1-[(2Z)-4-amino-2-buten-1-yl]-1H-benzimidazol-2-yl}methyl)-N-methyl-5,6,7,8-tetrahydro-8-quinolinamine as an off-white foam. ¹H NMR (D₂O): δ 8.48 (d, 1H), 8.18 (d, 1H), 7.71 (m, 2H), 7.60 (m, 1H), 7.50 (m, 2H), 5.73 (m, 2H), 5.11 (m, 2H), 4.48-4.29 (m, 3H), 3.82 (m, 2H), 2.88 (m, 2H), 2.22 (s, 3H), 2.18 (m, 1H), 2.09-1.91 (m, 2H), 1.74 (m, 1H). MS m/z 362 (M+1).

### d) N-({1-[(2Z)-4-(Dimethylamino)-2-buten-1-yl]-1H-benzimidazol-2-yl}methyl)-N-methyl-5,6,7,8-tetrahydro-8-quinolinamine

Reductive methylation of the hydrochloride salt of N-({1-[(2Z)-4-amino-2-buten-1-yl]-1H-benzimidazol-2-yl}methyl)-N-methyl-5,6,7,8-tetrahydro-8-quinolinamine (50 mg, 0.11 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 28 mg (68%) of N-({1-[(2Z)-4-(dimethylamino)-2-buten-1-yl]-1H-benzimidazol-2-yl}methyl)-N-methyl-5,6,7,8-tetrahydro-8-quinolinamine as a colorless oil. ¹H NMR (CD₃OD): δ 8.38 (d, 1H), 7.56 (m, 1H), 7.46 (m, 1H), 7.36 (m, 1H), 7.22 (m, 2H), 7.13 (m, 1H), 5.65 (m, 1H), 5.53 (m, 1H), 5.16 (m, 2H), 4.11-3.95 (m, 3H), 3.23 (d, 2H), 2.90-2.71 (m, 2H), 2.31 (s, 6H), 2.24 (s, 3H), 2.09 (m, 3H), 1.72 (m, 1H). MS m/z 390 (M+1).

### Example 25

### N-Methyl-N-({1-[(4-methyl-2-morpholinyl)methyl]-1H-benzimidazol-2-yl}methyl)-5,6,7,8-tetrahydro-8-quinolinamine

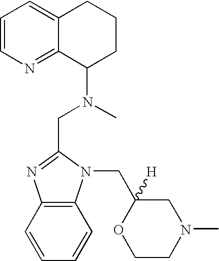

### a) t-Butyl 2-(chloromethyl)-4-morpholinecarboxylate

Reaction of t-butyl 2-(hydroxymethyl)-4-morpholinecarboxylate (0.75 g, 3.45 mmol, Tyger Scientific) with PS-triphenylphosphine and CCl₄ as described herein for the preparation of t-butyl 4-(chloromethyl)-1-piperidinecarboxylate, afforded 0.85 g (100%) of t-butyl 2-(chloromethyl)-4-morpholinecarboxylate as a pale yellow oil. ¹H NMR (CDCl₃): δ 4.03 (br s, 1H), 3.92 (m, 1H), 3.84 (br m, 1H), 3.64-3.42 (m, 4H), 2.96 (br m, 1H), 2.75 (br m, 1H), 1.47 (s, 9H).

### b) t-Butyl 2-[(2-{[methyl(5,6,7,8-tetrahydro-8-quinolinyl)amino]methyl}-1H-benzimidazol-1-yl)methyl]-4-morpholinecarboxylate

Reaction of N-(1H-benzimidazol-2-ylmethyl)-N-methyl-5,6,7,8-tetrahydro-8-quinolinamine (150 mg, 0.51 mmol) and t-butyl 2-(chloromethyl)-4-morpholinecarboxylate (0.36 g, 1.54 mmol) as described herein for the preparation of N-methyl-N-({1-[2-(1-piperidinyl)ethyl]-1H-benzimidazol-2-yl}methyl)-5,6,7,8-tetrahydro-8-quinolinamine, afforded 195 mg (77%) of t-butyl 2-[(2-{[methyl(5,6,7,8-tetrahydro-8-quinolinyl)amino]methyl}-1H-benzimidazol-1-yl)methyl]-4-morpholinecarboxylate as a light brown oil. The diastereomers were indistinguishable by analytical RP-HPLC, however, ¹H NMR analysis is consistent with a 1:1 diasteromer mixture. ¹H NMR (DMSO-d₆): δ 8.41 (m, 1H), 7.56-7.46 (m, 3H), 7.22-7.06 (m, 3H), 4.59-4.36 (m, 2H), 4.19-3.64 (m, 7H), 3.21 (m, 2H), 2.76-2.64 (m, 4H), 2.09, 2.02 (s, 3H total, 2 diastereomers), 1.92 (m, 2H), 1.63 (m, 1H), 1.32 (s, 9H). MS m/z 492 (M+1).

### c) N-Methyl-N-{[1-(2-morpholinylmethyl)-1H-benzimidazol-2-yl]methyl}-5,6,7,8-tetrahydro-8-quinolinamine

Reaction of t-butyl 2-[(2-{[methyl(5,6,7,8-tetrahydro-8-quinolinyl)amino]methyl}-1H-benzimidazol-1-yl)methyl]-4-morpholinecarboxylate (187 mg, 0.38 mmol) as described herein for the preparation of N-methyl-N-{[1-(4-piperidinylmethyl)-1H-benzimidazol-2-yl]methyl}-5,6,7,8-tetrahydro-8-quinolinamine, afforded 115 mg (77%) of N-methyl-N-{[1-(2-morpholinylmethyl)-1H-benzimidazol-2-yl]methyl}-5,6,7,8-tetrahydro-8-quinolinamine as a gold oil. The diastereomers were indistinguishable by analytical RP-HPLC, however, ¹H NMR analysis is consistent with a 1:1 diasteromer mixture. ¹H NMR (DMSO-d₆): δ 8.43 (m, 1H), 7.49 (m, 3H), 7.19-7.09 (m, 3H), 4.44-4.33 (m, 2H), 4.18-4.06 (m, 2H), 3.94-3.82 (m, 2H), 3.65-3.52 (m, 2H), 3.21 (m, 1H), 2.82-2.52 (m, 4H), 2.40-2.25 (m, 2H), 2.11, 2.03 (s, 3H total, 2 diastereomers), 1.95 (m, 2H), 1.64 (m, 1H). MS m/z 392 (M+1).

### d) N-Methyl-N-({1-[(4-methyl-2-morpholinyl)methyl]-1H-benzimidazol-2-yl}methyl)-5,6,7,8-tetrahydro-8-quinolinamine

Reductive methylation of N-methyl-N-{[1-(2-morpholinylmethyl)-1H-benzimidazol-2-yl]methyl}-5,6,7,8-tetrahydro-8-quinolinamine (53 mg, 0.14 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 48 mg (89%) of N-methyl-N-({1-[(4-methyl-2-morpholinyl)methyl]-1H-benzimidazol-2-yl}methyl)-5,6,7,8-tetrahydro-8-quinolinamine as an off-white foam, after flash chromatography (silica gel, 0 to 10% NH₄OH in acetonitrile). The diastereomers were indistinguishable by analytical RP-HPLC, however, ¹H NMR analysis is consistent with a 1:1 diasteromer mixture. ¹H NMR (DMSO-d₆): δ 8.43 (m, 1H), 7.49 (m, 3H), 7.20-7.09 (m, 3H), 4.52-4.33 (m, 2H), 4.18-4.06 (m, 2H), 3.93-3.57 (m, 4H), 3.25 (m, 1H), 2.82-2.64 (m, 3H), 2.11-2.03 (m, 6H), 1.96-1.83 (m, 4H), 1.71-1.57 (m, 2H). MS m/z 406 (M+1).

### Example 26

### N-Methyl-N-{[1-(2-pyridinylmethyl)-1H-benzimidazol-2-yl]methyl}-5,6,7,8-tetrahydro-8-quinolinamine

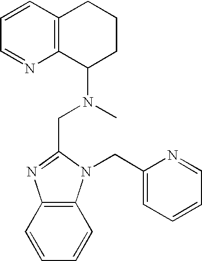

A mixture of N-(1H-benzimidazol-2-ylmethyl)-N-methyl-5,6,7,8-tetrahydro-8-quinolinamine (0.100 g, 0.342 mmol), 2-(chloromethyl)pyridine hydrochloride (0.112 g, 0.684 mmol), and K₂CO₃ (0.236 g, 1.71 mmol) in 6 mL of anhydrous DMF was stirred at RT for 18 hours and then heated to 70° C. for an additional 3 hours. The solution was cooled to RT and diluted with EtOAc. The resulting solution was washed with aqueous brine (3×), dried over Na₂SO₄, and concentrated to dryness at reduced pressure. The crude product was purified by flash chromatography (silica gel, gradient elution of dichloromethane to 85:15 dichloromethane/2M NH₃ in MeOH) to afford 87 mg (66%) of N-methyl-N-{[1-(2-pyridinylmethyl)-1H-benzimidazol-2-yl]methyl}-5,6,7,8-tetrahydro-8-quinolinamine as a tacky yellow foam. ¹H NMR (DMSO-d₆): δ 8.48 (d, 1H), 8.31 (d, 1H), 7.73 (t, 1H), 7.60 (m, 1H), 7.50-7.38 (m, 2H), 7.27 (m, 1H), 7.20-7.06 (m, 4H), 5.95 (d, 1H), 5.78 (d, 1H), 4.23 (d, 1H), 4.10 (d, 1H), 3.98 (m, 1H), 2.81-2.60 (m, 2H), 2.17 (s, 3H), 1.99-1.80 (m, 3H), 1.62 (m, 1H). MS m/z 384 (M+1).

### Example 27

### N-Methyl-N-{[1-(4-pyridinylmethyl)-1H-benzimidazol-2-yl]methyl}-5,6,7,8-tetrahydro-8-quinolinamine

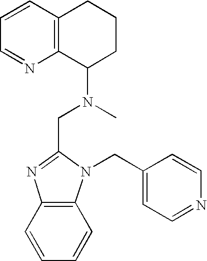

Alkylation of N-(1H-benzimidazol-2-ylmethyl)-N-methyl-5,6,7,8-tetrahydro-8-quinolinamine (0.100 g, 0.342 mmol) with 4-(chloromethyl)pyridine hydrochloride (0.112 g, 0.684 mmol) as described herein for the preparation of N-methyl-N-{[1-(3-pyridinylmethyl)-1H-benzimidazol-2-yl]methyl}-5,6,7,8-tetrahydro-8-quinolinamine followed by analogous chromatographic purification afforded 69 mg (53%) of N-methyl-N-{[1-(4-pyridinylmethyl)-1H-benzimidazol-2-yl]methyl}-5,6,7,8-tetrahydro-8-quinolinamine as a viscous yellow oil. ¹H NMR (DMSO-d₆): δ 8.50 (d, 2H), 8.31 (d, 1H), 7.63 (d, 1H), 7.49 (d, 1H), 7.43 (m, 1H), 7.24-7.09 (m, 3H), 7.03 (d, 2H), 5.90 (d, 1H), 5.79 (d, 1H), 4.20 (d, 1H), 4.08-3.91 (m, 2H), 2.81-2.60 (m, 2H), 2.14 (s, 3H), 1.93-1.79 (m, 3H), 1.62 (m, 1H). MS m/z 384 (M+1).

### Example 28

### 2-(2-{[Methyl(5,6,7,8-tetrahydro-8-quinolinyl)amino]methyl}-1H-benzimidazol-1-yl)-N-(4-pyridinylmethyl)acetamide

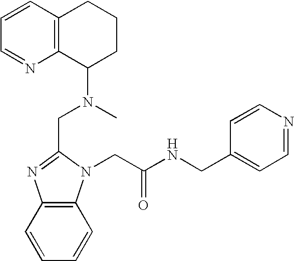

### a) Methyl (2-{[methyl(5,6,7,8-tetrahydro-8-quinolinyl)amino]methyl}-1H-benzimidazol-1-yl)acetate

A mixture of N-(1H-benzimidazol-2-ylmethyl)-N-methyl-5,6,7,8-tetrahydro-8-quinolinamine (0.100 g, 0.342 mmol), methyl bromoacetate (65 μL, 0.684 mmol), and K₂CO₃ (0.236 g, 1.71 mmol) in 5 mL of anhydrous DMF was heated to 70° C. with stirring for 1 hour. The mixture was cooled to RT and diluted with EtOAc. The resulting solution was washed with aqueous brine (3×), dried over Na₂SO₄, and concentrated to dryness at reduced pressure. The crude product was purified by flash chromatography (silica gel, gradient elution of chloroform to 9:1 chloroform/2M NH₃ in EtOH) to afford 73 mg (58%) of methyl (2-{[methyl(5,6,7,8-tetrahydro-8-quinolinyl)amino]methyl}-1H-benzimidazol-1-yl)acetate as a tacky yellow foam. ¹H NMR (DMSO-d₆): δ 8.43 (m, 1H), 7.63-7.45 (m, 3H), 7.29-7.13 (m, 3H), 5.69 (d, 1H), 5.41 (d, 1H), 4.19 (d, 1H), 4.07-3.96 (m, 2H), 3.70 (s, 3H), 2.90-2.64 (m, 2H), 2.13-1.81 (m, 6H), 1.71 (m, 1H). MS m/z 365 (M+1).

### b) 2-(2-{[Methyl(5,6,7,8-tetrahydro-8-quinolinyl)amino]methyl}-1H-benzimidazol-1-yl)-N-(4-pyridinylmethyl)acetamide

To a solution of 4-(aminomethyl)pyridine (20 μL, 0.193 mmol) in 3 mL of anhydrous dichloromethane contained in a screw cap sealed tube under an N₂ atmosphere was added 2M AlMe₃ in toluene (97 μL, 0.193 mmol). The resulting solution was stirred at RT for 15 minutes and then treated with methyl (2-{[methyl(5,6,7,8-tetrahydro-8-quinolinyl)amino]methyl}-1H-benzimidazol-1-yl)acetate (64 mg, 0.176 mmol) dissolved in 1 mL of anhydrous dichloromethane. The reaction vessel was sealed and the solution heated to 40° C. with stirring. After 18 hours the solution was cooled to RT and treated with an additional 97 μL portion of 2M AlMe₃ in toluene and 20 μL of 4-(aminomethyl)pyridine (pre-mixed as a solution in dichloromethane for 15 minutes in a separate vessel). After stirring for an additional 18 hours at 40° C. the solution was cooled to RT and quenched by addition of 1 mL of 1 N aqueous HCl. After stirring for 5 minutes the mixture was diluted with dichloromethane and stirred vigorously with addition of 10% aqueous Na₂CO₃. After stirring for 30 minutes the phases were separated. The dichloromethane solution was washed with saturated aqueous brine (1×), dried over Na₂SO₄, and concentrated to dryness at reduced pressure. The crude residue was purified by reverse phase HPLC (C8, gradient elution of H₂O/0.1% TFA to MeCN over 40 minutes). Fractions containing pure product (as determined by analytical HPLC) were combined and concentrated to dryness at reduced pressure. The residue was dissolved in EtOAc and the solution washed with 10% aqueous Na₂CO₃ (1×) followed by aqueous brine (1×). After drying over Na₂SO₄, the solution was concentrated to dryness to afford 44 mg (56%) of 2-(2-{[Methyl(5,6,7,8-tetrahydro-8-quinolinyl)amino]methyl}-1H-benzimidazol-1-yl)-N-(4-pyridinylmethyl)acetamide as a light yellow foam. ¹H NMR (DMSO-d₆): δ 9.06 (t, 1H), 8.49 (dd, 2H), 8.40 (d, 1H), 7.63-7.44 (m, 3H), 7.32-7.11 (m, 5H), 5.47 (d, 1H), 5.24 (d, 1H), 4.38 (d, 2H), 4.16-3.92 (m, 3H), 2.90-2.63 (m, 2H), 2.18-1.80 (m, 6H), 1.68 (m, 1H). MS m/z 441 (M+1).

### Example 29

### 2-(2-{[Methyl(5,6,7,8-tetrahydro-8-quinolinyl)amino]methyl}-1H-benzimidazol-1-yl)-N-(3-pyridinylmethyl)acetamide

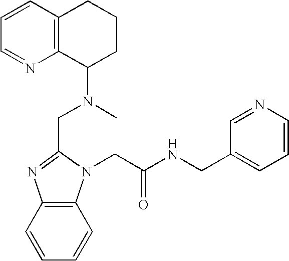

To a solution of 3-(aminomethyl)pyridine (42 μL, 0.411 mmol) in 3 mL of anhydrous 1,2-dichloroethane contained in a screw cap sealed tube under an N₂ atmosphere was added 2M AlMe₃ in toluene (0.21 mL, 0.41 mmol), After stirring at RT for 15 minutes, a solution of methyl (2-{[methyl(5,6,7,8-tetrahydro-8-quinolinyl)amino]methyl}-1H-benzimidazol-1-yl)acetate in 2 mL of anhydrous 1,2-dichloroethane was added. The reaction vessel was capped and the solution heated to 80° C. with stirring. After 2.5 hours, the solution was cooled to RT and quenched by addition of 1 mL of 1N aqueous HCl. Work-up and purification as described herein for 2-(2-{[Methyl(5,6,7,8-tetrahydro-8-quinolinyl)amino]methyl}-1H-benzimidazol-1-yl)-N-(4-pyridinylmethyl)acetamide afforded 21 mg (35%) of 2-(2-{[Methyl(5,6,7,8-tetrahydro-8-quinolinyl)amino]methyl}-1H-benzimidazol-1-yl)-N-(3-pyridinylmethyl)acetamide as a light yellow foam ¹H NMR (DMSO-d₆): δ 9.02 (t, 1H), 8.52-8.43 (m, 2H), 8.39 (d, 1H), 7.70-7.40 (m, 4H), 7.34 (m, 1H), 7.26-7.10 (m, 3H), 5.42 (d, 1H), 5.19 (d, 1H), 4.39 (d, 2H), 4.12-3.92 (m, 3H), 2.88-2.61 (m, 2H), 2.18-1.79 (m, 6H), 1.64 (m, 1H). MS m/z 441 (M+1).

### Example 30

### N-(3-Aminopropyl)-2-(2-{[methyl(5,6,7,8-tetrahydro-8-quinolinyl)amino]methyl}-1H-benzimidazol-1-yl)acetamide

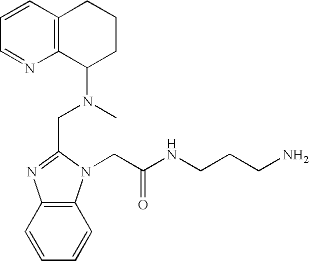

### a) (2-{[Methyl(5,6,7,8-tetrahydro-8-quinolinyl)amino]methyl}-1H-benzimidazol-1-yl)acetic acid

A solution of methyl (2-{[methyl(5,6,7,8-tetrahydro-8-quinolinyl)amino]methyl}-1H-benzimidazol-1-yl)acetate (0.100 g, 0.274 mmol) in 7 mL of 3:1 MeOH/water was treated with LiOH (13 mg, 0.548 mmol) and the resulting solution stirred at RT for 3 hours. The solution was then treated with 0.60 mL of 1N aqueous HCl and concentrated to dryness at reduced pressure. The residue was dissolved in 5 mL of EtOH and again concentrated to dryness at reduced pressure. The residue was then triturated with addition of EtOH and diethyl ether. A white suspension was produced which was stirred at RT for 15 minutes. The solid was collected by vacuum filtration and dried under vacuum to afford 89 mg (93%) of (2-{[methyl(5,6,7,8-tetrahydro-8-quinolinyl)amino]methyl}-1H-benzimidazol-1-yl)acetic acid as a white powder. ¹H NMR (DMSO-d₆): δ 8.69 (m, 1H), 8.20-7.30 (m, 6H), 5.42 (s, 2H), 4.97-4.50 (m, 3H), 3.06-2.23 (m, 6H), 2.20-1.98 (m, 2H), 1.83 (m, 1H). MS m/z 351 (M+1).

### b) 1,1-Dimethylethyl (3-{[(2-{[methyl(5,6,7,8-tetrahydro-8-quinolinyl)amino]methyl}-1H-benzimidazol-1-yl)acetyl]amino}propyl)carbamate

A solution of (2-{[methyl(5,6,7,8-tetrahydro-8-quinolinyl)amino]methyl}-1H-benzimidazol-1-yl)acetic acid (75 mg, 0.21 mmol), 1,1-dimethylethyl (3-aminopropyl)carbamate (75 mg, 0.43 mmol), N-[(dimethylamino)-1H-1,2,3-triazolo[4,5-b]pyridine-1-ylmethylene]-N-methylmethanaminium hexafluorophosphate N-oxide (0.163 g, 0.428 mmol), and N,N-diisopropylethylamine (0.11 mL, 0.64 mmol) in 5 mL of anhydrous DMF was stirred at RT for 18 hours. The solution was diluted with EtOAc, washed with saturated aqueous brine (3×), dried over Na₂SO₄, and concentrated to dryness at reduced pressure. The crude product was purified by flash chromatography (silica gel, gradient elution of chloroform to 9:1 chloroform/2M NH₃ in EtOH) to afford 72 mg (1,1-dimethylethyl (3-{[(2-{[methyl(5,6,7,8-tetrahydro-8-quinolinyl)amino]methyl}-1H-benzimidazol-1-yl)acetyl]amino}propyl)carbamate as a light yellow foam. ¹H NMR (DMSO-d₆): δ 8.51-8.39 (m, 2H), 7.62-7.51 (m, 2H), 7.45 (d, 1H), 7.32-7.13 (m, 3H), 6.80 (t, 1H), 5.33 (d, 1H), 5.08 (d, 1H), 4.11-3.90 (m, 3H), 3.19-3.02 (m, 2H), 3.00-2.64 (m, 4H), 2.20-1.86 (m, 6H), 1.78-1.36 (m, 12H). MS m/z 507 (M+1).

### c) N-(3-Aminopropyl)-2-(2-{[methyl(5,6,7,8-tetrahydro-8-quinolinyl)amino]methyl}-1H-benzimidazol-1-yl)acetamide

A solution of (1,1-dimethylethyl (3-{[(2-{[methyl(5,6,7,8-tetrahydro-8-quinolinyl)amino]methyl}-1H-benzimidazol-1-yl)acetyl]amino}propyl)carbamate (66 mg, 0.13 mmol) in 3 mL of anhydrous MeOH was treated with 1 mL of 4N HCl/dioxane and the resulting solution stirred at RT for 1 hour. The solution was concentrated to dryness at reduced pressure. The residue was dissolved in minimum EtOH and the solution stirred with addition of excess diethyl ether. A white suspension was produced that was stirred at RT for 30 minutes. The solid was collected by vacuum filtration and dried under vacuum to afford 60 mg (90%) of N-(3-Aminopropyl)-2-(2-{[methyl(5,6,7,8-tetrahydro-8-quinolinyl)amino]methyl}-1H-benzimidazol-1-yl)acetamide as the hydrochloride salt. ¹H NMR (DMSO-d₆): δ 9.19 (t, 1H), 8.78 (d, 1H), 8.36-7.71 (m, 5H), 7.62-7.48 (m, 2H), 5.36 (s, 2H), 4.90-4.47 (m, 3H), 3.23 (q, 2H), 3.02-2.71 (m, 4H), 2.58-2.27 (m, 5H), 2.21-1.98 (m, 2H), 1.97-1.70 (m, 3H). MS m/z 407 (M+1).

### Example 31

### N-(2-Aminoethyl)-2-(2-{[methyl(5,6,7,8-tetrahydro-8-quinolinyl)amino]methyl}-1H-benzimidazol-1-yl)acetamide

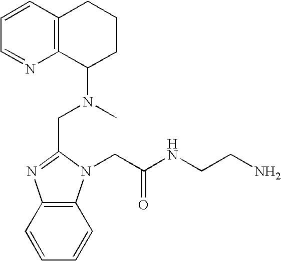

### a) 1,1-Dimethylethyl (2-{[(2-{[methyl(5,6,7,8-tetrahydro-8-quinolinyl)amino]methyl}-1H-benzimidazol-1-yl)acetyl]amino}ethyl)carbamate

Employing the method described herein for the preparation of 1,1-dimethylethyl (3-{[(2-{[methyl(5,6,7,8-tetrahydro-8-quinolinyl)amino]methyl}-1H-benzimidazol-1-yl)acetyl]amino}propyl)carbamate, 50 mg (0.14 mmol) of (2-{[methyl(5,6,7,8-tetrahydro-8-quinolinyl)amino]methyl}-1H-benzimidazol-1-yl)acetic acid was coupled with 1,1-dimethylethyl (2-aminoethyl)carbamate (46 mg, 0.29 mmol) to afford, following flash chromatography (silica gel, gradient elution of dichloromethane to 95:5 dichloromethane/2M NH₃ in MeOH), 35 mg (50%) of 1,1-dimethylethyl (2-{[(2-{[methyl(5,6,7,8-tetrahydro-8-quinolinyl)amino]methyl}-1H-benzimidazol-1-yl)acetyl]amino}ethyl)carbamate as a white foam. ¹H NMR (CDCl₃): δ 9.35 (br s, 1H), 8.42 (d, 1H), 7.70-7.58 (m, 2H), 7.50 (d, 1H), 7.32-7.12 (m, 3H), 5.75 (brs, 1H), 4.84 (d, 1H), 4.58 (d, 1H), 4.24 (m, 1H), 3.90 (d, 1H), 3.71 (d, 1H), 3.52 (m, 1H), 3.39-3.03 (m, 3H), 2.88 (m, 1H), 2.77 (m, 1H), 2.40-2.00 (m, 6H), 1.80 (m, 1H), 1.32 (s, 9H). MS m/z 493 (M+1).

### b) N-(2-Aminoethyl)-2-(2-{[methyl(5,6,7,8-tetrahydro-8-quinolinyl)amino]methyl}-1H-benzimidazol-1-yl)acetamide

A solution of 1,1-dimethylethyl (2-{[(2-{[methyl(5,6,7,8-tetrahydro-8-quinolinyl)amino]methyl}-1H-benzimidazol-1-yl)acetyl]amino}ethyl)carbamate (32 mg, 0.065 mmol) in 2 mL of anhydrous MeOH was treated with 2 mL of 4N HCl/dioxane and the resulting solution stirred at RT. After 1.5 hours the solution was concentrated to dryness at reduced pressure. The residue was shaken with a mixture of 8 mL of dichloromethane and 8 mL of 10% aqueous Na₂CO₃. The phases were separated. The dichloromethane solution was dried by passing through a hydrophobic separator tube (Alltech Associates, Deerfield, Ill., 60015) and the filtrate concentrated to dryness at reduced pressure to afford 21 mg (84%) of N-(2-aminoethyl)-2-(2-{[methyl(5,6,7,8-tetrahydro-8-quinolinyl)amino]methyl}-1H-benzimidazol-1-yl)acetamide as a viscous oil. ¹H NMR (CDCl₃): δ 9.33 (m, 1H), 8.41 (d, 1H), 7.70-7.60 (m, 2H), 7.45 (d, 1H), 7.31-7.18 (m, 2H), 7.12 (m, 1H), 5.06 (d, 1H), 4.70 (d, 1H), 4.13 (m, 1H), 3.92 (d, 1H), 3.82-3.73 (m, 2H), 3.61 (m, 1H), 3.41 (m, 1H), 3.18 (m, 1H), 2.93-2.66 (m, 3H), 2.32-2.13 (m, 4H), 2.11-1.67 (m, 4H). MS m/z 393 (M+1).

### Example 32

### N-Methyl-N-({1-[2-oxo-2-(1-piperazinyl)ethyl]-1H-benzimidazol-2-yl}methyl)-5,6,7,8-tetrahydro-8-quinolinamine

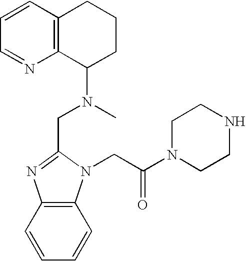

### a) 1,1-dimethylethyl 4-[(2-{[methyl(5,6,7,8-tetrahydro-8-quinolinyl)amino]methyl}-1H-benzimidazol-1-yl)acetyl]-1-piperazinecarboxylate

Employing the method described herein for the preparation of 1,1-dimethylethyl (3-{[(2-{[methyl(5,6,7,8-tetrahydro-8-quinolinyl)amino]methyl}-1H-benzimidazol-1-yl)acetyl]amino}propyl)carbamate, 50 mg (0.14 mmol) of (2-{[methyl(5,6,7,8-tetrahydro-8-quinolinyl)amino]methyl}-1H-benzimidazol-1-yl)acetic acid was coupled with 1,1-dimethylethyl 1-piperazinecarboxylate (53 mg, 0.29 mmol) to afford, following flash chromatography (silica gel, gradient elution of dichloromethane to 95:5 dichloromethane/2M NH₃ in MeOH), 39 mg (53%) of 1,1-dimethylethyl 4-[(2-{[methyl(5,6,7,8-tetrahydro-8-quinolinyl)amino]methyl}-1H-benzimidazol-1-yl)acetyl]-1-piperazinecarboxylate as a light yellow foam. ¹H NMR (CDCl₃): δ 8.37 (d, 1H), 7.69 (m, 1H), 7.38 (d, 1H), 7.23-7.15 (m, 3H), 7.06 (m, 1H), 6.12 (brs, 1H), 5.72 (d, 1H), 4.16-3.25 (m, 11H), 2.87-2.63 (m, 2H), 2.28-2.09 (m, 4H), 2.07-1.87 (m, 2H), 1.70 (m, 1H), 1.48 (s, 9H). MS m/z 519 (M+1).

### b) N-Methyl-N-({1-[2-oxo-2-(1-piperazinyl)ethyl]-1H-benzimidazol-2-yl}methyl)-5,6,7,8-tetrahydro-8-quinolinamine

Employing the method described herein for the preparation of N-(2-aminoethyl)-2-(2-{[methyl(5,6,7,8-tetrahydro-8-quinolinyl)amino]methyl}-1H-benzimidazol-1-yl)acetamide, 36 mg (0.069 mmol) of 1,1-dimethylethyl 4-[(2-{[methyl(5,6,7,8-tetrahydro-8-quinolinyl)amino]methyl}-1H-benzimidazol-1-yl)acetyl]-1-piperazinecarboxylate was subjected to HCl induced deprotection to afford 23 mg (79%) of N-methyl-N-({1-[2-oxo-2-(1-piperazinyl)ethyl]-1H-benzimidazol-2-yl}methyl)-5,6,7,8-tetrahydro-8-quinolinamine as a light yellow foam. ¹H NMR (CDCl₃): δ 8.39 (d, 1H), 7.69 (m, 1H), 7.36 (d, 1H), 7.23-7.16 (m, 3H), 7.03 (m, 1H), 5.98 (d, 1H), 5.71 (d, 1H), 4.07 (d, 1H), 4.01-3.92 (m, 2H), 3.86-3.58 (m, 3H), 3.48 (m, 1H), 2.97-2.63 (m, 5H), 2.33-2.07 (m, 6H), 2.06-1.88 (m, 2H), 1.70 (m, 1H). MS m/z 419 (M+1).

### Example 33

### 2-(2-{[Methyl(5,6,7,8-tetrahydro-8-quinolinyl)amino]methyl}-1H-benzimidazol-1-yl)-N-(2-pyridinylmethyl)acetamide

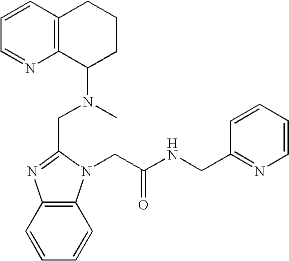

Employing the method described herein for the preparation of 1,1-dimethylethyl (3-{[(2-{[methyl(5,6,7,8-tetrahydro-8-quinolinyl)amino]methyl}-1H-benzimidazol-1-yl)acetyl]amino}propyl)carbamate, 77 mg (0.22 mmol) of (2-{[methyl(5,6,7,8-tetrahydro-8-quinolinyl)amino]methyl}-1H-benzimidazol-1-yl)acetic acid was coupled with 2-(aminomethyl)pyridine (48 mg, 0.44 mmol) to afford, following flash chromatography (silica gel, gradient elution of dichloromethane to 9:1 dichloromethane/2M NH₃ in MeOH), 61 mg (64%) of 2-(2-{[methyl(5,6,7,8-tetrahydro-8-quinolinyl)amino]methyl}-1H-benzimidazol-1-yl)-N-(2-pyridinylmethyl)acetamide as a light yellow foam. ¹H NMR (DMSO-d₆): δ 9.09 (t, 1H), 8.51 (m, 1H), 8.40 (m, 1H), 7.72 (t, 1H), 7.60 (d, 1H), 7.57-7.46 (m, 2H), 7.32-7.13 (m, 5H), 5.49 (d, 1H), 5.28 (d, 1H), 4.44 (d, 2H), 4.14-3.97 (m, 3H), 2.88-2.65 (m, 2H), 2.18-1.80 (m, 6H), 1.66 (m, 1H). MS m/z 441 (M+1).

### Example 34

### N-Methyl-N-({1-[(1-methyl-3-pyrrolidinyl)methyl]-1H-benzimidazol-2-yl}methyl)-5,6,7,8-tetrahydro-8-quinolinamine HCl salt

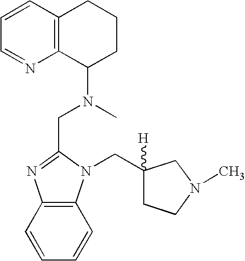

### a) N-Methyl-N-{[1-(3-pyrrolidinylmethyl)-1H-benzimidazol-2-yl]methyl}-5,6,7,8-tetrahydro-8-quinolinamine HCl salt

A solution of 1,1-dimethylethyl 3-[(2-{[methyl(5,6,7,8-tetrahydro-8-quinolinyl)amino]methyl}-1H-benzimidazol-1-yl)methyl]-1-pyrrolidinecarboxylate (40 mg, 0.084 mmol) in 2 mL of anhydrous MeOH was treated with 2 mL of 4N HCl/dioxane and the solution stirred at RT. After 30 minutes the solution was concentrated to dryness at reduced pressure. The residue was twice dissolved in MeOH and concentrated to dryness to afford 37 mg of N-methyl-N-{[1-(3-pyrrolidinylmethyl)-1H-benzimidazol-2-yl]methyl}-5,6,7,8-tetrahydro-8-quinolinamine as the HCl salt (90% yield based on tri-HCl salt). The diastereomers were indistinguishable by analytical RP-HPLC, however, ¹H NMR analysis is consistent with a 1:1 diastereomer mixture. ¹H NMR (CD₃OD): δ8.84 (d, 1H), 8.43 (d, 1H), 8.13-7.92 (m, 3H), 7.75-7.65 (m, 2H), 4.86-4.59 (m, 5H), 3.61-3.50 (m, 2H), 3.35-3.19 (m, 2H), 3.18-3.03 (m, 3H), 2.51 (m, 1H), 2.41-2.38 (m, 3H), 2.32-2.08 (m, 3H), 2.07-1.89 (m, 2H). MS m/z 376 (M+1).

### b) N-Methyl-N-({1-[(1-methyl-3-pyrrolidinyl)methyl]-1H-benzimidazol-2-yl}methyl)-5,6,7,8-tetrahydro-8-quinolinamine HCl salt

A solution of N-methyl-N-{[1-(3-pyrrolidinylmethyl)-1H-benzimidazol-2-yl]methyl}-5,6,7,8-tetrahydro-8-quinolinamine HCl salt (27 mg, 0.056 mmol based on tri-HCl salt) in 5 mL of anhydrous MeOH was stirred with addition of 0.15 mL of triethylamine. The resulting solution was concentrated to dryness at reduced pressure. The residue was dissolved in 5 mL of 1,2-dichloroethane and the solution treated with 37% aqueous formaldehyde (28 μL, 0.24 mmol) followed by NaBH(OAc)₃ (72 mg, 0.34 mmol). The resulting cloudy solution was stirred at RT for 2 hours. The solution was diluted with dichloromethane followed by 10% aqueous Na₂CO₃ and the mixture stirred vigorously for 20 minutes. The phases were separated. The organic solution was washed with 10% aqueous Na₂CO₃ (1×), saturated aqueous brine (1×), dried over Na₂SO₄, and concentrated to dryness at reduced pressure. The residue was dissolved in 2 mL of MeOH and the solution treated with 0.15 mL of 4N HCl/dioxane. The resulting solution was concentrated to dryness at reduced pressure. The residue was again dissolved in MeOH and concentrated to dryness to afford 26 mg N-methyl-N-({1-[(1-methyl-3-pyrrolidinyl)methyl]-1H-benzimidazol-2-yl}methyl)-5,6,7,8-tetrahydro-8-quinolinamine as the HCl salt (93% yield based on tri-HCl salt). The diastereomers were indistinguishable by analytical RP-HPLC, however, ¹H NMR analysis is consistent with a 1:1 diastereomer mixture. ¹H NMR (CD₃OD): δ 8.81 (d, 1H), 8.40 (d, 1H), 8.18-7.90 (m, 3H), 7.73-7.60 (m, 2H), 4.90-4.58 (m, 5H), 3.79 (m, 1H), 3.58 (m, 1H), 3.47-2.88 (m, 8H), 2.52 (m, 1H), 2.43-2.07 (m, 6H), 2.05-1.88 (m, 2H). MS m/z 376 (M+1).

### Example 35

### N-({1-[trans-4-(Dimethylamino)cyclohexyl]-1H-benzimidazol-2-yl}methyl)-N-methyl-5,6,7,8-tetrahydro-8-quinolinamine

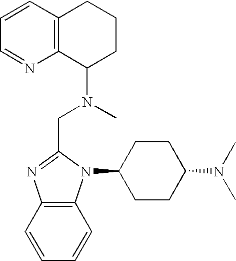

### a) 1,1-Dimethylethyl {trans-4-[(2-nitrophenyl)amino]cyclohexyl}carbamate

A mixture of 1,1-dimethylethyl (trans-4-aminocyclohexyl)carbamate (2.00 g, 9.33 mmol, Astatech, Inc., Philadelphia, Pa.), 1-chloro-2-nitrobenzene (2.21 g, 14.0 mmol), and K₂CO₃ (3.90 g, 28.0 mmol) in 25 mL of anhydrous DMF was heated to 120° C. with stirring. After 18 hours the reaction mixture was treated with an additional 2.2 g of 1-chloro-2-nitrobenzene followed by 3.0 g of K₂CO₃ and stirring at 120° C. continued. Following an additional 18 hour reaction period the mixture was cooled to RT and diluted with EtOAc. The solution was washed with saturated aqueous brine (5×), dried over Na₂SO₄, and concentrated to dryness at reduced pressure to afford a dark brown residue. The crude product was subjected to flash chromatography (silica gel, gradient elution of hexane to 1:1 hexane/EtOAc) to afford 1.04 g (33%) of 1,1-dimethylethyl {trans-4-[(2-nitrophenyl)amino]cyclohexyl}carbamate as a yellow solid. ¹H NMR (DMSO-d₆): δ 8.09 (d, 1H), 7.92 (d, 1H), 7.54 (t, 1H), 7.18 (d, 1H), 6.88 (d, 1H), 6.72 (t, 1H), 3.59 (m, 1H), 3.38-3.10 (m, 1H), 2.12-2.00 (m, 2H), 1.92-1.70 (m, 3H), 1.50-1.31 (m, 11H), 1.19 (m, 1H). MS m/z 358 (M+Na).

### b) 1,1-Dimethylethyl {trans-4-[(2-aminophenyl)amino]cyclohexyl}carbamate

A solution of 1,1-dimethylethyl {trans-4-[(2-nitrophenyl)amino]cyclohexyl}carbamate (0.50 g, 1.50 mmol) in 45 mL of MeOH was subjected to hydrogenation at 40 psi in the presence of 100 mg of 10% Pd on carbon. After 1 hour the reaction vessel was purged with nitrogen, catalyst removed by filtration through celite, and the filtrate concentrated to dryness at reduced pressure to afford 0.48 g (96%) of 1,1-dimethylethyl {trans-4-[(2-aminophenyl)amino]cyclohexyl}carbamate as a brown solid. ¹H NMR (DMSO-d₆): δ 6.80-6.62 (m, 1H), 6.53-6.30 (m, 4H), 4.43 (s, 2H), 4.04 (d, 1H), 3.28-3.00 (m, 2H), 2.02-1.90 (m, 2H), 1.85-1.65 (m, 3H), 1.42-1.06 (m, 12H). MS m/z 328 (M+Na).

### c) Phenylmethyl N-(5,6,7,8-tetrahydro-8-quinolinyl)glycinate

A solution of 6,7-dihydro-8(5H)-quinolinone (2.00 g, 13.6 mmol, J. Org. Chem., 2002, 67, 2197-2205), glycine benzyl ester (2.25 g, 13.6 mmol, free base prepared by dissolving the commercial HCl salt in 10% aqueous Na₂CO₃, extracting 4 times with EtOAc, drying the solution over Na₂SO₄, and concentrating at reduced pressure), and glacial acetic acid (1.60 mL, 27.2 mmol) in 40 mL of 1,2-dichloroethane was treated with NaBH(OAc)₃ (4.32 g, 20.4 mmol) by portion-wise addition over a 1 hour period.

After stirring at RT for 18 hours, the solution was diluted with an equal volume of 10% aqueous Na₂CO₃ and the mixture stirred vigorously for 30 minutes. The mixture was diluted with dichloromethane, stirred briefly, and the phases separated. The organic solution was washed once with saturated aqueous brine, dried over Na₂SO₄, and concentrated to dryness at reduced pressure to afford phenylmethyl N-(5,6,7,8-tetrahydro-8-quinolinyl)glycinate in quantitative yield as a yellow oil. ¹H NMR (CDCl₃): δ 8.39 (d, 1H), 7.44-7.27 (m, 6H), 7.06 (m, 1H), 5.19 (s, 2H), 3.80 (t, 1H), 3.65 (d, 2H), 2.88-2.67 (m, 3H), 2.16-1.94 (m, 2H), 1.87-1.64 (m, 2H). MS m/z 297 (M+1).

### d) Phenylmethyl N-methyl-N-(5,6,7,8-tetrahydro-8-quinolinyl)glycinate

Reductive methylation of phenylmethyl N-(5,6,7,8-tetrahydro-8-quinolinyl)glycinate (4.00 g, 13.5 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, followed by flash chromatography (silica gel, gradient elution of dichloromethane to 95:5 dichloromethane/2M NH₃ in MeOH) afforded 2.17 g (52%) of phenylmethyl N-methyl-N-(5,6,7,8-tetrahydro-8-quinolinyl)glycinate as a yellow-brown oil. ¹H NMR (CDCl₃): δ 8.41 (d, 1H), 7.40-7.26 (m, 6H), 7.04 (m, 1H), 5.15 (s, 2H), 4.02 (m, 1H), 3.66-3.46 (m, 2H), 2.86-2.60 (m, 2H), 2.47 (s, 3H), 2.14-1.80 (m, 3H), 1.68 (m, 1H). MS m/z 311 (M+1).

### e) N-Methyl-N-(5,6,7,8-tetrahydro-8-quinolinyl)glycine

A solution of phenylmethyl N-methyl-N-(5,6,7,8-tetrahydro-8-quinolinyl)glycinate (2.17 g, 6.98 mmol) in 35 mL of MeOH was subjected to catalytic hydrogenation at 50 psi in the presence of 10% Pd on carbon (0.22 g). After 2.5 hours the reaction vessel was purged with nitrogen, catalyst removed by filtration through celite, and the filtrate concentrated to dryness at reduced pressure to afford 1.36 g (89%) of N-methyl-N-(5,6,7,8-tetrahydro-8-quinolinyl)glycine as a light tan solid. ¹H NMR (DMSO-d₆): δ 8.43 (d, 1H), 7.62 (d, 1H), 7.30 (dd, 1H), 4.37 (m, 1H), 3.42 (d, 1H), 3.27 (d, 1H), 2.90-2.63 (m, 2H), 2.48 (s, 3H), 2.15 (m, 1H), 1.97 (m, 1H), 1.91-1.62 (m, 2H). MS m/z 221 (M+1).

### f) 1,1-Dimethylethyl {trans-4-[(2-{[N-methyl-N-(5,6,7,8-tetrahydro-8-quinolinyl)glycyl]amino}phenyl)amino]cyclohexyl}carbamate

A mixture of 1,1-dimethylethyl {trans-4-[(2-aminophenyl)amino]cyclohexyl}carbamate (0.10 g, 0.33 mmol) and N-methyl-N-(5,6,7,8-tetrahydro-8-quinolinyl)glycine (72 mg, 0.33 mmol) in 8 mL of anhydrous acetonitrile was treated with N,N-diisopropylethylamine (68 μL, 0.392 mmol) and gently warmed to dissolve the solid starting materials. After cooling to RT the solution was treated with bis(2-oxo-3-oxazolidinyl)phosphinic chloride (0.10 g, 0.39 mmol). After stirring at RT for 2.5 hours, the solution was diluted with EtOAc, washed with 10% aqueous Na₂CO₃ (2×), saturated aqueous brine (1×), dried over Na₂SO₄, and concentrated to dryness at reduced pressure to afford 1,1-dimethylethyl{trans-4-[(2-{[N-methyl-N-(5,6,7,8-tetrahydro-8-quinolinyl)glycyl]amino}phenyl)amino]cyclohexyl}carbamate in quantitative yield as a light brown foam. ¹H NMR (CDCl₃): δ 10.70 (s, 1H), 8.45 (d, 1H), 7.58-7.39 (m, 2H), 7.19-7.04 (m, 2H), 6.82-6.70 (m, 2H), 4.65-4.31 (m, 3H), 4.07 (m, 1H), 3.57-3.10 (m, 4H), 2.98-2.81 (m, 2H), 2.50 (s, 3H), 2.38-1.41 (m, 14H), 1.38-1.06 (m, 6H). MS m/z 508 (M+1).

### g) 1,1-Dimethylethyl [trans-4-(2-{[methyl(5,6,7,8-tetrahydro-8-quinolinyl)amino]methyl}-1H-benzimidazol-1-yl)cyclohexyl]carbamate

A solution of 1,1-dimethylethyl {trans-4-[(2-{[N-methyl-N-(5,6,7,8-tetrahydro-8-quinolinyl)glycyl]amino}phenyl)amino]cyclohexyl}carbamate (0.16 g, 0.32 mmol) in 8 mL of glacial acetic acid was heated to 70° C. with stirring. After 3 hours the solution was concentrated to dryness at reduced pressure and the residue dissolved in EtOAc. The solution was washed with 10% aqueous Na₂CO₃ (2×), saturated aqueous brine (1×), dried over Na₂SO₄, and concentrated to dryness at reduced pressure. The crude product was purified by reverse phase HPLC (C8, gradient elution of H₂O/0.1% TFA to MeCN over 40 minutes). Fractions containing pure product (as determined by analytical HPLC) were combined and concentrated to a volume of approximately 20 mL by rotary evaporation. The solution was treated with excess 10% aqueous Na₂CO₃ and the resulting mixture extracted with EtOAc (3×). The combined organic extracts were washed once with saturated aqueous brine, dried over Na₂SO₄ and concentrated to dryness at reduced pressure to afford 64 mg (42%) of 1,1-dimethylethyl [trans-4-(2-{[methyl(5,6,7,8-tetrahydro-8-quinolinyl)amino]methyl}-1H-benzimidazol-1-yl)cyclohexyl]carbamate as a light yellow foam. ¹H NMR (CDCl₃): δ 8.46 (d, 1H), 7.70 (m, 1H), 7.49 (m, 1H), 7.39 (d, 1H), 7.21-7.13 (m, 2H), 7.07 (m, 1H), 4.80 (m, 1H), 4.44 (m, 1H), 4.22 (d, 1H), 4.01-3.88 (m, 2H), 3.61 (m, 1H), 2.90-2.65 (m, 2H), 2.46-2.28 (m, 2H), 2.25-2.10 (m, 4H), 2.08-1.82 (m, 5H), 1.80-1.60 (m, 2H), 1.55-1.27 (m, 11H). MS m/z 490 (M+1).

### h) N-{[1-(trans-4-Aminocyclohexyl)-1H-benzimidazol-2-yl]methyl}-N-methyl-5,6,7,8-tetrahydro-8-quinolinamine

A solution of 1,1-dimethylethyl [trans-4-(2-{[methyl(5,6,7,8-tetrahydro-8-quinolinyl)amino]methyl}-1H-benzimidazol-1-yl)cyclohexyl]carbamate (60 mg, 0.12 mmol) in 1:1 trifluoroacetic acid/dichloromethane was stirred at RT for 2.5 hours and then concentrated to dryness at reduced pressure. The residue was dissolved in EtOAc. The solution was washed with 10% aqueous Na₂CO₃ (2×), saturated aqueous brine (1×), dried over Na₂SO₄ and concentrated to dryness at reduced pressure to afford 44 mg (92%) of N-{[1-(trans-4-aminocyclohexyl)-1H-benzimidazol-2-yl]methyl}-N-methyl-5,6,7,8-tetrahydro-8-quinolinamine as a light yellow foam. ¹H NMR (CDCl₃): δ 8.48 (d, 1H), 7.70 (m, 1H), 7.49 (m, 1H), 7.39 (d, 1H), 7.21-7.12 (m, 2H), 7.08 (m, 1H), 4.72 (m, 1H), 4.28 (d, 1H), 4.03-3.90 (m, 2H), 2.97-2.79 (m, 2H), 2.72 (m, 1H), 2.40-2.21 (m, 2H), 2.18 (s, 3H), 2.12-1.98 (m, 5H), 1.96-1.82 (m, 2H), 1.80-1.60 (m, 2H), 1.48-1.20 (m, 3H). MS m/z 390 (M+1).

### i) N-({1-[trans-4-(Dimethylamino)cyclohexyl]-1H-benzimidazol-2-yl}methyl)-N-methyl-5,6,7,8-tetrahydro-8-quinolinamine

A mixture of N-{[1-(trans-4-aminocyclohexyl)-1H-benzimidazol-2-yl]methyl}-N-methyl-5,6,7,8-tetrahydro-8-quinolinamine (38 mg, 0.098 mmol), 37% aqueous formaldehyde (40 μL, 0.49 mmol), and NaBH(OAc)₃ (0.10 g, 0.49 mmol) in 6 mL of anhydrous 1,2-dichloroethane was stirred at RT. After 18 hours the mixture was diluted with 10% aqueous Na₂CO₃ and stirred vigorously for 30 minutes. The mixture was diluted with dichloromethane and the phases separated. The organic solution was washed once with saturated aqueous brine, dried over Na₂SO₄ and concentrated to dryness at reduced pressure to afford 28 mg (68%) of N-({1-[trans-4-(dimethylamino)cyclohexyl]-1H-benzimidazol-2-yl}methyl)-N-methyl-5,6,7,8-tetrahydro-8-quinolinamine as a tacky, white foam. ¹H NMR (CDCl₃): δ 8.50 (d, 1H), 7.72 (m, 1H), 7.52 (m, 1H), 7.40 (d, 1H), 7.23-7.14 (m, 2H), 7.10 (m, 1H), 4.71 (m, 1H), 4.27 (d, 1H), 4.01-3.87 (m, 2H), 2.87 (m, 1H), 2.75 (m, 1H), 2.48-2.23 (m, 9H), 2.20 (s, 3H), 2.17-1.88 (m, 6H), 1.74 (m, 1H), 1.57-1.21 (m, 3H). MS m/z 418 (M+1).

### Example 36

### N-Methyl-N-({1-[2-(3-pyridinyl)ethyl]-1H-benzimidazol-2-yl}methyl-5,6,7,8-tetrahydro-8-quinolinamine

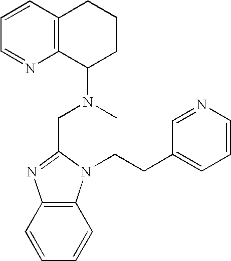

### a) 2-Nitro-N-[2-(3-pyridinyl)ethyl]aniline

A mixture of [2-(3-pyridinyl)ethyl]amine (0.39 g, 3.18 mmol), 1-chloro-2-nitrobenzene (1.00 g, 6.35 mmol), and K₂CO₃ (2.64 g, 19.1 mmol) in 8 mL of anhydrous DMF was heated to 120° C. with stirring. After 18 hours the mixture was cooled to RT and diluted with water. The resulting mixture was extracted with EtOAc (3×). The combined EtOAc extracts were washed with water (2×), saturated aqueous brine (1×), dried over Na₂SO₄ and concentrated to dryness at reduced pressure. The crude product was subjected to flash chromatography (silica gel, gradient elution from 1:1 hexane/EtOAc to EtOAc) to afford 0.61 g (79%) of 2-nitro-N-[2-(3-pyridinyl)ethyl]aniline as a yellow solid. ¹H NMR (DMSO-d₆): δ 8.56 (d, 1H), 8.48 (dd, 1H), 8.16 (m, 1H), 8.10 (d, 1H), 7.76 (m, 1H), 7.57 (m, 1H), 7.38 (m, 1H), 7.19 (d, 1H), 6.72 (t, 1H), 3.66 (m, 2H), 2.99 (t, 2H). MS m/z 244 (M+1).

### b) (2-Aminophenyl)[2-(3-pyridinyl)ethyl]amine

A solution of 2-nitro-N-[2-(3-pyridinyl)ethyl]aniline (0.59 g, 2.4 mmol) in 25 mL of MeOH was subjected to balloon hydrogenation in the presence of 50 mg of 10% Pd on carbon. After 4 hours the reaction vessel was purged with nitrogen, catalyst removed from the solution by filtration through celite, and the filtrate concentrated to dryness at reduced pressure to afford 0.47 g (90%) of (2-aminophenyl)[2-(3-pyridinyl)ethyl]amine as a purple-brown oil. ¹H NMR (DMSO-d₆): δ 8.55 (d, 1H), 8.45 (dd, 1H), 7.78 (m, 1H), 7.36 (m, 1H), 6.63-6.40 (m, 4H), 4.60-4.42 (m, 3H), 3.30 (m, 2H), 2.93 (t, 2H). MS m/z 214 (M+1).

### c) N²-Methyl-N¹-(2-{[2-(3-pyridinyl)ethyl]amino}phenyl)-N²-(5,6,7,8-tetrahydro-8-quinolinyl)glycinamide

Employing the method described herein for the preparation of 1,1-dimethylethyl {trans-4-[(2-{[N-methyl-N-(5,6,7,8-tetrahydro-8-quinolinyl)glycyl]amino}phenyl)amino]cyclohexyl}carbamate, (2-aminophenyl)[2-(3-pyridinyl)ethyl]amine (97 mg, 0.45 mmol) was coupled with N-methyl-N-(5,6,7,8-tetrahydro-8-quinolinyl)glycine (0.10 g, 0.45 mmol) to afford 0.164 g (88%) of N²-methyl-N¹-(2-{[2-(3-pyridinyl)ethyl]amino}phenyl)-N²-(5,6,7,8-tetrahydro-8-quinolinyl)glycinamide as a brown foam. ¹H NMR (DMSO-d₆): δ 10.45 (s, 1H), 8.50-8.30 (m, 3H), 7.68 (d, 1H), 5.59 (d, 1H), 7.44 (d, 1H), 7.39-7.18 (m, 2H), 7.08 (t, 1H), 6.72 (d, 1H), 6.70 (t, 1H), 5.09 (t, 1H), 3.99 (m, 1H), 3.51-3.38 (m, 2H), 3.30-3.12 (m, 2H), 2.99-2.66 (m, 4H), 2.38 (s, 3H), 2.15 (m, 1H), 2.05-1.60 (m, 3H).

### d) N-Methyl-N-({1-[2-(3-pyridinyl)ethyl]-1H-benzimidazol-2-yl}methyl)-5,6,7,8-tetrahydro-8-quinolinamine

Employing the method described herein for the preparation of 1,1-dimethylethyl [trans-4-(2-{[methyl(5,6,7,8-tetrahydro-8-quinolinyl)amino]methyl}-1H-benzimidazol-1-yl)cyclohexyl]carbamate, N²-methyl-N¹-(2-{[2-(3-pyridinyl)ethyl]amino}phenyl)-N²-(5,6,7,8-tetrahydro-8-quinolinyl)glycinamide (0.16 g, 0.39 mmol) was subjected to dehydration in glacial acetic acid to afford, following purification as described in the same example, 80 mg (52%) of N-methyl-N-({1-[2-(3-pyridinyl)ethyl]-1H-benzimidazol-2-yl}methyl)-5,6,7,8-tetrahydro-8-quinolinamine as a viscous, yellow oil. ¹H NMR (CDCl₃): δ 8.44 (d, 1H), 8.39 (d, 1H), 8.30 (s, 1H), 7.71 (m, 1H), 7.39 (d, 1H), 7.31-7.12 (m, 4H), 7.11-7.04 (m, 2H), 4.72 (t, 2H), 3.99 (t, 1H), 3.87 (d, 1H), 3.63 (d, 1H), 3.09 (t, 2H), 2.78 (m, 1H), 2.70 (m, 1H), 2.29 (s, 3H), 2.18-1.90 (m, 3H), 1.72 (m, 1H). MS m/z 398 (M+1).

### Example 37

### N-methyl-N-([1-[2-(2-pyridinyl)ethyl]-1H-benzimidazol-2-yl]methyl)-5,6,7,8-tetrahydro-8-quinolinamine

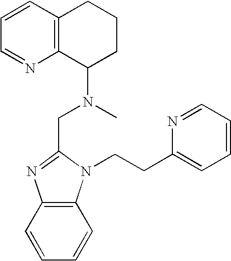

### a) 2-Nitro-N-[2-(2-pyridinyl)ethyl]aniline

Employing the procedure described herein for the preparation of 2-nitro-N-[2-(3-pyridinyl)ethyl]aniline, [2-(2-pyridinyl)ethyl]amine (0.39 g, 3.18 mmol) was reacted with 1-chloro-2-nitrobenzene (1.00 g, 6.35 mmol) to afford, following purification as described in the same example, 0.74 g (96%) of 2-nitro-N-[2-(2-pyridinyl)ethyl]aniline as an orange solid. ¹H NMR (DMSO-d₆): δ 8.58 (d, 1H), 8.38 (m, 1H), 8.09 (d, 1H), 7.79 (t, 1H), 7.58 (t, 1H), 7.39 (d, 1H), 7.28 (m, 1H), 7.13 (d, 1H), 6.71 (t, 1H), 3.75 (q, 2H), 3.14 (t, 2H). MS m/z 244 (M+1).

### b) (2-Aminophenyl)[2-(2-pyridinyl)ethyl]amine

Employing the procedure described herein for the preparation of (2-aminophenyl)[2-(3-pyridinyl)ethyl]amine, 2-nitro-N-[2-(2-pyridinyl)ethyl]anime (0.72 g, 3.0 mmol) was subjected to catalytic hydrogenation to afford 0.59 g (92%) of (2-aminophenyl)[2-(2-pyridinyl)ethyl]amine as a purple-brown solid. ¹H NMR (DMSO-d₆): δ 8.58 (d, 1H), 7.73 (t, 1H), 7.38 (d, 1H), 7.25 (m, 1H), 6.62-6.40 (m, 4H), 4.60 (t, 1H), 4.47 (s, 2H), 3.39 (q, 2H), 3.06 (t, 2H). MS m/z 214 (M+1).

### c) N²-Methyl-N¹-(2-{[2-(2-pyridinyl)ethyl]amino}phenyl)-N²-(5,6,7,8-tetrahydro-8-quinolinyl)glycinamide

Employing the method described herein for the preparation of 1,1-dimethylethyl {trans-4-[(2-{[N-methyl-N-(5,6,7,8-tetrahydro-8-quinolinyl)glycyl]amino}phenyl)amino]cyclohexyl}carbamate, (2-aminophenyl)[2-(2-pyridinyl)ethyl]amine (97 mg, 0.45 mmol) was coupled with N-methyl-N-(5,6,7,8-tetrahydro-8-quinolinyl)glycine (0.10 g, 0.45 mmol) to afford 0.176 g (94%) of N²-methyl-N¹-(2-{[2-(2-pyridinyl)ethyl]amino}phenyl)-N²-(5,6,7,8-tetrahydro-8-quinolinyl)glycinamide as a brown foam. ¹H NMR (DMSO-d₆): δ 10.38 (s, 1H), 8.48 (d, 1H), 8.37 (d, 1H), 7.70 (t, 1H), 7.59 (d, 1H), 7.40 (d, 1H), 7.30-7.16 (m, 3H), 7.08 (t, 1H), 6.82 (d, 1H), 6.67 (t, 1H), 5.18 (t, 1H), 4.00 (m, 1H), 3.59-3.43 (m, 2H), 3.28 (m, 2H), 3.12-2.67 (m, 4H), 2.38 (s, 3H), 2.12 (m, 1H), 2.04-1.60 (m, 3H). MS m/z 438 (M+Na).

### d) N-Methyl-N-({1-[2-(2-pyridinyl)ethyl]-1H-benzimidazol-2-yl}methyl)-5,6,7,8-tetrahydro-8-quinolinamine

Employing the method described herein for the preparation of 1,1-dimethylethyl [trans-4-(2-{[methyl(5,6,7,8-tetrahydro-8-quinolinyl)amino]methyl}-1H-benzimidazol-1-yl)cyclohexyl]carbamate, N²-methyl-N′-(2-{[2-(2-pyridinyl)ethyl]amino}phenyl)-N²-(5,6,7,8-tetrahydro-8-quinolinyl)glycinamide (0.17 g, 0.41 mmol) was subjected to dehydration in glacial acetic acid to afford, following purification as described in the same example, 48 mg (29%) of N-methyl-N-({1-[2-(2-pyridinyl)ethyl]-1H-benzimidazol-2-yl}methyl)-5,6,7,8-tetrahydro-8-quinolinamine as a viscous, yellow oil. ¹H NMR (CDCl₃): δ 8.53 (d, 1H), 8.38 (d, 1H), 7.68 (d, 1H), 7.41 (t, 1H), 7.33 (d, 1H), 7.23-7.07 (m, 4H), 7.02 (m, 1H), 6.80 (d, 1H), 4.86 (m, 1H), 4.76 (m, 1H), 4.05-3.93 (m, 2H), 3.80 (d, 1H), 3.20 (t, 2H), 2.80 (m, 1H), 2.71 (m, 1H), 2.32 (s, 3H), 2.18-1.93 (m, 3H), 1.72 (m, 1H). MS m/z 398 (M+H).

### Example 38

### N-({1-[3-(Dimethylamino)propyl]-1H-imidazol-2-yl}methyl)-N-methyl-5,6,7,8-tetrahydro-8-quinolinamine

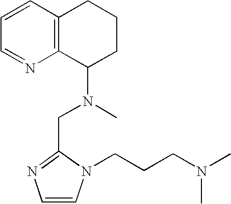

### a) 5,6,7,8-Tetrahydro-8-quinolinamine

A solution of 6,7-dihydro-8(5H)-quinolinone oxime (0.50 g, 3.1 mmol, Synthetic Communications, 2003, 33, 3497-3502) in 35 mL of MeOH was subjected to catalytic hydrogenation at 50 psi in the presence of 50 mg of 10% Pd on carbon. After 18 hours the reaction vessel was purged with nitrogen, catalyst removed by filtration through celite, and the filtrate concentrated to dryness at reduced pressure to afford 0.42 g (92%) of 5,6,7,8-tetrahydro-8-quinolinamine as a purple oil. ¹H NMR (CDCl₃): δ8.39 (d, 1H), 7.36 (d, 1H), 7.04 (m, 1H), 4.00 (t, 1H), 2.88-2.67 (m, 2H), 2.18 (m, 1H), 2.03-1.62 (m, 5H).

### b) N-(1H-Imidazol-2-ylmethyl)-5,6,7,8-tetrahydro-8-quinolinamine

A mixture of 1H-imidazole-2-carbaldehyde (78 mg, 0.81 mmol) in 10 mL of anhydrous MeOH was heated to reflux with stirring. To the hot mixture was added 5,6,7,8-tetrahydro-8-quinolinamine (0.10 g, 0.68 mmol) followed by trimethyl orthoformate (0.34 mL, 2.0 mmol). The solid aldehyde slowly dissolved affording a light yellow solution. After allowing the solution to cool to RT and stirring for an additional 1 hour, NaBH₄ (80 mg, 2.1 mmol) was added. After 30 minutes the solution was mixed with 2 mL of 10% aqueous Na₂CO₃, stirred vigorously of 10 minutes and then concentrated to dryness at by rotary evaporation. The solid residue was subjected to reverse phase HPLC (C8, gradient elution of H₂O/0.1% TFA to MeCN over 40 minutes). Fractions containing pure product (as determined by analytical HPLC) were combined and concentrated to dryness at reduced pressure to afford 0.21 g (56% yield based on tri-TFA salt) of N-(1H-imidazol-2-ylmethyl)-5,6,7,8-tetrahydro-8-quinolinamine as the TFA salt as a transparent, viscous oil. ¹H NMR (CD₃OD): δ 8.79 (d, 1H), 8.21 (d, 1H), 7.78 (dd, 1H), 7.52 (s, 2H), 4.50 (d, 1H), 4.36 (d, 1H), 4.28 (m, 1H), 3.01 (m, 2H), 2.46 (m, 1H), 2.15 (m, 1H), 1.99-1.77 (m, 2H). MS m/z 229 (M+H).

### c) N-(1H-Imidazol-2-ylmethyl)-N-methyl-5,6,7,8-tetrahydro-8-quinolinamine

A mixture of N-(1H-imidazol-2-ylmethyl)-5,6,7,8-tetrahydro-8-quinolinamine TFA salt (0.21 g, 0.37 mmol based on tri-TFA salt), 37% aqueous formalin (0.15 mL, 1.8 mmol), glacial acetic acid (0.35 mL, 3.7 mmol), and NaBH(OAc)₃ (0.62 g, 2.9 mmol) in 10 mL of THF was stirred at RT. After 18 hours 5 mL of 10% aqueous Na₂CO₃ was added and the resulting mixture stirred vigorously for 20 minutes. The mixture was then concentrated to dryness by rotary evaporation. The solid residue was subjected to reverse phase HPLC (C8, gradient elution of H₂O/0.1% TFA to MeCN over 40 minutes). Fractions containing pure product (as determined by analytical HPLC) were combined and concentrated to dryness at reduced pressure to afford 0.16 g (74% based on tri-TFA salt) of N-(1H-imidazol-2-ylmethyl)-N-methyl-5,6,7,8-tetrahydro-8-quinolinamine as the TFA salt as a transparent, viscous oil. ¹H NMR (CD₃OD): δ 8.88 (d, 1H), 8.30 (d, 1H), 7.86 (dd, 1H), 7.55 (s, 2H), 4.46 (dd, 1H), 4.32 (d, 1H), 4.13 (d, 1H), 3.01 (m, 2H), 2.30-2.13 (m, 5H), 2.09-1.81 (m, 2H). MS m/z 243 (M+H).

### d) 3-(2-{[Methyl(5,6,7,8-tetrahydro-8-quinolinyl)amino]methyl}-1H-imidazol-1-yl)propanenitrile

A mixture of N-(1H-imidazol-2-ylmethyl)-N-methyl-5,6,7,8-tetrahydro-8-quinolinamine TFA salt (0.16 g, 0.27 mmol based on tri-TFA salt), 3-bromopropanenitrile (0.11 g, 0.80 mmol), and K₂CO₃ (0.29 g, 2.1 mmol) in 10 mL of anhydrous DMF was heated to 80° C. with stirring. After 3.5 hours the mixture was treated with a second 0.11 g portion of 3-bromopropanenitrile and stirring at 80° C. continued for an additional 3 hours. After cooling to RT the mixture was diluted with EtOAc, washed with water (2×), saturated aqueous brine (2×), dried over Na₂SO₄, and concentrated to dryness at reduced pressure to afford 55 mg (71%) of 3-(2-{[methyl(5,6,7,8-tetrahydro-8-quinolinyl)amino]methyl}-1H-imidazol-1-yl)propanenitrile as a viscous yellow-brown oil. ¹H NMR (CD₃OD): δ 8.46 (d, 1H), 7.54 (d, 1H), 7.20 (m, 1H), 7.16 (s, 1H), 6.88 (s, 1H), 4.50 (m, 1H), 4.34 (m, 1H), 3.98 (t, 1H), 3.88 (d, 1H), 3.72 (d, 1H), 3.12-2.99 (m, 2H), 2.85 (m, 1H), 2.76 (m, 1H), 2.18-1.99 (m, 6H), 1.73 (m, 1H). MS m/z 296 (M+H).

### e) N-{[1-(3-Aminopropyl)-1H-imidazol-2-yl]methyl}-N-methyl-5,6,7,8-tetrahydro-8-quinolinamine

A solution of 3-(2-{[methyl(5,6,7,8-tetrahydro-8-quinolinyl)amino]methyl}-1H-imidazol-1-yl)propanenitrile (50 mg, 0.17 mmol) in 15 mL of 2M ammonia/MeOH was subjected to catalytic hydrogenation at 50 psi in the presence of Raney nickel. After 5 hours the reaction vessel was purged with nitrogen, catalyst removed by filtration through celite, and the filtrate concentrated to dryness at reduced pressure. The crude residue was purified by reverse phase HPLC (C8, gradient elution of H₂O/0.1% TFA to MeCN over 40 minutes). Fractions containing pure product (as determined by analytical HPLC) were combined and concentrated to dryness at reduced pressure to afford 46 mg (42% based on tri-TFA salt) of N-{[1-(3-aminopropyl)-1H-imidazol-2-yl]methyl}-N-methyl-5,6,7,8-tetrahydro-8-quinolinamine as the TFA salt as a transparent, viscous oil. ¹H NMR (CD₃OD): δ 8.93 (d, 1H), 8.37 (d, 1H), 7.90 (dd, 1H), 7.66 (s, 1H), 7.59 (s, 1H), 4.51 (dd, 1H), 4.42-4.33 (m, 3H), 4.24 (d, 1H), 3.13-3.00 (m, 4H), 2.33 (m, 1H), 2.29-2.17 (m, 6H), 2.07 (m, 1H), 1.92 (m, 1H). MS m/z 300 (M+H).

### f) N-({1-[3-(Dimethylamino)propyl]-1H-imidazol-2-yl}methyl)-N-methyl-5,6,7,8-tetrahydro-8-quinolinamine

A mixture of N-{[1-(3-aminopropyl)-1H-imidazol-2-yl]methyl}-N-methyl-5,6,7,8-tetrahydro-8-quinolinamine TFA salt (42 mg, 0.065 mmol based on tri-TFA salt), 37% aqueous formalin (32 μL, 0.39 mmol), glacial acetic acid (61 μL, 0.65 mmol), and NaBH(OAc)₃ (83 mg, 0.39 mmol) in 7 mL of THF was stirred at RT. After 18 hours the solution was diluted with 3 mL of 10% aqueous Na₂CO₃ and the resulting mixture stirred vigorously for 10 minutes. The mixture was then concentrated to dryness by rotary evaporation. The solid residue was purified by reverse phase HPLC (C8, gradient elution of H₂O/0.1% TFA to MeCN over 40 minutes). Fractions containing pure product (as determined by analytical HPLC) were combined and concentrated to dryness at reduced pressure to afford 45 mg (quantitative yield based on tri-TFA salt) of N-({1-[3-(dimethylamino)propyl]-1H-imidazol-2-yl}methyl)-N-methyl-5,6,7,8-tetrahydro-8-quinolinamine as the TFA salt as a viscous, transparent oil. ¹H NMR (CD₃OD): δ 8.92 (d, 1H), 8.33 (d, 1H), 7.88 (dd, 1H), 7.65 (s, 1H), 7.58 (s, 1H), 4.50 (m, 1H), 4.43-4.19 (m, 4H), 3.33-3.20 (m, 2H), 3.08-2.98 (m, 2H), 2.90 (s, 6H), 2.39-2.13 (m, 7H), 2.06 (m, 1H), 1.92 (m, 1H). MS m/z 328 (M+H).

### Example 39

### (8S)—N-Methyl-N-[(1-{[(3S)-1-methyl-3-piperidinyl]methyl}-1H-benzimidazol-2-yl)methyl]-5,6,7,8-tetrahydro-8-quinolinamine

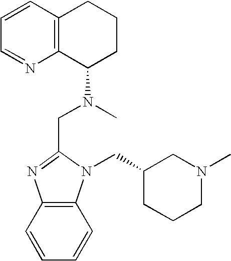

### a) 1,1-Dimethylethyl (3R)-3-{[(2-nitrophenyl)amino]methyl}-1-piperidinecarboxylate

A mixture of 1,1-dimethylethyl (3R)-3-(aminomethyl)-1-piperidinecarboxylate (3.50 g, 16.3 mmol, Ennova MedChem Group, Inc.), 1-fluoro-2-nitrobenzene (3.46 g, 24.5 mmol, Avocado Research Chemicals Ltd.), and K₂CO₃ (11.3 g, 81.5 mmol) in 40 mL of anhydrous acetonitrile was heated to reflux with stirring. After 5 hours the mixture was cooled to RT and filtered through a medium fritted funnel to remove solids. The filter cake was rinsed with an additional 40 mL portion of acetonitrile and the filtrate concentrated to dryness at reduced pressure. The crude oil was subjected to flash chromatography (silica gel, gradient elution of hexane to 6:4 hexane/EtOAc) to afford 1,1-dimethylethyl (3R)-3-{[(2-nitrophenyl)amino]methyl}-1-piperidinecarboxylate as a viscous, yellow oil in quantitative yield. ¹H NMR (DMSO-d₆): δ 8.19 (br s, 1H), 8.05 (d, 1H), 7.52 (t, 1H), 7.06 (d, 1H), 6.68 (t, 1H), 3.93-3.58 (m, 2H), 3.29-3.20 (m, 2H), 2.89-2.48 (m, 2H), 1.85-1.68 (m, 2H), 1.60 (m, 1H), 1.50-1.10 (br s, 11H). MS m/z 358 (M+Na).

### b) 1,1-Dimethylethyl (3R)-3-{[(2-aminophenyl)amino]methyl}-1-piperidinecarboxylate

A solution of 1,1-dimethylethyl (3R)-3-{[(2-nitrophenyl)amino]methyl}-1-piperidinecarboxylate (5.00 g, 14.9 mmol) in 150 mL of EtOH was subjected to balloon hydrogenation in the presence of 0.50 g of 10% Pd on carbon. After 18 hours the reaction vessel was purged with nitrogen, catalyst removed by filtration through celite, and the filtrate concentrated to dryness at reduced pressure to afford 4.38 g (96%) of 1,1-dimethylethyl (3R)-3-{[(2-aminophenyl)amino]methyl}-1-piperidinecarboxylate as a viscous brown oil. ¹H NMR (DMSO-d₆): δ 6.53-6.30 (m, 4H), 4.51-4.29 (m, 3H), 4.05-3.60 (m, 2H), 2.93-2.40 (m, 4H), 1.81 (m, 1H), 1.69 (m, 1H), 1.59 (m, 1H), 1.43-1.05 (m, 11H). MS m/z 306 (M+H).

### c) 1,1-Dimethylethyl (3R)-3-{[2-(chloromethyl)-1H-benzimidazol-1-yl]methyl}-1-piperidinecarboxylate

A solution of 1,1-dimethylethyl (3R)-3-{[(2-aminophenyl)amino]methyl}-1-piperidinecarboxylate (4.20 g, 13.8 mmol), 2-chloro-1,1,1-trimethoxyethane (6.40 g, 41.4 mmol, Aldrich), and p-toluenesulfonic acid (0.26 g, 1.4 mmol) in 70 mL of dichloromethane was stirred at RT. After 18 hours the solution was diluted with 100 mL of dichloromethane, washed twice with saturated aqueous NaHCO₃, dried over Na₂SO₄, and concentrated to dryness at reduced pressure. The crude product was purified by flash chromatography (silica gel, gradient elution of hexane to 6:4 hexane/EtOAc) to afford 4.71 g (94%) of 1,1-dimethylethyl (3R)-3-{[2-(chloromethyl)-1H-benzimidazol-1-yl]methyl}-1-piperidinecarboxylate as a light tan foam. ¹H NMR (DMSO-d₆): δ 7.67-7.58 (m, 2H), 7.28 (t, 1H), 7.23 (t, 1H), 5.06 (s, 2H), 4.28-4.13 (m, 2H), 3.79 (d, 1H), 3.72-3.38 (m, 1H), 2.80-2.58 (m, 2H), 2.05 (m, 1H), 1.72-1.54 (m, 2H), 1.50-0.97 (m, 11H). MS m/z 364 (M+H).

### d) (8S)—N-{(1S)-1-[4-(Methyloxy)phenyl]ethyl}-5,6,7,8-tetrahydro-8-quinolinamine

A solution of (S)-(−)-1-(4-methoxyphenyl)ethylamine (25.0 g, 166 mmol) and 6,7-dihydro-8(5H)-quinolinone (24.0 g, 166 mmol, J. Org. Chem., 2002, 67, 2197-2205) in dichloromethane was treated with glacial acetic acid (14.0 mL, 249 mmol) and sodium triacetoxyborohydride (53.0 g, 249 mmol). The reaction mixture was stirred at room temperature for 15 hours and then treated with sodium carbonate (106 g, 996 mmol) dissolved in water. The resulting mixture was stirred for 30 minutes and then diluted with dichloromethane. The phases were separated and the aqueous solution extracted with an additional portion of dichloromethane. The combined organic solutions were dried over MgSO₄ and concentrated to dryness at reduced pressure. The crude product was purified by flash chromatography (silica gel, gradient elution of dichloromethane to 97:3 dichloromethane/2M ammonia in MeOH) followed by recrystallization from hexane to afford 33 g (70%) of (8S)—N-{(1S)-1-[4-(methyloxy)phenyl]ethyl}-5,6,7,8-tetrahydro-8-quinolinamine as a white crystalline solid. ¹H-NMR (CDCl₃): δ 8.40 (m, 1H), 7.33 (m, 3H), 7.04 (m, 1H), 6.84 (d, 2H), 4.02 (m, 1H), 3.83-3.78 (m, 4H), 2.73-2.62 (m, 2H), 1.82 (m, 1H), 1.72 (m, 1H), 1.57 (m, 2H), 1.43 (d, 3H).

### e) (8S)—N-Methyl-N-{(1S)-1-[4-(methyloxy)phenyl]ethyl}-5,6,7,8-tetrahydro-8-quinolinamine

To a stirred mixture of (8S)—N-{(1S)-1-[4-(methyloxy)phenyl]ethyl}-5,6,7,8-tetrahydro-8-quinolinamine (5.00 g, 17.7 mmol), 37% aqueous formaldehyde (2.90 mL, 35.4 mmol), and glacial acetic acid (1.52 mL, 26.6 mmol) in 50 mL of 1,2-dichloroethane was added NaBH(OAc)₃ (5.64 g, 26.6 mmol). After stirring at RT for 2 hours the mixture was diluted with 50 mL of dichloromethane followed by 80 mL of 10% aqueous Na₂CO₃. The resulting mixture was stirred vigorously for 30 minutes and then the phases separated. The aqueous phase was extracted twice with dichloromethane. The combined organic solutions were washed with saturated aqueous brine, dried over Na₂SO₄, and concentrated to dryness at reduced pressure to afford (8S)—N-methyl-N-{(1S)-1-[4-(methyloxy)phenyl]ethyl}-5,6,7,8-tetrahydro-8-quinolinamine in quantitative yield as a yellow oil. ¹H-NMR (CDCl₃): δ 8.47 (d, 1H), 7.39 (d, 2H), 7.30 (d, 1H), 6.99 (dd, 1H), 6.84 (d, 2H), 4.42 (q, 1H), 3.97 (t, 1H), 3.78 (s, 3H), 2.79 (m, 1H), 2.61 (m, 1H), 2.05-1.78 (m, 6H), 1.57 (m, 1H), 1.37 (d, 3H). MS m/z 297 (M+H).

### f) (8S)—N-Methyl-5,6,7,8-tetrahydro-8-quinolinamine

A solution of (8S)—N-methyl-N-{(1S)-1-[4-(methyloxy)phenyl]ethyl}-5,6,7,8-tetrahydro-8-quinolinamine (5.48 g, 18.5 mmol) in 70 mL of 1:1 trifluoroacetic acid/dichloromethane was stirred at RT for 2.5 hours and then concentrated to dryness by rotary evaporation. The resulting purple syrup was partitioned between 0.5N aqueous HCl and EtOAc. The phases were separated, the aqueous solution washed with EtOAc (3×), and the EtOAc solutions discarded. The aqueous solution was treated with 5N aqueous NaOH to pH=12. The resulting oily mixture was extracted with dichloromethane (5×). The combined dichloromethane extracts were dried over Na₂SO₄ and concentrated to dryness at reduced pressure to afford 2.76 g (92%) of (8S)—N-methyl-5,6,7,8-tetrahydro-8-quinolinamine as a yellow oil. ¹H-NMR (CDCl₃): δ 8.37 (d, 1H), 7.34 (d, 1H), 7.03 (dd, 1H), 3.63 (t, 1H), 2.86-2.60 (m, 3H), 2.52 (s, 3H), 2.10 (m, 1H), 1.96 (m, 1H), 1.82-1.64 (m, 2H).

### g) 1,1-Dimethylethyl (3R)-3-{[2-({methyl[(8S)-5,6,7,8-tetrahydro-8-quinolinyl]amino}methyl)-1H-benzimidazol-1-yl]methyl}-1-piperidinecarboxylate

A solution of (8S)—N-methyl-5,6,7,8-tetrahydro-8-quinolinamine (2.30 g, 14.2 mmol), 1,1-dimethylethyl (3R)-3-{[2-(chloromethyl)-1H-benzimidazol-1-yl]methyl}-1-piperidinecarboxylate (4.70 g, 12.9 mmol), potassium iodide (0.322 g, 1.94 mmol), and N,N-diisopropylethylamine (4.5 mL, 26 mmol) in 80 mL of acetonitrile was heated to reflux with stirring. After 2 hours the solution was cooled to RT, concentrated to approximately 20 mL by rotary evaporation, and diluted with 200 mL of EtOAc. The resulting solution was washed with 10% aqueous Na₂CO₃ (1×), saturated aqueous brine (1×), dried over Na₂SO₄, and concentrated to dryness at reduced pressure. The crude product was purified by flash chromatography (silica gel, gradient elution of MeCN to 9:1 MeCN/NH₄OH) followed by recrystallization from hexane/EtOAc to afford 5.50 g (89%) of 1,1-dimethylethyl (3R)-3-{[2-({methyl[(8S)-5,6,7,8-tetrahydro-8-quinolinyl]amino}methyl)-1H-benzimidazol-1-yl]methyl}-1-piperidinecarboxylate as an off-white crystalline solid. ¹H NMR (DMSO-d₆): δ 8.44 (d, 1H), 7.59-7.47 (m, 3H), 7.23-7.07 (m, 3H), 4.36-4.12 (m, 3H), 3.99 (d, 1H), 3.92 (m, 1H), 3.78 (d, 1H), 3.68-3.35 (m, 1H), 2.86-2.40 (m, 5H), 2.13-1.86 (m, 6H), 1.75-1.49 (m, 3H), 1.43-0.90 (m, 11H). MS m/z 490 (M+H).

### h) (8S)—N-Methyl-N-({1-[(3S)-3-piperidinylmethyl]-1H-benzimidazol-2-yl}methyl)-5,6,7,8-tetrahydro-8-quinolinamine

A solution of 1,1-dimethylethyl (3R)-3-{[2-({methyl[(8S)-5,6,7,8-tetrahydro-8-quinolinyl]amino}methyl)-1H-benzimidazol-1-yl]methyl}-1-piperidinecarboxylate (0.50 g, 1.0 mmol) in 20 mL of 1:1 trifluoroacetic acid/dichloromethane was stirred at RT for 2 hours and concentrated to dryness at reduced pressure. The residue was dissolved in dichloromethane. The solution was washed with 10% aqueous Na₂CO₃ (2×), saturated aqueous brine (1×), dried over Na₂SO₄ and concentrated to dryness at reduced pressure to afford (8S)—N-methyl-N-({1-[(3S)-3-piperidinylmethyl]-1H-benzimidazol-2-yl}methyl)-5,6,7,8-tetrahydro-8-quinolinamine in quantitative yield. ¹H NMR (CD₃OD): δ 8.42 (d, 1H), 7.60-7.49 (m, 2H), 7.46 (d, 1H), 7.29-7.11 (m, 3H), 4.32-4.13 (m, 2H), 4.08 (d, 1H), 3.96-3.78 (m, 2H), 2.98-2.82 (m, 2H), 2.79 (m, 1H), 2.63 (d, 1H), 2.45 (m, 1H), 2.30-1.92 (m, 8H), 1.81-1.50 (m, 3H), 1.37 (m, 1H), 1.04 (m, 1H). MS m/z 390 (M+H).

### i) (8S)—N-Methyl-N-[(1-{[(3S)-1-methyl-3-piperidinyl]methyl}-1H-benzimidazol-2-yl)methyl]-5,6,7,8-tetrahydro-8-quinolinamine

A mixture of (8S)—N-methyl-N-({1-[(3S)-3-piperidinylmethyl]-1H-benzimidazol-2-yl}methyl)-5,6,7,8-tetrahydro-8-quinolinamine (0.14 g, 0.36 mmol), 37% aqueous formaldehyde (54 μL, 0.72 mmol), glacial acetic acid (31 μL, 0.54 mmol), and NaBH(OAc)₃ (0.11 g, 0.54 mmol) in 10 mL of 1,2-dichloroethane was stirred at RT. After 1.5 hours the mixture was concentrated to dryness at reduced pressure and the residue partitioned between dichloromethane and 10% aqueous Na₂CO₃. The phases were separated and the aqueous solution extracted with an additional portion of dichloromethane. The combined dichloromethane solutions were washed with saturated aqueous brine (1×), dried over Na₂SO₄, and concentrated to dryness at reduced pressure. The crude residue was purified by flash chromatography (silica gel, gradient elution of MeCN to 9:1 MeCN/NH₄OH) to afford 0.114 g (79%) of (8S)—N-methyl-N-[(1-{[(3S)-1-methyl-3-piperidinyl]methyl}-1H-benzimidazol-2-yl)methyl]-5,6,7,8-tetrahydro-8-quinolinamine as a tacky, white foam. ¹H NMR (CD₃OD): δ 8.43 (d, 1H), 7.59-7.52 (m, 2H), 7.46 (d, 1H), 7.29-7.17 (m, 3H), 4.34-4.18 (m, 2H), 4.07 (d, 1H), 3.96 (m, 2H), 2.92 (m, 1H), 2.83-2.67 (m, 2H), 2.42 (d, 1H), 2.30-1.99 (m, 10H), 1.92 (t, 1H), 1.82-1.36 (m, 5H), 0.92 (m, 1H). MS m/z 404 (M+H).

### Example 40

### (8S)—N-Methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine

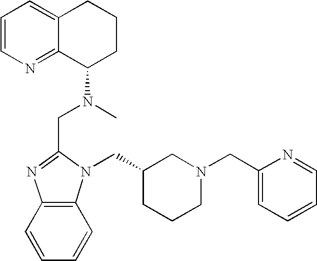

A solution of (8S)—N-methyl-N-({1-[(3S)-3-piperidinylmethyl]-1H-benzimidazol-2-yl}methyl)-5,6,7,8-tetrahydro-8-quinolinamine (50 mg, 0.13 mmol), 2-pyridinecarbaldehyde (18 μL, 0.19 mmol), and glacial acetic acid (15 μL, 0.26 mmol) in 4 mL of 1,2-dichloroethane was stirred at RT for 15 minutes then treated with NaBH(OAc)₃ (41 mg, 0.19 mmol). After stirring at RT for 2 hours the solution was diluted with 6 mL of dichloromethane followed by 10 mL of 10% aqueous Na₂CO₃. The resulting biphasic mixture was stirred vigorously for 20 minutes and then the phases allowed to separate. The organic solution was dried by passing through a hydrophobic separator tube (Alltech Associates, Deerfield, Ill., 60015). The filtrate was concentrated to dryness at reduced pressure. The crude product was purified by reverse phase HPLC (C8, gradient elution of H₂O/0.1% TFA to 1:1 H₂O/0.1% TFA:MeCN over 40 minutes). Fractions containing pure product (as determined by analytical HPLG) were combined and concentrated to a volume of approximately 20 mL by rotary evaporation. To this solution was added 50 mL of 10% aqueous Na₂CO₃. The resulting cloudy suspension was extracted with dichloromethane (3×). The combined organic extracts were washed with saturated aqueous brine (1×), dried over Na₂SO₄, and concentrated to dryness at reduced pressure to afford 35 mg (56%) of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine as a white foam. ¹H NMR (CD₃OD): δ 8.38 (m, 2H), 7.69 (t, 1H), 7.58-7.37 (m, 3H), 7.33 (d, 1H), 7.27-7.11 (m, 4H), 4.32-4.10 (m, 2H), 4.03 (d, 1H), 3.92-3.77 (m, 2H), 3.58-3.41 (m, 2H), 2.89 (m, 1H), 2.82-2.62 (m, 2H), 2.42 (d, 1H), 2.27-1.90 (m, 8H), 1.79-1.38 (m, 5H), 0.90 (m, 1H). MS m/z 481 (M+H).

### Example 41

### (8S)—N-Methyl-N-[(1-{[(3S)-1-(3-pyridinylmethyl)-3-piperidinyl]methyl}-1H-benzimidazol-2-yl)methyl]-5,6,7,8-tetrahydro-8-quinolinamine

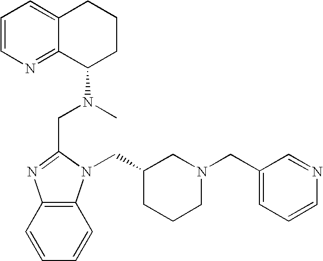

Employing the method described herein for the preparation of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (50 mg, 0.13 mmol) was subjected to reductive alkylation with 2-pyridinecarbaldehyde to afford, after work-up and purification as described in the same example, 43 mg (69%) of (8S)—N-methyl-N-[(1-{[(3S)-1-(3-pyridinylmethyl)-3-piperidinyl]methyl}-1H-benzimidazol-2-yl)methyl]-5,6,7,8-tetrahydro-8-quinolinamine as a white foam. ¹H NMR (CD₃OD): δ 8.43-8.28 (m, 3H), 7.64 (d, 1H), 7.59-7.37 (m, 3H), 7.35-7.09 (m, 4H), 4.32-4.16 (m, 2H), 4.04 (d, 1H), 3.91-3.76 (m, 2H), 3.43 (q, 2H), 2.89 (m, 1H), 2.82-2.61 (m, 2H), 2.42 (d, 1H), 2.29-1.87 (m, 8H), 1.79-1.36 (m, 5H), 0.90 (m, 1H). MS m/z 481 (M+H).

### Example 42

### (8S)—N-Methyl-N-[(1-{[(3S)-1-(4-pyridinylmethyl)-3-piperidinyl]methyl}-1H-benzimidazol-2-yl)methyl]-5,6,7,8-tetrahydro-8-quinolinamine

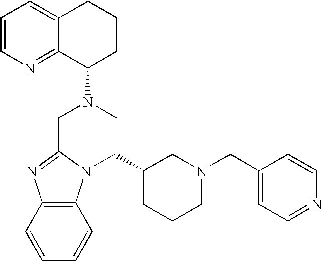

Employing the method described herein for the preparation of (8S)—N-methyl-N-[(1-{[(3S)-1-(2-pyridinylmethyl-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (61 mg, 0.16 mmol) was subjected to reductive alkylation with 4-pyridinecarbaldehyde to afford, after work-up and purification as described in the same example, 7.6 mg (10%) of (8S)—N-methyl-N-[(1-{[(3S)-1-(4-pyridinylmethyl)-3-piperidinyl]methyl}-1H-benzimidazol-2-yl)methyl]-5,6,7,8-tetrahydro-8-quinolinamine as a white foam. ¹H NMR (CD₃OD): δ 8.42-8.30 (m, 3H), 7.58-7.37 (m, 3H), 7.30-7.10 (m, 5H), 4.33-4.15 (m, 2H), 4.05 (d, 1H), 3.93-3.79 (m, 2H), 3.43 (q, 2H), 2.89 (m, 1H), 2.81-2.60 (m, 2H), 2.40 (d, 1H), 2.27-1.90 (m, 8H), 1.79-1.37 (m, 5H), 0.92 (m, 1H). MS m/z 481 (M+H).

### Example 43

### (8S)—N-Methyl-N-[(1-{[(3S)-1-(phenylmethyl)-3-piperidinyl]methyl}-1H-benzimidazol-2-yl)methyl]-5,6,7,8-tetrahydro-8-quinolinamine

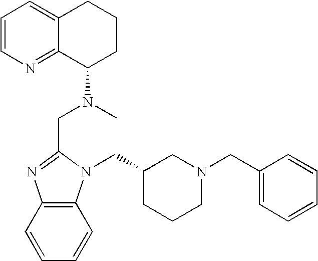

Employing the method described herein for the preparation of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (61 mg, 0.16 mmol) was subjected to reductive alkylation with benzaldehyde to afford, after work-up and purification as described in the same example, 47 mg (62%) of (8S)—N-methyl-N-[(1-{[(3S)-1-(phenylmethyl)-3-piperidinyl]methyl}-1H-benzimidazol-2-yl)methyl]-5,6,7,8-tetrahydro-8-quinolinamine as a white foam. ¹H NMR (CD₃OD): δ 8.38 (d, 1H), 7.53 (d, 1H), 7.48-7.36 (m, 2H), 7.29-7.08 (m, 8H), 4.30-4.12 (m, 2H), 4.01 (d, 1H), 3.90-3.71 (m, 2H), 3.45 (d, 1H), 3.32 (d, 1H), 2.89 (m, 1H), 2.82-2.67 (m, 2H), 2.43 (d, 1H), 2.28-1.87 (m, 8H), 1.79-1.35 (m, 5H), 0.83 (m, 1H). MS m/z 480 (M+H).

### Example 44

### (8S)—N-Methyl-N-[(1-{[(3S)-1-(2-methylpropyl)-3-piperidinyl]methyl}-1H-benzimidazol-2-yl)methyl]-5,6,7,8-tetrahydro-8-quinolinamine

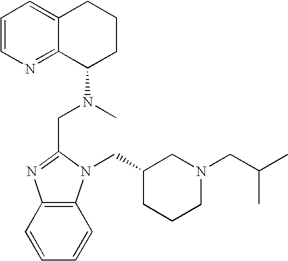

Employing the method described herein for the preparation of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (61 mg, 0.16 mmol) was subjected to reductive alkylation with isobutyraldehyde to afford, after work-up and purification as described in the same example, 49 mg (69%) of (8S)—N-methyl-N-[(1-{[(3S)-1-(2-methylpropyl)-3-piperidinyl]methyl}-1H-benzimidazol-2-yl)methyl]-5,6,7,8-tetrahydro-8-quinolinamine as a transparent, viscous oil. ¹H NMR (CD₃OD): δ 8.43 (d, 1H), 7.63-7.50 (m, 2H), 7.45 (d, 1H), 7.31-7.12 (m, 3H), 4.33-4.14 (m, 2H), 4.07 (d, 1H), 3.97-3.80 (m, 2H), 2.91 (m, 1H), 2.87-2.65 (m, 2H), 2.43 (d, 1H), 2.32-1.37 (m, 16H), 0.97-0.70 (m, 7H). MS m/z 446 (M+H).

### Example 45

### (8S)—N-[(1-{[(3S)-1-(1H-Imidazol-2-ylmethyl)-3-piperidinyl]methyl}-1H-benzimidazol-2-yl)methyl]-N-methyl-5,6,7,8-tetrahydro-8-quinolinamine

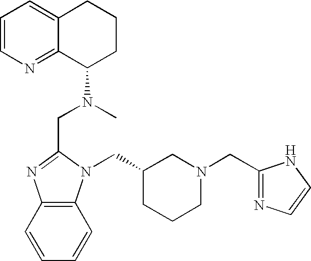

A mixture of (8S)—N-methyl-N-({1-[(3S)-3-piperidinylmethyl]-1H-benzimidazol-2-yl}methyl)-5,6,7,8-tetrahydro-8-quinolinamine (50 mg, 0.13 mmol), 1H-imidazole-2-carbaldehyde (18 mg, 0.19 mmol), and trimethyl orthoformate (42 μL, 0.38 mmol) in 5 mL of anhydrous MeOH was heated to reflux with stirring for 25 minutes and then allowed to cool to RT. To the resulting solution was added NaBH₄ (30 mg, 0.79 mmol). After stirring at RT for 2 hours, the solution was concentrated to dryness at reduced pressure. The residue was partitioned between dichloromethane and 10% aqueous Na₂CO₃. The phases were separated and the aqueous solution extracted with an additional portion of dichloromethane. The combined dichloromethane solutions were dried by passing through a hydrophobic separator tube (Alltech Associates, Deerfield, Ill., 60015) and the filtrate concentrated to dryness at reduced pressure. The crude product was subjected to HPLC purification followed by free-basing of the TFA salt as described herein for the preparation of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine to afford 19 mg (32%) of (8S)—N-[(1-{[(3S)-1-(1H-imidazol-2-ylmethyl)-3-piperidinyl]methyl}-1H-benzimidazol-2-yl)methyl]-N-methyl-5,6,7,8-tetrahydro-8-quinolinamine as a white foam. ¹H NMR (CD₃OD): δ 8.38 (d, 1H), 7.58 (d, 1H), 7.50 (d, 1H), 7.44 (d, 1H), 7.30-7.13 (m, 3H), 6.94 (s, 2H), 4.29 (dd, 1H), 4.17 (dd, 1H), 4.05 (d, 1H), 3.90-3.80 (m, 2H), 3.50 (s, 2H), 2.92 (m, 1H), 2.83-2.65 (m, 2H), 2.49 (d, 1H), 2.29-1.92 (m, 8H), 1.82-1.37 (m, 5H), 0.79 (m, 1H). MS m/z 470 (M+H).

### Example 46

### 2-((3S)-3-{[2-({Methyl[(8S)-5,6,7,8-tetrahydro-8-quinolinyl]amino}methyl)-1H-benzimidazol-1-yl]methyl}-1-piperidinyl)ethanol

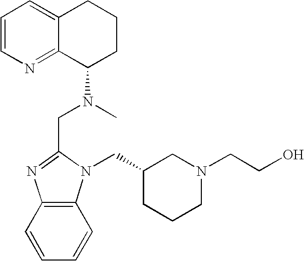

A solution of (8S)—N-methyl-N-({1-[(3S)-3-piperidinylmethyl]-1H-benzimidazol-2-yl}methyl)-5,6,7,8-tetrahydro-8-quinolinamine (50 mg, 0.13 mmol), {[(tert-butyl)(dimethyl)silyl]oxy}acetaldehyde (36 μL, 0.19 mmol), and glacial acetic acid (15 μL, 0.26 mmol) in 4 mL of 1,2-dichloroethane was stirred at RT for 15 minutes and then treated with NaBH(OAc)₃ (41 mg, 0.19 mmol). After stirring at RT for 2 hours, the cloudy solution was diluted with 6 mL of dichloromethane followed by 10 mL of 10% aqueous Na₂CO₃. The mixture was stirred vigorously for 20 minutes and then the phases allowed to separate. The organic solution was dried by passing through a hydrophobic separator tube (Alltech Associates, Deerfield, Ill., 60015) and the filtrate concentrated to dryness at reduced pressure. The residue was dissolved in 5 mL of anhydrous THF and the solution treated with of 1 M tetrabutylammonium fluoride in THF (0.25 mL, 0.25 mmol). After stirring at RT for 2 hours the solution was concentrated to dryness by rotary evaporation and the resulting residue dissolved in dichloromethane. The solution was washed with 10% aqueous Na₂CO₃, dried by passage through a hydrophobic separator tube, and concentrated to dryness at reduced pressure. The crude product was purified by reverse phase HPLC (C8, gradient elution of H₂O/0.1% TFA to MeCN over 40 minutes). Fractions containing pure product (as determined by analytical HPLC) were combined and concentrated to a volume of approximately 20 mL by rotary evaporation. The solution was treated with excess 10% aqueous Na₂CO₃ and the resulting mixture extracted with dichloromethane (3×). The combined organic extracts were washed once with saturated aqueous brine, dried over Na₂SO₄ and concentrated to dryness at reduced pressure to afford 31 mg (55%) of 2-((3S)-3-{[2-({methyl[(8S)-5,6,7,8-tetrahydro-8-quinolinyl]amino}methyl)-1H-benzimidazol-1-yl]methyl}-1-piperidinyl)ethanol as a white foam. ¹H NMR (CD₃OD): δ 8.42 (d, 1H), 7.60-7.50 (m, 2H), 7.44 (d, 1H), 7.29-7.13 (m, 3H), 4.34-4.13 (m, 2H), 4.06 (d, 1H), 3.95-3.80 (m, 2H), 3.52 (t, 2H), 2.99-2.70 (m, 3H), 2.55 (d, 1H), 2.46-2.29 (m, 2H), 2.27-1.89 (m, 8H), 1.82-1.38 (m, 5H), 0.87 (m, 1H). MS m/z 434 (M+H).

### Example 47

### 3-((3S)-3-{[2-({Methyl[(8S)-5,6,7,8-tetrahydro-8-quinolinyl]amino}methyl)-1H-benzimidazol-1-yl]methyl}1-piperidinyl)-1-propanol

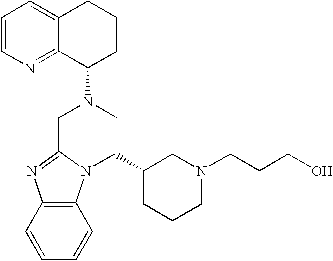

To a stirred solution of 3-{[(tert-butyl)(dimethyl)silyl]oxy}-1-propanol (0.200 g, 1.05 mmol) in 20 mL of dichloromethane was added IBX polystyrene resin (2.25 g, 3.15 mmol @ 1.40 mmol/g, Novabiochem). After gently stirring the mixture at RT for 18 hours, the resin was removed by filtration through a medium fritted funnel. The resin was rinsed with 3 additional portions of dichloromethane and the filtrate concentrated to approximately 5 mL by rotary evaporation. The solution was diluted with 12 mL of 1,2-dichloroethane and treated with (8S)—N-methyl-N-({1-[(3S)-3-piperidinylmethyl]-1H-benzimidazol-2-yl}methyl)-5,6,7,8-tetrahydro-8-quinolinamine (0.100 g, 0.257 mmol), glacial acetic acid (88 μL, 1.5 mmol), and NaBH(OAc)₃ (0.273 g, 1.29 mmol). The cloudy solution was stirred at RT for 3 hours and then diluted with dichloromethane followed by 10% aqueous Na₂CO₃. The resulting mixture was stirred vigorously for 30 minutes and the phases allowed to separate. The organic solution was dried by passing through a hydrophobic separator tube (Alltech Associates, Deerfield, Ill., 60015), and then concentrated to dryness at reduced pressure. The residue was dissolved in 5 mL of anhydrous THF and the solution treated with 1 M tetrabutylammonium fluoride in THF (0.50 mL, 0.50 mmol). After stirring at RT for 2 hours the solution was concentrated to dryness and the residue dissolved in dichloromethane. The solution was washed with 10% aqueous Na₂CO₃ (1×), saturated aqueous brine (1×), dried over Na₂SO₄, and concentrated to dryness at reduced pressure. The crude product was purified by flash chromatography (silica gel, gradient elution of MeCN to 85:15 MeCN/NH₄OH) to afford 56 mg (49%) of 3-((3S)-3-{[2-({methyl[(8S)-5,6,7,8-tetrahydro-8-quinolinyl]amino}methyl)-1H-benzimidazol-1-yl]methyl}-1-piperidinyl)-1-propanol as a white foam. ¹H NMR (CD₃OD): δ 8.45 (d, 1H), 7.61-7.52 (m, 2H), 7.47 (d, 1H), 7.30-7.17 (m, 3H), 4.36-4.18 (m, 2H), 4.09 (d, 1H), 3.96-3.83 (m, 2H), 3.51 (t, 2H), 2.99-2.72 (m, 3H), 2.52 (d, 1H), 2.39-2.31 (m, 2H), 2.29-2.19 (m, 4H), 2.18-1.98 (m, 3H), 1.91 (t, 1H), 1.77 (m, 1H), 1.72-1.36 (m, 6H), 0.91 (m, 1H). MS m/z 448 (M+H).

### Example 48

### N-{[1-({3-[(Dimethylamino)methyl]phenyl}methyl)-1H-benzimidazol-2-yl]methyl}-N-methyl-5,6,7,8-tetrahydro-8-quinolinamine

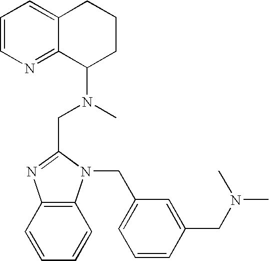

### a) 3-[(2-{[Methyl(5,6,7,8-tetrahydro-8-quinolinyl)amino]methyl}-1H-benzimidazol-1-yl)methyl]benzonitrile

Reaction of N-(1H-benzimidazol-2-ylmethyl)-N-methyl-5,6,7,8-tetrahydro-8-quinolinamine (207 mg, 0.71 mmol) and 3-cyanobenzyl bromide (208 mg, 1.06 mmol) as described herein for the preparation of N-methyl-N-{[1-(3-pyridinylmethyl)-1H-benzimidazol-2-yl]methyl}-5,6,7,8-tetrahydro-8-quinolinamine, afforded 202 mg (70%) of 3-[(2-{[methyl(5,6,7,8-tetrahydro-8-quinolinyl)amino]methyl}-1H-benzimidazol-1-yl)methyl]benzonitrile as a brown oil. ¹H NMR (DMSO-d₆): δ 8.27 (d, 1H), 7.70 (m, 1H), 7.58 (m, 2H), 7.49-7.37 (m, 3H), 7.29 (m, 1H), 7.11 (m, 3H), 5.74 (m, 2H), 4.21-3.92 (m, 4H), 2.64 (m, 2H), 2.09 (s, 3H), 1.82 (m, 3H). MS m/z 408 (M+1).

### b) N-[(1-{[3-(Aminomethyl)phenyl]methyl}-1H-benzimidazol-2-yl)methyl]-N-methyl-5,6,7,8-tetrahydro-8-quinolinamine

Reduction of 3-[(2-{[methyl(5,6,7,8-tetrahydro-8-quinolinyl)amino]methyl}-1H-benzimidazol-1-yl)methyl]benzonitrile (198 mg, 0.49 mmol) as herein described for the preparation of N-[(1-{[4-(aminomethyl)phenyl]methyl}-1H-benzimidazol-2-yl)methyl]-N-methyl-5,6,7,8-tetrahydro-8-quinolinamine, afforded 95 mg (48%) of N-[(1-{[3-(aminomethyl)phenyl]methyl}-1H-benzimidazol-2-yl)methyl]-N-methyl-5,6,7,8-tetrahydro-8-quinolinamine as a sticky white foam, after flash chromatography (silica gel, gradient elution of acetonitrile to 9:1 acetonitrile/NH₄OH), followed by reverse phase HPLC purification (C8, 0 to 70% acetonitrile in H₂O/0.1% TFA). ¹H NMR (DMSO-d₆): δ 8.31 (d, 1H), 7.55 (m, 1H), 7.43 (m, 1H), 7.31 (m, 1H), 7.18-7.10 (m, 6H), 6.83 (m, 1H), 5.64 (m, 2H), 4.18-4.01 (m, 2H), 3.91 (t, 1H), 3.60 (s, 2H), 2.65 (m, 2H), 2.11 (s, 3H), 1.88 (m, 3H), 1.57 (m, 1H). MS m/z 412 (M+1).

### c) N-{[1-({3-[(Dimethylamino)methyl]phenyl}methyl)-1H-benzimidazol-2-yl]methyl}-N-methyl-5,6,7,8-tetrahydro-8-quinolinamine

Reductive methylation of N-[(1-{[3-(aminomethyl)phenyl]methyl}-1H-benzimidazol-2-yl)methyl]-N-methyl-5,6,7,8-tetrahydro-8-quinolinamine (37 mg, 0.09 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 9 mg (23%) of N-{[1-({3-[(dimethylamino)methyl]phenyl}methyl)-1H-benzimidazol-2-yl]methyl}-N-methyl-5,6,7,8-tetrahydro-8-quinolinamine as a yellow oil. ¹H NMR (DMSO-d₆): δ 8.29 (m, 1H), 7.55 (m, 1H), 7.42 (m, 1H), 7.30 (m, 1H), 7.20-7.03 (m, 6H), 6.89 (m, 1H), 5.65 (m, 2H), 4.15-3.89 (m, 3H), 2.64 (m, 2H), 2.10 (s, 3H), 2.02 (s, 6H), 1.85 (m, 4H), 1.57 (m, 2H). MS m/z 440 (M+1).

### Example 49

### N-({1-[6-(Dimethylamino)hexyl]-1H-benzimidazol-2-yl}methyl)-N-methyl-5,6,7,8-tetrahydro-8-quinolinamine

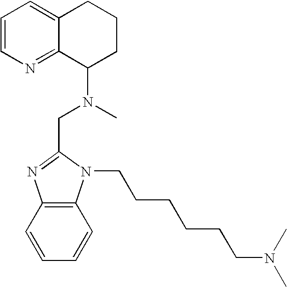

### a) 6-(2-{[Methyl(5,6,7,8-tetrahydro-8-quinolinyl)amino]methyl}-1H-benzimidazol-1-yl)hexanenitrile

Reaction of N-(1H-benzimidazol-2-ylmethyl)-N-methyl-5,6,7,8-tetrahydro-8-quinolinamine (203 mg, 0.69 mmol) and 6-bromohexanenitrile (138 μL, 1.04 mmol) as described herein for the preparation of N-methyl-N-{[1-(3-pyridinylmethyl)-1H-benzimidazol-2-yl]methyl}-5,6,7,8-tetrahydro-8-quinolinamine, afforded 103 mg (38%) of 6-(2-{[methyl(5,6,7,8-tetrahydro-8-quinolinyl)amino]methyl}-1H-benzimidazol-1-yl)hexanenitrile as a brown oil. ¹H NMR (DMSO-d₆): δ 8.43 (d, 1H), 7.50 (m, 3H), 7.14 (m, 3H), 4.32 (m, 2H), 4.21-3.99 (m, 2H), 3.94 (m, 1H), 2.78-2.64 (m, 2H), 2.46 (m, 2H), 2.07 (s, 3H), 1.95 (m, 3H), 1.73-1.50 (m, 5H), 1.34 (m, 2H). MS m/z 388 (M+1).

### b) N-{[1-(6-Aminohexyl)-1H-benzimidazol-2-yl]methyl}-N-methyl-5,6,7,8-tetrahydro-8-quinolinamine

Reduction of 6-(2-{[methyl(5,6,7,8-tetrahydro-8-quinolinyl)amino]methyl}-1H-benzimidazol-1-yl)hexanenitrile (103 mg, 0.27 mmol) as herein described for the preparation of N-[(1-{[4-(aminomethyl)phenyl]methyl}-1H-benzimidazol-2-yl)methyl]-N-methyl-5,6,7,8-tetrahydro-8-quinolinamine, afforded 77 mg (74%) of N-{[1-(6-aminohexyl)-1H-benzimidazol-2-yl]methyl}-N-methyl-5,6,7,8-tetrahydro-8-quinolinamine as a brown oil, after flash chromatography (silica gel, gradient elution of acetonitrile to 9:1 acetonitrile/NH₄OH). ¹H NMR (DMSO-d₆): δ 8.42 (d, 1H), 7.49 (m, 3H), 7.14 (m, 3H), 4.28-3.29 (m, 5H), 2.78-2.66 (m, 2H), 2.07 (s, 3H), 1.94 (m, 3H), 1.65 (m, 3H), 1.41 (m, 1H), 1.25 (m, 7H). MS m/z 392 (M+1).

### c) N-({1-[6-(Dimethylamino)hexyl]-1H-benzimidazol-2-yl}methyl)-N-methyl-5,6,7,8-tetrahydro-8-quinolinamine

Reductive methylation of N-{[1-(6-aminohexyl)-1H-benzimidazol-2-yl]methyl}-N-methyl-5,6,7,8-tetrahydro-8-quinolinamine (55 mg, 0.14 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 27 mg (46%) of N-({1-[6-(dimethylamino)hexyl]-1H-benzimidazol-2-yl}methyl)-N-methyl-5,6,7,8-tetrahydro-8-quinolinamine as a yellow oil, after reverse phase HPLC purification (C8, 0 to 70% acetonitrile in H₂O/0.1% TFA). ¹H NMR (DMSO-d₆): δ 8.42 (d, 1H), 7.49 (m, 3H), 7.13 (m, 3H), 4.30-3.91 (m, 5H), 2.82-2.66 (m, 2H), 2.09-2.03 (m, 11H), 1.94 (m, 3H), 1.65 (m, 3H), 1.31-1.22 (m, 6H). MS m/z 420 (M+1).

### Example 50

### N-{[1-({2-[(Dimethylamino)methyl]phenyl}methyl)-1H-benzimidazol-2-yl]methyl}-N-methyl-5,6,7,8-tetrahydro-8-quinolinamine

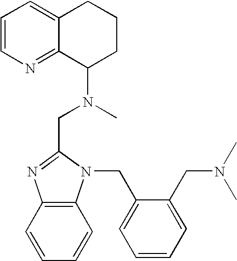

### a) 2-[(2-{[Methyl(5,6,7,8-tetrahydro-8-quinolinyl)amino]methyl}-1H-benzimidazol-1-yl)methyl]benzonitrile

Reaction of N-(1H-benzimidazol-2-ylmethyl)-N-methyl-5,6,7,8-tetrahydro-8-quinolinamine (240 mg, 0.82 mmol) and 2-cyanobenzyl bromide (241 mg, 1.23 mmol) as described herein for the preparation of N-methyl-N-{[1-(3-pyridinylmethyl)-1H-benzimidazol-2-yl]methyl}-5,6,7,8-tetrahydro-8-quinolinamine, afforded 182 mg (54%) of 2-[(2-{[methyl(5,6,7,8-tetrahydro-8-quinolinyl)amino]methyl}-1H-benzimidazol-1-yl)methyl]benzonitrile as a brown oil. ¹H NMR (DMSO-d₆): δ 8.22 (d, 1H), 7.89 (m, 1H), 7.61 (m, 1H), 7.50-7.40 (m, 3H), 7.25-7.04 (m, 4H), 6.53 (m, 1H), 5.96 (m, 2H), 4.18-3.83 (m, 3H), 2.63 (m, 2H), 2.06 (s, 3H), 1.79 (m, 3H), 1.54 (m, 1H). MS m/z 408 (M+1).

### b) N-[(1-{[2-(Aminomethyl)phenyl]methyl}-1H-benzimidazol-2-yl)methyl]-N-methyl-5,6,7,8-tetrahydro-8-quinolinamine

Reduction of 2-[(2-{[methyl(5,6,7,8-tetrahydro-8-quinolinyl)amino]methyl}-1H-benzimidazol-1-yl)methyl]benzonitrile (180 mg, 0.44 mmol) as herein described for the preparation of N-[(1-{[4-(aminomethyl)phenyl]methyl}-1H-benzimidazol-2-yl)methyl]-N-methyl-5,6,7,8-tetrahydro-8-quinolinamine, afforded 89 mg (49%) of N-[(1-{[2-(aminomethyl)phenyl]methyl}-1H-benzimidazol-2-yl)methyl]-N-methyl-5,6,7,8-tetrahydro-8-quinolinamine as a sticky white foam, after flash chromatography (silica gel, gradient elution of acetonitrile to 9:1 acetonitrile/NH₄OH), followed by reverse phase HPLC purification (C8, 0 to 70% acetonitrile in H₂O/0.1% TFA). ¹H NMR (DMSO-d₆): δ 8.27 (d, 1H), 7.59 (m, 1H), 7.40 (m, 2H), 7.22-7.05 (m, 5H), 6.96 (t, 1H), 6.10 (d, 1H), 5.80 (q, 2H), 4.14-3.81 (m, 5H), 2.60 (m, 2H), 2.06 (s, 3H), 1.79 (m, 3H), 1.52 (m, 1H). MS m/z 412 (M+1).

### c) N-{[1-({2-[(Dimethylamino)methyl]phenyl}methyl)-1H-benzimidazol-2-yl]methyl}-N-methyl-5,6,7,8-tetrahydro-8-quinolinamine

Reductive methylation of N-[(1-{[2-(aminomethyl)phenyl]methyl}-1H-benzimidazol-2-yl)methyl]-N-methyl-5,6,7,8-tetrahydro-8-quinolinamine (62 mg, 0.15 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 35 mg (53%) of N-{[1-({2-[(dimethylamino)methyl]phenyl}methyl)-1H-benzimidazol-2-yl]methyl}-N-methyl-5,6,7,8-tetrahydro-8-quinolinamine as a white foam, after reverse phase HPLC purification (C8, 0 to 70% acetonitrile in H₂O/0.1% TFA). ¹H NMR (DMSO-d₆): δ 8.29 (d, 1H), 7.59 (m, 1H), 7.40 (m, 1H), 7.25 (m, 1H), 7.16-6.99 (m, 6H), 6.14 (d, 1H), 5.82 (m, 2H), 4.16-3.97 (m, 2H), 3.83 (m, 1H), 3.51 (q, 2H), 2.62 (m, 2H), 2.19 (s, 6H), 2.07 (s, 3H), 1.79 (m, 3H), 1.52 (m, 1H). MS m/z 440 (M+1).

### Example 51

### N-[4-(2-{[Methyl(5,6,7,8-tetrahydro-8-quinolinyl)amino]methyl}-1H-benzimidazol-1-yl)butyl]methanesulfonamide

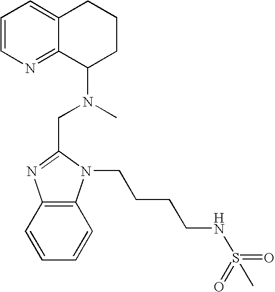

To a solution of N-{[1-(4-aminobutyl)-1H-benzimidazol-2-yl]methyl}-N-methyl-5,6,7,8-tetrahydro-8-quinolinamine (30 mg, 0.083 mmol) in dichloromethane was added N,N-diisopropylethylamine (43 μL, 0.25 mmol) and methanesulfonyl chloride (6 μL, 0.083 mmol). After stirring at RT for 1 h, saturated aqueous NaHCO₃ was added. The mixture was filtered through a hydrophobic frit. The aqueous layer was rinsed with CH₂Cl₂ (3×) and filtered. The combined organic layers were concentrated and purified using reverse phase HPLC (C8, 0 to 100% MeOH in H₂O/0.1% TFA). The resulting product was partitioned between CH₂Cl₂ and saturated aqueous NaHCO₃. The aqueous layer was extracted with CH₂Cl₂ again, and the organic layers were combined. After drying over Na₂SO₄, the solvent was removed in vacuo to afford 8.4 mg (23%) of N-[4-(2-{[methyl(5,6,7,8-tetrahydro-8-quinolinyl)amino]methyl}-1H-benzimidazol-1-yl)butyl]methanesulfonamide as a pale yellow oil. ¹H NMR (CD₃OD): δ 8.42 (m, 1H), 7.51 (m, 3H), 7.22 (m, 3H), 4.36 (m, 2H), 4.10-3.88 (m, 3H), 3.01 (t, 2H), 2.87 (m, 1H), 2.84 (s, 3H), 2.75 (m, 1H), 2.24 (s, 3H), 2.20-2.05 (m, 3H), 1.76 (m, 3H), 1.47 (m, 2H). MS m/z 442 (M+1).

### Example 52

### N-{[1-(3-Aminopropyl)-1H-benzimidazol-2-yl]methyl}-N-(2-phenylethyl)-5,6,7,8-tetrahydro-8-quinolinamine

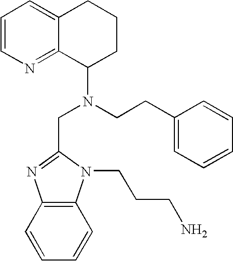

### a) N-(1H-Benzimidazol-2-ylmethyl)-N-(2-phenylethyl)-5,6,7,8-tetrahydro-8-quinolinamine

Reaction of N-(1H-benzimidazol-2-ylmethyl)-5,6,7,8-tetrahydro-8-quinolinamine (272 mg, 0.98 mmol) and phenylacetaldehyde (0.15 mL, 1.27 mmol) as described herein for the preparation of 3-(2-{[ethyl(5,6,7,8-tetrahydro-8-quinolinyl)amino]methyl}-1H-benzimidazol-1-yl)propanenitrile, afforded 276 mg (74%) of N-(1H-benzimidazol-2-ylmethyl)-N-(2-phenylethyl)-5,6,7,8-tetrahydro-8-quinolinamine as a light orange foam. ¹H NMR (DMSO-d₆): δ 12.34 (s, 1H), 8.46 (d, 1H), 7.47 (m, 3H), 7.17-7.00 (m, 8H), 4.20-4.00 (m, 3H), 2.91-2.54 (m, 6H), 2.06 (m, 1H), 1.90-1.77 (m, 2H), 1.62 (m, 1H). MS m/z 383 (M+1).

### b) 3-(2-{[(2-Phenylethyl)(5,6,7,8-tetrahydro-8-quinolinyl)amino]methyl}-1H-benzimidazol-1-yl)propanenitrile

Reaction of N-(1H-benzimidazol-2-ylmethyl)-N-(2-phenylethyl)-5,6,7,8-tetrahydro-8-quinolinamine (140 mg, 0.37 mmol) and 3-bromopropionitrile (91 μL, 1.10 mmol) as described herein for the preparation of N-methyl-N-{[1-(3-pyridinylmethyl)-1H-benzimidazol-2-yl]methyl}-5,6,7,8-tetrahydro-8-quinolinamine, afforded 150 mg (94%) of 3-(2-{[(2-phenylethyl)(5,6,7,8-tetrahydro-8-quinolinyl)amino]methyl}-1H-benzimidazol-1-yl)propanenitrile as a reddish-brown oil. ¹H NMR (DMSO-d₆): δ 8.44 (m, 1H), 7.60 (m, 2H), 7.45 (m, 1H), 7.21-7.04 (m, 6H), 6.88 (m, 2H), 4.74 (m, 1H), 4.55 (m, 1H), 4.24-4.06 (m, 3H), 3.23 (t, 2H), 2.86 (s, 2H), 2.77-2.51 (m, 4H), 2.07 (m, 1H), 1.88 (m, 2H), 1.62 (m, 1H). MS m/z 436 (M+1).

### c) N-{[1-(3-Aminopropyl)-1H-benzimidazol-2-yl]methyl}-N-(2-phenylethyl)-5,6,7,8-tetrahydro-8-quinolinamine

Reduction of 3-(2-{[(2-phenylethyl)(5,6,7,8-tetrahydro-8-quinolinyl)amino]methyl}-1H-benzimidazol-1-yl)propanenitrile (150 mg, 0.34 mmol) as described herein for the preparation of N-{[1-(3-aminopropyl)-1H-benzimidazol-2-yl]methyl}-N-methyl-5,6,7,8-tetrahydro-8-quinolinamine, afforded 89 mg (59%) of N-{[1-(3-aminopropyl)-1H-benzimidazol-2-yl]methyl}-N-(2-phenylethyl)-5,6,7,8-tetrahydro-8-quinolinamine as a yellow oil. ¹H NMR (DMSO-d₆): δ 8.42 (d, 1H), 7.56-7.44 (m, 3H), 7.19-7.03 (m, 6H), 6.89 (m, 2H), 4.35 (m, 2H), 4.28-4.17 (m, 2H), 4.10 (m, 1H), 2.84-2.69 (m, 2H), 2.65 (m, 2H), 2.51 (m, 2H), 1.97-1.76 (m, 7H), 1.60 (m, 1H). MS m/z 440 (M+1).

### Example 53

### N-{[1-(4-Aminobutyl)-1H-benzimidazol-2-yl}methyl]-N-(2-phenylethyl)-5,6,7,8-tetrahydro-8-quinolinamine

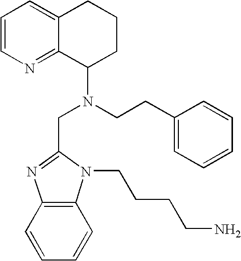

### a) 4-(2-{[(2-Phenylethyl)(5,6,7,8-tetrahydro-8-quinolinyl)amino]methyl}-1H-benzimidazol-1-yl)butanenitrile

Reaction of N-(1H-benzimidazol-2-ylmethyl)-N-(2-phenylethyl)-5,6,7,8-tetrahydro-8-quinolinamine (123 mg, 0.32 mmol) and 4-bromobutanenitrile (96 μL, 0.96 mmol) as described herein for the preparation of N-methyl-N-{[1-(3-pyridinylmethyl)-1H-benzimidazol-2-yl]methyl}-5,6,7,8-tetrahydro-8-quinolinamine, afforded 120 mg (83%) of 4-(2-{[(2-phenylethyl)(5,6,7,8-tetrahydro-8-quinolinyl)amino]methyl}-1H-benzimidazol-1-yl)butanenitrile as a brown oil. ¹H NMR (DMSO-d₆): δ 8.43 (d, 1H), 7.58-7.43 (m, 3H), 7.22-7.03 (m, 6H), 6.87 (m, 2H), 4.35 (m, 2H), 4.23 (m, 2H), 4.10 (m, 2H), 2.81-2.40 (m, 6H), 2.17-2.04 (m, 3H), 1.87 (m, 3H), 1.60 (m, 1H). MS m/z 450 (M+1).

### b) N-{[1-(4-Aminobutyl)-1H-benzimidazol-2-yl]methyl}-N-(2-phenylethyl)-5,6,7,8-tetrahydro-8-quinolinamine

Reduction of 4-(2-{[(2-phenylethyl)(5,6,7,8-tetrahydro-8-quinolinyl)amino]methyl}-1H-benzimidazol-1-yl)butanenitrile (120 mg, 0.27 mmol) as described herein for the preparation of N-{[1-(3-aminopropyl)-1H-benzimidazol-2-yl]methyl}-N-methyl-5,6,7,8-tetrahydro-8-quinolinamine, afforded 65 mg (54%) of N-{[1-(4-aminobutyl)-1H-benzimidazol-2-yl]methyl}-N-(2-phenylethyl)-5,6,7,8-tetrahydro-8-quinolinamine as a pale yellow oil. ¹H NMR (DMSO-d₆): δ 8.42 (d, 1H), 7.55 (m, 1H), 7.46 (m, 2H), 7.19-7.03 (m, 6H), 6.89 (m, 2H), 4.29-4.14 (m, 4H), 4.07 (m, 1H), 2.83-2.62 (m, 5H), 2.50 (m, 2H), 1.97 (m, 2H), 1.88 (m, 2H), 1.73 (m, 2H), 1.60 (m, 1H), 1.31 (m, 2H). MS m/z 454 (M+1).

### Example 54

### N-{[1-(4-Aminobutyl)-1H-benzimidazol-2-yl]methyl}-N-(3-methylbutyl)-5,6,7,8-tetrahydro-8-quinolinamine

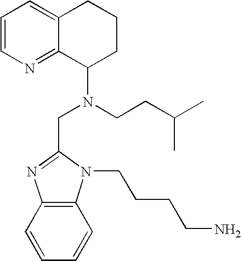

### a) 4-(2-{[(3-Methylbutyl)(5,6,7,8-tetrahydro-8-quinolinyl)amino]methyl}-H-benzimidazol-1-yl)butanenitrile

Reaction of N-(1H-benzimidazol-2-ylmethyl)-N-(3-methylbutyl)-5,6,7,8-tetrahydro-8-quinolinamine (167 mg, 0.48 mmol) and 4-bromobutanenitrile (0.14 mL, 1.44 mmol) as described herein for the preparation of N-methyl-N-{[1-(3-pyridinylmethyl)-1H-benzimidazol-2-yl]methyl}-5,6,7,8-tetrahydro-8-quinolinamine, afforded 57 mg (29%) of 4-(2-{[(3-methylbutyl)(5,6,7,8-tetrahydro-8-quinolinyl)amino]methyl}-1H-benzimidazol-1-yl)butanenitrile as a brown oil. ¹H NMR (DMSO-d₆): δ 8.43 (d, 1H), 7.53 (m, 2H), 7.44 (m, 1H), 7.21-7.11 (m, 3H), 4.47 (m, 2H), 4.14-3.97 (m, 3H), 2.75-2.39 (m, 6H), 2.20-2.01 (m, 3H), 1.87 (m, 2H), 1.58 (m, 1H), 1.39 (m, 1H), 1.10 (m, 2H), 0.58 (m, 6H). MS m/z 416 (M+1).

### b) N-{[1-(4-Aminobutyl)-1H-benzimidazol-2-yl]methyl}-N-(3-methylbutyl)-5,6,7,8-tetrahydro-8-quinolinamine

Reduction of 4-(2-{[(3-methylbutyl)(5,6,7,8-tetrahydro-8-quinolinyl)amino]methyl}-H-benzimidazol-1-yl)butanenitrile (57 mg, 0.14 mmol) as described herein for the preparation of N-{[1-(3-aminopropyl)-1H-benzimidazol-2-yl]methyl}-N-methyl-5,6,7,8-tetrahydro-8-quinolinamine, afforded 32 mg (56%) of N-{[1-(4-aminobutyl)-1H-benzimidazol-2-yl]methyl}-N-(3-methylbutyl)-5,6,7,8-tetrahydro-8-quinolinamine as a pale yellow oil. ¹H NMR (DMSO-d₆): δ 8.42 (d, 1H), 7.48 (m, 3H), 7.13 (m, 3H), 4.48-4.32 (m, 2H), 4.16-3.94 (m, 3H), 2.75-2.41 (m, 6H), 1.97-1.81 (m, 3H), 1.73 (m, 2H), 1.58 (m, 1H), 1.44 (m, 1H), 1.31 (m, 2H), 1.13 (m, 2H), 0.60 (m, 6H). MS m/z 420 (M+1).

### Example 55

### N-{[1-(4-Aminobutyl)-1H-benzimidazol-2-yl]methyl}-N-(Phenylmethyl)-5,6,7,8-tetrahydro-8-quinolinamine

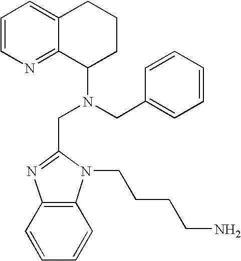

### a) 4-(2-{[(Phenylmethyl)(5,6,7,8-tetrahydro-8-quinolinyl)amino]methyl}-1H-benzimidazol-1-yl)butanenitrile

Reaction of N-(1H-benzimidazol-2-ylmethyl)-N-(phenylmethyl)-5,6,7,8-tetrahydro-8-quinolinamine (131 mg, 0.36 mmol) and 4-bromobutanenitrile (106 μL, 1.07 mmol) as described herein for the preparation of N-methyl-N-{[1-(3-pyridinylmethyl)-1H-benzimidazol-2-yl]methyl}-5,6,7,8-tetrahydro-8-quinolinamine, afforded 110 mg (71%) of 4-(2-{[(phenylmethyl)(5,6,7,8-tetrahydro-8-quinolinyl)amino]methyl}-1H-benzimidazol-1-yl)butanenitrile as a brown oil. ¹H NMR (DMSO-d₆): δ 8.51 (d, 1H), 7.52 (m, 1H), 7.45 (m, 2H), 7.32 (m, 2H), 7.24 (m, 2H), 7.15 (m, 4H), 4.40 (m, 1H), 4.19-3.86 (m, 5H), 3.70-3.53 (m, 2H), 2.78-2.49 (m, 2H), 2.42-2.29 (m, 2H), 2.08-1.80 (m, 4H), 1.49 (m, 1H). MS m/z 436 (M+1).

### b) N-{[1-(4-Aminobutyl)-1H-benzimidazol-2-yl]methyl}-N-(phenylmethyl)-5,6,7,8-tetrahydro-8-quinolinamine

Reduction of 4-(2-{[(phenylmethyl)(5,6,7,8-tetrahydro-8-quinolinyl)amino]methyl}-1H-benzimidazol-1-yl)butanenitrile (110 mg, 0.25 mmol) as described herein for the preparation of N-{[1-(3-aminopropyl)-1H-benzimidazol-2-yl]methyl}-N-methyl-5,6,7,8-tetrahydro-8-quinolinamine, afforded 57 mg (51%) of N-{[1-(4-aminobutyl)-1H-benzimidazol-2-yl]methyl}-N-(phenylmethyl)-5,6,7,8-tetrahydro-8-quinolinamine as an off-white foam. ¹H NMR (DMSO-d₆): δ 8.50 (m, 1H), 7.51 (m, 1H), 7.43 (m, 2H), 7.34 (m, 2H), 7.24 (m, 2H), 7.18-7.07 (m, 4H), 4.35 (m, 1H), 4.21-3.87 (m, 4H), 3.72-3.55 (m, 2H), 2.76-2.59 (m, 2H), 2.40 (m, 2H), 2.03-1.87 (m, 3H), 1.52 (m, 3H), 1.12 (m, 2H). MS m/z 440 (M+1).

### Example 56

### N-({1-[4-(Dimethylamino)-2-butyn-1-yl}-1H-benzimidazol-2-yl]methyl)-N-methyl-5,6,7,8-tetrahydro-8-quinolinamine

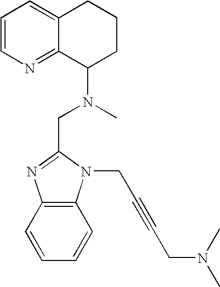

### a) t-Butyl (4-chloro-2-butyn-1-yl)carbamate

Reaction of the hydrochloride salt of (4-chloro-2-butyn-1-yl)amine (1.53 g, 10.9 mmol) as described herein for the preparation of t-butyl [(2Z)-4-chloro-2-buten-1-yl]carbamate afforded 2.11 g (95%) of t-butyl (4-chloro-2-butyn-1-yl)carbamate as a colorless oil. ¹H NMR (CDCl₃): δ 4.68 (br s, 1H), 4.13 (m, 2H), 3.98 (m, 2H), 1.45 (s, 9H).

### b) t-Butyl [4-(2-{[methyl(5,6,7,8-tetrahydro-8-quinolinyl)amino]methyl}-1H-benzimidazol-1-yl)-2-butyn-1-yl]carbamate

Reaction of N-(1H-benzimidazol-2-ylmethyl)-N-methyl-5,6,7,8-tetrahydro-8-quinolinamine (150 mg, 0.51 mmol) and t-butyl (4-chloro-2-butyn-1-yl)carbamate (0.42 g, 2.05 mmol) as described herein for the preparation of N-methyl-N-{[1-(3-pyridinylmethyl)-1H-benzimidazol-2-yl]methyl}-5,6,7,8-tetrahydro-8-quinolinamine, afforded 109 mg (46%) of t-butyl [4-(2-{[methyl(5,6,7,8-tetrahydro-8-quinolinyl)amino]methyl}-1H-benzimidazol-1-yl)-2-butyn-1-yl]carbamate as an off-white foam after flash chromatography (silica gel, gradient elution of dichloromethane to 9:1 dichloromethane/2M NH₃ in MeOH), followed by reverse phase HPLC (C8, 0 to 70% acetonitrile in H₂O/0.1% TFA). ¹H NMR (DMSO-d₆): δ 8.43 (d, 1H), 7.54 (m, 2H), 7.47 (m, 1H), 7.17 (m, 4H), 5.59-5.37 (m, 2H), 4.09 (m, 2H), 3.95 (m, 1H), 3.64 (m, 2H), 2.81-2.64 (m, 2H), 2.09 (s, 3H), 2.00-1.88 (m, 3H), 1.64 (m, 1H), 1.31 (s, 9H). MS m/z 460 (M+1).

### c) N-{[1-(4-Amino-2-butyn-1-yl)-1H-benzimidazol-2-yl]methyl}-N-methyl-5,6,7,8-tetrahydro-8-quinolinamine

Reaction of t-butyl [4-(2-{[methyl(5,6,7,8-tetrahydro-8-quinolinyl)amino]methyl}-1H-benzimidazol-1-yl)-2-butyn-1-yl]carbamate (109 mg, 0.24 mmol) as described herein for the preparation of N-methyl-N-({1-[2-(2-piperidinyl)ethyl]-1H-benzimidazol-2-yl}methyl)-5,6,7,8-tetrahydro-8-quinolinamine, afforded 120 mg of the hydrochloride salt of N-{[1-(4-amino-2-butyn-1-yl)-1H-benzimidazol-2-yl]methyl}-N-methyl-5,6,7,8-tetrahydro-8-quinolinamine as a tan solid. ¹H NMR (D₂O): δ 8.49 (m, 1H), 8.21 (m, 1H), 7.79 (m, 1H), 7.73 (m, 2H), 7.54 (m, 2H), 5.29 (s, 2H), 4.53-4.37 (m, 3H), 3.70 (s, 2H), 2.88 (m, 2H), 2.21 (m, 4H), 2.10-1.92 (m, 2H), 1.75 (m, 1H). MS m/z 360 (M+1).

### d) N-({1-[4-(Dimethylamino)-2-butyn-1-yl]-1H-benzimidazol-2-yl}methyl)-N-methyl-5,6,7,8-tetrahydro-8-quinolinamine

Reductive methylation of the hydrochloride salt of N-{[1-(4-amino-2-butyn-1-yl)-1H-benzimidazol-2-yl]methyl}-N-methyl-5,6,7,8-tetrahydro-8-quinolinamine (47 mg, 0.13 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 25 mg (64%) of N-({1-[4-(dimethylamino)-2-butyn-1-yl]-1H-benzimidazol-2-yl}methyl)-N-methyl-5,6,7,8-tetrahydro-8-quinolinamine as a colorless oil. ¹H NMR (CD₃OD): δ 8.42 (d, 1H), 7.53 (m, 3H), 7.30-7.15 (m, 3H), 5.60-5.30 (m, 2H), 4.06-3.92 (m, 3H), 3.29 (m, 1H), 3.15 (s, 2H), 2.92-2.72 (m, 2H), 2.27 (s, 3H), 2.14-2.04 (m, 8H), 1.74 (m, 1H). MS m/z 388 (M+1).

### Example 57

### N-Methyl-N-([1-[3-(4-morpholinyl)propyl]-1H-benzimidazol-2-yl]methyl)-5,6,7,8-tetrahydro-8-quinolinamine

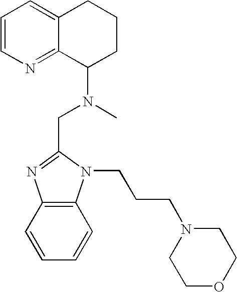

### a) 4-(3-Chloropropyl)morpholine

A solution of morpholine (2 mL, 23.0 mmol) in N,N-dimethylformamide (15 mL) was treated with potassium carbonate (4.75 g, 34.4 mmol) and 1-chloro-3-iodopropane (3.7 mL, 34.4 mmol). After 16 h, the reaction mixture was partitioned between EtOAc and H₂O. The aqueous layer was washed with EtOAc (15×). The combined organic layers were dried (Na₂SO₄) and concentrated. ¹H NMR indicated a very large amount of DMF still remaining, so the product was taken up in EtOAc and washed with H₂O. The organic layer was washed with brine, dried (Na₂SO₄) and concentrated. The resulting pale yellow oil was taken up in Et₂O and treated with 4N HCl in dioxane to precipitate the product, affording 1.45 g (32%) of the hydrochloride salt of 4-(3-chloropropyl)morpholine as a white solid. ¹H NMR (D₂O): δ 3.94 (br m, 2H), 3.70 (br m, 2H), 3.53 (m, 2H), 3.15 (br m, 2H), 3.19 (m, 2H), 3.10 (br m, 2H), 2.08 (m, 2H).

### b) N-Methyl-N-({1-[3-(4-morpholinyl)propyl]-1H-benzimidazol-2-yl}methyl)-5,6,7,8-tetrahydro-8-quinolinamine

Reaction of N-(1H-benzimidazol-2-ylmethyl)-N-methyl-5,6,7,8-tetrahydro-8-quinolinamine (60 mg, 0.21 mmol) and the hydrochloride salt of 4-(3-chloropropyl)morpholine (0.12 g, 62 mmol) as described herein for the preparation of N-methyl-N-({1-[2-(1-piperidinyl)ethyl]-1H-benzimidazol-2-yl}methyl)-5,6,7,8-tetrahydro-8-quinolinamine, afforded 29 mg (34%) of N-methyl-N-({1-[3-(4-morpholinyl)propyl]-1H-benzimidazol-2-yl}methyl)-5,6,7,8-tetrahydro-8-quinolinamine as a pale yellow oil after flash chromatography (silica gel, gradient elution of acetonitrile to 9:1 acetonitrile/NH₄OH), followed by reverse phase HPLC purification (C8, 0 to 70% acetonitrile in H₂O/0.1% TFA). ¹H NMR (DMSO-d₆): δ 8.42 (d, 1H), 7.50 (m, 3H), 7.14 (m, 3H), 4.35 (m, 2H), 4.16 (m, 2H), 3.96 (m, 1H), 3.50 (m, 4H), 2.77-2.64 (m, 2H), 2.29-2.16 (m, 6H), 2.07 (s, 3H), 1.96-1.85 (m, 5H), 1.62 (m, 1H). MS m/z 420 (M+1).

### Example 58

### N-({1-[(2E)-4-Amino-2-buten-1-yl]-1H-benzimidazol-2-yl}methyl)-N-methyl-5,6,7,8-tetrahydro-8-quinolinamine

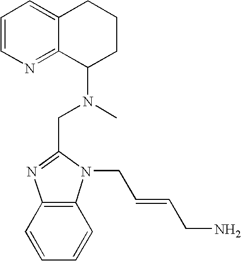

### a) 2-[(2E)-4-(2-{[Methyl(5,6,7,8-tetrahydro-8-quinolinyl)amino]methyl}-1H-benzimidazol-1-yl)-2-buten-1-yl]-1H-isoindole-1,3(2H)-dione

Reaction of N-(1H-benzimidazol-2-ylmethyl)-N-methyl-5,6,7,8-tetrahydro-8-quinolinamine (175 mg, 0.60 mmol) and 2-[(2E)-4-bromo-2-buten-1-yl]-1H-isoindole-1,3(2H)-dione (0.42 g, 1.50 mmol, prepared as described in J. Med. Chem. 1996, 39, 149-157) as described herein for the preparation of t-butyl [(2Z)-4-(2-{[methyl(5,6,7,8-tetrahydro-8-quinolinyl)amino]methyl}-1H-benzimidazol-1-yl)-2-buten-1-yl]carbamate afforded 72 mg (24%) of 2-[(2E)-4-(2-{[methyl(5,6,7,8-tetrahydro-8-quinolinyl)amino]methyl}-1H-benzimidazol-1-yl)-2-buten-1-yl]-1H-isoindole-1,3(2H)-dione as a brown oil. ¹H NMR (DMSO-d₆): δ 8.35 (d, 1H), 7.81 (m, 4H), 7.52 (m, 1H), 7.41 (m, 2H), 7.11 (m, 3H), 5.75-5.59 (m, 2H), 4.93 (m, 2H), 4.15-3.98 (m, 4H), 3.84 (m, 1H), 2.58 (m, 2H), 1.89 (s, 3H), 1.82-1.47 (m, 4H). MS m/z 492 (M+1).

### b) N-({1-[(2E)-4-Amino-2-buten-1-yl]-1H-benzimidazol-2-yl}methyl)-N-methyl-5,6,7,8-tetrahydro-8-quinolinamine

A solution of 2-[(2E)-4-(2-{[methyl(5,6,7,8-tetrahydro-8-quinolinyl)amino]methyl}-1H-benzimidazol-1-yl)-2-buten-1-yl]-1H-isoindole-1,3(2H)-dione (72 mg, 0.15 mmol) in ethanol (10 mL) was treated with hydrazine hydrate (0.5 mL, 10.3 mmol) and stirred at RT for 3 h. The reaction mixture was poured into saturated aqueous NaHCO₃ and extracted with CH₂Cl₂. The combined organic extracts were washed with brine, dried (Na₂SO₄) and concentrated. Flash chromatography (silica gel, gradient elution of acetonitrile to 9:1 acetonitrile/NH₄OH) afforded 37 mg (70%) of N-({1-[(2E)-4-amino-2-buten-1-yl]-1H-benzimidazol-2-yl}methyl)-N-methyl-5,6,7,8-tetrahydro-8-quinolinamine as a colorless oil. ¹H NMR (CD₃OD): δ 8.38 (d, 1H), 7.56 (m, 1H), 7.49 (m, 1H), 7.40 (m, 1H), 7.19 (m, 3H), 5.64 (m, 1H), 5.42 (m, 1H), 5.01 (m, 2H), 4.05-3.87 (m, 3H), 3.11 (d, 2H), 2.91-2.71 (m, 2H), 2.25 (s, 3H), 2.15-2.04 (m, 3H), 1.72 (m, 1H). MS m/z 362 (M+1).

### Example 59

### N-Methyl-N-({1-[3-(1-methyl-2-piperidinyl)propyl]-1H-benzimidazol-2-yl}methyl)-5,6,7,8-tetrahydro-8-quinolinamine

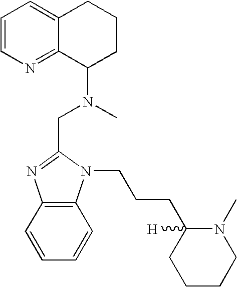

### a) 3-(2-Piperidinyl)-1-propanol

A solution of 2-(2-pyridinyl)propanol (1.5 mL, 11.7 mmol) in ethanol (45 mL) and concentrated HCl (0.96 mL, 11.7 mmol) was subjected to catalytic hydrogenation at 60 psi in the presence of 120 mg of 10% PtO₂. After stirring overnight, the reaction vessel was purged with nitrogen, catalyst removed by filtration through celite, and the filtrate concentrated to dryness at reduced pressure to afford 2.40 g of the hydrochloride salt of 3-(2-piperidinyl)-1-propanol as an off-white solid. ¹H NMR (DMSO-d₆): δ 3.38 (m, 2H), 3.32 (m, 1H), 3.16 (m, 1H), 2.91 (m, 1H), 2.78 (m, 1H), 1.81 (m, 1H), 1.73-1.23 (m, 8H).

### b) t-Butyl 2-(3-hydroxypropyl)-1-piperidinecarboxylate

A solution of the hydrochloride salt of 3-(2-piperidinyl)-1-propanol (2.09 g, 11.7 mmol) in THF (60 mL) and H₂O (5 mL) was treated with N,N-diisopropylethylamine (4.1 mL, 23.4 mmol) and di-t-butyl dicarbonate (4.34 g, 19.9 mmol). After stirring at RT for 18 h, the reaction was poured into 10% aqueous citric acid and extracted with EtOAc (2×). The combined organic extracts were washed with saturated aqueous NaHCO₃, brine, dried over Na₂SO₄ and concentrated to a pale yellow oil. Flash chromatography (silica gel, gradient elution of 0 to 100% EtOAc in hexanes) afforded 3.35 g (quant.) of t-butyl 2-(3-hydroxypropyl)-1-piperidinecarboxylate as a colorless oil. ¹H NMR (CDCl₃): δ 4.24 (br s, 1H), 3.94 (m, 1H), 3.67 (m, 2H), 2.73 (m, 1H), 1.80-1.28 (m, 20H).

### c) t-Butyl 2-(3-chloropropyl)-1-piperidinecarboxylate

Reaction of t-butyl 2-(3-hydroxypropyl)-1-piperidinecarboxylate (1.56 g, 6.4 mmol) with PS-triphenylphosphine and CCl₄ as described herein for the preparation of t-butyl 4-(chloromethyl)-1-piperidinecarboxylate, afforded 1.64 g (98%) of t-butyl 2-(3-chloropropyl)-1-piperidinecarboxylate as an opaque white oil. ¹H NMR (CDCl₃): δ 4.23 (m, 1H), 3.97 (m, 1H), 3.56 (m, 2H), 2.73 (t, 1H), 1.92-1.28 (m, 19H).

### d) t-Butyl 2-[3-(2-{[methyl(5,6,7,8-tetrahydro-8-quinolinyl)amino]methyl}-1H-benzimidazol-1-yl)propyl]-1-piperidinecarboxylate

Reaction of N-(1H-benzimidazol-2-ylmethyl)-N-methyl-5,6,7,8-tetrahydro-8-quinolinamine (184 mg, 0.63 mmol) and t-butyl 2-(3-chloropropyl)-1-piperidinecarboxylate (0.66 g, 2.52 mmol) as described herein for the preparation of N-methyl-N-({1-[2-(1-piperidinyl)ethyl]-1H-benzimidazol-2-yl}methyl)-5,6,7,8-tetrahydro-8-quinolinamine, afforded 104 mg (32%) of t-butyl 2-[3-(2-{[methyl(5,6,7,8-tetrahydro-8-quinolinyl)amino]methyl}-1H-benzimidazol-1-yl)propyl]-1-piperidinecarboxylate as a tan foam after flash chromatography (silica gel, gradient elution of dichloromethane to 9:1 dichloromethane/2 N NH₃ in MeOH). The diastereomers were indistinguishable by analytical RP-HPLC, however, ¹H NMR analysis is consistent with a 1:1 diasteromer mixture. ¹H NMR (DMSO-d₆): δ 8.43 (d, 1H), 7.49 (m, 3H), 7.14 (m, 3H), 4.29-3.96 (m, 6H), 3.75 (m, 1H), 2.77-2.59 (m, 3H), 2.07 (m, 3H), 1.95 (m, 3H), 1.64-1.45 (m, 9H), 1.25 (m, 11H). MS m/z 518 (M+1).

### e) N-Methyl-N-({1-[3-(2-piperidinyl)propyl]-1H-benzimidazol-2-yl}methyl)-5,6,7,8-tetrahydro-8-quinolinamine

Reaction of t-butyl 2-[3-(2-{[methyl(5,6,7,8-tetrahydro-8-quinolinyl)amino]methyl}-1H-benzimidazol-1-yl)propyl]-1-piperidinecarboxylate (104 mg, 0.20 mmol) as described herein for the preparation of N-methyl-N-{[1-(4-piperidinylmethyl)-1H-benzimidazol-2-yl]methyl}-5,6,7,8-tetrahydro-8-quinolinamine, afforded 75 mg (89%) of N-methyl-N-({1-[3-(2-piperidinyl)propyl]-1H-benzimidazol-2-yl}methyl)-5,6,7,8-tetrahydro-8-quinolinamine as a gold oil after flash chromatography (silica gel, gradient elution of acetonitrile to 9:1 acetonitrile/NH₄OH). The diastereomers were indistinguishable by analytical RP-HPLC, however, ¹H NMR analysis is consistent with a 1:1 diasteromer mixture. ¹H NMR (DMSO-d₆): δ 8.42 (d, 1H), 7.49 (m, 3H), 7.13 (m, 3H), 4.29-3.99 (m, 4H), 3.92 (m, 1H), 2.84-2.64 (m, 3H), 2.41-2.27 (m, 2H), 2.06 (s, 3H), 1.94 (m, 3H), 1.69 (m, 4H), 1.44 (m, 2H), 1.20 (m, 4H), 0.87 (m, 1H). MS m/z 418 (M+1).

### f) N-Methyl-N-({1-[3-(1-methyl-2-piperidinyl)propyl]-1H-benzimidazol-2-yl}methyl)-5,6,7,8-tetrahydro-8-quinolinamine

Reductive methylation of N-methyl-N-({1-[3-(2-piperidinyl)propyl]-1H-benzimidazol-2-yl}methyl)-5,6,7,8-tetrahydro-8-quinolinamine (35 mg, 0.084 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 35 mg (97%) of N-methyl-N-({1-[3-(1-methyl-2-piperidinyl)propyl]-1H-benzimidazol-2-yl}methyl)-5,6,7,8-tetrahydro-8-quinolinamine as a colorless oil. The diastereomers were indistinguishable by analytical RP-HPLC, however, ¹H NMR analysis is consistent with a 1:1 diasteromer mixture. ¹H NMR (CD₃OD): δ 8.42 (m, 1H), 7.51 (m, 3H), 7.22 (m, 3H), 4.38 (m, 2H), 4.09-3.89 (m, 3H), 2.92-2.74 (m, 3H), 2.24 (s, 3H), 2.20-2.06 (m, 7H), 1.87-1.47 (m, 9H), 1.27-1.12 (m, 3H). MS m/z 432 (M+1).

### Example 60

### N-({1-[3-(Dimethylamino)propyl]-1H-benzimidazol-2-yl}methyl)-N-(1-methylethyl)-5,6,7,8-tetrahydro-8-quinolinamine

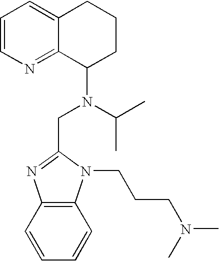

### a) N-(1H-Benzimidazol-2-ylmethyl)-N-(1-methylethyl)-5,6,7,8-tetrahydro-8-quinolinamine

A mixture of N-(1H-benzimidazol-2-ylmethyl)-5,6,7,8-tetrahydro-8-quinolinamine (0.58 g, 2.09 mmol), acetone (0.18 mL, 2.51 mmol), glacial acetic acid (0.36 mL, 6.27 mmol), and NaBH(OAc)₃ (0.89 g, 4.18 mmol) in 5 mL of 1,2-dichloroethane was stirred at RT for 5 h. The solution was partitioned between dichoromethane and saturated aqueous NaHCO₃. The aqueous layer was extracted again with dichloromethane. The combined organic layers was washed once with saturated aqueous NaHCO₃, once with aqueous brine, dried over Na₂SO₄, and concentrated to dryness at reduced pressure. The crude product was dissolved in MeOH and stirred with an equal volume of 6N aqueous HCl. After 0.5 h the solution was poured into a separatory funnel containing H₂O and EtOAc. To this was added 10% aqueous Na₂CO₃, followed by 20 mL of 5 N NaOH. The aqueous layer was extracted with EtOAc (3×). The combined EtOAc extracts were washed once with 10% aqueous Na₂CO₃, once with aqueous brine, dried over Na₂SO₄, and concentrated to dryness at reduced pressure. The crude product was purified by flash chromatography (silica gel, gradient elution of dichloromethane to 9:1 dichloromethane/2M NH₃ in MeOH) to afford 0.40 g (60%) of N-(1H-benzimidazol-2-ylmethyl)-N-(1-methylethyl)-5,6,7,8-tetrahydro-8-quinolinamine as an off-white foam. ¹H NMR (DMSO-d₆): δ 13.14 (s, 1H), 8.61 (d, 1H), 7.49 (m, 3H), 7.21 (m, 1H), 7.06 (m, 2H), 4.05-3.90 (m, 3H), 2.96 (m, 1H), 2.80 (m, 1H), 2.64 (m, 1H), 2.09 (m, 1H), 1.87 (m, 2H), 1.61 (m, 1H), 1.05 (d, 3H), 0.92 (d, 3H). MS m/z 321 (M+1).

### b) 3-(2-{[(1-Methylethyl)(5,6,7,8-tetrahydro-8-quinolinyl)amino]methyl}-1H-benzimidazol-1-yl)propanenitrile

Reaction of N-(1H-benzimidazol-2-ylmethyl)-N-(1-methylethyl)-5,6,7,8-tetrahydro-8-quinolinamine (54 mg, 0.17 mmol) and 3-bromopropionitrile (42 μL, 0.51 mmol) as described herein for the preparation of N-methyl-N-{[1-(3-pyridinylmethyl)-1H-benzimidazol-2-yl]methyl}-5,6,7,8-tetrahydro-8-quinolinamine, afforded 34 mg (54%) of 3-(2-{[(1-methylethyl)(5,6,7,8-tetrahydro-8-quinolinyl)amino]methyl}-1H-benzimidazol-1-yl)propanenitrile as a gold solid. ¹H NMR (DMSO-d₆): δ 8.38 (d, 1H), 7.60 (m, 1H), 7.53 (m, 1H), 7.37 (m, 1H), 7.20-7.05 (m, 3H), 4.94 (m, 1H), 4.67 (m, 1H), 4.22-4.07 (m, 2H), 3.90 (m, 1H), 3.35 (m, 2H), 2.76 (m, 2H), 2.59 (m, 1H), 2.10 (m, 1H), 1.90 (m, 2H), 1.53 (m, 1H), 1.02 (m, 6H). MS m/z 374 (M+1).

### c) N-{[1-(3-Aminopropyl)-1H-benzimidazol-2-yl]methyl}-N-(1-methylethyl)-5,6,7,8-tetrahydro-8-quinolinamine

Reduction of 3-(2-{[(1-methylethyl)(5,6,7,8-tetrahydro-8-quinolinyl)amino]methyl}-1H-benzimidazol-1-yl)propanenitrile (140 mg, 0.37 mmol) as described herein for the preparation of N-{[1-(3-aminopropyl)-1H-benzimidazol-2-yl]methyl}-N-methyl-5,6,7,8-tetrahydro-8-quinolinamine, afforded 104 mg (73%) of N-{[1-(3-aminopropyl)-1H-benzimidazol-2-yl]methyl}-N-(1-methylethyl)-5,6,7,8-tetrahydro-8-quinolinamine as a yellow oil. ¹H NMR (DMSO-d₆): δ 8.39 (d, 1H), 7.48 (m, 2H), 7.35 (m, 1H), 7.16-7.05 (m, 3H), 4.49 (m, 2H), 4.18-3.97 (m, 3H), 2.84 (m, 1H), 2.73 (m, 1H), 2.60-2.49 (m, 2H), 1.97-1.74 (m, 6H), 1.54 (m, 1H), 0.98 (m, 6H). MS m/z 378 (M+1).

### d) N-({1-[3-(Dimethylamino)propyl]-1H-benzimidazol-2-yl}methyl)-N-(1-methylethyl)-5,6,7,8-tetrahydro-8-quinolinamine

Reductive methylation of N-{[1-(3-aminopropyl)-1H-benzimidazol-2-yl]methyl}-N-(1-methylethyl)-5,6,7,8-tetrahydro-8-quinolinamine (66 mg, 0.17 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 50 mg (70%) of N-({1-[3-(dimethylamino)propyl]-1H-benzimidazol-2-yl}methyl)-N-(1-methylethyl)-5,6,7,8-tetrahydro-8-quinolinamine as a yellow oil. ¹H NMR (DMSO-d₆): δ 8.40 (m, 1H), 7.52 (m, 1H), 7.45 (m, 1H), 7.37 (m, 1H), 7.18-7.06 (m, 3H), 4.45 (m, 2H), 4.21-4.06 (m, 2H), 3.99 (m, 1H), 2.89-2.52 (m, 3H), 2.11-2.08 (m, 8H), 2.03-1.82 (m, 5H), 1.56 (m, 1H), 1.00 (m, 6H). MS m/z 406 (M+1).

### Example 61

### N-[3-(2-{[Methyl(5,6,7,8-tetrahydro-8-quinolinyl)amino]methyl}-1H-benzimidazol-1-yl)propyl]guanidine

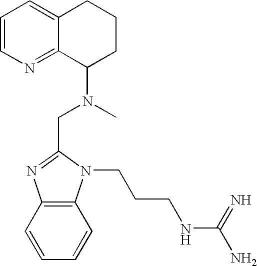

### a) Bis(1,1-dimethylethyl) ((E)-{[3-(2-{[methyl(5,6,7,8-tetrahydro-8-quinolinyl)amino]methyl}-1H-benzimidazol-1-yl)propyl]amino}methylylidene)biscarbamate

A solution of N-{[1-(3-aminopropyl)-1H-benzimidazol-2-yl]methyl}-N-methyl-5,6,7,8-tetrahydro-8-quinolinamine (87 mg, 0.25 mmol) in THF (5 mL) was treated with N,N′ di-boc-1H-pyrazole-1-carboxamidine (73 mg, 0.24 mmol). After stirring at RT for 18 h, the entire reaction mixture was purified by flash chromatography (silica gel, gradient elution of acetonitrile to 94:6 acetonitrile/NH₄OH) to afford 126 mg (86%) of bis(1,1-dimethylethyl) ((E)-{[3-(2-{[methyl(5,6,7,8-tetrahydro-8-quinolinyl)amino]methyl}-1H-benzimidazol-1-yl)propyl]amino}methylylidene)biscarbamate as a white foam. ¹H NMR (DMSO-d₆): δ11.44 (s, 1H), 8.43 (d, 1H), 8.35 (m, 1H), 7.50 (m, 3H), 7.14 (m, 3H), 4.35 (m, 2H), 4.21-4.04 (m, 2H), 3.96 (m, 1H), 3.36 (m, 2H), 2.78-2.63 (m, 3H), 2.04-1.88 (m, 7H), 1.61 (m, 1H), 1.40 (s, 9H), 1.35 (s, 9H). MS m/z 592 (M+1).

### b) N-[3-(2-{[Methyl(5,6,7,8-tetrahydro-8-quinolinyl)amino]methyl}-1H-benzimidazol-1-yl)propyl]guanidine

A solution of bis(1,1-dimethylethyl) ((E)-{[3-(2-{[methyl(5,6,7,8-tetrahydro-8-quinolinyl)amino]methyl}-1H-benzimidazol-1-yl)propyl]amino}methylylidene)biscarbamate (55 mg, 0.093 mmol) in anhydrous methanol (2 mL) was treated with 4 N HCl in dioxane (2 mL). After stirring for 24 h, the reaction was concentrated under reduced pressure. Evaporation with ethanol (3×), followed by evaporation with hexane (3×) afforded 52 mg of the hydrochloride salt of N-[3-(2-{[methyl(5,6,7,8-tetrahydro-8-quinolinyl)amino]methyl}-1H-benzimidazol-1-yl)propyl]guanidine as a white solid. ¹H NMR (D₂O): δ 8.47 (m, 1H), 8.18 (m, 1H), 7.68 (m, 3H), 7.50 (m, 2H), 4.46-4.27 (m, 5H), 3.15 (t, 2H), 2.88 (m, 2H), 2.20 (m, 4H), 2.08 (m, 3H), 1.96 (m, 1H), 1.74 (m, 1H). MS m/z 392 (M+1).

### Example 62

### N-[3-(2-{[Methyl(5,6,7,8-tetrahydro-8-quinolinyl)amino]methyl}-1H-benzimidazol-1-yl)propyl]benzenesulfonamide

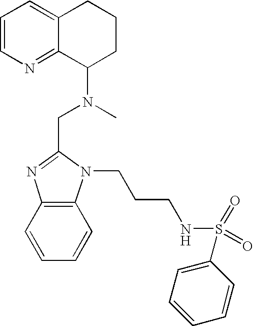

A solution of N-{[1-(3-aminopropyl)-1H-benzimidazol-2-yl]methyl}-N-methyl-5,6,7,8-tetrahydro-8-quinolinamine (112 mg, 0.32 mmol) in dichloromethane (3 mL) was treated with N,N-diisopropylethylamine (0.17 mL, 0.96 mmol) and phenylsulfonyl chloride (45 μL, 0.35 mmol). After stirring at RT for 0.5 h, saturated aqueous NaHCO₃ was added. The mixture was filtered through a hydrophobic frit. The aqueous layer was rinsed with CH₂Cl₂ (1×) and filtered. The combined organic layers were concentrated and purified by flash chromatography (silica gel, gradient elution of acetonitrile to 95:5 acetonitrile/NH₄OH) to afford 62 mg (39%) of N-[3-(2-{[methyl(5,6,7,8-tetrahydro-8-quinolinyl)amino]methyl}-1H-benzimidazol-1-yl)propyl]benzenesulfonamide as an off-white foam. ¹H NMR (DMSO-d₆): δ 8.35 (d, 1H), 7.85 (t, 1H), 7.73 (m, 2H), 7.63-7.42 (m, 6H), 7.14 (m, 3H), 4.29 (m, 2H), 4.14-3.93 (m, 3H), 2.83-2.63 (m, 4H), 1.98 (s, 3H), 1.93-1.81 (m, 5H), 1.62 (m, 1H). MS m/z 490 (M+1).

### Example 63

### N-[3-(2-{[Methyl(5,6,7,8-tetrahydro-8-quinolinyl)amino]methyl}-1H-benzimidazol-1-yl)propyl]methanesulfonamide

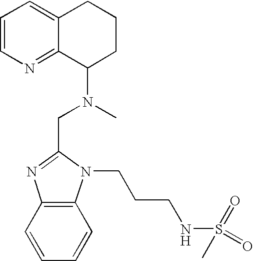

Reaction of N-{[1-(3-aminopropyl)-1H-benzimidazol-2-yl]methyl}-N-methyl-5,6,7,8-tetrahydro-8-quinolinamine (137 mg, 0.39 mmol) and methanesulfonyl chloride (33 μL, 0.43 mmol) as described herein for the preparation of N-[3-(2-{[methyl(5,6,7,8-tetrahydro-8-quinolinyl)amino]methyl}-1H-benzimidazol-1-yl)propyl]benzenesulfonamide, afforded 114 mg (68%) of N-[3-(2-{[methyl(5,6,7,8-tetrahydro-8-quinolinyl)amino]methyl}-1H-benzimidazol-1-yl)propyl]methanesulfonamide as a cream foam. ¹H NMR (DMSO-d₆): δ 8.44 (d, 1H), 7.51 (m, 3H), 7.30 (t, 1H), 7.20-7.10 (m, 3H), 4.38 (t, 2H), 4.19-4.00 (m, 3H), 3.01 (m, 2H), 2.87 (s, 3H), 2.81-2.64 (m, 2H), 2.05 (s, 3H), 1.93 (m, 5H), 1.64 (m, 1H). MS m/z 428 (M+1).

### Example 64

### N-Methyl-N-[(1-{3-[(3-methylbutyl)amino]propyl}-1H-benzimidazol-2-yl)methyl]-5,6,7,8-tetrahydro-8-quinolinamine

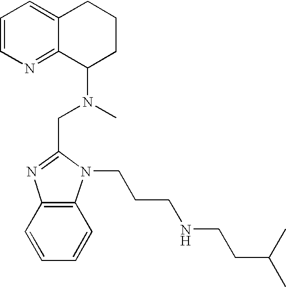

To a solution of N-{[1-(3-aminopropyl)-1H-benzimidazol-2-yl]methyl}-N-methyl-5,6,7,8-tetrahydro-8-quinolinamine (107 mg, 0.31 mmol) in anhydrous methanol (5 mL) was added isovaleraldehyde (49 μL, 0.46 mmol) and trimethyl orthoformate (0.10 mL, 0.92 mmol). After stirring at rt for 0.5 h, the reaction was treated with sodium borohydride (35 mg, 0.92 mmol). After 0.25 h, the reaction mixture was concentrated under reduced pressure. The residue was taken up in chloroform and washed with 1 N NaOH and brine, then dried over Na₂SO₄ and concentrated. Flash chromatography (silica gel, gradient elution of acetonitrile to 9:1 acetonitrile/NH₄OH) afforded 97 mg (76%) of N-methyl-N-[(1-{3-[(3-methylbutyl)amino]propyl}-1H-benzimidazol-2-yl)methyl]-5,6,7,8-tetrahydro-8-quinolinamine as a pale yellow oil. ¹H NMR (DMSO-d₆): δ 8.42 (d, 1H), 7.50 (m, 3H), 7.13 (m, 3H), 4.37 (m, 2H), 4.21-3.97 (m, 3H), 2.80-2.64 (m, 2H), 2.43 (m, 3H), 2.06 (s, 3H), 1.99-1.79 (m, 6H), 1.65-1.50 (m, 2H), 1.23 (m, 2H), 0.80 (d, 6H). MS m/z 420 (M+1).

### Example 65

### N-[(1-[3-[Bis(3-methylbutyl)amino]propyl]-1H-benzimidazol-2-yl)methyl]-N-methyl-5,6,7,8-tetrahydro-8-quinolinamine

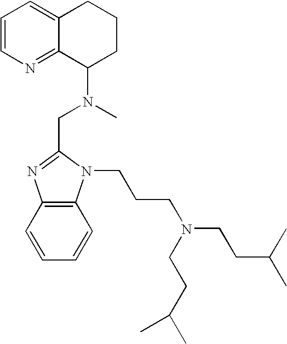

A mixture of N-{[1-(3-aminopropyl)-1H-benzimidazol-2-yl]methyl}-N-methyl-5,6,7,8-tetrahydro-8-quinolinamine (88 mg, 0.25 mmol), isovaleraldehyde (59 μL, 0.55 mmol), NaBH(OAc)₃ (0.21 g, 1.00 mmol) and AcOH (72 μL, 1.26 mmol) in anhydrous 1,2-dichloroethane (5 mL) was allowed to stir at RT for 18 h. The reaction was partitioned between CH₂Cl₂ and saturated aqueous NaHCO₃. The aqueous layer was extracted again with CH₂Cl₂. The combined organic layers were washed with brine, dried (Na₂SO₄) and concentrated under reduced pressure. The crude product was purified by flash chromatography (silica gel, gradient elution of acetonitrile to 9:1 acetonitrile/NH₄OH) to afford 108 mg (88%) of N-[(1-{3-[bis(3-methylbutyl)amino]propyl}-1H-benzimidazol-2-yl)methyl]-N-methyl-5,6,7,8-tetrahydro-8-quinolinamine as a yellow oil. ¹H NMR (DMSO-d₆): δ 8.43 (m, 1H), 7.55-7.45 (m, 3H), 7.15 (m, 3H), 4.31 (m, 2H), 4.23-4.05 (m, 2H), 3.98 (m, 1H), 2.82-2.66 (m, 2H), 2.31 (m, 6H), 2.08 (s, 3H), 1.96 (m, 3H), 1.80 (m, 2H), 1.64 (m, 1H), 1.49 (m, 2H), 1.20 (m, 4H), 0.80 (d, 12H). MS m/z 490 (M+1).

### Example 66

### N-({1-[3-(Dimethylamino)-2,2-dimethylpropyl]-1H-benzimidazol-2-yl}methyl)-N-(3-methylbutyl)-5,6,7,8-tetrahydro-8-quinolinamine

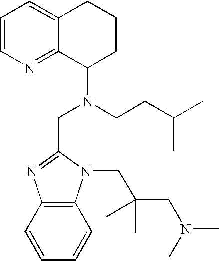

### a) 1,1-Dimethylethyl {2,2-dimethyl-3-[(2-nitrophenyl)amino]propyl}carbamate

Reaction of 1,1-dimethylethyl (3-amino-2,2-dimethylpropyl)carbamate (1.46 g, 7.22 mmol, Tyger Scientific) as described herein for the preparation of t-butyl 4-[(2-nitrophenyl)amino]-1-piperidinecarboxylate afforded 1.71 g (73%) of 1,1-dimethylethyl {2,2-dimethyl-3-[(2-nitrophenyl)amino]propyl}carbamate as an orange oil. ¹H NMR (CDCl₃): δ 8.31 (m, 1H), 8.17 (m, 1H), 7.42 (m, 1H), 6.87 (m, 1H), 6.63 (m, 1H), 4.65 (m, 1H), 3.13 (m, 4H), 1.43 (s, 9H), 1.04 (s, 6H). MS m/z 346 (M+1).

### b) 1,1-Dimethylethyl {3-[(2-aminophenyl)amino]-2,2-dimethylpropyl}carbamate

A solution of 1,1-dimethylethyl{2,2-dimethyl-3-[(2-nitrophenyl)amino]propyl}carbamate (1.71 g, 5.29 mmol) in EtOH was subjected to catalytic hydrogenation at 45 psi in the presence of 0.17 g of 10% Pd on charcoal. After 4 h the reaction vessel was purged with nitrogen, catalyst removed by filtration through celite, and the filtrate concentrated to dryness at reduced pressure to afford 1.51 g (97%) of 1,1-dimethylethyl {3-[(2-aminophenyl)amino]-2,2-dimethylpropyl}carbamate as a brown oil. ¹H NMR (CDCl₃): δ 6.87-6.69 (m, 4H), 4.85 (m, 1H), 3.61 (br s, 2H), 3.17 (d, 2H), 2.95 (s, 2H), 1.48 (s, 9H), 1.04 (s, 6H). MS m/z 316 (M+Na).

### c) 1,1-Dimethylethyl {2,2-dimethyl-3-[(2-{[({[(phenylmethyl)oxy]carbonyl}amino)acetyl]amino}phenyl)amino]propyl}carbamate

Reaction of 1,1-dimethylethyl {3-[(2-aminophenyl)amino]-2,2-dimethylpropyl}carbamate (1.51 g, 5.15 mmol) as previously described herein for the preparation of t-butyl 4-({2-[(N-{[(phenylmethyl)oxy]carbonyl}glycyl)amino]phenyl}amino)-1-piperidinecarboxylate afforded 2.10 g (84%) of 1,1-dimethylethyl {2,2-dimethyl-3-[(2-{[({[(phenylmethyl)oxy]carbonyl}amino)acetyl]amino}phenyl)amino]propyl}carbamate as a sticky white foam after flash chromatography (silica gel, gradient elution of 0 to 100% EtOAc in hexanes). ¹H NMR (DMSO-d₆): δ 9.24 (s, 1H), 7.55 (m, 1H), 7.34 (m, 5H), 6.95 (m, 3H), 6.67 (m, 1H), 6.50 (m, 1H), 5.03 (m, 2H), 4.80 (m, 1H), 3.83 (d, 2H), 2.84 (m, 4H), 1.35 (s, 9H), 0.82 (s, 6H). MS m/z 485 (M+1).

### d) 1,1-Dimethylethyl (2,2-dimethyl-3-{2-[({[(phenylmethyl)oxy]carbonyl}amino)methyl]-1H-benzimidazol-1-yl}propyl)carbamate

A solution of 1,1-dimethylethyl {2,2-dimethyl-3-[(2-{[({[(phenylmethyl)oxy]carbonyl}amino)acetyl]amino}phenyl)amino]propyl}carbamate (2.52 g, 5.20 mmol) in glacial acetic acid (65 mL) was heated to 80° C. for 9 h. The reaction was cooled to RT and concentrated under reduced pressure. The residue was taken up in EtOAc and washed with 10% aqueous Na₂CO₃ (2×), brine (1×), dried over Na₂SO₄, then concentrated under reduced pressure. The resulting tan foam was purified by flash chromatography (silica gel, gradient elution of 0 to 100% EtOAc in hexanes) to afford 1.83 g (75%) of 1,1-dimethylethyl (2,2-dimethyl-3-{2-[({[(phenylmethyl)oxy]carbonyl}amino)methyl]-1H-benzimidazol-1-yl}propyl)carbamate as a white foam. ¹H NMR (DMSO-d₆): δ 7.87 (t, 1H), 7.53 (m, 2H), 7.33 (m, 3H), 7.29 (m, 1H), 7.16 (m, 3H), 7.03 (m, 1H), 5.02 (s, 2H), 4.46 (d, 2H), 4.09 (s, 2H), 2.93 (m, 2H), 1.36 (s, 9H), 0.81 (s, 6H). MS m/z 467 (M+1).

### e) 1,1-Dimethylethyl {3-[2-(aminomethyl)-1H-benzimidazol-1-yl]-2,2-dimethylpropyl}carbamate

Reaction of 1,1-dimethylethyl (2,2-dimethyl-3-{2-[({[(phenylmethyl)oxy]carbonyl}amino)methyl]-1H-benzimidazol-1-yl}propyl)carbamate (1.83 g, 3.92 mmol) as previously described herein for the preparation of 1,1-dimethylethyl {3-[(2-aminophenyl)amino]-2,2-dimethylpropyl}carbamate afforded 1.25 g (96%) of 1,1-dimethylethyl {3-[2-(aminomethyl)-1H-benzimidazol-1-yl]-2,2-dimethylpropyl}carbamate as a sticky white foam. ¹H NMR (DMSO-d₆): δ 7.51 (m, 2H), 7.13 (m, 2H), 7.04 (t, 1H), 4.07 (s, 2H), 3.90 (s, 2H), 2.92 (d, 2H), 1.95 (br s, 2H), 1.36 (s, 9H), 0.80 (s, 6H). MS m/z 333 (M+1).

### f) 1,1-Dimethylethyl (2,2-dimethyl-3-{2-[(5,6,7,8-tetrahydro-8-quinolinylamino)methyl]-1H-benzimidazol-1-yl}propyl)carbamate

Reaction of 1,1-dimethylethyl {3-[2-(aminomethyl)-1H-benzimidazol-1-yl]-2,2-dimethylpropyl}carbamate (0.70 g, 2.10 mmol) as previously described herein for the preparation of t-butyl 4-{2-[(5,6,7,8-tetrahydro-8-quinolinylamino)methyl]-1H-benzimidazol-1-yl}-1-piperidinecarboxylate afforded 0.86 g (88%) of 1,1-dimethylethyl (2,2-dimethyl-3-{2-[(5,6,7,8-tetrahydro-8-quinolinylamino)methyl]-1H-benzimidazol-1-yl}propyl)carbamate as an orange oil after flash chromatography (silica gel, gradient elution of acetonitrile to 94:6 acetonitrile/NH₄OH). ¹H NMR (DMSO-d₆): δ 8.38 (d, 1H), 7.53 (m, 3H), 7.16 (m, 3H), 7.07 (t, 1H), 4.16-4.00 (m, 4H), 3.78 (m, 1H), 3.17 (br s, 1H), 2.94 (d, 2H), 2.75 (m, 2H), 2.10 (m, 1H), 1.90 (m, 1H), 1.69 (m, 2H), 1.36 (s, 9H), 0.82 (s, 6H). MS m/z 464 (M+1).

### g) 1,1-Dimethylethyl [2,2-dimethyl-3-(2-{[(3-methylbutyl)(5,6,7,8-tetrahydro-8-quinolinyl)amino]methyl}-1H-benzimidazol-1-yl)propyl]carbamate

Reaction of 1,1-dimethylethyl (2,2-dimethyl-3-{2-[(5,6,7,8-tetrahydro-8-quinolinylamino)methyl]-1H-benzimidazol-1-yl}propyl)carbamate (139 mg, 0.30 mmol) and isovaleraldehyde (48 μL, 0.45 mmol) as herein described for the preparation of 3-(2-{[ethyl(5,6,7,8-tetrahydro-8-quinolinyl)amino]methyl}-1H-benzimidazol-1-yl)propanenitrile, afforded 147 mg (92%) of 1,1-dimethylethyl [2,2-dimethyl-3-(2-{[(3-methylbutyl)(5,6,7,8-tetrahydro-8-quinolinyl)amino]methyl}-1H-benzimidazol-1-yl)propyl]carbamate as an off-white foam. ¹H NMR (DMSO-d₆): δ 8.43 (d, 1H), 7.52 (m, 2H), 7.44 (m, 1H), 7.29-7.09 (m, 3H), 7.04 (t, 1H), 4.47-4.32 (m, 2H), 4.22-4.10 (m, 2H), 3.92 (m, 1H), 2.91 (d, 2H), 2.79-2.62 (m, 2H), 1.97-1.84 (m, 3H), 1.60 (m, 1H), 1.37-1.34 (m, 11H), 0.92-0.83 (m, 3H), 0.77 (s, 3H), 0.75 (s, 3H), 0.49 (m, 6H). MS m/z 534 (M+1).

### h) N-{[1-(3-Amino-2,2-dimethylpropyl)-1H-benzimidazol-2-yl]methyl}-N-(3-methylbutyl)-5,6,7,8-tetrahydro-8-quinolinamine

Deprotection of 1,1-dimethylethyl [2,2-dimethyl-3-(2-{[(3-methylbutyl)(5,6,7,8-tetrahydro-8-quinolinyl)amino]methyl}-1H-benzimidazol-1-yl)propyl]carbamate (147 mg, 0.27 mmol) as described herein for the preparation of N-{[1-(3-azetidinylmethyl)-1H-benzimidazol-2-yl]methyl}-N-methyl-5,6,7,8-tetrahydro-8-quinolinamine, afforded 104 mg (87%) of N-{[1-(3-amino-2,2-dimethylpropyl)-1H-benzimidazol-2-yl]methyl}-N-(3-methylbutyl)-5,6,7,8-tetrahydro-8-quinolinamine as a yellow oil after flash chromatography (silica gel, gradient elution of acetonitrile to 9:1 acetonitrile/NH₄OH). ¹H NMR (DMSO-d₆): δ 8.40 (d, 1H), 7.57 (m, 1H), 7.50 (m, 1H), 7.42 (m, 1H), 7.10 (m, 3H), 4.35 (s, 2H), 4.25-4.10 (m, 2H), 3.92 (m, 1H), 2.77-2.60 (m, 2H), 2.43-2.30 (m, 4H), 1.89 (m, 3H), 1.56 (m, 1H), 1.36 (m, 1H), 0.93 (m, 2H), 0.73 (s, 6H), 0.51 (m, 6H). MS m/z 434 (M+1).

### i) N-({1-[3-(Dimethylamino)-2,2-dimethylpropyl]-1H-benzimidazol-2-yl}methyl)-N-(3-methylbutyl)-5,6,7,8-tetrahydro-8-quinolinamine

Reductive methylation of N-{[1-(3-amino-2,2-dimethylpropyl)-1H-benzimidazol-2-yl]methyl}-N-(3-methylbutyl)-5,6,7,8-tetrahydro-8-quinolinamine (60 mg, 0.14 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 37 mg (58%) of N-({1-[3-(dimethylamino)-2,2-dimethylpropyl]-1H-benzimidazol-2-yl}methyl)-N-(3-methylbutyl)-5,6,7,8-tetrahydro-8-quinolinamine as a yellow oil. ¹H NMR (DMSO-d₆): δ 8.41 (d, 1H), 7.53-7.43 (m, 3H), 7.17-7.08 (m, 3H), 4.39 (m, 2H), 4.24-4.08 (m, 2H), 3.86 (m, 1H), 2.74-2.62 (m, 2H), 2.43 (m, 2H), 2.26 (s, 6H), 2.17 (s, 2H), 1.92 (m, 3H), 1.56 (m, 1H), 1.40 (m, 1H), 0.99 (m, 2H), 0.78 (s, 3H), 0.77 (s, 3H), 0.54 (m, 6H). MS m/z 462 (M+1).

### Example 67

### N-({1-[3-(Dimethylamino)-2,2-dimethylproyl]-1H-benzimidazol-2-yl}methyl)-N-methyl-5,6,7,8-tetrahydro-8-quinolinamine

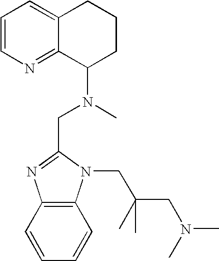

### a) 1,1-Dimethylethyl [2,2-dimethyl-3-(2-{[methyl(5,6,7,8-tetrahydro-8-quinolinyl)amino]methyl}-1H-benzimidazol-1-yl)propyl]carbamate

Reductive methylation of 1,1-dimethylethyl (2,2-dimethyl-3-{2-[(5,6,7,8-tetrahydro-8-quinolinylamino)methyl]-1H-benzimidazol-1-yl}propyl)carbamate (0.62 g, 1.34 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 0.59 g (92%) of 1,1-dimethylethyl [2,2-dimethyl-3-(2-{[methyl(5,6,7,8-tetrahydro-8-quinolinyl)amino]methyl}-1H-benzimidazol-1-yl)propyl]carbamate as a peach-colored foam after flash chromatography (silica gel, gradient elution of acetonitrile to 9:1 acetonitrile/NH₄OH). ¹H NMR (DMSO-d₆): δ 8.44 (m, 1H), 7.52 (m, 3H), 7.19-7.07 (m, 4H), 4.36 (m, 2H), 4.26-4.09 (m, 2H), 3.88 (m, 1H), 2.89 (m, 2H), 2.83-2.65 (m, 2H), 1.97-1.93 (m, 6H), 1.63 (m, 1H), 1.32 (s, 9H), 0.76 (s, 3H), 0.73 (s, 3H). MS m/z 478 (M+1).

### b) N-{[1-(3-Amino-2,2-dimethylpropyl)-1H-benzimidazol-2-yl]methyl}-N-methyl-5,6,7,8-tetrahydro-8-quinolinamine

Deprotection of 1,1-dimethylethyl [2,2-dimethyl-3-(2-{[methyl(5,6,7,8-tetrahydro-8-quinolinyl)amino]methyl}-1H-benzimidazol-1-yl)propyl]carbamate (0.59 g, 1.23 mmol) as described herein for the preparation of N-{[1-(3-azetidinylmethyl)-1H-benzimidazol-2-yl]methyl}-N-methyl-5,6,7,8-tetrahydro-8-quinolinamine, afforded 379 mg (81%) of N-{[1-(3-amino-2,2-dimethylpropyl)-1H-benzimidazol-2-yl]methyl}-N-methyl-5,6,7,8-tetrahydro-8-quinolinamine as a yellow oil after flash chromatography (silica gel, gradient elution of acetonitrile to 9:1 acetonitrile/NH₄OH). ¹H NMR (DMSO-d₆): δ 8.41 (d, 1H), 7.58-7.47 (m, 3H), 7.18-7.07 (m, 3H), 4.36-4.08 (m, 4H), 3.88 (m, 1H), 2.80-2.63 (m, 2H), 2.33 (m, 2H), 1.98 (s, 3H), 1.92 (m, 2H), 1.63 (m, 2H), 0.70 (s, 6H). MS m/z 378 (M+1).

### c) N-({1-[3-(Dimethylamino)-2,2-dimethylpropyl]-1H-benzimidazol-2-yl}methyl)-N-methyl-5,6,7,8-tetrahydro-8-quinolinamine

Reductive methylation of N-{[1-(3-amino-2,2-dimethylpropyl)-1H-benzimidazol-2-yl]methyl}-N-methyl-5,6,7,8-tetrahydro-8-quinolinamine (51 mg, 0.13 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 43 mg (78%) of N-({1-[3-(dimethylamino)-2,2-dimethylpropyl]-1H-benzimidazol-2-yl}methyl)-N-methyl-5,6,7,8-tetrahydro-8-quinolinamine as a yellow oil. ¹H NMR (DMSO-d₆): δ 8.43 (m, 1H), 7.52 (m, 3H), 7.20-7.09 (m, 3H), 4.37-4.05 (m, 4H), 3.85 (m, 1H), 2.83-2.65 (m, 2H), 2.25 (s, 6H), 2.12 (s, 2H), 2.01-1.92 (m, 6H), 1.64 (m, 1H), 0.78 (s, 3H), 0.70 (s, 3H). MS m/z 406 (M+1).

### Example 68

### N-[2,2-Dimethyl-3-(2-{[methyl(5,6,7,8-tetrahydro-8-quinolinyl)amino]methyl}-1H-benzimidazol-1-yl)propyl]quanidine

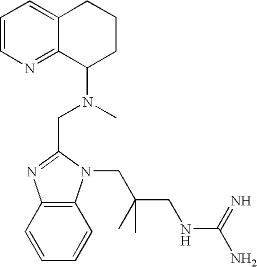

### a) Bis(1,1-dimethylethyl) ((Z)-{[2,2-dimethyl-3-(2-{[methyl(5,6,7,8-tetrahydro-8-quinolinyl)amino]methyl}-1H-benzimidazol-1-yl)propyl]amino}methylylidene)biscarbamate

Reaction of N-{[1-(3-amino-2,2-dimethylpropyl)-1H-benzimidazol-2-yl]methyl}-N-methyl-5,6,7,8-tetrahydro-8-quinolinamine (48 mg, 0.13 mmol) as described herein for the preparation of bis(1,1-dimethylethyl) ((E)-{[3-(2-{[methyl(5,6,7,8-tetrahydro-8-quinolinyl)amino]methyl}-1H-benzimidazol-1-yl)propyl]amino}methylylidene)biscarbamate afforded 61 mg (78%) of bis(1,1-dimethylethyl) ((Z)-{[2,2-dimethyl-3-(2-{[methyl(5,6,7,8-tetrahydro-8-quinolinyl)amino]methyl}-1H-benzimidazol-1-yl)propyl]amino}methylylidene)biscarbamate as a yellow oil. ¹H NMR (DMSO-d₆): δ 11.48 (s, 1H), 8.44 (m, 1H), 8.33 (m, 1H), 7.56-7.48 (m, 3H), 7.14 (m, 3H), 4.40 (m, 2H), 4.28-4.13 (m, 2H), 3.83 (m, 1H), 3.40-3.26 (m, 2H), 2.77-2.59 (m, 2H), 1.95 (s, 3H), 1.89 (m, 3H), 1.61 (m, 1H), 1.39 (s, 9H), 1.37 (s, 9H), 0.85 (s, 6H). MS m/z 620 (M+1).

### b) N-[2,2-Dimethyl-3-(2-{[methyl(5,6,7,8-tetrahydro-8-quinolinyl)amino]methyl}-1H-benzimidazol-1-yl)propyl]guanidine

Deprotection of bis(1,1-dimethylethyl) ((Z)-{[2,2-dimethyl-3-(2-{[methyl(5,6,7,8-tetrahydro-8-quinolinyl)amino]methyl}-1H-benzimidazol-1-yl)propyl]amino}methylylidene)biscarbamate (60 mg, 0.097 mmol) as described herein for the preparation of N-{[1-(3-azetidinylmethyl)-1H-benzimidazol-2-yl]methyl}-N-methyl-5,6,7,8-tetrahydro-8-quinolinamine, afforded 29 mg (71%) of N-[2,2-dimethyl-3-(2-{[methyl(5,6,7,8-tetrahydro-8-quinolinyl)amino]methyl}-1H-benzimidazol-1-yl)propyl]guanidine as a tan solid. ¹H NMR (DMSO-d₆): δ 8.42 (d, 1H), 7.51 (m, 4H), 7.15 (m, 4H), 4.40 (m, 2H), 4.28-4.12 (m, 2H), 3.90 (m, 1H), 3.08 (d, 2H), 2.76-2.64 (m, 3H), 1.97 (s, 3H), 1.92 (m, 3H), 1.62 (m, 1H), 1.20 (m, 1H), 0.84 (s, 6H). MS m/z 420 (M+1).

### Example 69

### N-[(1-{2,2-Dimethyl-3-[(3-methylbutyl)amino]propyl}-1H-benzimidazol-2-yl)methyl]-N-methyl-5,6,7,8-tetrahydro-8-quinolinamine

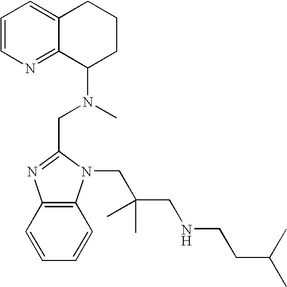

Reaction of N-{[1-(3-amino-2,2-dimethylpropyl)-1H-benzimidazol-2-yl]methyl}-N-methyl-5,6,7,8-tetrahydro-8-quinolinamine (49 mg, 0.13 mmol) as described herein for the preparation of N-methyl-N-[(1-{3-[(3-methylbutyl)amino]propyl}-1H-benzimidazol-2-yl)methyl]-5,6,7,8-tetrahydro-8-quinolinamine afforded 50 mg (86%) of N-[(1-{2,2-dimethyl-3-[(3-methylbutyl)amino]propyl}-1H-benzimidazol-2-yl)methyl]-N-methyl-5,6,7,8-tetrahydro-8-quinolinamine as a colorless oil. ¹H NMR (DMSO-d₆): δ 8.41 (d, 1H), 7.59 (m, 1H), 7.49 (m, 2H), 7.12 (m, 3H), 4.39-4.10 (m, 4H), 3.92 (m, 1H), 2.80-2.64 (m, 2H), 2.44 (m, 1H), 2.21 (m, 2H), 1.97 (s, 3H), 1.91 (m, 3H), 1.59 (m, 2H), 1.38 (m, 1H), 1.28 (m, 2H), 0.81 (m, 6H), 0.75 (d, 6H). MS m/z 448 (M+1).

### Example 70

### N-({1-[2-(1H-Imidazol-1-yl)ethyl]-1H-benzimidazol-2-yl}methyl)-N-methyl-5,6,7,8-tetrahydro-8-quinolinamine

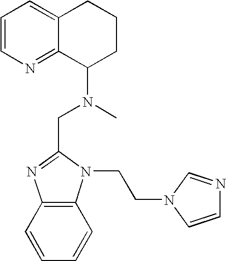

### a) [2-(1H-Imidazol-1-yl)ethyl]amine

Imidazole (8.10 g, 119 mmol), 2-chloroethylamine monohydrochloride (15.2 g, 131 mmol), tetrabutylammonium hydrogensulfate (1.62 g, 4.8 mmol) and sodium hydroxide (17.1 g, 428 mmol) were combined with 100 mL acetonitrile and heated under reflux for 21 h. The reaction mixture was cooled and filtered. The filtrate was concentrated to a pale yellow oil. Flash chromatography (silica gel, gradient elution of acetonitrile to 9:1 acetonitrile/NH₄OH) afforded 4.52 g (34%) of [2-(1H-imidazol-1-yl)ethyl]amine as a pale yellow oil. ¹H NMR (DMSO-d₆): δ 7.58 (s, 1H), 7.17 (s, 1H), 6.83 (s, 1H), 3.86 (t, 2H), 2.79 (t, 2H), 2.10 (brs, 2H).

### b) N-[2-(1H-Imidazol-1-yl)ethyl]-2-nitroaniline

Reaction of [2-(1H-imidazol-1-yl)ethyl]amine (0.465 g, 4.18 mmol) as described herein for the preparation of t-butyl 4-[(2-nitrophenyl)amino]-1-piperidinecarboxylate afforded 0.32 g (33%) of N-[2-(1H-imidazol-1-yl)ethyl]-2-nitroaniline as a gold solid. ¹H NMR (DMSO-d₆): δ 8.10 (t, 1H), 8.03 (dd, 1H), 7.61 (s, 1H), 7.49 (m, 1H), 7.20 (s, 1H), 7.05 (m, 1H), 6.86 (s, 1H), 6.68 (m, 1H), 4.23 (t, 2H), 3.72 (q, 2H). MS m/z 233 (M+1).

### c) N-[2-(1H-Imidazol-1-yl)ethyl]-1,2-benzenediamine

Reaction of N-[2-(1H-imidazol-1-yl)ethyl]-2-nitroaniline (0.32 g, 1.38 mmol) as described herein for the preparation of 1,1-dimethylethyl {3-[(2-aminophenyl)amino]-2,2-dimethylpropyl}carbamate afforded 0.27 g (96%) of N-[2-(1H-imidazol-1-yl)ethyl]-1,2-benzenediamine as a brown/purple oil. ¹H NMR (DMSO-d₆): δ 7.61 (s, 1H), 7.19 (s, 1H), 6.84 (s, 1H), 6.53-6.39 (m, 4H), 4.51 (t, 1H), 4.43 (s, 2H), 4.13 (t, 2H), 3.32 (q, 2H). MS m/z 203 (M+1).

### d) N¹-(2-{[2-(1H-Imidazol-1-yl)ethyl]amino}phenyl)-N²-methyl-N²-(5,6,7,8-tetrahydro-8-quinolinyl)glycinamide

Reaction of N-[2-(1H-imidazol-1-yl)ethyl]-1,2-benzenediamine (0.209 g, 1.03 mmol) and N-methyl-N-(5,6,7,8-tetrahydro-8-quinolinyl)glycine (0.228 g, 1.03 mmol) as previously described herein for the preparation of t-butyl 4-({2-[(N-{[(phenylmethyl)oxy]carbonyl}glycyl)amino]phenyl}amino)-1-piperidinecarboxylate afforded 0.261 g (62%) of N²-(2-{[2-(1H-imidazol-1-yl)ethyl]amino}phenyl)-N²-methyl-N²-(5,6,7,8-tetrahydro-8-quinolinyl)glycinamide as a brown oil. ¹H NMR (DMSO-d₆): δ10.35 (s, 1H), 8.29 (d, 1H), 7.53 (m, 2H), 7.37 (m, 1H), 7.18 (m, 1H), 7.12 (s, 1H), 7.02 (m, 1H), 6.78 (m, 2H), 6.65 (t, 1H), 5.11 (t, 1H), 4.16-3.93 (m, 3H), 3.44 (m, 2H), 3.20 (m, 2H), 2.80-2.66 (m, 2H), 2.32 (s, 3H), 2.10 (m, 1H), 1.91 (m, 1H), 1.84-1.67 (m, 2H). MS m/z 405 (M+1).

### e) N-({1-[2-(1H-Imidazol-1-yl)ethyl]-1H-benzimidazol-2-yl}methyl)-N-methyl-5,6,7,8-tetrahydro-8-quinolinamine

Reaction of N¹-(2-{[2-(1H-imidazol-1-yl)ethyl]amino}phenyl)-N²-methyl-N²-(5,6,7,8-tetrahydro-8-quinolinyl)glycinamide (0.261 g, 0.65 mmol) as described herein for the preparation of 1,1-dimethylethyl (2,2-dimethyl-3-{2-[({[(phenylmethyl)oxy]carbonyl}amino)methyl]-1H-benzimidazol-1-yl}propyl)carbamate afforded 0.213 g (86%) of N-({1-[2-(1H-imidazol-1-yl)ethyl]-1H-benzimidazol-2-yl}methyl)-N-methyl-5,6,7,8-tetrahydro-8-quinolinamine as a tan foam after flash chromatography (silica gel, gradient elution of acetonitrile to 9:1 acetonitrile/NH₄OH). ¹H NMR (DMSO-d₆): δ 8.38 (m, 1H), 7.51 (m, 2H), 7.32 (m, 2H), 7.17-7.10 (m, 3H), 6.99 (s, 1H), 6.79 (s, 1H), 4.74 (m, 2H), 4.50 (m, 2H), 3.99-3.76 (m, 3H), 2.80-2.65 (m, 2H), 2.04 (m, 4H), 1.90 (m, 2H), 1.64 (m, 1H). MS m/z 387 (M+1).

### Example 71

### N-Methyl-N-({1-[2-(1-methyl-1H-imidazol-5-yl)ethyl]-1H-benzimidazol-2-yl}methyl)-5,6,7,8-tetrahydro-8-quinolinamine

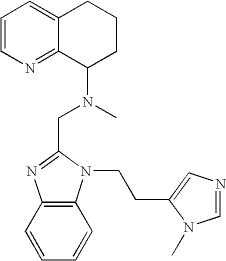

### a) N-[2-(1-Methyl-1H-imidazol-5-yl)ethyl]-2-nitroaniline

Reaction of 3-methylhistamine dihydrochloride (151 mg, 0.76 mmol) as described herein for the preparation of t-butyl 4-[(2-nitrophenyl)amino]-1-piperidinecarboxylate afforded 39 mg (21%) of N-[2-(1-methyl-1H-imidazol-5-yl)ethyl]-2-nitroaniline as an orange oil. ¹H NMR (DMSO-d₆): δ 8.16 (t, 1H), 8.04 (d, 1H), 7.51 (m, 2H), 7.07 (m, 1H), 6.73 (s, 1H), 6.67 (m, 1H), 3.57 (m, 5H), 2.88 (t, 2H). MS m/z 247 (M+1).

### b) N-[2-(1-Methyl-1H-imidazol-5-yl)ethyl]-1,2-benzenediamine

Reaction of N-[2-(1-methyl-1H-imidazol-5-yl)ethyl]-2-nitroaniline (100 mg, 0.41 mmol) as described herein for the preparation of 1,1-dimethylethyl {3-[(2-aminophenyl)amino]-2,2-dimethylpropyl}carbamate afforded 84 mg (95%) of N-[2-(1-methyl-1H-imidazol-5-yl)ethyl]-1,2-benzenediamine as a yellow oil. ¹H NMR (DMSO-d₆): δ 7.46 (s, 1H), 6.72 (s, 1H), 6.52-6.37 (m, 4H), 4.49 (t, 1H), 4.42 (s, 2H), 3.51 (s, 3H), 3.22 (m, 2H), 2.79 (m, 2H). MS m/z 217 (M+1).

### c) N²-Methyl-N¹-(2-{[2-(1-methyl-1H-imidazol-5-yl)ethyl]amino}phenyl)-N²-(5,6,7,8-tetrahydro-8-quinolinyl)glycinamide

Reaction of N-[2-(1-methyl-1H-imidazol-5-yl)ethyl]-1,2-benzenediamine (84 mg, 0.39 mmol) as previously described herein for the preparation of N¹-(2-{[2-(1H-imidazol-1-yl)ethyl]amino}phenyl)-N²-methyl-N²-(5,6,7,8-tetrahydro-8-quinolinyl)glycinamide afforded 85 mg (52%) of N²-methyl-N¹-(2-{[2-(1-methyl-1H-imidazol-5-yl)ethyl]amino}phenyl)-N²-(5,6,7,8-tetrahydro-8-quinolinyl)glycinamide as a gold oil. ¹H NMR (DMSO-d₆): δ 10.37 (s, 1H), 8.29 (d, 1H), 7.51 (m, 1H), 7.41 (s, 1H), 7.34 (m, 1H), 7.17 (m, 1H), 7.01 (m, 1H), 6.74 (m, 1H), 6.61 (m, 2H), 5.01 (t, 1H), 3.93 (m, 1H), 3.44 (s, 3H), 3.35-3.16 (m, 4H), 2.81-2.64 (m, 4H), 2.28 (s, 3H), 2.08 (m, 1H), 1.90 (m, 1H), 1.83-1.65 (m, 2H). MS m/z 419 (M+1).

### d) N-Methyl-N-({1-[2-(1-methyl-1H-imidazol-5-yl)ethyl]-1H-benzimidazol-2-yl}methyl)-5,6,7,8-tetrahydro-8-quinolinamine

Reaction of N²-methyl-N¹-(2-{[2-(1-methyl-1H-imidazol-5-yl)ethyl]amino}phenyl)-N²-(5,6,7,8-tetrahydro-8-quinolinyl)glycinamide (85 mg, 0.20 mmol) as described herein for the preparation of 1,1-dimethylethyl (2,2-dimethyl-3-{2-[({[(phenylmethyl)oxy]carbonyl}amino)methyl]-1H-benzimidazol-1-yl}propyl)carbamate afforded 65 mg (80%) of N-methyl-N-({1-[2-(1-methyl-1H-imidazol-5-yl)ethyl]-1H-benzimidazol-2-yl}methyl)-5,6,7,8-tetrahydro-8-quinolinamine as a white foam after flash chromatography (silica gel, gradient elution of acetonitrile to 9:1 acetonitrile/NH₄OH). ¹H NMR (DMSO-d₆): δ 8.34 (m, 1H), 7.55 (m, 1H), 7.45 (m, 2H), 7.39 (m, 1H), 7.15 (m, 3H), 6.63 (s, 1H), 4.60 (m, 2H), 4.08-3.90 (m, 3H), 3.36 (s, 3H), 3.04 (m, 2H), 2.77-2.62 (m, 2H), 2.06 (s, 3H), 1.91 (m, 3H), 1.61 (m, 1H). MS m/z 401 (M+1).

### Example 72

### N-Methyl-N-({1-[2-(1-methyl-1H-imidazol-4-yl)ethyl]-1H-benzimidazol-2-yl}methyl)-5,6,7,8-tetrahydro-8-quinolinamine

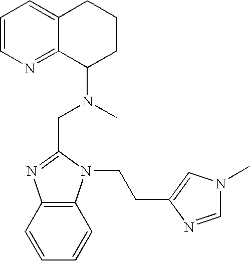

### a) N-[2-(1-Methyl-1H-imidazol-4-yl)ethyl]-2-nitroaniline

Reaction of 1-methylhistamine dihydrochloride (108 mg, 0.55 mmol) as described herein for the preparation of t-butyl 4-[(2-nitrophenyl)amino]-1-piperidinecarboxylate afforded 57 mg (43%) of N-[2-(1-methyl-1H-imidazol-4-yl)ethyl]-2-nitroaniline as an orange oil. ¹H NMR (DMSO-d₆): δ 8.27 (t, 1H), 8.03 (d, 1H), 7.51 (m, 2H), 7.04 (m, 1H), 6.93 (s, 1H), 6.66 (m, 1H), 3.57 (s, 3H), 3.52 (m, 2H), 2.78 (t, 2H). MS m/z 247 (M+1).

### b) N-[2-(1-Methyl-1H-imidazol-4-yl)ethyl]-1,2-benzenediamine

Reaction of N-[2-(1-methyl-1H-imidazol-4-yl)ethyl]-2-nitroaniline (57 mg, 0.23 mmol) as described herein for the preparation of 1,1-dimethylethyl {3-[(2-aminophenyl)amino]-2,2-dimethylpropyl}carbamate afforded 44 mg (88%) of N-[2-(1-methyl-1H-imidazol-4-yl)ethyl]-1,2-benzenediamine as a brown oil. ¹H NMR (DMSO-d₆): δ 7.43 (s, 1H), 6.88 (s, 1H), 6.52-6.36 (m, 4H), 4.39 (br m, 3H), 3.55 (s, 3H), 3.17 (m, 2H), 2.69 (m, 2H). MS m/z 217 (M+1).

### c) N²-Methyl-N¹-(2-{[2-(1-methyl-1H-imidazol-4-yl)ethyl]amino}phenyl)-N²-(5,6,7,8-tetrahydro-8-quinolinyl)glycinamide

Reaction of N-[2-(1-methyl-1H-imidazol-4-yl)ethyl]-1,2-benzenediamine (44 mg, 0.20 mmol) as previously described herein for the preparation of N¹-(2-{[2-(1H-imidazol-1-yl)ethyl]amino}phenyl)-N²-methyl-N¹-(5,6,7,8-tetrahydro-8-quinolinyl)glycinamide afforded 59 mg (69%) of N²-methyl-N¹-(2-{[2-(1-methyl-1H-imidazol-4-yl)ethyl]amino}phenyl)-N²-(5,6,7,8-tetrahydro-8-quinolinyl)glycinamide as a gold oil. ¹H NMR (DMSO-d₆): δ 10.31 (s, 1H), 8.29 (d, 1H), 7.50 (m, 1H), 7.39 (s, 1H), 7.31 (m, 1H), 7.15 (m, 1H), 6.99 (t, 1H), 6.80 (s, 1H), 6.69 (m, 1H), 6.58 (t, 1H), 5.10 (m, 1H), 3.99 (m, 2H), 3.50 (s, 3H), 3.23 (m, 3H), 2.82-2.61 (m, 4H), 2.31 (s, 3H), 2.07 (m, 1H), 1.93-1.61 (m, 3H). MS m/z 419 (M+1).

### d) N-Methyl-N-({1-[2-(1-methyl-1H-imidazol-4-yl)ethyl]-1H-benzimidazol-2-yl}methyl)-5,6,7,8-tetrahydro-8-quinolinamine

Reaction of N²-methyl-N¹-(2-{[2-(1-methyl-1H-imidazol-4-yl)ethyl]amino}phenyl)-N²-(5,6,7,8-tetrahydro-8-quinolinyl)glycinamide (59 mg, 0.14 mmol) as described herein for the preparation of 1,1-dimethylethyl (2,2-dimethyl-3-{2-[({[(phenylmethyl)oxy]carbonyl}amino)methyl]-1H-benzimidazol-1-yl}propyl)carbamate afforded 41 mg (73%) of N-methyl-N-({1-[2-(1-methyl-1H-imidazol-4-yl)ethyl]-1H-benzimidazol-2-yl}methyl)-5,6,7,8-tetrahydro-8-quinolinamine as a tan foam after flash chromatography (silica gel, gradient elution of acetonitrile to 9:1 acetonitrile/NH₄OH). ¹H NMR (DMSO-d₆): δ 8.41 (m, 1H), 7.50 (m, 3H), 7.39 (m, 1H), 7.14 (m, 3H), 6.70 (s, 1H), 4.54 (m, 2H), 4.10-3.96 (m, 3H), 3.52 (s, 3H), 2.88 (m, 2H), 2.80-2.63 (m, 2H), 2.08 (s, 3H), 1.95 (m, 3H), 1.63 (m, 1H). MS m/z 401 (M+1).

### Example 73

### N-[(1-{4-[(Dimethylamino)methyl]phenyl}-1H-benzimidazol-2-yl)methyl]-N-methyl-5,6,7,8-tetrahydro-8-quinolinamine

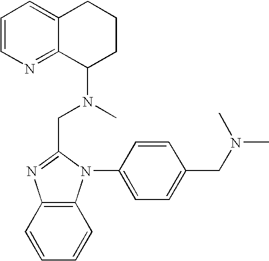

### a) 4-(2-{[Methyl(5,6,7,8-tetrahydro-8-quinolinyl)amino]methyl}-1H-benzimidazol-1-yl)benzonitrile

A mixture of N-(1H-benzimidazol-2-ylmethyl)-N-methyl-5,6,7,8-tetrahydro-8-quinolinamine (179 mg, 0.61 mmol), 4-fluorobenzonitrile (222 mg, 1.84 mmol) and cesium carbonate (1.00 g, 3.06 mmol) in anhydrous DMF (10 mL) was heated to 80° C. After 18 h, the reaction was cooled to RT and partitioned between EtOAc and water. The aqueous layer was extracted again with EtOAc. The combined organic layers were washed with brine, dried over Na₂SO₄ and concentrated. Flash chromatography (silica gel, gradient elution of acetonitrile to 9:1 acetonitrile/NH₄OH) afforded 180 mg (75%) of 4-(2-{[methyl(5,6,7,8-tetrahydro-8-quinolinyl)amino]methyl}-1H-benzimidazol-1-yl)benzonitrile as a yellow oil. ¹H NMR (DMSO-d₆): δ 8.35 (m, 1H), 8.02 (m, 2H), 7.88 (m, 2H), 7.68 (m, 1H), 7.41 (m, 1H), 7.27-7.11 (m, 4H), 4.26-4.12 (m, 2H), 3.63 (m, 1H), 2.57 (m, 2H), 1.95 (s, 3H), 1.67-1.38 (m, 4H). MS m/z 394 (M+1).

### b) N-({1-[4-(Aminomethyl)phenyl]-1H-benzimidazol-2-yl}methyl)-N-methyl-5,6,7,8-tetrahydro-8-quinolinamine

Reduction of 4-(2-{[methyl(5,6,7,8-tetrahydro-8-quinolinyl)amino]methyl}-1H-benzimidazol-1-yl)benzonitrile (175 mg, 0.44 mmol) as described herein for the preparation of N-{[1-(3-aminopropyl)-1H-benzimidazol-2-yl]methyl}-N-methyl-5,6,7,8-tetrahydro-8-quinolinamine, afforded 55 mg (31%) of N-({1-[4-(aminomethyl)phenyl]-1H-benzimidazol-2-yl}methyl)-N-methyl-5,6,7,8-tetrahydro-8-quinolinamine as an off-white foam. ¹H NMR (DMSO-d₆): δ 8.32 (m, 1H), 7.64 (m, 1H), 7.50 (m, 4H), 7.40 (m, 1H), 7.19 (m, 2H), 7.10 (m, 2H), 4.03 (m, 2H), 3.83 (s, 2H), 3.66 (m, 1H), 2.56 (m, 2H), 2.03 (s, 3H), 1.69-1.44 (m, 4H). MS m/z 398 (M+1).

### c) N-[(1-{4-[(Dimethylamino)methyl]phenyl}-1H-benzimidazol-2-yl)methyl]-N-methyl-5,6,7,8-tetrahydro-8-quinolinamine

Reductive methylation of N-({1-[4-(aminomethyl)phenyl]-1H-benzimidazol-2-yl}methyl)-N-methyl-5,6,7,8-tetrahydro-8-quinolinamine (40 mg, 0.10 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 14 mg (33%) of N-[(1-{4-[(dimethylamino)methyl]phenyl}-1H-benzimidazol-2-yl)methyl]-N-methyl-5,6,7,8-tetrahydro-8-quinolinamine as a colorless oil. ¹H NMR (CD₃OD): δ 8.28 (m, 1H), 7.67 (m, 1H), 7.54-7.41 (m, 5H), 7.26 (m, 2H), 7.11 (m, 2H), 4.12-3.94 (m, 2H), 3.68 (m, 1H), 3.30 (m, 2H), 2.63 (m, 2H), 2.31 (s, 6H), 2.15 (s, 3H), 1.79 (m, 2H), 1.67 (m, 1H), 1.52 (m, 1H). MS m/z 426 (M+1).

### Example 74

### N-[(1-{2-[(Dimethylamino)methyl]phenyl}-1H-benzimidazol-2-yl)methyl]-N-methyl-5,6,7,8-tetrahydro-8-quinolinamine

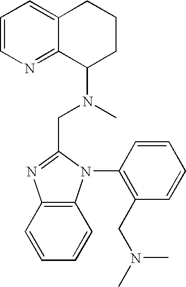

### a) 2-(2-{[Methyl(5,6,7,8-tetrahydro-8-quinolinyl)amino]methyl}-1H-benzimidazol-1-yl)benzonitrile

Reaction of N-(1H-benzimidazol-2-ylmethyl)-N-methyl-5,6,7,8-tetrahydro-8-quinolinamine (111 mg, 0.38 mmol) and 2-fluorobenzonitrile (0.12 mL, 1.14 mmol) as described herein for the preparation of 4-(2-{[methyl(5,6,7,8-tetrahydro-8-quinolinyl)amino]methyl}-1H-benzimidazol-1-yl)benzonitrile afforded 103 mg (69%) of 2-(2-{[methyl(5,6,7,8-tetrahydro-8-quinolinyl)amino]methyl}-1H-benzimidazol-1-yl)benzonitrile as a yellow oil. ¹H NMR analysis is consistent with a 1:1 mixture of rotamers. ¹H NMR (DMSO-d₆): δ 8.32 (m, 1H), 8.11-7.81 (m, 2H), 7.72-7.55 (m, 3H), 7.39-6.95 (m, 5H), 4.38-3.81 (m, 2H), 3.43 (m, 1H), 2.49 (m, 2H), 2.06, 1.88 (s, 3H total, 2 rotamers), 1.57-1.11 (m, 4H). MS m/z 394 (M+1).

### b) N-({1-[2-(Aminomethyl)phenyl]-1H-benzimidazol-2-yl}methyl)-N-methyl-5,6,7,8-tetrahydro-8-quinolinamine

Reduction of 2-(2-{[methyl(5,6,7,8-tetrahydro-8-quinolinyl)amino]methyl}-1H-benzimidazol-1-yl)benzonitrile (100 mg, 0.25 mmol) as described herein for the preparation of N-{[1-(3-aminopropyl)-1H-benzimidazol-2-yl]methyl}-N-methyl-5,6,7,8-tetrahydro-8-quinolinamine, afforded 79 mg (78%) of N-({1-[2-(aminomethyl)phenyl]-1H-benzimidazol-2-yl}methyl)-N-methyl-5,6,7,8-tetrahydro-8-quinolinamine as a gold oil. ¹H NMR analysis is consistent with a 1:1 mixture of rotamers. ¹H NMR (DMSO-d₆): δ 8.31 (m, 1H), 7.75 (m, 1H), 7.66 (m, 1H), 7.55 (m, 1H), 7.42-7.08 (m, 6H), 6.85 (m, 1H), 4.03 (m, 2H), 3.91-3.67 (m, 1H), 3.56-3.41 (m, 1H), 3.29 (m, 1H), 3.24 (m, 1H), 2.53 (m, 2H), 2.17, 1.99 (s, 3H total, 2 rotamers), 1.64-1.37 (m, 3H). MS m/z 398 (M+1).

### c) N-[(1-{2-[(Dimethylamino)methyl]phenyl}-1H-benzimidazol-2-yl)methyl]-N-methyl-5,6,7,8-tetrahydro-8-quinolinamine

Reductive methylation of N-({1-[2-(aminomethyl)phenyl]-1H-benzimidazol-2-yl}methyl)-N-methyl-5,6,7,8-tetrahydro-8-quinolinamine (62 mg, 0.16 mmol) as described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 51 mg (77%) of N-[(1-{2-[(dimethylamino)methyl]phenyl}-1H-benzimidazol-2-yl)methyl]-N-methyl-5,6,7,8-tetrahydro-8-quinolinamine as a colorless oil. ¹H NMR analysis is consistent with a 1:1 mixture of rotamers. ¹H NMR (DMSO-d₆): δ 8.31 (m, 1H), 7.68-7.52 (m, 3H), 7.46-7.22 (m, 3H), 7.20-7.09 (m, 3H), 6.82 (m, 1H), 3.99-3.48 (m, 3H), 3.14-2.65 (m, 2H), 2.52 (m, 2H), 2.10 (m, 3H), 1.95 (s, 3H), 1.86 (s, 3H), 1.63-1.36 (m, 4H). MS m/z 426 (M+1).

### Example 75

### (8S)—N-Methyl-N-[(1-{[(3R)-1-methyl-3-piperidinyl]methyl}-1H-benzimidazol-2-yl)methyl]-5,6,7,8-tetrahydro-8-quinolinamine

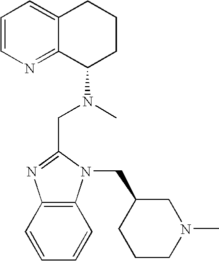

### a) 1,1-Dimethylethyl (3S)-3-{[(2-nitrophenyl)amino]methyl}-1-piperidinecarboxylate

Reaction of 1,1-dimethylethyl (3S)-3-(aminomethyl)-1-piperidinecarboxylate (1.95 g, 9.10 mmol, Ennova MedChem Group, Inc.) as described herein for the preparation of 1,1-dimethylethyl (3R)-3-{[(2-nitrophenyl)amino]methyl}-1-piperidinecarboxylate afforded 1,1-dimethylethyl (3S)-3-{[(2-nitrophenyl)amino]methyl}-1-piperidinecarboxylate as a yellow-orange oil in quantitative yield. ¹H NMR (CDCl₃): δ 8.18-8.10 (m, 2H), 7.42 (m, 1H), 6.82 (m, 1H), 6.64 (m, 1H), 3.97 (m, 1H), 3.82 (m, 1H), 3.28-3.13 (m, 2H), 2.93 (m, 1H), 2.77 (m, 1H), 1.92 (m, 2H), 1.70 (m, 1H), 1.52-1.42 (m, 10H), 1.40-1.22 (m, 1H). MS m/z 358 (M+Na).

### b) 1,1-Dimethylethyl (3S)-3-{[(2-aminophenyl)amino]methyl}-1-piperidinecarboxylate

Reduction of 1,1-dimethylethyl (3S)-3-{[(2-nitrophenyl)amino]methyl}-1-piperidinecarboxylate (3.16 g, 9.42 mmol) as described herein for the preparation of 1,1-dimethylethyl {3-[(2-aminophenyl)amino]-2,2-dimethylpropyl}carbamate afforded 1,1-dimethylethyl (3S)-3-{[(2-aminophenyl)amino]methyl}-1-piperidinecarboxylate as a brown oil in quantitative yield. ¹H NMR (CDCl₃): δ 6.81 (m, 1H), 6.73-6.63 (m, 3H), 4.03 (br m, 1H), 3.83 (m, 1H), 3.05-2.67 (m, 8H), 1.94-1.85 (m, 2H), 1.66 (m, 1H), 1.45 (m, 9H), 1.29 (m, 1H). MS m/z 328 (M+Na).

### c) 1,1-Dimethylethyl (3S)-3-{[2-(chloromethyl)-1H-benzimidazol-1-yl]methyl}-1-piperidinecarboxylate

Reaction of 1,1-dimethylethyl (3S)-3-{[(2-aminophenyl)amino]methyl}-1-piperidinecarboxylate (2.33 g, 7.60 mmol) as described herein for the preparation of 1,1-dimethylethyl (3R)-3-{[2-(chloromethyl)-1H-benzimidazol-1-yl]methyl}-1-piperidinecarboxylate afforded 2.37 g (85%) of 1,1-dimethylethyl (3S)-3-{[2-(chloromethyl)-1H-benzimidazol-1-yl]methyl}-1-piperidinecarboxylate as a light pinkish-brown foam. ¹H NMR (CDCl₃): δ 7.80 (m, 1H), 7.36 (m, 3H), 4.93 (m, 2H), 4.25 (m, 1H), 4.12 (m, 1H), 3.82 (m, 2H), 2.95 (m, 1H), 2.79 (m, 1H), 2.21 (m, 1H), 1.69 (m, 2H), 1.39-1.24 (m, 1H). MS m/z 364 (M+1).

### d) 1,1-Dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate

A solution of 1,1-dimethylethyl (3S)-3-{[2-(chloromethyl)-1H-benzimidazol-1-yl]methyl}-1-piperidinecarboxylate (0.46 g, 1.26 mmol), (8S)—N-methyl-5,6,7,8-tetrahydro-8-quinolinamine (0.21 g, 1.26 mmol), potassium iodide (31 mg, 0.15 mmol), and N,N-diisopropylethylamine (0.44 mL, 2.53 mmol) in 20 mL of acetonitrile was heated to 65° C. with stirring. Two additional 20 mg portions of (8S)—N-methyl-5,6,7,8-tetrahydro-8-quinolinamine were added. After 7 hours the solution was cooled to RT and concentrated. The residue was partitioned between EtOAc and saturated aqueous NaHCO₃. The aqueous layer was extracted with an additional portion of EtOAc. The combined organic layers were washed with saturated aqueous brine (1×), dried over Na₂SO₄, and concentrated to dryness at reduced pressure. The crude product was purified by flash chromatography (silica gel, gradient elution of MeCN to 95:5 MeCN/NH₄OH) to afford 0.38 g (61%) of 1,1-dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate as a white solid. ¹H NMR (DMSO-d₆): δ 8.49 (d, 1H), 7.58 (m, 3H), 7.21 (m, 3H), 4.27 (m, 3H), 4.07-3.80 (m, 3H), 3.57 (br m, 1H), 2.76 (m, 3H), 2.45 (m, 1H), 2.12 (m, 4H), 2.01 (m, 3H), 1.64 (m, 3H), 1.21 (m, 11H). MS m/z 490 (M+1).

### e) (8S)—N-Methyl-N-({1-[(3R)-3-piperidinylmethyl]-1H-benzimidazol-2-yl}methyl)-5,6,7,8-tetrahydro-8-quinolinamine

Deprotection of 1,1-dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate (0.16 g, 0.33 mmol) as described herein for the preparation of (8S)—N-methyl-N-({1-[(3S)-3-piperidinylmethyl]-1H-benzimidazol-2-yl}methyl)-5,6,7,8-tetrahydro-8-quinolinamine afforded (8S)—N-methyl-N-({1-[(3R)-3-piperidinylmethyl]-1H-benzimidazol-2-yl}methyl)-5,6,7,8-tetrahydro-8-quinolinamine as a tan foam in quantitative yield. ¹H NMR (CD₃OD): δ 8.56 (d, 1H), 7.65 (m, 2H), 7.54 (m, 1H), 7.31 (m, 3H), 4.51 (m, 1H), 4.30 (m, 1H), 4.12-3.85 (m, 3H), 3.11 (m, 1H), 2.90 (m, 4H), 2.63 (t, 1H), 2.43 (m, 1H), 2.33 (s, 3H), 2.20 (m, 3H), 1.95-1.79 (m, 3H), 1.66 (m, 1H), 1.39 (m, 1H). MS m/z 390 (M+1).

### f) (8S)—N-Methyl-N-[(1-{[(3R)-1-methyl-3-piperidinyl]methyl}-1H-benzimidazol-2-yl)methyl]-5,6,7,8-tetrahydro-8-quinolinamine

Reductive methylation of (8S)—N-methyl-N-({1-[(3R)-3-piperidinylmethyl]-1H-benzimidazol-2-yl}methyl)-5,6,7,8-tetrahydro-8-quinolinamine (102 mg, 0.26 mmol) as described herein for the preparation of (8S)—N-methyl-N-[(1-{[(3S)-1-methyl-3-piperidinyl]methyl}-1H-benzimidazol-2-yl)methyl]-5,6,7,8-tetrahydro-8-quinolinamine afforded 76 mg (72%) of (8S)—N-methyl-N-[(1-{[(3R)-1-methyl-3-piperidinyl]methyl}-1H-benzimidazol-2-yl)methyl]-5,6,7,8-tetrahydro-8-quinolinamine as a colorless oil. ¹H NMR (CD₃OD): δ 8.43 (d, 1H), 7.55 (m, 2H), 7.45 (m, 1H), 7.23 (m, 3H), 4.26 (d, 2H), 4.08-3.84 (m, 3H), 2.94-2.74 (m, 3H), 2.48 (m, 1H), 2.25-2.02 (m, 10H), 1.92-1.44 (m, 6H), 1.27 (m, 1H). MS m/z 404 (M+1).

### Example 76

### (8R)—N-Methyl-N-[(1-{[(3S)-1-methyl-3-piperidinyl]methyl}-1H-benzimidazol-2-yl)methyl]-5,6,7,8-tetrahydro-8-quinolinamine

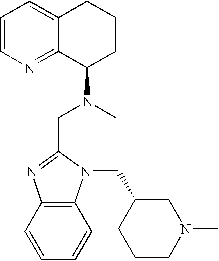

### a) (8R)—N-{(1R)-1-[4-(Methyloxy)phenyl]ethyl}-5,6,7,8-tetrahydro-8-quinolinamine

A solution of (R)-1-(4-methoxyphenyl)ethylamine (10.51 g, 69.5 mmol) and 6,7-dihydro-8(5H)-quinolinone (10.13 g, 68.8 mmol, J. Org. Chem., 2002, 67, 2197-2205) in 1,2-dichloroethane was treated with glacial acetic acid (5.9 mL, 103 mmol) and sodium triacetoxyborohydride (21.9 g, 103 mmol). The reaction mixture was stirred at room temperature for 18 hours and then treated with 10% aqueous sodium carbonate. The resulting mixture was extracted with dichloromethane (2×). The combined organic layers were washed with brine, dried over Na₂SO₄ and concentrated to dryness at reduced pressure. The crude product was purified by flash chromatography (silica gel, gradient elution of dichloromethane to 94:6 dichloromethane/2M ammonia in MeOH) followed by recrystallization from hexane to afford 11.69 g (60%) of (8R)—N-{(1R)-1-[4-(methyloxy)phenyl]ethyl}-5,6,7,8-tetrahydro-8-quinolinamine as a brown crystalline solid. ¹H-NMR (CDCl₃): δ 8.40 (d, 1H), 7.37 (m, 3H), 7.07 (m, 1H), 6.86 (m, 2H), 4.09 (br s, 1H), 3.85-3.80 (m, 4H), 2.78-2.63 (m, 3H), 1.88-1.48 (m, 7H). MS m/z 283 (M+1).

### b) (8R)—N-Methyl-N-{(1R)-1-[4-(methyloxy)phenyl]ethyl}-5,6,7,8-tetrahydro-8-quinolinamine

Reaction of (8R)—N-{(1R)-1-[4-(methyloxy)phenyl]ethyl}-5,6,7,8-tetrahydro-8-quinolinamine (1.09 g, 3.86 mmol) as described herein for the preparation of (8S)—N-methyl-N-{(1S)-1-[4-(methyloxy)phenyl]ethyl}-5,6,7,8-tetrahydro-8-quinolinamine afforded (8R)—N-methyl-N-{(1R)-1-[4-(methyloxy)phenyl]ethyl}-5,6,7,8-tetrahydro-8-quinolinamine in quantitative yield as a pale yellow oil. ¹H-NMR (CDCl₃): δ 8.46 (d, 1H), 7.40 (d, 2H), 7.29 (m, 1H), 7.00 (m, 1H), 6.83 (d, 2H), 4.43 (m, 1H), 3.98 (m, 1H), 3.78 (s, 3H), 2.78 (m, 1H), 2.61 (m, 1H), 2.01-1.85 (m, 6H), 1.56 (m, 1H), 1.37 (d, 3H).

### c) (8R)—N-Methyl-5,6,7,8-tetrahydro-8-quinolinamine

Reaction of (8R)—N-methyl-N-{(1R)-1-[4-(methyloxy)phenyl]ethyl}-5,6,7,8-tetrahydro-8-quinolinamine (1.14 g, 3.85 mmol) as described herein for the preparation of (8S)—N-methyl-N-({1-[(3S)-3-piperidinylmethyl]-1H-benzimidazol-2-yl}methyl)-5,6,7,8-tetrahydro-8-quinolinamine afforded 0.60 g (97%) of (8R)—N-methyl-5,6,7,8-tetrahydro-8-quinolinamine as a yellow oil. ¹H NMR (CDCl₃): δ 8.39 (d, 1H), 7.38 (m, 1H), 7.08 (m, 1H), 3.74-3.64 (m, 2H), 2.86-2.71 (m, 2H), 2.56 (s, 3H), 2.16 (m, 1H), 2.00 (m, 1H), 1.85-1.72 (m, 2H).

### d) 1,1-Dimethylethyl (3R)-3-{[2-({methyl[(8R)-5,6,7,8-tetrahydro-8-quinolinyl]amino}methyl)-1H-benzimidazol-1-yl]methyl}-1-piperidinecarboxylate

Reaction of 1,1-dimethylethyl (3R)-3-{[2-(chloromethyl)-1H-benzimidazol-1-yl]methyl}-1-piperidinecarboxylate (330 mg, 0.91 mmol) and (8R)—N-methyl-5,6,7,8-tetrahydro-8-quinolinamine (0.16 g, 1.00 mmol) as described herein for the preparation of 1,1-dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate afforded 0.31 g (70%) of 1,1-dimethylethyl (3R)-3-{[2-({methyl[(8R)-5,6,7,8-tetrahydro-8-quinolinyl]amino}methyl)-1H-benzimidazol-1-yl]methyl}-1-piperidinecarboxylate as a white solid. ¹H NMR (DMSO-d₆): δ 8.49 (d, 1H), 7.58 (m, 3H), 7.21 (m, 3H), 4.25 (m, 3H), 4.07-3.80 (m, 3H), 3.57 (br m, 1H), 2.76 (m, 3H), 2.45 (m, 1H), 2.13 (m, 4H), 2.01 (m, 3H), 1.64 (m, 3H), 1.21 (m, 11H). MS m/z 490 (M+1).

### e) (8R)—N-Methyl-N-({1-[(3S)-3-piperidinylmethyl]-1H-benzimidazol-2-yl}methyl)-5,6,7,8-tetrahydro-8-quinolinamine

Deprotection of 1,1-dimethylethyl (3R)-3-{[2-({methyl[(8R)-5,6,7,8-tetrahydro-8-quinolinyl]amino}methyl)-1H-benzimidazol-1-yl]methyl}-1-piperidinecarboxylate (0.15 g, 0.31 mmol) as described herein for the preparation of (8S)—N-methyl-N-({1-[(3S)-3-piperidinylmethyl]-1H-benzimidazol-2-yl}methyl)-5,6,7,8-tetrahydro-8-quinolinamine afforded (8R)—N-methyl-N-({1-[(3S)-3-piperidinylmethyl]-1H-benzimidazol-2-yl}methyl)-5,6,7,8-tetrahydro-8-quinolinamine as an off-white foam in quantitative yield. ¹H NMR (CD₃OD): δ 8.56 (d, 1H), 7.66-7.50 (m, 3H), 7.30 (m, 3H), 4.53 (m, 1H), 4.31 (m, 1H), 4.16-3.85 (m, 3H), 3.12 (m, 1H), 3.04-2.82 (m, 4H), 2.67 (m, 1H), 2.47 (m, 1H), 2.34 (s, 3H), 2.18 (m, 3H), 1.93-1.34 (m, 5H). MS m/z 390 (M+1).

### f) (8R)—N-Methyl-N-[(1-{[(3S)-1-methyl-3-piperidinyl]methyl}-1H-benzimidazol-2-yl)methyl]-5,6,7,8-tetrahydro-8-quinolinamine

Reductive methylation of (8R)—N-methyl-N-({1-[(3S)-3-piperidinylmethyl]-1H-benzimidazol-2-yl}methyl)-5,6,7,8-tetrahydro-8-quinolinamine (92 mg, 0.24 mmol) as described herein for the preparation of (8S)—N-methyl-N-[(1-{[(3S)-1-methyl-3-piperidinyl]methyl}-1H-benzimidazol-2-yl)methyl]-5,6,7,8-tetrahydro-8-quinolinamine afforded 81 mg (85%) of (8R)—N-methyl-N-[(1-{[(3S)-1-methyl-3-piperidinyl]methyl}-1H-benzimidazol-2-yl)methyl]-5,6,7,8-tetrahydro-8-quinolinamine as a colorless oil. ¹H NMR (CD₃OD): δ 8.43 (d, 1H), 7.55 (m, 2H), 7.45 (m, 1H), 7.23 (m, 3H), 4.26 (d, 2H), 4.08-3.84 (m, 3H), 2.96-2.73 (m, 3H), 2.48 (m, 1H), 2.24-2.00 (m, 10H), 1.91-1.44 (m, 6H), 1.27 (m, 1H). MS m/z 404 (M+1).

### Example 77

### (8R)—N-Methyl-N-[(1-{[(3R)-1-methyl-3-piperidinyl]methyl}-1H-benzimidazol-2-yl)methyl]-5,6,7,8-tetrahydro-8-quinolinamine

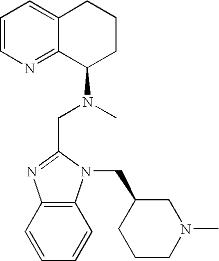

### a) 1,1-Dimethylethyl (3S)-3-{[2-({methyl[(8R)-5,6,7,8-tetrahydro-8-quinolinyl]amino}methyl)-1H-benzimidazol-1-yl]methyl}-1-piperidinecarboxylate

Reaction of 1,1-dimethylethyl (3S)-3-{[2-(chloromethyl)-1H-benzimidazol-1-yl]methyl}-1-piperidinecarboxylate (316 mg, 0.87 mmol) and (8R)—N-methyl-5,6,7,8-tetrahydro-8-quinolinamine (0.14 g, 0.87 mmol) as described herein for the preparation of 1,1-dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate afforded 0.36 g (85%) of 1,1-dimethylethyl (3S)-3-{[2-({methyl[(8R)-5,6,7,8-tetrahydro-8-quinolinyl]amino}methyl)-1H-benzimidazol-1-yl]methyl}-1-piperidinecarboxylate as an off-white solid. ¹H NMR (DMSO-d₆): δ 8.49 (d, 1H), 7.58 (m, 3H), 7.21 (m, 3H), 4.37-3.80 (m, 6H), 3.54 (br m, 1H), 2.76 (m, 3H), 2.54 (m, 1H), 2.05-1.97 (m, 7H), 1.60 (m, 3H), 1.27 (m, 11H). MS m/z 490 (M+1).

### b) (8R)—N-Methyl-N-({1-[(3R)-3-piperidinylmethyl]-1H-benzimidazol-2-yl}methyl)-5,6,7,8-tetrahydro-8-quinolinamine

Deprotection of 1,1-dimethylethyl (3S)-3-{[2-({methyl[(8R)-5,6,7,8-tetrahydro-8-quinolinyl]amino}methyl)-1H-benzimidazol-1-yl]methyl}-1-piperidinecarboxylate (0.17 g, 0.35 mmol) as described herein for the preparation of (8S)—N-methyl-N-({1-[(3S)-3-piperidinylmethyl]-1H-benzimidazol-2-yl}methyl)-5,6,7,8-tetrahydro-8-quinolinamine afforded (8R)—N-methyl-N-({1-[(3R)-3-piperidinylmethyl]-1H-benzimidazol-2-yl}methyl)-5,6,7,8-tetrahydro-8-quinolinamine as an off-white foam in quantitative yield. ¹H NMR (CD₃OD): δ 8.43 (d, 1H), 7.55 (m, 2H), 7.47 (m, 1H), 7.23 (m, 3H), 4.27 (m, 2H), 4.12-3.86 (m, 3H), 3.00-2.68 (m, 4H), 2.54 (m, 1H), 2.30-2.04 (m, 8H), 1.69 (m, 3H), 1.42 (m, 1H), 1.11 (m, 1H). MS m/z 390 (M+1).

### c) (8R)—N-Methyl-N-[(1-{[(3R)-1-methyl-3-piperidinyl]methyl}-1H-benzimidazol-2-yl)methyl]-5,6,7,8-tetrahydro-8-quinolinamine

Reductive methylation of (8R)—N-methyl-N-({1-[(3R)-3-piperidinylmethyl]-1H-benzimidazol-2-yl}methyl)-5,6,7,8-tetrahydro-8-quinolinamine (126 mg, 0.32 mmol) as described herein for the preparation of (8S)—N-methyl-N-[(1-{[(3S)-1-methyl-3-piperidinyl]methyl}-1H-benzimidazol-2-yl)methyl]-5,6,7,8-tetrahydro-8-quinolinamine afforded 107 mg (82%) of (8R)—N-methyl-N-[(1-{[(3R)-1-methyl-3-piperidinyl]methyl}-1H-benzimidazol-2-yl)methyl]-5,6,7,8-tetrahydro-8-quinolinamine as a colorless oil. ¹H NMR (CD₃OD): δ 8.43 (d, 1H), 7.55 (t, 2H), 7.45 (m, 1H), 7.23 (m, 3H), 4.26 (m, 2H), 4.10-3.85 (m, 3H), 2.96-2.71 (m, 3H), 2.41 (m, 1H), 2.24-2.00 (m, 10H), 1.92-1.43 (m, 6H), 1.27 (m, 1H). MS m/z 404 (M+1).

### Example 78

### (8R)—N-Methyl-N-[(1-{[(3R)-1-(3-methylbutyl)-3-piperidinyl]methyl}-1H-benzimidazol-2-yl)methyl]-5,6,7,8-tetrahydro-8-quinolinamine

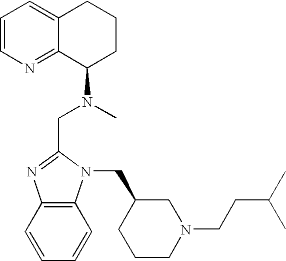

A solution of (8R)—N-methyl-N-({1-[(3R)-3-piperidinylmethyl]-1H-benzimidazol-2-yl}methyl)-5,6,7,8-tetrahydro-8-quinolinamine (44 mg, 0.11 mmol) in 1,2-dichloroethane (3 mL) was treated with isovaleraldehyde (18 μL, 0.17 mmol), NaBH(OAc)₃ (48 mg, 0.22 mmol) and glacial acetic acid (19 μL, 0.34 mmol). After 4 h, the reaction was diluted with dichloromethane, 10% aqueous Na₂CO₃ and brine and shaken well. The mixture was filtered through a hydrophobic frit. The aqueous layer was washed with dichloromethane and filtered. The combined organic layers were concentrated under reduced pressure. The crude product was purified by flash chromatography (silica gel, gradient elution of acetonitrile to 9:1 acetonitrile/NH₄OH) to afford 38 mg (74%) of (8R)—N-methyl-N-[(1-{[(3R)-1-(3-methylbutyl)-3-piperidinyl]methyl}-1H-benzimidazol-2-yl)methyl]-5,6,7,8-tetrahydro-8-quinolinamine as a colorless oil. ¹H NMR (CD₃OD): δ 8.44 (d, 1H), 7.56 (m, 2H), 7.46 (m, 1H), 7.23 (m, 3H), 4.33-4.19 (m, 2H), 4.11-3.87 (m, 3H), 2.92 (m, 1H), 2.77 (m, 2H), 2.49 (m, 1H), 2.29-2.00 (m, 9H), 1.90 (t, 1H), 1.76 (m, 1H), 1.64 (m, 1H), 1.57-1.39 (m, 4H), 1.24 (m, 2H), 0.94-0.83 (m, 7H). MS m/z 460 (M+1).

### Example 79

(8R)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine.

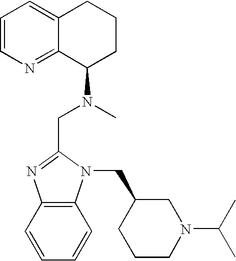

A solution of (8R)—N-methyl-N-({1-[(3R)-3-piperidinylmethyl]-1H-benzimidazol-2-yl}methyl)-5,6,7,8-tetrahydro-8-quinolinamine (44 mg, 0.11 mmol) in 1,2-dichloroethane (3 mL) was treated with acetone (11 μL, 0.17 mmol), NaBH(OAc)₃ (48 mg, 0.22 mmol) and glacial acetic acid (19 μL, 0.34 mmol). After 24 h, additional acetone (50 μL) and NaBH(OAc)₃ (100 mg) were added. After an additional 24 h, the reaction was diluted with dichloromethane, 10% aqueous Na₂CO₃ and brine and shaken well. The mixture was filtered through a hydrophobic frit. The aqueous layer was washed with dichloromethane and filtered. The combined organic layers were concentrated under reduced pressure. The crude product was purified by flash chromatography (silica gel, gradient elution of acetonitrile to 9:1 acetonitrile/NH₄OH) to afford 39 mg (80%) of (8R)—N-methyl-N-[(1-{[(3R)-1-(1-methylethyl)-3-piperidinyl]methyl}-1H-benzimidazol-2-yl)methyl]-5,6,7,8-tetrahydro-8-quinolinamine as a pale yellow oil. ¹H NMR (CD₃OD): δ8.44 (m, 1H), 7.55 (m, 2H), 7.45 (m, 1H), 7.22 (m, 3H), 4.31-4.17 (m, 2H), 4.09-3.83 (m, 3H), 2.90 (m, 1H), 2.77 (m, 2H), 2.56 (m, 1H), 2.43 (m, 1H), 2.24-1.99 (m, 9H), 1.77-1.62 (m, 3H), 1.51-1.38 (m, 2H), 0.89 (m, 6H). MS m/z 432 (M+1).

### Example 80

### (8S)—N-Methyl-N-[(1-{[(3R)-1-(3-methylbutyl)-3-piperidinyl]methyl}-1H-benzimidazol-2-yl)methyl]-5,6,7,8-tetrahydro-8-quinolinamine

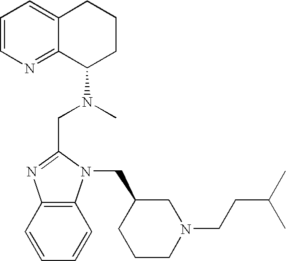

Reaction of (8S)—N-methyl-N-({1-[(3R)-3-piperidinylmethyl]-1H-benzimidazol-2-yl}methyl)-5,6,7,8-tetrahydro-8-quinolinamine (40 mg, 0.10 mmol) as described herein for the preparation of (8R)—N-methyl-N-[(1-{[(3R)-1-(3-methylbutyl)-3-piperidinyl]methyl}-1H-benzimidazol-2-yl)methyl]-5,6,7,8-tetrahydro-8-quinolinamine afforded 33 mg (70%) of (8S)—N-methyl-N-[(1-{[(3R)-1-(3-methylbutyl)-3-piperidinyl]methyl}-1H-benzimidazol-2-yl)methyl]-5,6,7,8-tetrahydro-8-quinolinamine as a colorless oil. ¹H NMR (CD₃OD): δ 8.42 (d, 1H), 7.56 (m, 2H), 7.46 (m, 1H), 7.24 (m, 3H), 4.27 (m, 2H), 4.11-3.86 (m, 3H), 2.92 (m, 1H), 2.78 (m, 2H), 2.56 (m, 1H), 2.30-2.01 (m, 9H), 1.90 (m, 1H), 1.76 (m, 1H), 1.65-1.42 (m, 5H), 1.26 (m, 2H), 0.85 (m, 7H). MS m/z 460 (M+1).

### Example 81

### (8S)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine

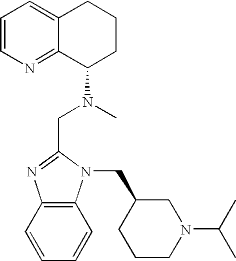

Reaction of (8S)—N-methyl-N-({1-[(3R)-3-piperidinylmethyl]-1H-benzimidazol-2-yl}methyl)-5,6,7,8-tetrahydro-8-quinolinamine (40 mg, 0.10 mmol) as described herein for the preparation of (8R)—N-methyl-N-[(1-{[(3R)-1-(1-methylethyl)-3-piperidinyl]methyl}-1H-benzimidazol-2-yl)methyl]-5,6,7,8-tetrahydro-8-quinolinamine afforded 33 mg (75%) of (8S)—N-methyl-N-[(1-{[(3R)-1-(1-methylethyl)-3-piperidinyl]methyl}-1H-benzimidazol-2-yl)methyl]-5,6,7,8-tetrahydro-8-quinolinamine as a pale yellow oil. ¹H NMR (CD₃OD): δ 8.42 (d, 1H), 7.55 (m, 2H), 7.46 (m, 1H), 7.23 (m, 3H), 4.24 (m, 2H), 4.09-3.85 (m, 3H), 2.89 (m, 1H), 2.77 (m, 2H), 2.57 (m, 2H), 2.24-2.01 (m, 9H), 1.82-1.38 (m, 5H), 0.95 (m, 6H). MS m/z 432 (M+1).

### Example 82

### (8R)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine

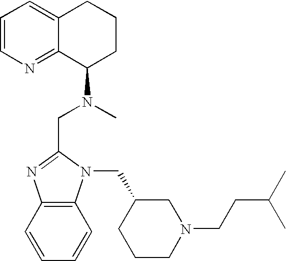

Reaction of (8R)—N-methyl-N-({1-[(3S)-3-piperidinylmethyl]-1H-benzimidazol-2-yl}methyl)-5,6,7,8-tetrahydro-8-quinolinamine (40 mg, 0.10 mmol) as described herein for the preparation of (8R)—N-methyl-N-[(1-{[(3R)-1-(3-methylbutyl)-3-piperidinyl]methyl}-1H-benzimidazol-2-yl)methyl]-5,6,7,8-tetrahydro-8-quinolinamine afforded 33 mg (70%) of (8R)—N-methyl-N-[(1-{[(3S)-1-(3-methylbutyl)-3-piperidinyl]methyl}-1H-benzimidazol-2-yl)methyl]-5,6,7,8-tetrahydro-8-quinolinamine as a colorless oil. ¹H NMR (CD₃OD): δ 8.43 (d, 1H), 7.56 (m, 2H), 7.46 (m, 1H), 7.23 (m, 3H), 4.27 (m, 2H), 4.11-3.86 (m, 3H), 2.91 (m, 1H), 2.80 (m, 2H), 2.55 (m, 1H), 2.27-2.02 (m, 9H), 1.90 (m, 1H), 1.76 (m, 1H), 1.65-1.40 (m, 5H), 1.25 (m, 2H), 0.85 (m, 7H). MS m/z 460 (M+1).

### Example 83

### (8R)—N-Methyl-N-[1-{[(3S)-1-(1-methylethyl)-3-piperidinyl]methyl}-1H-benzimidazol-2-yl)methyl]-5,6,7,8-tetrahydro-8-quinolinamine

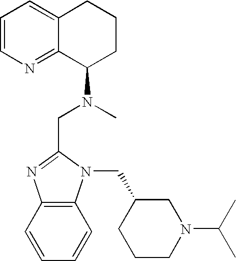

Reaction of (8R)—N-methyl-N-({1-[(3S)-3-piperidinylmethyl]-1H-benzimidazol-2-yl}methyl)-5,6,7,8-tetrahydro-8-quinolinamine (40 mg, 0.10 mmol) as described herein for the preparation of (8R)—N-methyl-N-[(1-{[(3R)-1-(1-methylethyl)-3-piperidinyl]methyl}-1H-benzimidazol-2-yl)methyl]-5,6,7,8-tetrahydro-8-quinolinamine afforded 40 mg (91%) of (8R)—N-methyl-N-[(1-{[(3S)-1-(1-methylethyl)-3-piperidinyl]methyl}-1H-benzimidazol-2-yl)methyl]-5,6,7,8-tetrahydro-8-quinolinamine as a pale yellow oil. ¹H NMR (CD₃OD): δ 8.44 (d, 1H), 7.58-7.47 (m, 3H), 7.23 (m, 3H), 4.30 (m, 2H), 4.06-3.85 (m, 3H), 3.02-2.74 (m, 5H), 2.42-2.05 (m, 10H), 1.76 (m, 2H), 1.56 (m, 2H), 1.08 (m, 6H). MS m/z 432 (M+1).

### Example 84

### (8S)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine

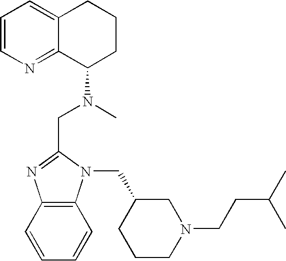

Reaction of (8S)—N-methyl-N-({1-[(3S)-3-piperidinylmethyl]-1H-benzimidazol-2-yl}methyl)-5,6,7,8-tetrahydro-8-quinolinamine (44 mg, 0.11 mmol) as described herein for the preparation of (8R)—N-methyl-N-[(1-{[(3R)-1-(3-methylbutyl)-3-piperidinyl]methyl}-1H-benzimidazol-2-yl)methyl]-5,6,7,8-tetrahydro-8-quinolinamine afforded 43 mg (83%) of (8S)—N-methyl-N-[(1-{[(3S)-1-(3-methylbutyl)-3-piperidinyl]methyl}-1H-benzimidazol-2-yl)methyl]-5,6,7,8-tetrahydro-8-quinolinamine as a colorless oil. ¹H NMR (CD₃OD): δ 8.44 (d, 1H), 7.56 (m, 2H), 7.46 (m, 1H), 7.23 (m, 3H), 4.26 (m, 2H), 4.11-3.87 (m, 3H), 2.91 (m, 1H), 2.79 (m, 2H), 2.49 (m, 1H), 2.25-2.02 (m, 9H), 1.89 (m, 1H), 1.75 (m, 1H), 1.65 (m, 1H), 1.57-1.39 (m, 4H), 1.23 (m, 2H), 0.94-0.83 (m, 7H). MS m/z 460 (M+1).

### Example 85

### (8S)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine

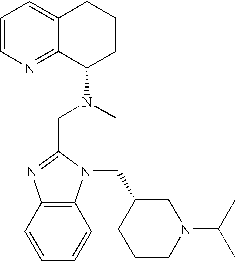

Reaction of (8S)—N-methyl-N-({1-[(3S)-3-piperidinylmethyl]-1H-benzimidazol-2-yl}methyl)-5,6,7,8-tetrahydro-8-quinolinamine (44 mg, 0.11 mmol) as described herein for the preparation of (8R)—N-methyl-N-[(1-{[(3R)-1-(1-methylethyl)-3-piperidinyl]methyl}-1H-benzimidazol-2-yl)methyl]-5,6,7,8-tetrahydro-8-quinolinamine afforded 39 mg (80%) of (8S)—N-methyl-N-[(1-{[(3S)-1-(1-methylethyl)-3-piperidinyl]methyl}-1H-benzimidazol-2-yl)methyl]-5,6,7,8-tetrahydro-8-quinolinamine as a pale yellow oil. ¹H NMR (CD₃OD): δ 8.44 (d, 1H), 7.56 (m, 2H), 7.45 (m, 1H), 7.24 (m, 3H), 4.24 (m, 2H), 4.09-3.83 (m, 3H), 2.89 (m, 1H), 2.75 (m, 2H), 2.58-2.42 (m, 2H), 2.29-2.16 (m, 5H), 2.06 (m, 4H), 1.75-1.63 (m, 3H), 1.52-1.39 (m, 2H), 0.95-0.85 (m, 6H). MS m/z 432 (M+1).

### Example 86

### (3S)-3-{[2-({Methyl[(8R)-5,6,7,8-tetrahydro-8-quinolinyl]amino}methyl)-1H-benzimidazol-1-yl]methyl}-1-piperidinecarboximidamide

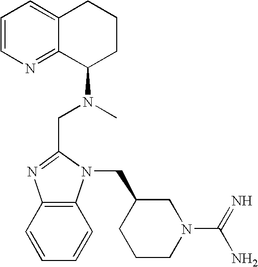

### a) Bis(1,1-dimethylethyl) [(E)-((3S)-3-{[2-({methyl[(8R)-5,6,7,8-tetrahydro-8-quinolinyl]amino}methyl)-1H-benzimidazol-1-yl]methyl}-1-piperidinyl)methylylidene]biscarbamate

Reaction of (8R)—N-methyl-N-({1-[(3R)-3-piperidinylmethyl]-1H-benzimidazol-2-yl}methyl)-5,6,7,8-tetrahydro-8-quinolinamine (47 mg, 0.12 mmol) as described herein for the preparation of bis(1,1-dimethylethyl) ((E)-{[3-(2-{[methyl(5,6,7,8-tetrahydro-8-quinolinyl)amino]methyl}-1H-benzimidazol-1-yl)propyl]amino}methylylidene)biscarbamate afforded 54 mg (71%) of bis(1,1-dimethylethyl) [(E)-((3S)-3-{[2-({methyl[(8R)-5,6,7,8-tetrahydro-8-quinolinyl]amino}methyl)-1H-benzimidazol-1-yl]methyl}-1-piperidinyl)methylylidene]biscarbamate as a colorless oil. ¹H NMR (CD₃OD): δ 8.43 (d, 1H), 7.53 (m, 3H), 7.23 (m, 3H), 4.28-3.76 (m, 7H), 2.94-2.75 (m, 3H), 2.58 (t, 1H), 2.24-2.06 (m, 7H), 1.76-1.58 (m, 3H), 1.37 (m, 19H), 1.16 (m, 2H). MS m/z 632 (M+1).

### b) (3S)-3-{[2-({Methyl[(8R)-5,6,7,8-tetrahydro-8-quinolinyl]amino}methyl)-1H-benzimidazol-1-yl]methyl}-1-piperidinecarboximidamide

Deprotection of bis(1,1-dimethylethyl) [(E)-((3S)-3-{[2-({methyl[(8R)-5,6,7,8-tetrahydro-8-quinolinyl]amino}methyl)-1H-benzimidazol-1-yl]methyl}-1-piperidinyl)methylylidene]biscarbamate (44 mg, 0.07 mmol) as described herein for the preparation of N-{[1-(3-azetidinylmethyl)-1H-benzimidazol-2-yl]methyl}-N-methyl-5,6,7,8-tetrahydro-8-quinolinamine, afforded (3S)-3-{[2-({methyl[(8R)-5,6,7,8-tetrahydro-8-quinolinyl]amino}methyl)-1H-benzimidazol-1-yl]methyl}-1-piperidinecarboximidamide as an off-white foam in quantitative yield. ¹H NMR (CD₃OD): δ 8.45 (d, 1H), 7.64 (m, 1H), 7.56 (m, 2H), 7.36-7.20 (m, 3H), 4.40 (m, 2H), 4.27-4.03 (m, 3H), 3.81 (m, 2H), 3.11-2.79 (m, 4H), 2.38-2.12 (m, 7H), 1.86-1.34 (m, 8H). MS m/z 432 (M+1).

### Example 87

### (3S)-3-{[2-({Methyl[(8S)-5,6,7,8-tetrahydro-8-quinolinyl]amino}methyl)-1H-benzimidazol-1-yl]methyl}-1-piperidinecarboximidamide

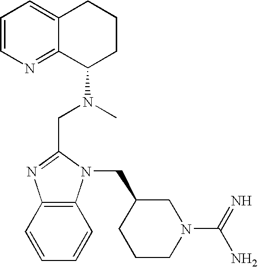

### a) Bis(1,1-dimethylethyl) [(E)-((3S)-3-{[2-({methyl[(8S)-5,6,7,8-tetrahydro-8-quinolinyl]amino}methyl)-1H-benzimidazol-1-yl]methyl}-1-piperidinyl)methylylidene]biscarbamate

Reaction of (8S)—N-methyl-N-({1-[(3R)-3-piperidinylmethyl]-1H-benzimidazol-2-yl}methyl)-5,6,7,8-tetrahydro-8-quinolinamine (42 mg, 0.14 mmol) as described herein for the preparation of bis(1,1-dimethylethyl)((E)-{[3-(2-{[methyl(5,6,7,8-tetrahydro-8-quinolinyl)amino]methyl}-1H-benzimidazol-1-yl)propyl]amino}methylylidene)biscarbamate afforded 62 mg (73%) of bis(1,1-dimethylethyl) [(E)-((3S)-3-{[2-({methyl[(8S)-5,6,7,8-tetrahydro-8-quinolinyl]amino}methyl)-1H-benzimidazol-1-yl]methyl}-1-piperidinyl)methylylidene]biscarbamate as a colorless oil. ¹H NMR (CD₃OD): δ 8.45 (d, 1H), 7.54 (m, 3H), 7.23 (m, 3H), 4.29 (m, 2H), 4.12-3.79 (m, 5H), 2.88-2.66 (m, 4H), 2.24-2.07 (m, 7H), 1.75-1.53 (m, 3H), 1.38 (m, 19H), 1.11 (m, 2H). MS m/z 632 (M+1).

### b) (3S)-3-{[2-({Methyl[(8S)-5,6,7,8-tetrahydro-8-quinolinyl]amino}methyl)-1H-benzimidazol-1-yl]methyl}-1-piperidinecarboximidamide

Deprotection of bis(1,1-dimethylethyl) [(E)-((3S)-3-{[2-({methyl[(8S)-5,6,7,8-tetrahydro-8-quinolinyl]amino}methyl)-1H-benzimidazol-1-yl]methyl}-1-piperidinyl)methylylidene]biscarbamate (51 mg, 0.08 mmol) as described herein for the preparation of N-{[1-(3-azetidinylmethyl)-1H-benzimidazol-2-yl]methyl}-N-methyl-5,6,7,8-tetrahydro-8-quinolinamine, afforded (3S)-3-{[2-({methyl[(8S)-5,6,7,8-tetrahydro-8-quinolinyl]amino}methyl)-1H-benzimidazol-1-yl]methyl}-1-piperidinecarboximidamide as an off-white solid in quantitative yield. ¹H NMR (CD₃OD): δ 8.51 (d, 1H), 7.68-7.54 (m, 3H), 7.36-7.24 (m, 3H), 4.42-3.84 (m, 7H), 3.14-2.79 (m, 4H), 2.46-2.12 (m, 7H), 1.86-1.26 (m, 8H). MS m/z 432 (M+1).

### Example 88

### (3R)-3-{[2-({Methyl[(8R)-5,6,7,8-tetrahydro-8-quinolinyl]amino}methyl)-1H-benzimidazol-1-yl]methyl}-1-piperidinecarboximidamide

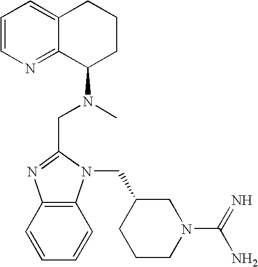

### a) Bis(1,1-dimethylethyl) [(E)-((3R)-3-{[2-({methyl[(8R)-5,6,7,8-tetrahydro-8-quinolinyl]amino}methyl)-1H-benzimidazol-1-yl]methyl}-1-piperidinyl)methylylidene]biscarbamate

Reaction of (8R)—N-methyl-N-({1-[(3S)-3-piperidinylmethyl]-1H-benzimidazol-2-yl}methyl)-5,6,7,8-tetrahydro-8-quinolinamine (43 mg, 0.14 mmol) as described herein for the preparation of bis(1,1-dimethylethyl)((E)-{[3-(2-{[methyl(5,6,7,8-tetrahydro-8-quinolinyl)amino]methyl}-1H-benzimidazol-1-yl)propyl]amino}methylylidene)biscarbamate afforded 71 mg (82%) of bis(1,1-dimethylethyl) [(E)-((3R)-3-{[2-({methyl[(8R)-5,6,7,8-tetrahydro-8-quinolinyl]amino}methyl)-1H-benzimidazol-1-yl]methyl}-1-piperidinyl)methylylidene]biscarbamate as a colorless oil. ¹H NMR (CD₃OD): δ 8.45 (d, 1H), 7.54 (m, 3H), 7.23 (m, 3H), 4.29 (m, 2H), 4.13-3.79 (m, 5H), 2.89-2.66 (m, 4H), 2.24-2.01 (m, 7H), 1.75-1.54 (m, 3H), 1.38 (m, 19H), 1.11 (m, 2H). MS m/z 632 (M+1).

### b) (3R)-3-{[2-({Methyl[(8R)-5,6,7,8-tetrahydro-8-quinolinyl]amino}methyl)-1H-benzimidazol-1-yl]methyl}-1-piperidinecarboximidamide

Deprotection of bis(1,1-dimethylethyl) [(E)-((3R)-3-{[2-({methyl[(8R)-5,6,7,8-tetrahydro-8-quinolinyl]amino}methyl)-1H-benzimidazol-1-yl]methyl}-1-piperidinyl)methylylidene]biscarbamate (54 mg, 0.085 mmol) as described herein for the preparation of N-{[1-(3-azetidinylmethyl)-1H-benzimidazol-2-yl]methyl}-N-methyl-5,6,7,8-tetrahydro-8-quinolinamine, afforded (3R)-3-{[2-({methyl[(8R)-5,6,7,8-tetrahydro-8-quinolinyl]amino}methyl)-1H-benzimidazol-1-yl]methyl}-1-piperidinecarboximidamide as an off-white solid in quantitative yield. ¹H NMR (CD₃OD): δ 8.51 (d, 1H), 7.65-7.54 (m, 3H), 7.36-7.24 (m, 3H), 4.40-3.84 (m, 7H), 3.14-2.79 (m, 4H), 2.45-2.12 (m, 7H), 1.83-1.29 (m, 8H). MS m/z 432 (M+1).

### Example 89

### (3R)-3-{[2-({Methyl[(8S)-5,6,7,8-tetrahydro-8-quinolinyl]amino}methyl)-1H-benzimidazol-1-yl]methyl}-1-piperidinecarboximidamide

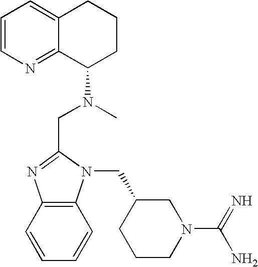

### a) Bis(1,1-dimethylethyl) [(E)-((3R)-3-{[2-({methyl[(8S)-5,6,7,8-tetrahydro-8-quinolinyl]amino}methyl)-1H-benzimidazol-1-yl]methyl}-1-piperidinyl)methylylidene]biscarbamate

Reaction of (8S)—N-methyl-N-({1-[(3S)-3-piperidinylmethyl]-1H-benzimidazol-2-yl}methyl)-5,6,7,8-tetrahydro-8-quinolinamine (56 mg, 0.18 mmol) as described herein for the preparation of bis(1,1-dimethylethyl)((E)-{[3-(2-{[methyl(5,6,7,8-tetrahydro-8-quinolinyl)amino]methyl}-1H-benzimidazol-1-yl)propyl]amino}methylylidene)biscarbamate afforded 92 mg (80%) of bis(1,1-dimethylethyl) [(E)-((3R)-3-{[2-({methyl[(8S)-5,6,7,8-tetrahydro-8-quinolinyl]amino}methyl)-1H-benzimidazol-1-yl]methyl}-1-piperidinyl)methylylidene]biscarbamate as a white foam. ¹H NMR (CD₃OD): δ 8.43 (d, 1H), 7.53 (m, 3H), 7.23 (m, 3H), 4.27 (m, 2H), 4.17-3.77 (m, 5H), 2.95-2.75 (m, 3H), 2.58 (m, 1H), 2.24-2.07 (m, 7H), 1.76-1.61 (m, 3H), 1.37 (m, 19H), 1.16 (m, 2H). MS m/z 632 (M+1).

### b) (3R)-3-{[2-({Methyl[(8S)-5,6,7,8-tetrahydro-8-quinolinyl]amino}methyl)-1H-benzimidazol-1-yl]methyl}-1-piperidinecarboximidamide

Deprotection of bis(1,1-dimethylethyl) [(E)-((3R)-3-{[2-({methyl[(8S)-5,6,7,8-tetrahydro-8-quinolinyl]amino}methyl)-1H-benzimidazol-1-yl]methyl}-1-piperidinyl)methylylidene]biscarbamate (90 mg, 0.014 mmol) as described herein for the preparation of N-{[1-(3-azetidinylmethyl)-1H-benzimidazol-2-yl]methyl}-N-methyl-5,6,7,8-tetrahydro-8-quinolinamine, afforded (3R)-3-{[2-({methyl[(8S)-5,6,7,8-tetrahydro-8-quinolinyl]amino}methyl)-1H-benzimidazol-1-yl]methyl}-1-piperidinecarboximidamide as an off-white solid in quantitative yield. ¹H NMR (CD₃OD): δ 8.44 (d, 1H), 7.65-7.54 (m, 3H), 7.36-7.20 (m, 3H), 4.40 (m, 2H), 4.23-3.98 (m, 3H), 3.81 (m, 2H), 3.11-2.79 (m, 4H), 2.38-2.11 (m, 8H), 1.86-1.29 (m, 7H). MS m/z 432 (M+1).

### Example 90

### (3R)—N-Cyano-3-{[2-({methyl[(8S)-5,6,7,8-tetrahydro-8-quinolinyl]amino}methyl)-1H-benzimidazol-1-yl]methyl}-1-propyl-1-piperidinecarboximidamide

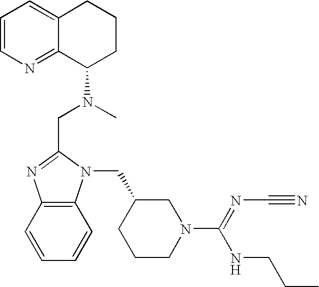

### a) Phenyl (3R)—N-cyano-3-{[2-({methyl[(8S)-5,6,7,8-tetrahydro-8-quinolinyl]amino}methyl)-1H-benzimidazol-1-yl]methyl}-1-piperidinecarboximidoate

A solution of (8S)—N-methyl-N-({1-[(3S)-3-piperidinylmethyl]-1H-benzimidazol-2-yl}methyl)-5,6,7,8-tetrahydro-8-quinolinamine (170 mg, 0.44 mmol) in isopropanol (10 mL) was treated with diphenyl cyanocarbonimidate (104 mg, 0.44 mmol). After stirring at RT overnight, the reaction mixture was concentrated. The crude product was purified by flash chromatography (silica gel, gradient elution of acetonitrile to 95:5 acetonitrile/NH₄OH) to afford 227 mg (97%) of phenyl (3R)—N-cyano-3-{[2-({methyl[(8S)-5,6,7,8-tetrahydro-8-quinolinyl]amino}methyl)-1H-benzimidazol-1-yl]methyl}-1-piperidinecarboximidoate as a colorless oil. ¹H NMR (CD₃OD): δ 8.42 (m, 1H), 7.58-7.15 (m, 10H), 6.72 (br m, 1H), 4.32-3.91 (m, 7H), 3.08-2.70 (m, 4H), 2.10 (m, 7H), 1.81-1.24 (m, 5H). MS m/z 534 (M+1).

### b) (3R)—N-Cyano-3-{[2-({methyl[(8S)-5,6,7,8-tetrahydro-8-quinolinyl]amino}methyl)-1H-benzimidazol-1-yl]methyl}-N′-propyl-1-piperidinecarboximidamide

A solution of phenyl (3R)—N-cyano-3-{[2-({methyl[(8S)-5,6,7,8-tetrahydro-8-quinolinyl]amino}methyl)-1H-benzimidazol-1-yl]methyl}-1-piperidinecarboximidoate (50 mg, 0.094 mmol) in isopropanol (3 mL) was treated with propylamine (0.15 mL) and heated in a sealed tube for 4 h. The reaction mixture was concentrated and purified by flash chromatography (silica gel, gradient elution of acetonitrile to 9:1 acetonitrile/NH₄OH) to afford 46 mg (98%) of (3R)—N-cyano-3-{[2-({methyl[(8S)-5,6,7,8-tetrahydro-8-quinolinyl]amino}methyl)-1H-benzimidazol-1-yl]methyl}-N′-propyl-1-piperidinecarboximidamide as an off-white foam. ¹H NMR (CD₃OD): δ 8.41 (d, 1H), 7.58-7.48 (m, 3H), 7.23 (m, 3H), 4.36-4.22 (m, 2H), 4.17-3.91 (m, 3H), 3.74 (m, 2H), 3.19 (m, 3H), 2.93 (m, 2H), 2.77 (m, 1H), 2.60 (m, 1H), 2.24-2.07 (m, 7H), 1.72 (m, 2H), 1.58 (m, 1H), 1.43 (m, 3H), 1.20 (m, 1H), 0.77 (t, 3H). MS m/z 499 (M+1).

### Example 91

### (3R)—N-Cyano-3-{[2-({methyl[(8S)-5,6,7,8-tetrahydro-8-quinolinyl]amino}methyl)-1H-benzimidazol-1-yl]methyl}-1-piperidinecarboximidamide

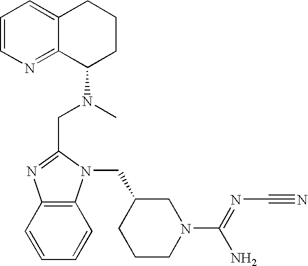

Reaction of phenyl (3R)—N-cyano-3-{[2-({methyl[(8S)-5,6,7,8-tetrahydro-8-quinolinyl]amino}methyl)-1H-benzimidazol-1-yl]methyl}-1-piperidinecarboximidoate (50 mg, 0.094 mmol) and ammonia (3.0 mL, 2.0 M in iPrOH) as described herein for the preparation of (3R)—N-cyano-3-{[2-({methyl[(8S)-5,6,7,8-tetrahydro-8-quinolinyl]amino}methyl)-1H-benzimidazol-1-yl]methyl}-N′-propyl-1-piperidinecarboximidamide except that no additional isopropanol was used as solvent afforded 38 mg (88%) of (3R)—N-cyano-3-{[2-({methyl[(8S)-5,6,7,8-tetrahydro-8-quinolinyl]amino}methyl)-1H-benzimidazol-1-yl]methyl}-1-piperidinecarboximidamide as an off-white solid. ¹H NMR (CD₃OD): δ 8.42 (d, 1H), 7.59-7.46 (m, 3H), 7.24 (m, 3H), 4.33-3.84 (m, 8H), 2.94-2.75 (m, 3H), 2.58 (m, 1H), 2.25-2.07 (m, 8H), 1.75 (m, 1H), 1.59 (m, 2H), 1.37 (m, 1H), 1.18 (m, 1H). MS m/z 457 (M+1).

### Example 92

### (3R)—N′-Cyano-N,N-dimethyl-3-{[2-({methyl[(8S)-5,6,7,8-tetrahydro-8-quinolinyl]amino}methyl)-1H-benzimidazol-1-yl]methyl}-1-piperidinecarboximidamide

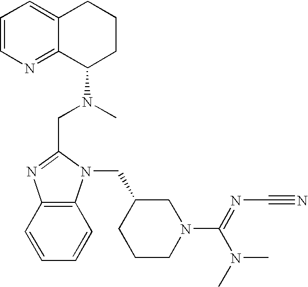

Reaction of phenyl (3R)—N-cyano-3-{[2-({methyl[(8S)-5,6,7,8-tetrahydro-8-quinolinyl]amino}methyl)-1H-benzimidazol-1-yl]methyl}-1-piperidinecarboximidoate (50 mg, 0.094 mmol) and dimethylamine (0.2 mL, 2.0 M in THF) as described herein for the preparation of (3R)—N-cyano-3-{[2-({methyl[(8S)-5,6,7,8-tetrahydro-8-quinolinyl]amino}methyl)-1H-benzimidazol-1-yl]methyl}-N′-propyl-1-piperidinecarboximidamide afforded 18 mg (40%) of (3R)—N-cyano-3-{[2-({methyl[(8S)-5,6,7,8-tetrahydro-8-quinolinyl]amino}methyl)-1H-benzimidazol-1-yl]methyl}-1-piperidinecarboximidamide as an off-white solid after repurification by reverse phase HPLC. ¹H NMR (CD₃OD): δ 8.42 (d, 1H), 7.59-7.49 (m, 3H), 7.23 (m, 3H), 4.29 (m, 2H), 4.16-3.90 (m, 3H), 3.62-3.29 (m, 3H), 2.96 (m, 2H), 2.77 (s, 6H), 2.67 (m, 1H), 2.23 (m, 5H), 2.06 (m, 2H), 1.77-1.43 (m, 4H), 1.15 (m, 1H). MS m/z 485 (M+1).

### Example 93

### (8S)—N-[(1-{[(3S)-1-Methyl-3-piperidinyl]methyl}-1H-benzimidazol-2-yl)methyl]-N-propyl-5,6,7,8-tetrahydro-8-quinolinamine

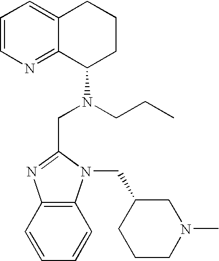

### a) (8S)—N-Propyl-5,6,7,8-tetrahydro-8-quinolinamine

Reaction of (8S)—N-{(1S)-1-[4-(methyloxy)phenyl]ethyl}-5,6,7,8-tetrahydro-8-quinolinamine (1.02 g, 3.6 mmol), and propionaldehyde (0.52 mL, 7.2 mmol) as described herein for the preparation of (8S)—N-methyl-N-{(1S)-1-[4-(methyloxy)phenyl]ethyl}-5,6,7,8-tetrahydro-8-quinolinamine afforded a yellow oil. Deprotection of the crude product as described herein for the preparation of (8R)—N-methyl-5,6,7,8-tetrahydro-8-quinolinamine afforded 0.53 g (77%) of (8S)—N-propyl-5,6,7,8-tetrahydro-8-quinolinamine as a straw-colored oil. ¹H-NMR (CDCl₃): δ 8.44 (d, 1H), 7.41 (d, 1H), 7.10 (m, 1H), 3.83 (m, 1H), 2.90-2.67 (m, 4H), 2.24-2.02 (m, 2H), 1.88-1.58 (m, 4H), 1.02 (t, 3H). MS m/z 191 (M+H).

### b) 1,1-Dimethylethyl (3R)-3-{[2-({propyl[(8S)-5,6,7,8-tetrahydro-8-quinolinyl]amino}methyl)-1H-benzimidazol-1-yl]methyl}-1-piperidinecarboxylate

Reaction of 1,1-dimethylethyl (3R)-3-{[2-(chloromethyl)-1H-benzimidazol-1-yl]methyl}-1-piperidinecarboxylate (0.61 g, 1.68 mmol) and (8S)—N-propyl-5,6,7,8-tetrahydro-8-quinolinamine (0.32 g, 1.68 mmol) as described herein for the preparation of 1,1-dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate afforded 0.55 g (63%) of 1,1-dimethylethyl (3R)-3-{[2-({propyl[(8S)-5,6,7,8-tetrahydro-8-quinolinyl]amino}methyl)-1H-benzimidazol-1-yl]methyl}-1-piperidinecarboxylate as an off-white foam. ¹H NMR (CD₃OD): δ 8.41 (d, 1H), 7.56 (m, 1H), 7.44 (m, 2H), 7.23 (m, 2H), 7.11 (m, 1H), 4.45-4.29 (m, 2H), 4.11 (s, 2H), 4.04 (m, 1H), 3.87 (m, 1H), 2.86 (m, 1H), 2.71 (m, 2H), 2.60-2.34 (m, 3H), 2.08 (m, 4H), 1.68 (m, 3H), 1.37-1.12 (m, 14H), 0.72 (t, 3H). MS m/z 518 (M+1).

### c) (8S)—N-({1-[(3S)-3-Piperidinylmethyl]-1H-benzimidazol-2-yl}methyl)-N-propyl-5,6,7,8-tetrahydro-8-quinolinamine

Deprotection of 1,1-dimethylethyl (3R)-3-{[2-({propyl[(8S)-5,6,7,8-tetrahydro-8-quinolinyl]amino}methyl)-1H-benzimidazol-1-yl]methyl}-1-piperidinecarboxylate (0.55 g, 1.06 mmol) as described herein for the preparation of (8S)—N-methyl-N-({1-[(3S)-3-piperidinylmethyl]-1H-benzimidazol-2-yl}methyl)-5,6,7,8-tetrahydro-8-quinolinamine afforded (8S)—N-({1-[(3S)-3-piperidinylmethyl]-1H-benzimidazol-2-yl}methyl)-N-propyl-5,6,7,8-tetrahydro-8-quinolinamine as an off-white foam in quantitative yield. ¹H NMR (CD₃OD): δ 8.40 (d, 1H), 7.55 (d, 1H), 7.44 (m, 2H), 7.23 (m, 2H), 7.12 (m, 1H), 4.34 (m, 2H), 4.10-4.03 (m, 3H), 2.95-2.84 (m, 2H), 2.75-2.43 (m, 5H), 2.11 (m, 5H), 1.68 (m, 3H), 1.39 (m, 3H), 1.15 (m, 1H), 0.74 (t, 3H). MS m/z 418 (M+1).

### d) (8S)—N-[(1-{[(3S)-1-Methyl-3-piperidinyl]methyl}-1H-benzimidazol-2-yl)methyl]-N-propyl-5,6,7,8-tetrahydro-8-quinolinamine

Reductive methylation of (8S)—N-({1-[(3S)-3-piperidinylmethyl]-1H-benzimidazol-2-yl}methyl)-N-propyl-5,6,7,8-tetrahydro-8-quinolinamine (50 mg, 0.12 mmol) as described herein for the preparation of (8S)—N-methyl-N-[(1-{[(3S)-1-methyl-3-piperidinyl]methyl}-1H-benzimidazol-2-yl)methyl]-5,6,7,8-tetrahydro-8-quinolinamine afforded 45 mg (88%) of (8S)—N-[(1-{[(3S)-1-methyl-3-piperidinyl]methyl}-1H-benzimidazol-2-yl)methyl]-N-propyl-5,6,7,8-tetrahydro-8-quinolinamine as a yellow oil. ¹H NMR (CD₃OD): δ 8.39 (d, 1H), 7.55 (m, 1H), 7.43 (m, 2H), 7.22 (m, 2H), 7.11 (m, 1H), 4.38 (m, 2H), 4.09-4.05 (m, 3H), 2.85 (m, 1H), 2.73 (m, 2H), 2.62-2.43 (m, 3H), 2.13-2.05 (m, 7H), 1.92 (t, 1H), 1.70-1.36 (m, 7H), 1.00 (m, 1H), 0.73 (t, 3H). MS m/z 432 (M+1).

### Example 94

### (8S)—N-[(1-{[(3S)-1-Methyl-3-piperidinyl]methyl}-1H-benzimidazol-2-yl)methyl]-N-(2-methylpropyl)-5,6,7,8-tetrahydro-8-quinolinamine

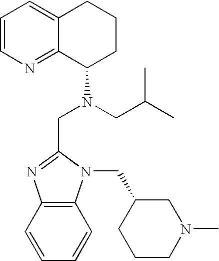

### a) (8S)—N-(2-Methylpropyl)-5,6,7,8-tetrahydro-8-quinolinamine

Reaction of (8S)—N-{(1S)-1-[4-(methyloxy)phenyl]ethyl}-5,6,7,8-tetrahydro-8-quinolinamine (1.06 g, 3.75 mmol), and isobutyraldehyde (0.68 mL, 7.5 mmol) as described herein for the preparation of (8S)—N-methyl-N-{(1S)-1-[4-(methyloxy)phenyl]ethyl}-5,6,7,8-tetrahydro-8-quinolinamine afforded a yellow oil. Deprotection of the crude product as described herein for the preparation of (8R)—N-methyl-5,6,7,8-tetrahydro-8-quinolinamine afforded 0.48 g (62%) of (8S)—N-(2-methylpropyl)-5,6,7,8-tetrahydro-8-quinolinamine as a straw-colored oil. ¹H-NMR (CDCl₃): δ 8.44 (d, 1H), 7.41 (d, 1H), 7.10 (m, 1H), 3.80 (m, 1H), 2.82 (m, 2H), 2.60 (d, 2H), 2.23-2.02 (m, 2H), 1.82 (m, 3H), 1.03 (d, 3H), 1.01 (d, 3H). MS m/z 205 (M+H).

### b) 1,1-Dimethylethyl (3R)-3-{[2-({(2-methylpropyl)[(8S)-5,6,7,8-tetrahydro-8-quinolinyl]amino}methyl)-1H-benzimidazol-1-yl]methyl}-1-piperidinecarboxylate

Reaction of 1,1-dimethylethyl (3R)-3-{[2-(chloromethyl)-1H-benzimidazol-1-yl]methyl}-1-piperidinecarboxylate (0.61 g, 1.66 mmol) and (8S)—N-(2-methylpropyl)-5,6,7,8-tetrahydro-8-quinolinamine (0.34 g, 1.66 mmol) as described herein for the preparation of 1,1-dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate afforded 0.51 g (58%) of 1,1-dimethylethyl (3R)-3-{[2-({(2-methylpropyl)[(8S)-5,6,7,8-tetrahydro-8-quinolinyl]amino}methyl)-1H-benzimidazol-1-yl]methyl}-1-piperidinecarboxylate as a white foam. ¹H NMR (CD₃OD): δ 8.45 (d, 1H), 7.58 (d, 1H), 7.47 (m, 2H), 7.25 (m, 2H), 7.14 (m, 1H), 4.58 (m, 1H), 4.35 (m, 1H), 4.16-4.02 (m, 3H), 3.85 (m, 1H), 2.87-2.68 (m, 3H), 2.41-2.29 (m, 3H), 2.12-2.01 (m, 4H), 1.80-1.67 (m, 3H), 1.42-1.10 (m, 13H), 0.71 (m, 6H). MS m/z 532 (M+1).

### c) (8S)—N-(2-Methylpropyl)-N-({1-[(3S)-3-piperidinylmethyl]-1H-benzimidazol-2-yl}methyl)-5,6,7,8-tetrahydro-8-quinolinamine

Deprotection of 1,1-dimethylethyl (3R)-3-{[2-({(2-methylpropyl)[(8S)-5,6,7,8-tetrahydro-8-quinolinyl]amino}methyl)-1H-benzimidazol-1-yl]methyl}-1-piperidinecarboxylate (0.51 g, 0.95 mmol) as described herein for the preparation of (8S)—N-methyl-N-({1-[(3S)-3-piperidinylmethyl]-1H-benzimidazol-2-yl}methyl)-5,6,7,8-tetrahydro-8-quinolinamine afforded (8S)—N-(2-methylpropyl)-N-({1-[(3S)-3-piperidinylmethyl]-1H-benzimidazol-2-yl}methyl)-5,6,7,8-tetrahydro-8-quinolinamine as a white foam in quantitative yield. ¹H NMR (CD₃OD): δ 8.45 (d, 1H), 7.57 (m, 1H), 7.47 (m, 2H), 7.24 (m, 2H), 7.14 (m, 1H), 4.55-4.35 (m, 2H), 4.17-4.02 (m, 3H), 2.96-2.81 (m, 2H), 2.75-2.66 (m, 2H), 2.52-2.31 (m, 3H), 2.20-2.01 (m, 5H), 1.69 (m, 3H), 1.42 (m, 2H), 1.23 (m, 1H), 0.71 (m, 6H). MS m/z 432 (M+1).

### d) (8S)—N-[(1-{[(3S)-1-Methyl-3-piperidinyl]methyl}-1H-benzimidazol-2-yl)methyl]-N-(2-methylpropyl)-5,6,7,8-tetrahydro-8-quinolinamine

Reductive methylation of (8S)—N-(2-methylpropyl)-N-({1-[(3S)-3-piperidinylmethyl]-1H-benzimidazol-2-yl}methyl)-5,6,7,8-tetrahydro-8-quinolinamine (49 mg, 0.11 mmol) as described herein for the preparation of (8S)—N-methyl-N-[(1-{[(3S)-1-methyl-3-piperidinyl]methyl}-1H-benzimidazol-2-yl)methyl]-5,6,7,8-tetrahydro-8-quinolinamine afforded 45 mg (89%) of (8S)—N-[(1-{[(3S)-1-methyl-3-piperidinyl]methyl}-1H-benzimidazol-2-yl)methyl]-N-(2-methylpropyl)-5,6,7,8-tetrahydro-8-quinolinamine as a colorless oil. ¹H NMR (CD₃OD): δ 8.43 (d, 1H), 7.56 (m, 1H), 7.46 (m, 2H), 7.23 (m, 2H), 7.12 (m, 1H), 4.57 (m, 1H), 4.38 (m, 1H), 4.16-4.01 (m, 3H), 2.87-2.69 (m, 3H), 2.45-2.29 (m, 3H), 2.19-1.93 (m, 8H), 1.73-1.38 (m, 6H), 1.09 (m, 1H), 0.70 (m, 6H). MS m/z 446 (M+1).

### Example 95

### 2-{[(1-{[(3S)-1-Methyl-3-piperidinyl]methyl}-1H-benzimidazol-2-yl)methyl][(8S)-5,6,7,8-tetrahydro-8-quinolinyl]amino}ethanol

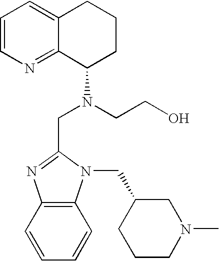

### a) (8S)—N-(2-{[(1,1-Dimethylethyl)(dimethyl)silyl]oxy}ethyl)-5,6,7,8-tetrahydro-8-quinolinamine

Reaction of (8S)—N-{(1S)-1-[4-(methyloxy)phenyl]ethyl}-5,6,7,8-tetrahydro-8-quinolinamine (1.35 g, 4.78 mmol), and (tert-butyldimethylsilyloxy)acetaldehyde (1.82 mL, 9.56 mmol) as described herein for the preparation of (8S)—N-methyl-N-{(1S)-1-[4-(methyloxy)phenyl]ethyl}-5,6,7,8-tetrahydro-8-quinolinamine afforded a yellow oil. Deprotection of the crude product as described herein for the preparation of (8R)—N-methyl-5,6,7,8-tetrahydro-8-quinolinamine afforded 0.52 g (35%) of (8S)—N-(2-{[(1,1-dimethylethyl)(dimethyl)silyl]oxy}ethyl)-5,6,7,8-tetrahydro-8-quinolinamine as an orange-brown oil. ¹H-NMR (CDCl₃): δ 8.42 (d, 1H), 7.41 (d, 1H), 7.10 (m, 1H), 3.84 (m, 3H), 2.93 (m, 2H), 2.81 (m, 2H), 2.21 (m, 1H), 2.06 (m, 1H), 1.81 (m, 2H), 0.97 (s, 9H), 0.12 (s, 6H). MS m/z 307 (M+H).

### b) 1,1-Dimethylethyl (3R)-3-{[2-({(2-{[(1,1-dimethylethyl)(dimethyl)silyl]oxy}ethyl)[(8S)-5,6,7,8-tetrahydro-8-quinolinyl]amino}methyl)-1H-benzimidazol-1-yl]methyl}-1-piperidinecarboxylate

Reaction of 1,1-dimethylethyl (3R)-3-{[2-(chloromethyl)-1H-benzimidazol-1-yl]methyl}-1-piperidinecarboxylate (0.62 g, 1.70 mmol) and (8S)—N-(2-{[(1,1-dimethylethyl)(dimethyl)silyl]oxy}ethyl)-5,6,7,8-tetrahydro-8-quinolinamine (0.52 g, 1.70 mmol) as described herein for the preparation of 1,1-dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate afforded 0.40 g (37%) of 1,1-dimethylethyl (3R)-3-{[2-({(2-{[(1,1-dimethylethyl)(dimethyl)silyl]oxy}ethyl)[(8S)-5,6,7,8-tetrahydro-8-quinolinyl]amino}methyl)-1H-benzimidazol-1-yl]methyl}-1-piperidinecarboxylate as a pale yellow foam. ¹H NMR (CD₃OD): δ 8.42 (d, 1H), 7.57 (m, 1H), 7.46 (m, 2H), 7.24 (m, 2H), 7.12 (m, 1H), 4.45-4.09 (m, 5H), 3.88 (m, 1H), 3.43 (m, 1H), 2.90-2.70 (m, 5H), 2.45 (m, 1H), 2.17-2.03 (m, 4H), 1.69 (m, 3H), 1.35-1.05 (m, 13H), 0.73 (s, 9H), −0.18 (s, 6H). MS m/z 634 (M+1).

### c) 2-{({1-[(3S)-3-Piperidinylmethyl]-1H-benzimidazol-2-yl}methyl)[(8S)-5,6,7,8-tetrahydro-8-quinolinyl]amino}ethanol

Deprotection of 1,1-dimethylethyl (3R)-3-{[2-({(2-{[(1,1-dimethylethyl)(dimethyl)silyl]oxy}ethyl)[(8S)-5,6,7,8-tetrahydro-8-quinolinyl]amino}methyl)-1H-benzimidazol-1-yl]methyl}-1-piperidinecarboxylate (395 mg, 0.62 mmol) as described herein for the preparation of (8S)—N-methyl-N-({1-[(3S)-3-piperidinylmethyl]-1H-benzimidazol-2-yl}methyl)-5,6,7,8-tetrahydro-8-quinolinamine afforded 2-{({1-[(3S)-3-piperidinylmethyl]-1H-benzimidazol-2-yl}methyl)[(8S)-5,6,7,8-tetrahydro-8-quinolinyl]amino}ethanol as an off-white foam in quantitative yield. ¹H NMR (CD₃OD): δ 8.36 (d, 1H), 7.55 (d, 1H), 7.42 (m, 2H), 7.24 (m, 2H), 7.07 (m, 1H), 4.46-4.29 (m, 2H), 4.18 (m, 2H), 4.00 (m, 1H), 3.55-3.39 (m, 2H), 2.96-2.72 (m, 6H), 2.50 (m, 1H), 2.26-2.04 (m, 5H), 1.67 (m, 3H), 1.43 (m, 1H), 1.26 (m, 1H). MS m/z 420 (M+1).

### d) 2-{[(1-{[(3S)-1-Methyl-3-piperidinyl]methyl}-1H-benzimidazol-2-yl)methyl][(8S)-5,6,7,8-tetrahydro-8-quinolinyl]amino}ethanol

Reductive methylation of 2-{({1-[(3S)-3-piperidinylmethyl]-1H-benzimidazol-2-yl}methyl)[(8S)-5,6,7,8-tetrahydro-8-quinolinyl]amino}ethanol (54 mg, 0.13 mmol) as described herein for the preparation of (8S)—N-methyl-N-[(1-{[(3S)-1-methyl-3-piperidinyl]methyl}-1H-benzimidazol-2-yl)methyl]-5,6,7,8-tetrahydro-8-quinolinamine afforded 44 mg (79%) of 2-{[(1-{[(3S)-1-methyl-3-piperidinyl]methyl}-1H-benzimidazol-2-yl)methyl][(8S)-5,6,7,8-tetrahydro-8-quinolinyl]amino}ethanol as a colorless oil. ¹H NMR (CD₃OD): δ 8.35 (d, 1H), 7.55 (m, 1H), 7.41 (m, 2H), 7.22 (m, 2H), 7.07 (m, 1H), 4.48 (m, 1H), 4.33 (m, 1H), 4.17 (m, 2H), 3.98 (m, 1H), 3.54 (m, 1H), 3.37 (m, 1H), 2.84-2.66 (m, 5H), 2.52 (m, 1H), 2.23-2.17 (m, 5H), 2.03 (m, 3H), 1.69-1.48 (m, 5H), 1.12 (m, 1H). MS m/z 434 (M+1).

### Example 96

### 3-{[(1-{[(3S)-1-Methyl-3-piperidinyl]methyl}-1H-benzimidazol-2-yl)methyl][(8S)-5,6,7,8-tetrahydro-8-quinolinyl]amino}-1-propanol

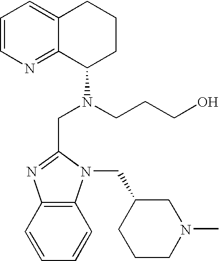

### a) 3-[(8S)-5,6,7,8-Tetrahydro-8-quinolinylamino]-1-propanol

A solution of 3-{(tert-butyldimethylsilyl)oxy}propanol (2.5 mL, 11.7 mmol) in dichloromethane (20 mL) was treated with IBX polystyrene resin (12.55 g, 1.4 mmol/g, Novabiochem) and allowed to stir at RT overnight. The resin was removed by filtration and the filtrate was cooled to 0° C. To this solution was added (8S)—N-{(1S)-1-[4-(methyloxy)phenyl]ethyl}-5,6,7,8-tetrahydro-8-quinolinamine (1.10 g, 3.91 mmol), NaBH(OAc)₃ (1.24 g, 5.87 mmol), and acetic acid (2.2 mL, 39.1 mmol). After 2 h the reaction was treated with 10% aqueous sodium carbonate and stirred for 2 h. The layers were separated and the aqueous layer was extracted with CH₂Cl₂. The combined organic layers were washed with brine, dried over Na₂SO₄ and concentrated to a yellow oil. Deprotection of the crude product as described herein for the preparation of (8R)—N-methyl-5,6,7,8-tetrahydro-8-quinolinamine afforded 0.45 g (56%) of 3-[(8S)-5,6,7,8-tetrahydro-8-quinolinylamino]-1-propanol as a yellow oil. ¹H-NMR (CDCl₃): δ 8.37 (d, 1H), 7.37 (m, 1H), 7.07 (m, 1H), 3.80 (m, 2H), 3.70 (m, 1H), 3.11 (m, 1H), 2.92-2.70 (m, 3H), 2.13 (m, 1H), 1.96 (m, 1H), 1.87-1.69 (m, 4H). MS m/z 207 (M+H).

### b) 1,1-Dimethylethyl (3R)-3-{[2-({(3-hydroxypropyl)[(8S)-5,6,7,8-tetrahydro-8-quinolinyl]amino}methyl)-1H-benzimidazol-1-yl]methyl}-1-piperidinecarboxylate

Reaction of 1,1-dimethylethyl (3R)-3-{[2-(chloromethyl)-1H-benzimidazol-1-yl]methyl}-1-piperidinecarboxylate (0.69 g, 1.90 mmol) and 3-[(8S)-5,6,7,8-tetrahydro-8-quinolinylamino]-1-propanol (0.37 g, 1.79 mmol) as described herein for the preparation of 1,1-dimethylethyl (3S)-3-{[2-({methyl[(8S)-5,6,7,8-tetrahydro-8-quinolinyl]amino}methyl)-1H-benzimidazol-1-yl]methyl}-1-piperidinecarboxylate afforded 0.62 g (65%) of 1,1-dimethylethyl (3R)-3-{[2-({(3-hydroxypropyl)[(8S)-5,6,7,8-tetrahydro-8-quinolinyl]amino}methyl)-1H-benzimidazol-1-yl]methyl}-1-piperidinecarboxylate as a white foam. ¹H NMR (CD₃OD): δ 8.39 (m, 1H), 7.57 (m, 1H), 7.45 (m, 2H), 7.24 (m, 2H), 7.12 (m, 1H), 4.47 (m, 1H), 4.27 (m, 1H), 4.05 (m, 3H), 3.84 (m, 1H), 3.54 (m, 1H), 3.42 (m, 1H), 2.84-2.65 (m, 5H), 2.31-1.99 (m, 5H), 1.69 (m, 4H), 1.51-1.07 (m, 13H). MS m/z 534 (M+1).

### c) 3-{({1-[(3S)-3-Piperidinylmethyl]-1H-benzimidazol-2-yl}methyl)[(8S)-5,6,7,8-tetrahydro-8-quinolinyl]amino}-1-propanol

Deprotection of 1,1-dimethylethyl (3R)-3-{[2-({(3-hydroxypropyl)[(8S)-5,6,7,8-tetrahydro-8-quinolinyl]amino}methyl)-1H-benzimidazol-1-yl]methyl}-1-piperidinecarboxylate (710 mg, 1.33 mmol) as described herein for the preparation of (8S)—N-methyl-N-({1-[(3S)-3-piperidinylmethyl]-1H-benzimidazol-2-yl}methyl)-5,6,7,8-tetrahydro-8-quinolinamine afforded 3-{({1-[(3S)-3-piperidinylmethyl]-1H-benzimidazol-2-yl}methyl)[(8S)-5,6,7,8-tetrahydro-8-quinolinyl]amino}-1-propanol as a white foam in quantitative yield. ¹H NMR (CD₃OD): δ 8.40 (d, 1H), 7.57 (m, 1H), 7.44 (m, 2H), 7.24 (m, 2H), 7.12 (m, 1H), 4.47-4.24 (m, 2H), 4.05 (m, 3H), 3.54 (m, 1H), 3.42 (m, 1H), 2.95-2.82 (m, 3H), 2.75-2.63 (m, 3H), 2.46 (m, 1H), 2.20-2.07 (m, 5H), 1.72 (m, 4H), 1.54-1.41 (m, 2H), 1.20 (m, 1H). MS m/z 434 (M+1).

### d) 3-{[(1-{[(3S)-1-Methyl-3-piperidinyl]methyl}-1H-benzimidazol-2-yl)methyl][(8S)-5,6,7,8-tetrahydro-8-quinolinyl]amino}-1-propanol

Reductive methylation 3-{({1-[(3S)-3-piperidinylmethyl]-1H-benzimidazol-2-yl}methyl)[(8S)-5,6,7,8-tetrahydro-8-quinolinyl]amino}-1-propanol (50 mg, 0.12 mmol) as described herein for the preparation of (8S)—N-methyl-N-[(1-{[(3S)-1-methyl-3-piperidinyl]methyl}-1H-benzimidazol-2-yl)methyl]-5,6,7,8-tetrahydro-8-quinolinamine afforded 43 mg (83%) of 3-{[(1-{[(3S)-1-methyl-3-piperidinyl]methyl}-1H-benzimidazol-2-yl)methyl][(8S)-5,6,7,8-tetrahydro-8-quinolinyl]amino}-1-propanol as a colorless oil. ¹H NMR (CD₃OD): δ 8.38 (d, 1H), 7.56 (m, 1H), 7.44 (m, 2H), 7.23 (m, 2H), 7.11 (m, 1H), 4.47 (m, 1H), 4.26 (m, 1H), 4.03 (m, 3H), 3.53 (m, 1H), 3.40 (m, 1H), 2.86-2.63 (m, 5H), 2.38 (m, 1H), 2.16-2.08 (m, 7H), 1.90 (m, 1H), 1.74-1.46 (m, 7H), 1.04 (m, 1H). MS m/z 448 (M+1).

### Example 97

### (8S)—N-[(1-{[(3S)-1-(1-Methylethyl)-3-piperidinyl]methyl}-1H-benzimidazol-2-yl)methyl]-N-propyl-5,6,7,8-tetrahydro-8-quinolinamine

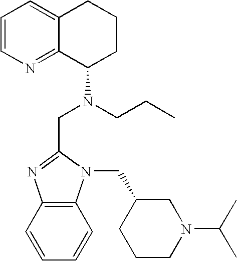

A solution of (8S)—N-({1-[(3S)-3-piperidinylmethyl]-1H-benzimidazol-2-yl}methyl)-N-propyl-5,6,7,8-tetrahydro-8-quinolinamine (56.5 mg, 0.14 mmol) in 1,2-dichloroethane (4 mL) was treated with acetone (50 μL, 0.68 mmol), NaBH(OAc)₃ (172 mg, 0.81 mmol) and glacial acetic acid (23 μL, 0.34 mmol). After 18 h the reaction was diluted with dichloromethane, 10% aqueous Na₂CO₃ and brine and shaken well. The mixture was filtered through a hydrophobic frit. The aqueous layer was washed with dichloromethane and filtered. The combined organic layers were concentrated under reduced pressure. The crude product was purified by flash chromatography (silica gel, gradient elution of acetonitrile to 9:1 acetonitrile/NH₄OH) to afford 53 mg (85%) of (8S)—N-[(1-{[(3S)-1-(1-methylethyl)-3-piperidinyl]methyl}-1H-benzimidazol-2-yl)methyl]-N-propyl-5,6,7,8-tetrahydro-8-quinolinamine as a colorless oil. ¹H NMR (CD₃OD): δ 8.42 (d, 1H), 7.56 (d, 1H), 7.44 (m, 2H), 7.23 (m, 2H), 7.13 (m, 1H), 4.46-4.31 (m, 2H), 4.09 (m, 3H), 2.90-2.46 (m, 7H), 2.12 (m, 5H), 1.72-1.34 (m, 7H), 1.01-0.91 (m, 7H), 0.74 (t, 3H). MS m/z 460 (M+1).

### Example 98

### (8S)—N-[(1-{[(3S)-1-(1-Methylethyl)-3-piperidinyl]methyl}-1H-benzimidazol-2-yl)methyl]-N-(2-methylpropyl)-5,6,7,8-tetrahydro-8-quinolinamine

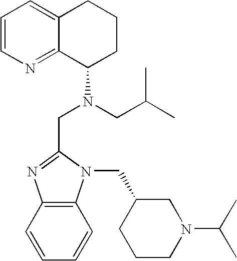

Reaction of (8S)—N-(2-methylpropyl)-N-({1-[(3S)-3-piperidinylmethyl]-1H-benzimidazol-2-yl}methyl)-5,6,7,8-tetrahydro-8-quinolinamine (60 mg, 0.14 mmol) as described herein for the preparation of (8S)—N-[(1-{[(3S)-1-(1-methylethyl)-3-piperidinyl]methyl}-1H-benzimidazol-2-yl)methyl]-N-propyl-5,6,7,8-tetrahydro-8-quinolinamine afforded 56 mg (86%) of (8S)—N-[(1-{[(3S)-1-(1-methylethyl)-3-piperidinyl]methyl}-1H-benzimidazol-2-yl)methyl]-N-(2-methylpropyl)-5,6,7,8-tetrahydro-8-quinolinamine as a colorless oil. ¹H NMR (CD₃OD): δ 8.46 (d, 1H), 7.58 (d, 1H), 7.48 (m, 2H), 7.25 (m, 2H), 7.15 (m, 1H), 4.60 (m, 1H), 4.39 (m, 1H), 4.16-4.03 (m, 3H), 2.88-2.01 (m, 12H), 1.77-1.64 (m, 4H), 1.46 (m, 2H), 1.12 (m, 1H), 0.94 (m, 6H), 0.71 (m, 6H). MS m/z 474 (M+1).

### Example 99

### 2-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}ethanol

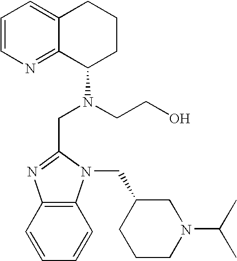

Reaction of 2-{({1-[(3S)-3-piperidinylmethyl]-1H-benzimidazol-2-yl}methyl)[(8S)-5,6,7,8-tetrahydro-8-quinolinyl]amino}ethanol (57 mg, 0.14 mmol) as described herein for the preparation of (8S)—N-[(1-{[(3S)-1-(1-methylethyl)-3-piperidinyl]methyl}-1H-benzimidazol-2-yl)methyl]-N-propyl-5,6,7,8-tetrahydro-8-quinolinamine afforded 42 mg (67%) of 2-{[(1-{[(3S)-1-(1-methylethyl)-3-piperidinyl]methyl}-1H-benzimidazol-2-yl)methyl][(8S)-5,6,7,8-tetrahydro-8-quinolinyl]amino}ethanol as a white foam. ¹H NMR (CD₃OD): δ 8.37 (m, 1H), 7.56 (m, 1H), 7.43 (m, 2H), 7.23 (m, 2H), 7.08 (m, 1H), 4.51 (m, 1H), 4.34 (m, 1H), 4.25-4.13 (m, 2H), 4.00 (m, 1H), 3.56 (m, 1H), 3.41 (m, 1H), 2.87-2.67 (m, 7H), 2.37-2.25 (m, 3H), 2.06 (m, 3H), 1.67 (m, 3H), 1.51 (m, 1H), 1.17 (m, 1H), 0.98 (m, 6H). MS m/z 462 (M+1).

### Example 100

### 3-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}-1-propanol

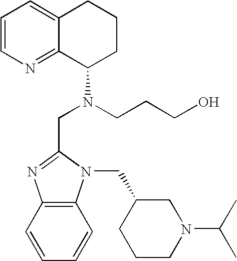

Reaction of 3-{({1-[(3S)-3-piperidinylmethyl]-1H-benzimidazol-2-yl}methyl)[(8S)-5,6,7,8-tetrahydro-8-quinolinyl]amino}-1-propanol (54 mg, 0.12 mmol) as described herein for the preparation of (8S)—N-[(1-{[(3S)-1-(1-methylethyl)-3-piperidinyl]methyl}-1H-benzimidazol-2-yl)methyl]-N-propyl-5,6,7,8-tetrahydro-8-quinolinamine afforded 25 mg (42%) of 2-{[(1-{[(3S)-1-(1-methylethyl)-3-piperidinyl]methyl}-1H-benzimidazol-2-yl)methyl][(8S)-5,6,7,8-tetrahydro-8-quinolinyl]amino}ethanol as a white foam. ¹H NMR (CD₃OD): δ 8.41 (d, 1H), 7.57 (d, 1H), 7.45 (m, 2H), 7.24 (m, 2H), 7.13 (m, 1H), 4.51 (m, 1H), 4.28 (m, 1H), 4.04 (m, 3H), 3.54 (m, 1H), 3.42 (m, 1H), 2.91-2.51 (m, 7H), 2.14 (m, 5H), 1.77-1.62 (m, 5H), 1.49 (m, 2H), 1.08 (m, 1H), 0.93 (m, 6H). MS m/z 476 (M+1).

### Example 101

### (8S)—N-{[1-({(3S)-1-[3-(Dimethylamino)-2,2-dimethylpropyl]-3-piperidinyl}methyl)-1H-benzimidazol-2-yl]methyl}-N-methyl-5,6,7,8-tetrahydro-8-quinolinamine

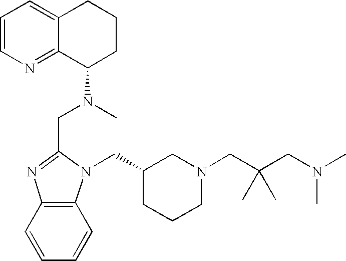

In a manner similar to the procedure described herein for the synthesis of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (50 mg, 0.13 mmol) was subjected to reductive alkylation with 3-(dimethylamino)-2,2-dimethylpropanal (33 mg, 0.26 mmol) followed by reverse phase HPLC purification to afford 39 mg (61%) of (8S)—N-{[1-({(3S)-1-[3-(dimethylamino)-2,2-dimethylpropyl]-3-piperidinyl}methyl)-1H-benzimidazol-2-yl]methyl}-N-methyl-5,6,7,8-tetrahydro-8-quinolinamine as a viscous oil. ¹H NMR (CD₃OD): δ 8.47 (d, 1H), 7.60-7.53 (m, 2H), 7.46 (d, 1H), 7.32-7.17 (m, 3H), 4.27-4.15 (m, 2H), 4.09 (d, 1H), 3.97-3.83 (m, 2H), 2.93 (m, 1H), 2.79 (m, 1H), 2.70 (d, 1H), 2.32 (d, 1H), 2.29-2.17 (m, 4H), 2.16-1.84 (m, 14H), 1.83-1.40 (m, 5H), 0.86 (m, 1H), 0.68 (s, 3H), 0.67 (s, 3H). MS m/z 503 (M+1).

### Example 102

### (8S)—N-Methyl-N-[(1-{[(3S)-1-(2-thienylmethyl)-3-Piperidinyl]methyl}-1H-benzimidazol-2-yl)methyl]-5,6,7,8-tetrahydro-8-quinolinamine

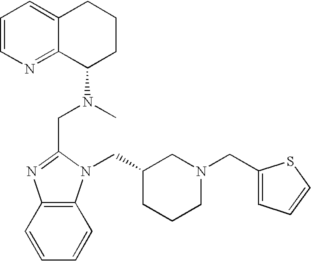

In a manner similar to the procedure described herein for the synthesis of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (50 mg, 0.13 mmol) was subjected to reductive alkylation with 2-thiophenecarbaldehyde (29 mg, 0.26 mmol) followed by reverse phase HPLC purification to afford 34 mg (54%) of (8S)—N-methyl-N-[(1-{[(3S)-1-(2-thienylmethyl)-3-piperidinyl]methyl}-1H-benzimidazol-2-yl)methyl]-5,6,7,8-tetrahydro-8-quinolinamine as a viscous oil. ¹H NMR (CD₃OD): δ 8.39 (d, 1H), 7.57 (d, 1H), 7.50-7.43 (m, 2H), 7.33-7.14 (m, 4H), 6.93 (m, 1H), 6.74 (m, 1H), 4.28 (dd, 1H), 4.19 (dd, 1H), 4.04 (d, 1H), 3.89 (t, 1H), 3.81 (d, 1H), 3.68 (d, 1H), 3.58 (d, 1H), 2.92 (m, 1H), 2.83-2.67 (m, 2H), 2.47 (d, 1H), 2.29-2.18 (m, 4H), 2.17-1.93 (m, 4H), 1.76 (m, 1H), 1.68-1.37 (m, 4H), 0.82 (m, 1H). MS m/z 486 (M+1).

### Example 103

### (8S)—N-Methyl-N-[(1-{[(3S)-1-(1,3-thiazol-2-ylmethyl)-3-piperidinyl]methyl}-1H-benzimidazol-2-yl)methyl]-5,6,7,8-tetrahydro-8-quinolinamine

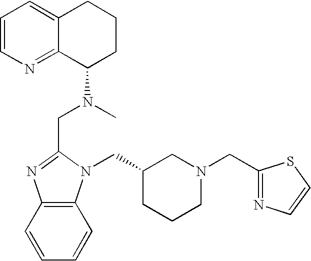

In a manner similar to the procedure described herein for the synthesis of (8S)—N-methyl-N-[(1-{([(3S)-1-(2-pyridinylmethyl)-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (50 mg, 0.13 mmol) was subjected to reductive alkylation with 2-thiazolecarbaldehyde (29 mg, 0.26 mmol) followed by reverse phase HPLC purification to afford 22 mg (35%) of (8S)—N-methyl-N-[(1-{[(3S)-1-(1,3-thiazol-2-ylmethyl)-3-piperidinyl]methyl}-1H-benzimidazol-2-yl)methyl]-5,6,7,8-tetrahydro-8-quinolinamine as a viscous oil ¹H NMR (CD₃OD): δ 8.42 (d, 1H), 7.67 (d, 1H), 7.58-7.43 (m, 4H), 7.29-7.17 (m, 3H), 4.33-4.17 (m, 2H), 4.06 (d, 1H), 3.94-3.68 (m, 4H), 2.93 (m, 1H), 2.85-2.70 (m, 2H), 2.50 (m, 1H), 2.28-1.94 (m, 8H), 1.82-1.60 (m, 3H), 1.56-1.40 (m, 2H), 0.83 (m, 1H). MS m/z 487 (M+1).

### Example 104

### (8S)—N-Methyl-N-{[1-({(3S)-1-[(1-methyl-1H-pyrrol-2-yl)methyl]-3-piperidinyl}methyl)-1H-benzimidazol-2-yl]methyl}-5,6,7,8-tetrahydro-8-quinolinamine

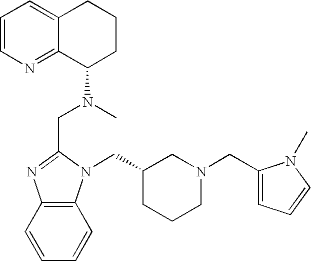

In a manner similar to the procedure described herein for the synthesis of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (50 mg, 0.13 mmol) was subjected to reductive alkylation with 1-methyl-1H-pyrrole-2-carbaldehyde (28 mg, 0.26 mmol) followed by reverse phase HPLC purification to afford 5 mg (8%) of (8S)—N-methyl-N-{[1-({(3S)-1-[(1-methyl-1H-pyrrol-2-yl)methyl]-3-piperidinyl}methyl)-1H-benzimidazol-2-yl]methyl}-5,6,7,8-tetrahydro-8-quinolinamine as a viscous oil. ¹H NMR (CD₃OD): δ 8.39 (d, 1H), 7.57 (d, 1H), 7.52 (d, 1H), 7.43 (d, 1H), 7.30-7.16 (m, 3H), 6.53 (m, 1H), 5.89 (m, 1H), 5.79 (m, 1H), 4.30-4.13 (m, 2H), 4.04 (d, 1H), 3.87 (t, 1H), 3.80 (d, 1H), 3.50 (s, 3H), 3.40 (d, 1H), 3.26 (d, 1H), 2.93 (m, 1H), 2.85-2.72 (m, 2H), 2.51 (d, 1H), 2.29-2.18 (m, 4H), 2.16-1.83 (m, 5H), 1.78 (m, 1H), 1.61 (m, 1H), 1.52-1.35 (m, 2H), 0.83 (m, 1H). MS m/z 483 (M+1).

### Example 105

### (8S)—N-{[1-({(3S)-1-[2-(Dimethylamino)ethyl]-3-piperidinyl}methyl)-1H-benzimidazol-2-yl]methyl}-N-methyl-5,6,7,8-tetrahydro-8-quinolinamine

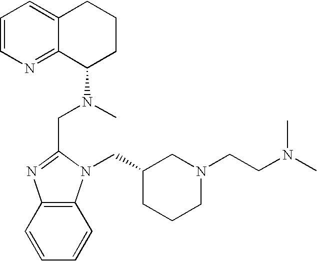

### a) 1,1-Dimethylethyl [2-((3S)-3-{[2-({methyl[(8S)-5,6,7,8-tetrahydro-8-quinolinyl]amino}methyl)-1H-benzimidazol-1-yl]methyl}-1-piperidinyl)ethyl]carbamate

In a manner similar to the procedure described herein for the synthesis of (8S)—N-methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine (73 mg, 0.19 mmol) was subjected to reductive alkylation with N-boc-2-aminoacetaldehyde (45 mg, 0.28 mmol) followed by reverse phase HPLC purification to afford 23 mg (23%) of 1,1-dimethylethyl [2-((3S)-3-{[2-({methyl[(8S)-5,6,7,8-tetrahydro-8-quinolinyl]amino}methyl)-1H-benzimidazol-1-yl]methyl}-1-piperidinyl)ethyl]carbamate as a white foam. ¹H NMR (CD₃OD): δ 8.46 (d, 1H), 7.61-7.53 (m, 2H), 7.48 (d, 1H), 7.32-7.18 (m, 3H), 4.37-4.13 (m, 2H), 4.10 (d, 1H), 3.97-3.85 (m, 2H), 3.06 (t, 2H), 2.95 (m, 1H), 2.86-2.72 (m, 2H), 2.52 (d, 1H), 2.38-1.90 (m, 10H), 1.78 (m, 1H), 1.69-1.56 (m, 2H), 1.55-1.37 (m, 11H), 0.90 (m, 1H). MS m/z 533 (M+1).

### b) (8S)—N-[(1-{[(3S)-1-(2-Aminoethyl)-3-piperidinyl]methyl}-1H-benzimidazol-2-yl)methyl]-N-methyl-5,6,7,8-tetrahydro-8-quinolinamine

In a manner similar to the procedure described herein for the synthesis of (8S)—N-methyl-N-({1-[(3S)-3-piperidinylmethyl]-1H-benzimidazol-2-yl}methyl)-5,6,7,8-tetrahydro-8-quinolinamine, 1,1-dimethylethyl [2-((3S)-3-{[2-({methyl[(8S)-5,6,7,8-tetrahydro-8-quinolinyl]amino}methyl)-1H-benzimidazol-1-yl]methyl}-1-piperidinyl)ethyl]carbamate (21 mg, 0.039 mmol) was subjected to TFA deprotection followed by aqueous Na₂CO₃ free basing to afford 16 mg (94%) of (8S)—N-[(1-{[(3S)-1-(2-aminoethyl)-3-piperidinyl]methyl}-1H-benzimidazol-2-yl)methyl]-N-methyl-5,6,7,8-tetrahydro-8-quinolinamine as a clear viscous oil. ¹H NMR (CD₃OD): δ 8.44 (d, 1H), 7.61-7.52 (m, 2H), 7.48 (d, 1H), 7.32-7.17 (m, 3H), 4.36-4.18 (m, 2H), 4.07 (d, 1H), 3.98-3.83 (m, 2H), 2.93 (m, 1H), 2.86-2.74 (m, 2H), 2.70-2.58 (m, 2H), 2.48 (d, 1H), 2.39-1.98 (m, 9H), 1.93 (t, 1H), 1.83-1.39 (m, 5H), 0.92 (m, 1H). MS m/z 433 (M+1).

### c) (8S)—N-{[1-({(3S)-1-[2-(Dimethylamino)ethyl]-3-piperidinyl}methyl)-1H-benzimidazol-2-yl]methyl}-N-methyl-5,6,7,8-tetrahydro-8-quinolinamine

Reductive methylation of (8S)—N-[(1-{[(3S)-1-(2-aminoethyl)-3-piperidinyl]methyl}-1H-benzimidazol-2-yl)methyl]-N-methyl-5,6,7,8-tetrahydro-8-quinolinamine (9 mg, 0.021 mmol) in a manner similar to the procedure described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 8 mg (83%) of (8S)—N-{[1-({(3S)-1-[2-(dimethylamino)ethyl]-3-piperidinyl}methyl)-1H-benzimidazol-2-yl]methyl}-N-methyl-5,6,7,8-tetrahydro-8-quinolinamine as a clear viscous oil. ¹H NMR (CD₃OD): δ 8.43 (m, 1H), 7.60-7.51 (m, 2H), 7.46 (d, 1H), 7.30-7.15 (m, 3H), 4.36-4.15 (m, 2H), 4.07 (d, 1H), 3.97-3.82 (m, 2H), 2.91 (m, 1H), 2.84-2.69 (m, 2H), 2.49 (d, 1H), 2.40-1.86 (m, 18H), 1.82-1.36 (m, 5H), 0.91 (m, 1H). MS m/z 461 (M+1).

### Example 106

### (8S)—N-Methyl-N-({1-[((3S)-1-{[(2S)-1-methyl-2-pyrrolidinyl]methyl}-3-Piperidinyl)methyl]-1H-benzimidazol-2-yl}methyl)-5,6,7,8-tetrahydro-8-quinolinamine

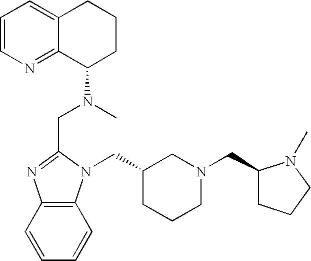

### a) (8S)—N-Methyl-N-{[1-({(3S)-1-[(2S)-2-pyrrolidinylmethyl]-3-piperidinyl}methyl)-1H-benzimidazol-2-yl]methyl}-5,6,7,8-tetrahydro-8-quinolinamine

In a manner similar to the procedure described herein for the synthesis of (8S)—N-[(1-{[(3S)-1-(2-aminoethyl)-3-piperidinyl]methyl}-1H-benzimidazol-2-yl)methyl]-N-methyl-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine was subjected to reductive alkylation with 1,1-dimethylethyl (2S)-2-formyl-1-pyrrolidinecarboxylate followed by TFA induced cleavage of the BOC group to afford (8S)—N-methyl-N-{[1-({(3S)-1-[(2S)-2-pyrrolidinylmethyl]-3-piperidinyl}methyl)-1H-benzimidazol-2-yl]methyl}-5,6,7,8-tetrahydro-8-quinolinamine as a light yellow foam in 79% overall yield. ¹H NMR (CD₃OD): δ 8.45 (d, 1H), 7.59-7.52 (m, 2H), 7.48 (d, 1H), 7.30-7.18 (m, 3H), 4.33-4.18 (m, 2H), 4.08 (d, 1H), 3.96-3.84 (m, 2H), 3.12 (m, 1H), 2.99-2.89 (m, 2H), 2.86-2.72 (m, 3H), 2.59 (d, 1H), 2.38-2.18 (m, 6H), 2.16-1.94 (m, 4H), 1.88-1.59 (m, 5H), 1.57-1.40 (m, 3H), 1.31 (m, 1H), 0.89 (m, 1H). MS m/z 473 (M+1).

### b) (8S)—N-Methyl-N-({1-[((3S)-1-{[(2S)-1-methyl-2-pyrrolidinyl]methyl}-3-piperidinyl)methyl]-1H-benzimidazol-2-yl}methyl)-5,6,7,8-tetrahydro-8-quinolinamine

Reductive methylation of (8S)—N-methyl-N-{[1-({(3S)-1-[(2S)-2-pyrrolidinylmethyl]-3-piperidinyl}methyl)-1H-benzimidazol-2-yl]methyl}-5,6,7,8-tetrahydro-8-quinolinamine (25 mg, 0.053 mmol) in a manner similar to the procedure described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 26 mg (100%) of (8S)—N-methyl-N-({1-[((3S)-1-{[(2S)-1-methyl-2-pyrrolidinyl]methyl}-3-piperidinyl)methyl]-1H-benzimidazol-2-yl}methyl)-5,6,7,8-tetrahydro-8-quinolinamine as a viscous oil. ¹H NMR (CD₃OD): δ 8.43 (d, 1H), 7.59-7.52 (m, 2H), 7.44 (d, 1H), 7.28-7.16 (m, 3H), 4.32-4.17 (m, 2H), 4.06 (d, 1H), 3.96-3.81 (m, 2H), 2.98-2.86 (m, 2H), 2.84 (m, 2H), 2.48-2.36 (m, 2H), 2.29-1.96 (m, 13H), 1.95-1.71 (m, 3H), 1.69-1.24 (m, 7H), 0.91 (m, 1H). MS m/z 487 (M+1).

### Example 107

### (8S)—N-Methyl-N-({1-[((3S)-1-{[(2S)-1-(1-methylethyl)-2-Pyrrolidinyl]methyl}-3-piperidinyl)methyl]-1H-benzimidazol-2-yl}methyl)-5,6,7,8-tetrahydro-8-quinolinamine

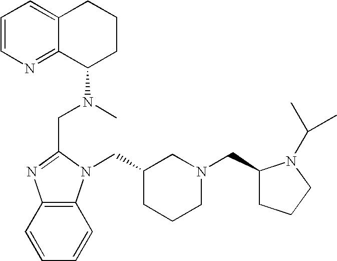

In a manner similar to the procedure described herein for the synthesis of (8R)—N-methyl-N-[(1-{[(3R)-1-(1-methylethyl)-3-piperidinyl]methyl}-1H-benzimidazol-2-yl)methyl]-5,6,7,8-tetrahydro-8-quinolinamine, (8S)—N-methyl-N-{[1-({(3S)-1-[(2S)-2-pyrrolidinylmethyl]-3-piperidinyl}methyl)-1H-benzimidazol-2-yl]methyl}-5,6,7,8-tetrahydro-8-quinolinamine (20 mg, 0.042 mmol) was subjected to reductive alkylation with acetone to afford 16 mg (73%) (8S)—N-methyl-N-({1-[((3S)-1-{[(2S)-1-(1-methylethyl)-2-pyrrolidinyl]methyl}-3-piperidinyl)methyl]-1H-benzimidazol-2-yl}methyl)-5,6,7,8-tetrahydro-8-quinolinamine as a viscous yellow oil. ¹H NMR (CD₃OD): δ 8.45 (d, 1H), 7.60-7.52 (m, 2H), 7.44 (d, 1H), 7.30 (m, 3H), 4.33-4.16 (m, 2H), 4.06 (d, 1H), 3.96-3.80 (m, 2H), 2.98-2.67 (m, 6H), 2.50-1.95 (m, 11H), 1.92-1.38 (m, 10H), 1.06-0.75 (m, 7H). MS m/z 515 (M+1).

### Example 108

### (8S)—N-Methyl-N-([1-[((3S)-1-{[(2R)-1-methyl-2-pyrrolidinyl]methyl}-3-piperidinyl)methyl]-1H-benzimidazol-2-yl]methyl)-5,6,7,8-tetrahydro-8-quinolinamine

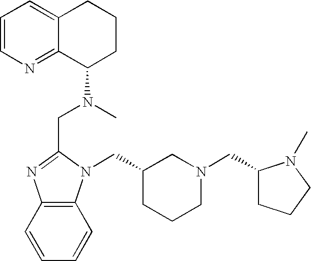

### a) (8S)—N-Methyl-N-{[1-({(3S)-1-[(2R)-2-pyrrolidinylmethyl]-3-piperidinyl}methyl)-1H-benzimidazol-2-yl]methyl}-5,6,7,8-tetrahydro-8-quinolinamine

In a manner similar to the procedure described herein for the synthesis of (8S)—N-[(1-{[(3S)-1-(2-aminoethyl)-3-piperidinyl]methyl}-1H-benzimidazol-2-yl)methyl]-N-methyl-5,6,7,8-tetrahydro-8-quinolinamine, (8S)—N-methyl-N-({1-[(3S)-3-piperidinylmethyl]-1H-benzimidazol-2-yl}methyl)-5,6,7,8-tetrahydro-8-quinolinamine was subjected to reductive alkylation with 1,1-dimethylethyl (2R)-2-formyl-1-pyrrolidinecarboxylate followed by TFA induced cleavage of the BOC group to afford (8S)—N-methyl-N-{[1-({(3S)-1-[(2R)-2-pyrrolidinylmethyl]-3-piperidinyl}methyl)-1H-benzimidazol-2-yl]methyl}-5,6,7,8-tetrahydro-8-quinolinamine as a white foam in 82% overall yield. ¹H NMR (CD₃OD): δ 8.43 (d, 1H), 7.59-7.52 (m, 2H), 7.45 (d, 1H), 7.29-7.16 (m, 3H), 4.33-4.15 (m, 2H), 4.05 (d, 1H), 3.95-3.81 (m, 2H), 3.18 (m, 1H), 2.98-2.70 (m, 5H), 2.41 (d, 1H), 2.28-1.94 (m, 9H), 1.90-1.38 (m, 9H), 1.29 (m, 1H), 0.91 (m, 1H). MS m/z 473 (M+1).

### b) (8S)—N-Methyl-N-({1-[((3S)-1-{[(2R)-1-methyl-2-pyrrolidinyl]methyl}-3-piperidinyl)methyl]-1H-benzimidazol-2-yl}methyl)-5,6,7,8-tetrahydro-8-quinolinamine

Reductive methylation of (8S)—N-methyl-N-{[1-({(3S)-1-[(2R)-2-pyrrolidinylmethyl]-3-piperidinyl}methyl)-1H-benzimidazol-2-yl]methyl}-5,6,7,8-tetrahydro-8-quinolinamine (25 mg, 0.053 mmol) in a manner similar to the procedure described herein for the preparation of N-methyl-N-({1-[(1-methyl-3-piperidinyl)methyl]-1H-benzimidazol-2-yl}methyl)-5,6,7,8-tetrahydro-8-quinolinamine, afforded 24 mg (92%) of (8S)—N-methyl-N-({1-[((3S)-1-{[(2R)-1-methyl-2-pyrrolidinyl]methyl}-3-piperidinyl)methyl]-1H-benzimidazol-2-yl}methyl)-5,6,7,8-tetrahydro-8-quinolinamine as a viscous oil. ¹H NMR (CD₃OD): δ 8.43 (d, 1H), 7.59-7.51 (m, 2H), 7.43 (d, 1H), 7.29-7.16 (m, 3H), 4.33-4.17 (m, 2H), 4.06 (d, 1H), 3.94-3.81 (m, 2H), 2.97-2.86 (m, 2H), 2.85-2.70 (m, 2H), 2.43 (d, 1H), 2.36-1.97 (m, 14H), 1.94-1.82 (m, 2H), 1.76 (m, 1H), 1.70-1.58 (m, 3H), 1.57-1.31 (m, 4H), 0.92 (m, 1H). MS m/z 487 (M+1).

### Example 109

### (8S)—N-Methyl-N-({1-[((3S)-1-{[(2R)-1-(1-methylethyl)-2-pyrrolidinyl]methyl}-3-piperidinyl)methyl]-1H-benzimidazol-2-yl}methyl)-5,6,7,8-tetrahydro-8-quinolinamine

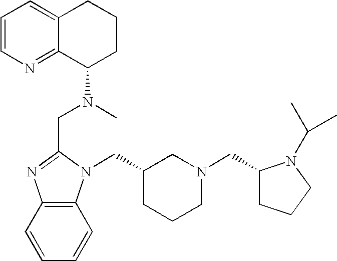

In a manner similar to the procedure described herein for the synthesis of (8R)—N-methyl-N-[(1-{[(3R)-1-(1-methylethyl)-3-piperidinyl]methyl}-1H-benzimidazol-2-yl)methyl]-5,6,7,8-tetrahydro-8-quinolinamine, (8S)—N-methyl-N-{[1-({(3S)-1-[(2R)-2-pyrrolidinylmethyl]-3-piperidinyl}methyl)-1H-benzimidazol-2-yl]methyl}-5,6,7,8-tetrahydro-8-quinolinamine (28 mg, 0.059 mmol) was subjected to reductive alkylation with acetone to afford 26 mg (87%) (8S)—N-methyl-N-({1-[((3S)-1-{[(2R)-1-(1-methylethyl)-2-pyrrolidinyl]methyl}-3-piperidinyl)methyl]-1H-benzimidazol-2-yl}methyl)-5,6,7,8-tetrahydro-8-quinolinamine as a viscous yellow oil. ¹H NMR (CD₃OD): δ 8.44 (d, 1H), 7.59-7.51 (m, 2H), 7.45 (d, 1H), 7.29-7.15 (m, 3H), 4.33-4.18 (m, 2H), 4.05 (d, 1H), 3.97-3.80 (m, 2H), 2.97-2.60 (m, 6H), 2.52-2.37 (m, 2H), 2.29-1.84 (m, 10H), 1.82-1.36 (m, 9H), 1.04-0.85 (m, 7H). MS m/z 515 (M+1).

### Example 110

### (8S)—N-{[1-(3-Aminopropyl)-1H-benzimidazol-2-yl]methyl}-N-methyl-5,6,7,8-tetrahydro-8-quinolinamine

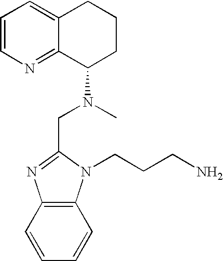

Employing a reaction sequence similar to that described herein for the preparation of (8S)—N-methyl-N-({1-[(3S)-3-piperidinylmethyl]-1H-benzimidazol-2-yl}methyl)-5,6,7,8-tetrahydro-8-quinolinamine, tert-butyl (3-aminopropyl)carbamate was converted, in 5 steps, to (8S)—N-{[1-(3-aminopropyl)-1H-benzimidazol-2-yl]methyl}-N-methyl-5,6,7,8-tetrahydro-8-quinolinamine which was obtained as as a viscous yellow oil in 60% overall yield. ¹H NMR (CD₃OD): δ 8.46 (d, 1H), 7.63-7.48 (m, 3H), 7.32-7.17 (m, 3H), 4.56 (m, 1H), 4.39 (m, 1H), 4.11-3.96 (m, 2H), 3.84 (d, 1H), 2.89 (m, 1H), 2.83-2.57 (m, 3H), 2.28 (s, 3H), 2.20-1.96 (m, 5H), 1.76 (m, 1H). MS m/z 350 (M+1).

### Example 111

### (8S)—N-({1-[3-(Dimethylamino)propyl]-1H-benzimidazol-2-yl}methyl)-N-methyl-5,6,7,8-tetrahydro-8-quinolinamine

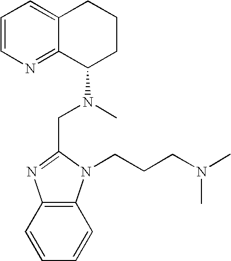

Reductive methylation of (8S)—N-{[1-(3-aminopropyl)-1H-benzimidazol-2-yl]methyl}-N-methyl-5,6,7,8-tetrahydro-8-quinolinamine (0.10 g, 0.0.29 mmol) in a manner similar to the procedure described herein for the preparation of N-methyl-N-({1-[(1-methyl-3-piperidinyl)methyl]-1H-benzimidazol-2-yl}methyl)-5,6,7,8-tetrahydro-8-quinolinamine, followed by flash chromatography (silica gel, gradient elution of MeCN to 95:5 MeCN/NH₄OH) afforded 75 mg (69%) of (8S)—N-({1-[3-(dimethylamino)propyl]-1H-benzimidazol-2-yl}methyl)-N-methyl-5,6,7,8-tetrahydro-8-quinolinamine as a viscous oil. ¹H NMR (CD₃OD): δ 8.45 (d, 1H), 7.62-7.47 (m, 3H), 7.32-7.16 (m, 3H), 4.50-4.33 (m, 2H), 4.08 (d, 1H), 4.03-3.91 (m, 2H), 2.90 (m, 1H), 2.77 (m, 1H), 2.30-2.02 (m, 14H), 1.97-1.84 (m, 2H), 1.77 (m, 1H). MS m/z 378 (M+1).

### Example 112

### (8S)—N-Methyl-N-[(1-{3-[(2-methylpropyl)amino]propyl}-1H-benzimidazol-2-yl)methyl]-5,6,7,8-tetrahydro-8-quinolinamine

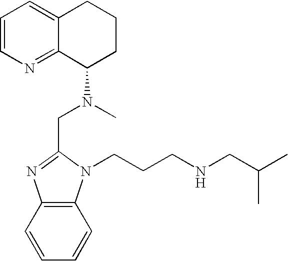

In a manner similar to the procedure described herein for the synthesis of N-methyl-N-[(1-{3-[(3-methylbutyl)amino]propyl}-1H-benzimidazol-2-yl)methyl]-5,6,7,8-tetrahydro-8-quinolinamine, (8S)—N-{[1-(3-aminopropyl)-1H-benzimidazol-2-yl]methyl}-N-methyl-5,6,7,8-tetrahydro-8-quinolinamine (0.10 g, 0.29 mmol) was subjected to reductive alkylation with isobutyraldehyde followed by flash chromatography (silica gel, gradient elution of MeCN to 95:5 MeCN/NH₄OH) to afford 62 mg (53%) of (8S)—N-methyl-N-[(1-{3-[(2-methylpropyl)amino]propyl}-1H-benzimidazol-2-yl)methyl]-5,6,7,8-tetrahydro-8-quinolinamine as a viscous oil. ¹H NMR (CD₃OD): δ 8.43 (d, 1H), 7.62-7.48 (m, 3H), 7.31-7.16 (m, 3H), 4.53-4.36 (m, 2H), 4.11-3.98 (m, 2H), 3.90 (d, 1H), 2.89 (m, 1H), 2.78 (m, 1H), 2.52 (t, 2H), 2.37 (d, 2H), 2.28 (s, 3H), 2.22-1.93 (m, 5H), 1.82-1.64 (m, 2H), 0.93-0.84 (m, 6H). MS m/z 406 (M+1).

### Example 113

### (8S)—N-Methyl-N[(1-{3-[(1-methylethyl)amino]propyl}-1H-benzimidazol-2-yl)methyl]-5,6,7,8-tetrahydro-8-quinolinamine

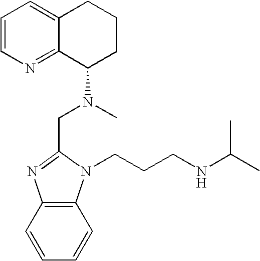

In a manner similar to the procedure described herein for the synthesis of N-methyl-N-[(1-{3-[(3-methylbutyl)amino]propyl}-1H-benzimidazol-2-yl)methyl]-5,6,7,8-tetrahydro-8-quinolinamine, (8S)—N-{[1-(3-aminopropyl)-1H-benzimidazol-2-yl]methyl}-N-methyl-5,6,7,8-tetrahydro-8-quinolinamine (0.10 g, 0.29 mmol) was subjected to reductive alkylation with acetone followed by flash chromatography (silica gel, gradient elution of MeCN to 95:5 MeCN/NH₄OH) to afford 54 mg (48%) of (8S)—N-methyl-N-[(1-{3-[(1-methylethyl)amino]propyl}-1H-benzimidazol-2-yl)methyl]-5,6,7,8-tetrahydro-8-quinolinamine as a viscous oil. ¹H NMR (CD₃OD): δ 8.44 (d, 1H), 7.58 (d, 1H), 7.56-7.48 (m, 2H), 7.32-7.16 (m, 3H), 4.54-4.39 (m, 2H), 4.10-3.98 (m, 2H), 3.90 (d, 1H), 2.90 (m, 1H), 2.83-2.70 (m, 2H), 2.52 (t, 2H), 2.27 (s, 3H), 2.21-2.03 (m, 3H), 2.01-1.88 (m, 2H), 1.77 (m, 1H), 1.05-0.97 (m, 6H). MS m/z 392 (M+1).

### Example 114

### (8S)—N-[(1-{3-[(1H-Imidazol-2-ylmethyl)amino]propyl}-1H-benzimidazol-2-yl)methyl]-N-methyl-5,6,7,8-tetrahydro-8-quinolinamine

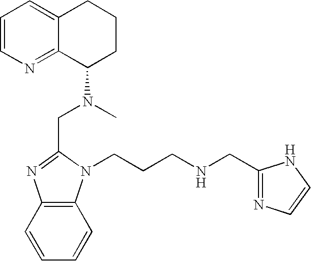

In a manner similar to the procedure described herein for the synthesis of (8S)—N-[(1-{[(3S)-1-(1H-imidazol-2-ylmethyl)-3-piperidinyl]methyl}-1H-benzimidazol-2-yl)methyl]-N-methyl-5,6,7,8-tetrahydro-8-quinolinamine, (8S)—N-{[1-(3-aminopropyl)-1H-benzimidazol-2-yl]methyl}-N-methyl-5,6,7,8-tetrahydro-8-quinolinamine (0.10 g, 0.29 mmol) was subjected to reductive alkylation with 1H-imidazole-2-carbaldehyde followed by flash chromatography (silica gel, gradient elution of MeCN to 95:5 MeCN/NH₄OH) to afford 60 mg (48%) of (8S)—N-[(1-{3-[(1H-imidazol-2-ylmethyl)amino]propyl}-1H-benzimidazol-2-yl)methyl]-N-methyl-5,6,7,8-tetrahydro-8-quinolinamine as an off-white solid. ¹H NMR (CD₃OD): δ 8.36 (d, 1H), 7.57 (d, 1H), 7.52-7.44 (m, 2H), 7.29-7.10 (m, 3H), 6.98 (s, 2H), 4.50-4.33 (m, 2H), 4.05 (d, 1H), 3.96 (m, 1H), 3.89 (d, 1H), 3.78 (s, 2H), 2.86 (m, 1H), 2.73 (m, 1H), 2.48 (t, 2H), 2.24 (s, 3H), 2.19-2.00 (m, 3H), 1.98-1.84 (m, 2H), 1.72 (m, 1H). MS m/z 430 (M+1).

### BIOLOGICAL SECTION

### Fusion Assay

### Plasmid Generation

The complete coding sequences of HIV-1 tat (GenBank Accession No. X07861) and rev (GenBank Accession No. M34378) were cloned into pcDNA3.1 expression vectors containing G418 and hygromycin resistance genes, respectively. The complete coding sequence of the HIV-1 (HXB2 strain) gp160 envelope gene (nucleotide bases 6225-8795 of GenBank Accession No. K03455) was cloned into plasmid pCRII-TOPO. The three HIV genes were additionally inserted into the baculovirus shuttle vector, pFastBacMam1, under the transcriptional control of the CMV promoter. A construction of the pHIV-1 LTR containing mutated NFkB sequences linked to the luciferase reporter gene was prepared by digesting pcDNA3.1, containing the G418 resistance gene, with Nru I and Bam HI to remove the CMV promoter. LTR-luc was then cloned into the Nru I/Bam HI sites of the plasmid vector. Plasmid preparations were performed after the plasmids were amplified in Escherichia coli strain DH5-alpha. The fidelity of the inserted sequences was confirmed by double-strand nucleotide sequencing using an ABI Prism Model 377 automated sequencer.

### BacMam Baculovirus Generation

Recombinant BacMam baculoviruses were constructed from pFastBacMam shuttle plasmids by using the bacterial cell-based Bac-to-Bac system. Viruses were propagated in Sf9 (Spodoptera frugiperda) cells cultured in Hink's TNM-FH Insect media supplemented with 10% (v/v) fetal bovine serum and 0.1% (v/v) pluronic F-68 according to established protocols.

### Cell Culture

Human osteosarcoma (HOS) cells that naturally express human CXCR4 were transfected with human CCR5, human CD4 and the pHIV-LTR-luciferase plasmid using FuGENE 6 transfection reagent. Single cells were isolated and grown under selection condition in order to generate a stable HOS (hCXCR4/hCCR5/hCD4/pHIV-LTR-luciferase) clonal cell line. The cells were maintained in Dulbeccos modified Eagles media supplemented with 10% fetal calf serum (FCS), G418 (400 ug/ml), puromycin (1 ug/ml), mycophenolic acid (40 ug/ml), xanthine (250 ug/ml) and hypoxanthine (13.5 ug/ml) to maintain a selection pressure for cells expressing the LTR-luciferase, hCCR5 and hCD4, respectively. Human embryonic kidney (HEK-293) cells stably transfected to express the human macrophage scavenging receptor (Class A, type 1; GenBank Accession No. D90187), were maintained in DMEM/F-12 media (1:1) supplemented with 10% FCS and 1.5 ug/ml puromycin. The expression of this receptor by the HEK-293 cells enhances their ability to stick to tissue culture treated plasticware.

### Transduction of HEK-293 Cells

HEK-293 cells were harvested using enzyme-free cell dissociation buffer. The cells were resuspended in DMEM/F-12 media supplemented with 10% FCS and 1.5 ug/ml and counted. Tranductions were performed by direct addition of BacMam baculovirus containing insect cell media to cells. The cells were simultaneously transduced with BacMam baculovirus expressing HIV-1 tat, HIV-1 rev and HIV-1 gp160 (from the HXB2 HIV strain). Routinely an MOI of 10 of each virus was added to the media containing the cells. 2 mM butyric acid was also added to the cells at this stage to increase protein expression in transduced cells. The cells were subsequently mixed and seeded into a flask at 30 million cells per T225. The cells were incubated at 37° C., 5% CO₂, 95% humidity for 24 h to allow for protein expression.

### Cell/Cell Fusion Assay Format

HEK and HOS cells were harvested in DMEM/F-12 media containing 2% FCS and DMEM media containing 2% FCS, respectively, with no selection agents added. Compounds were plated as 1 ul spots in 100% DMSO on a 96-well CulturPlate plates. HOS cells (50 ul) were added first to the wells, followed immediately by the HEK cells (50 ul). The final concentration of each cell type was 20,000 cells per well. Following these additions, the cells were returned to a tissue culture incubator (37° C.; 5% CO₂/95% air) for an additional 24 h.

### Measurement of Luciferase Production

Following the 24 h incubation, total cellular luciferase activity was measured using the LucLite Plus assay kit (Packard, Meridien, Conn.). In brief, 100 ul of this reagent was added to each well. The plates were sealed and mixed. The plates were dark adapted for approximately 10 min prior to the luminescence being read on a Packard TopCount.

### Functional Assay

### Cell Culture

Human embryonic kidney (HEK-293) cells were maintained and harvested as described above. Cells were plated in 96-well, black clear bottom, poly-lysine coated plates at a concentration of 40,000 cells per well in a final volume of 100 ul containing human CXCR4BacMam (MOI=25) and Gqi5 BacMam (MOI=12.5). The cells were incubated at 37° C., 5% CO₂, 95% humidity for 24 h to allow for protein expression.

### Functional FLIPR Assay

After the required incubation time the cells were washed once with 50 ul of fresh serum-free DMEM/F12 media containing probenicid. 50 ul of dye solution was then added to the cells (Calcium Plus Assay Kit Dye; Molecular Devices) was dissolved in 200 ml of the above probenicid/BSA containing media and incubated for 1 h. Cell plates were transferred to a Fluorometric Imaging Plate Reader (FLIPR). Upon addition the effect of the compounds on the change in [Ca²⁺]ᵢ was examined to determine if the compounds were agonists or antagonists (ability to block SDF-1 alpha activity) at the CXCR4 receptor. IC₅₀ values are determined and pK_{b} values are calculated using the Leff and Dougall equation: K_{B}═IC₅₀/((2+([agonist]/EC₅₀̂b)̂1/b−1) Where IC₅₀ is that defined by the antagonist concentration-response curve [agonist)] is the EC₈₀ concentration of agonist used EC₅₀ is that defined by the agonist concentration-response curve b is the slope of the agonist concentration-response curve.

### HOS HIV-1 Infectivity Assay

### HIV Virus Preparation

Compounds were profiled against two HIV-1 viruses, the M-tropic (CCR5 utilizing) Ba-L strain and the T-tropic (CXCR4 utilizing) IIIB strain. Both viruses were propagated in human peripheral blood lymphocytes. Compounds were tested for there ability to block infection of the HOS cell line (expressing hCXCR4/hCCR5/hCD4/pHIV-LTR-luciferase) by either HIV-1 Ba-L or HIV-1 IIIB. Compound cytotoxicity was also examined in the absence of virus addition.

### HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS and non-essential amino acid to a concentration of 60,000 cells/ml. The cells were plated into 96-well plates (100 ul per well) and the plates were placed in a tissue culture incubator (37° C.; 5% CO₂/95% air) for a period of 24 h.

Subsequently, 50 ul of the desired drug solution (4 times the final concentration) was added to each well and the plates were returned to the tissue culture incubator (37° C.; 5% CO₂/95% air) for 1 h. Following this incubation 50 ul of diluted virus was added to each well (approximately 2 million RLU per well of virus). The plates were returned to the tissue culture incubator (37° C.; 5% CO₂/95% air) and were incubated for a further 96 h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

TABLE 1

| | | | Fusion | | HOS |
|---|---|---|---|---|---|
| | | Functional | assay | Cytotox | (3B) |
| Example | Structure | assay (pIC50) | (pIC50) | (pIC50) | (uM) |
| 3 | 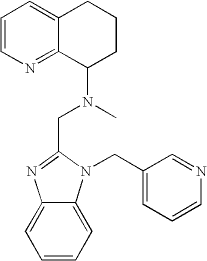 | 6.40 (n = 1) | 5.75 (n = 1) | <4.00(n = 1) | 0.574(n = 3) |
| 4 | 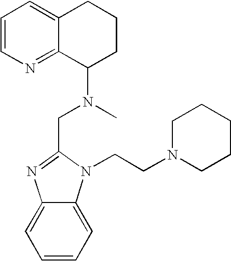 | 7.22 (n = 1) | 6.86 (n = 2) | <4.00(n = 1) | 0.156(n = 2) |
| 5 | 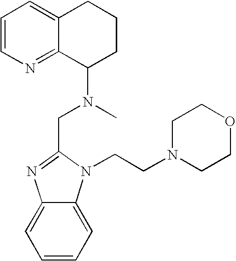 | 6.91 (n = 1) | 6.70 (n = 2) | <4.00(n = 1) | 0.225(n = 2) |
| 6 | 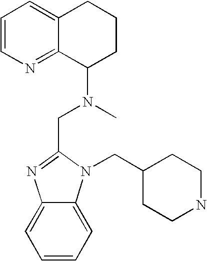 | 6.55 (n = 1) | 6.62 (n = 2) | <4.00(n = 1) | 0.090(n = 2) |
| 7 | 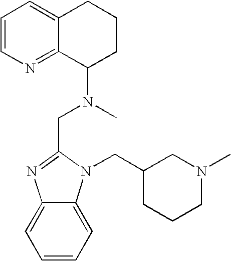 | 8.09 (n = 1) | 7.68 (n = 2) | <4.00(n = 1) | 0.019(n = 2) |
| 8d | 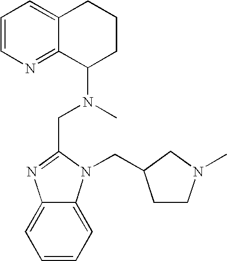 | 7.13 (n = 1) | 7.13 (n = 2) | <4.00(n = 1) | 0.057(n = 2) |
| 9 | 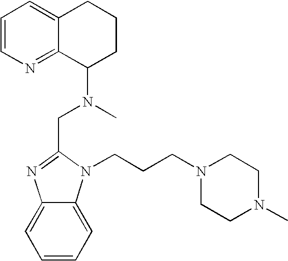 | 6.87 (n = 1) | 6.78 (n = 2) | <4.00(n = 1) | 0.093(n = 2) |
| 10e | 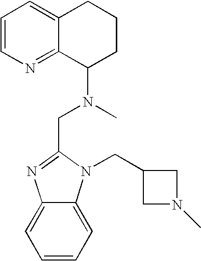 | 6.93 (n = 1) | 7.04 (n = 1) | <4.00(n = 1) | 0.066(n = 2) |
| 11i | 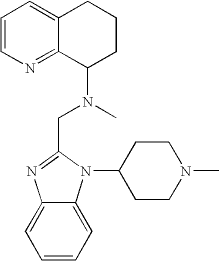 | 7.43 | 7.10 | <4.00 | 0.082(n = 1) |
| 12 | 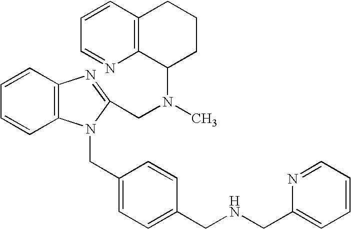 | 6.65 (n = 1) | 6.24 (n = 1) | <4.00(n = 1) | 0.376(n = 2) |
| 13 | 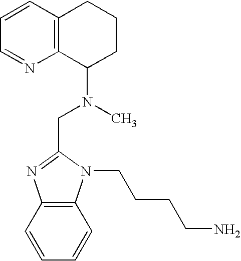 | 6.52 (n = 1) | 6.90 (n = 1) | <4.00(n = 1) | 0.131(n = 2) |
| 14 | 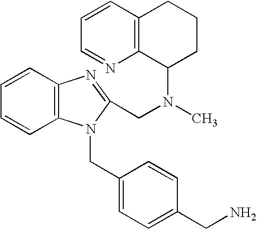 | 6.55 (n = 1) | 6.15 (n = 1) | <4.00(n = 1) | 0.985(n = 2) |
| 15c | 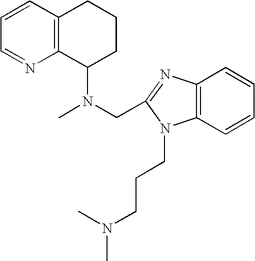 | 7.77 (n = 1) | 7.78 (n = 2) | <4.00(n = 1) | 0.023(n = 18) |
| 16 | 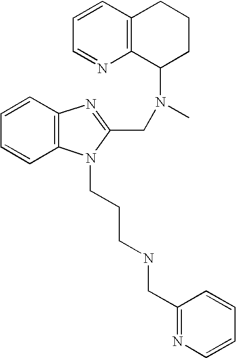 | 7.23 (n = 1) | 6.83 (n = 2) | <4.00(n = 1) | 0.121(n = 2) |
| 17 | 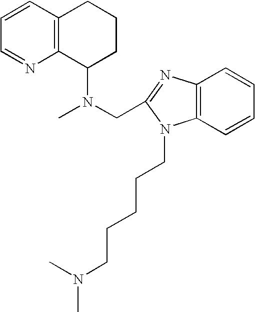 | 6.69 (n = 1) | 6.49 (n = 2) | <4.00(n = 1) | 0.251(n = 2) |
| 18 | 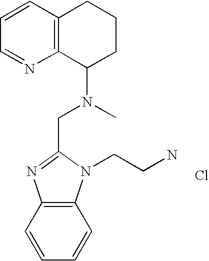 | 6.78 (n = 1) | 6.50 (n = 2) | <4.00(n = 1) | 0.212(n = 2) |
| 19 | 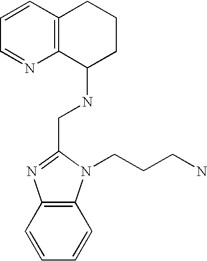 | 6.63 (n = 1) | 6.29 (n = 2) | <4.00(n = 1) | 0.090(n = 2) |
| 20 | 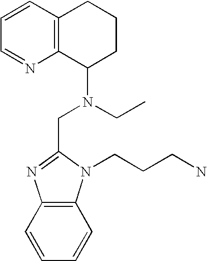 | 7.55 (n = 1) | 7.25 (n = 2) | <4.00(n = 1) | 0.021(n = 2) |
| 21 | 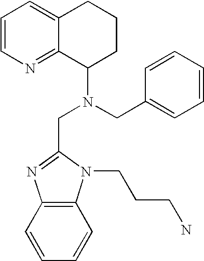 | 6.54 (n = 1) | 5.88 (n = 2) | <4.00(n = 1) | 0.595(n = 2) |
| 22 | 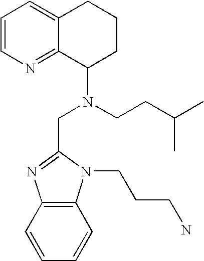 | 7.25 (n = 1) | 6.80 (n = 2) | <4.00(n = 1) | 0.113(n = 2) |
| 23e | 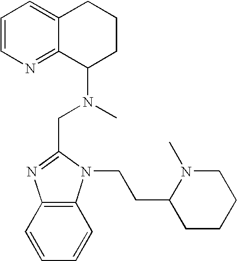 | 7.76 (n = 1) | 7.42 (n = 2) | <4.00 | 0.033(n = 2) |
| 24 | 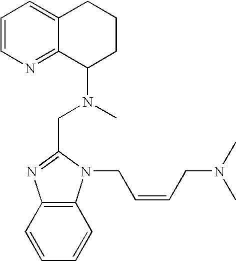 | 7.81 (n = 1) | 7.69 (n = 2) | <4.00(n = 1) | 0.025(n = 1) |
| 25 | 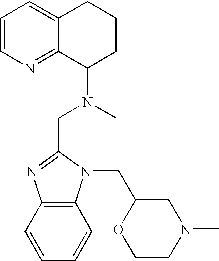 | 7.53 (n = 1) | 7.42 (n = 1) | <4.00(n = 1) | 0.185(n = 2) |

Compounds of the present invention demonstrate desired potency. For example, a preferable potency demonstrated by compounds of the present invention is below 100 nM. Moreover, compounds of the present invention are believed to provide a desired pK profile. Compounds active in HOS assay were also active in the fusion assay. Compounds exhibited separation between activity and cytotoxicicty in the described assays.

Activity of various compounds of the present invention are included in Table 2.

TABLE 2

| Example | Structure | Activity Level* |
|---|---|---|
| 3 | 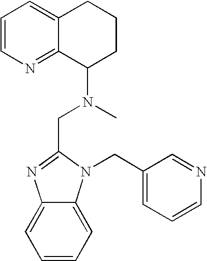 | C |
| 4 | 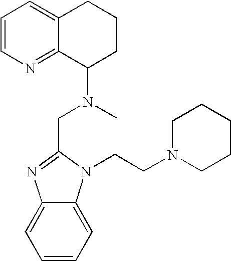 | B |
| 5 | 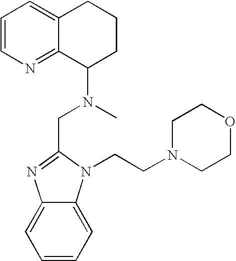 | B |
| 6 | 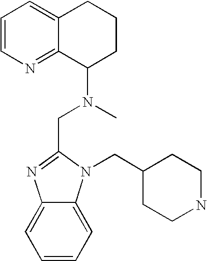 | A |
| 7 | 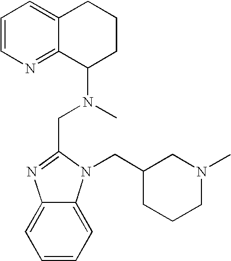 | A |
| 8c | 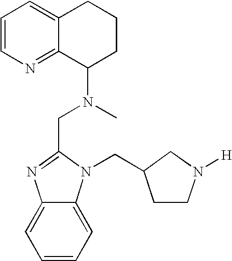 | A |
| 8d | 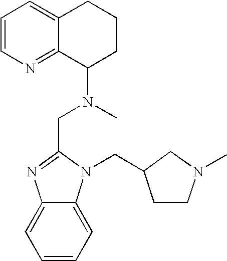 | A |
| 9 | 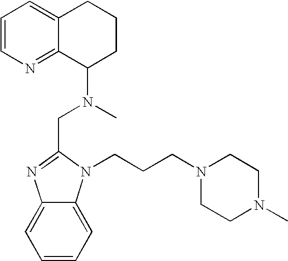 | A |
| 10d | 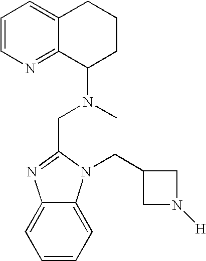 | A |
| 10e | 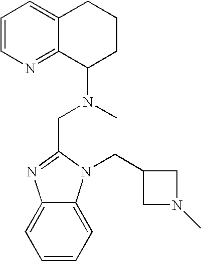 | A |
| 11h | 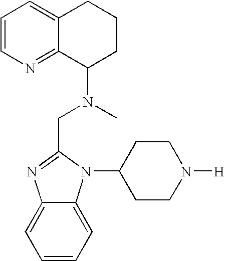 | A |
| 11i | 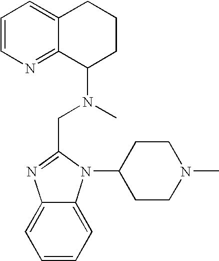 | A |
| 12 | 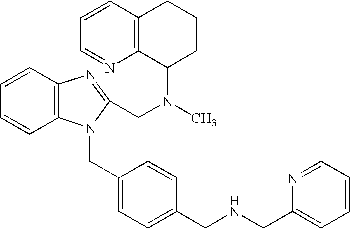 | B |
| 13 | 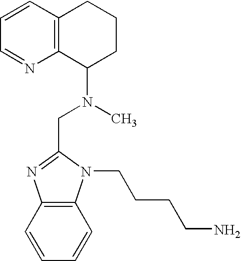 | B |
| 14 | 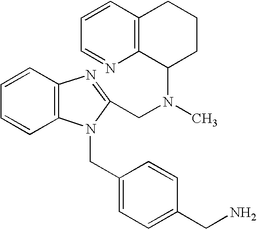 | C |
| 15b | 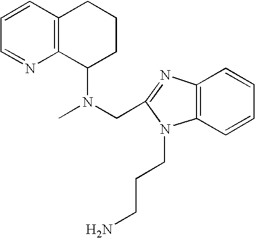 | A |
| 15c | 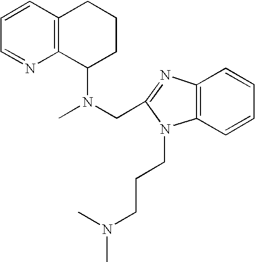 | A |
| 16 | 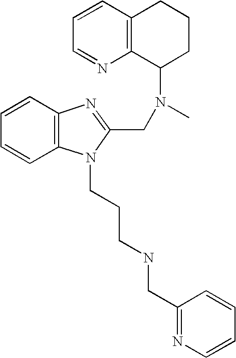 | B |
| 17 | 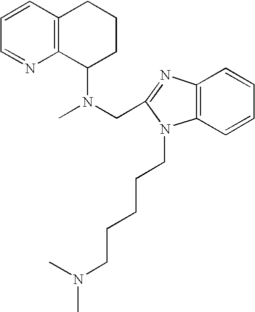 | B |
| 18 | 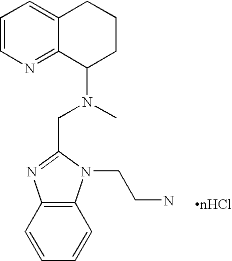 | B |
| 19 | 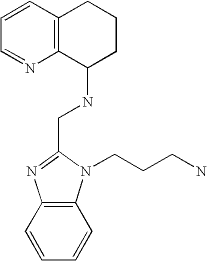 | A |
| 20 | 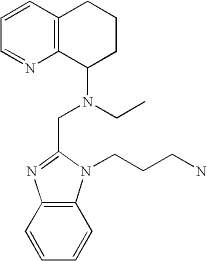 | A |
| 21 | 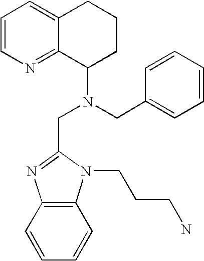 | C |
| 22 | 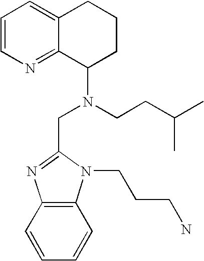 | B |
| 23d | 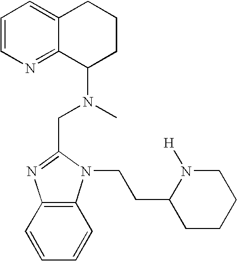 | A |
| 23e | 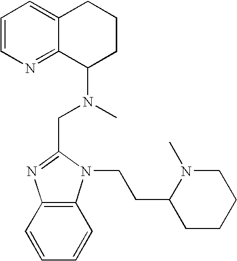 | A |
| 24 | 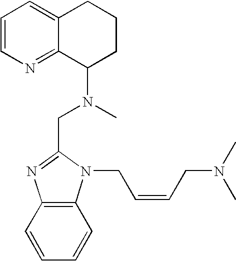 | A |
| 25 | 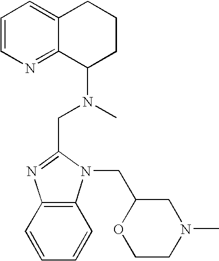 | B |
| 26 | 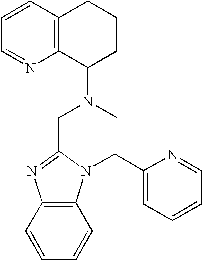 | C |
| 27 | 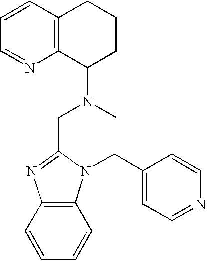 | B |
| 28 | 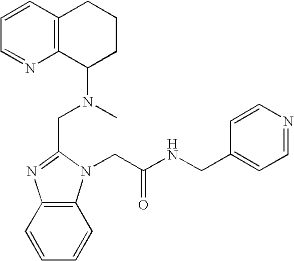 | B |
| 29 | 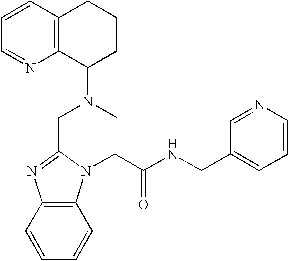 | A |
| 30 | 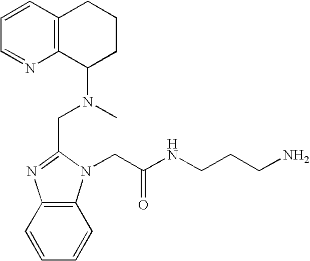 | B |
| 31 | 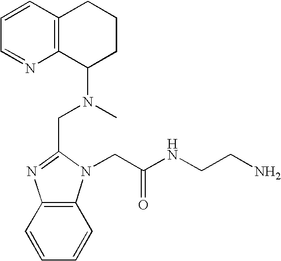 | B |
| 32 | 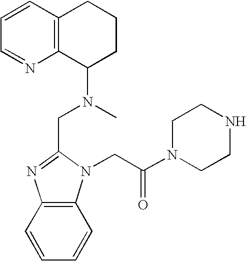 | C |
| 33 | 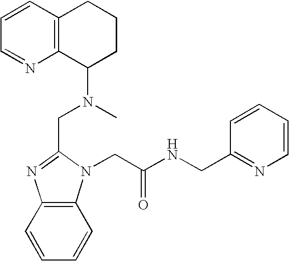 | B |
| 34 | 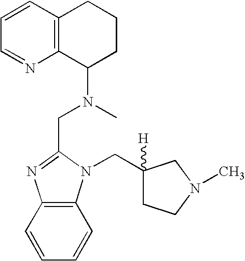 | A |
| 35 | 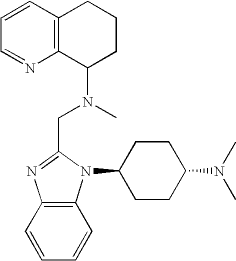 | B |
| 36 | 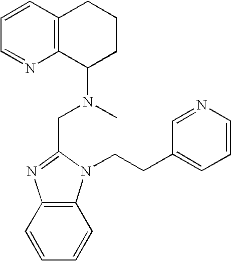 | B |
| 37 | 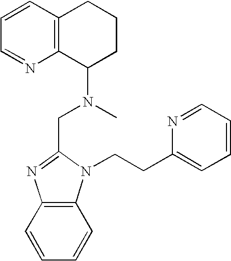 | B |
| 38 | 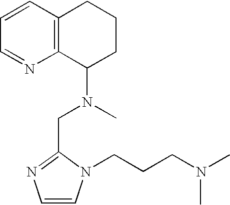 | C |
| 39i | 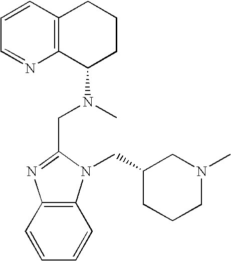 | A |
| 39h | 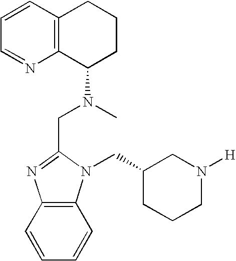 | A |
| 40 | 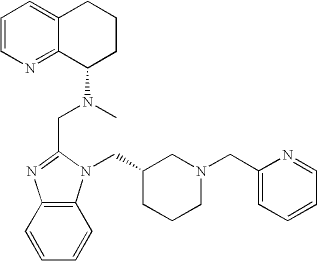 | A |
| 41 | 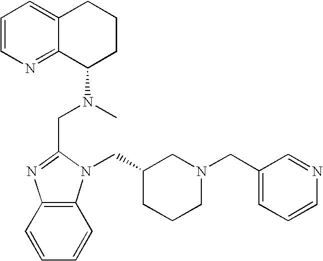 | A |
| 42 | 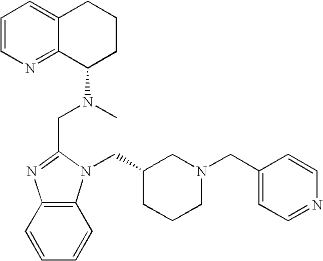 | A |
| 43 | 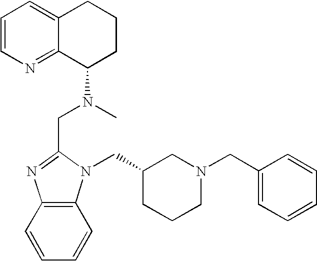 | A |
| 44 | 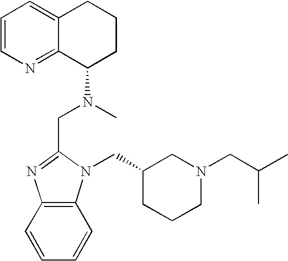 | A |
| 45 | 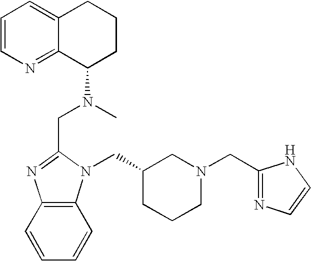 | A |
| 46 | 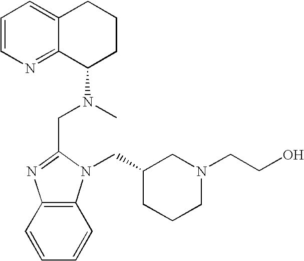 | A |
| 47 | 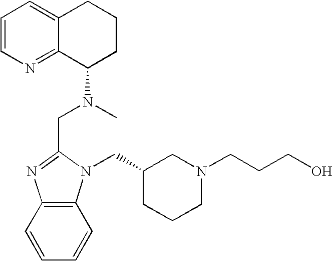 | A |
| 48 | 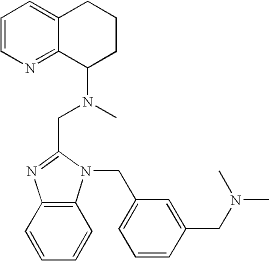 | B |
| 49 | 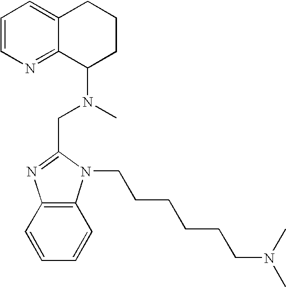 | B |
| 50 | 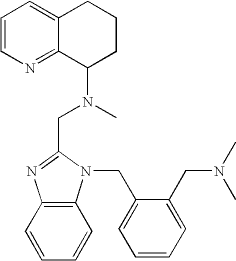 | B |
| 51 | 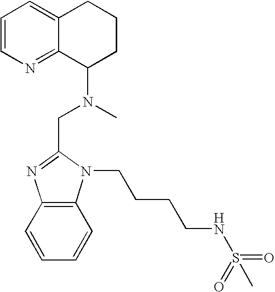 | B |
| 52 | 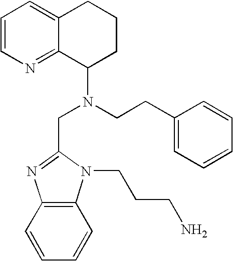 | C |
| 53 | 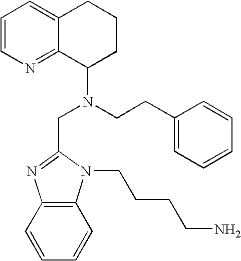 | C |
| 54 | 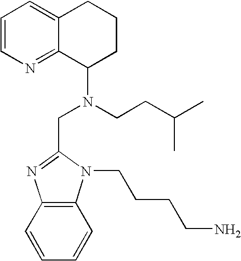 | B |
| 55 | 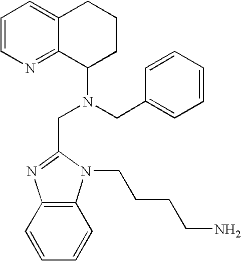 | C |
| 56 | 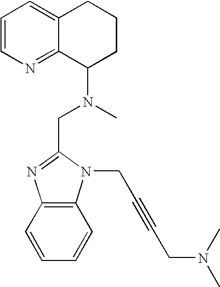 | B |
| 57 | 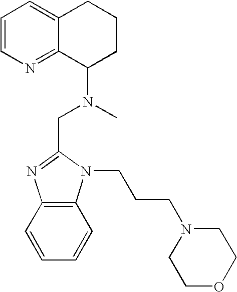 | B |
| 58 | 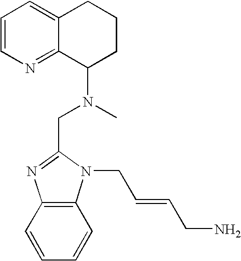 | B |
| 59 | 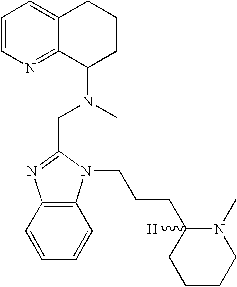 | B |
| 60 | 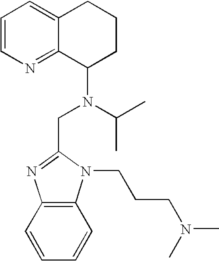 | A |
| 61 | 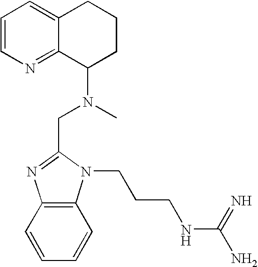 | A |
| 62 | 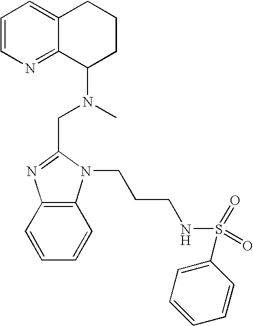 | B |
| 63 | 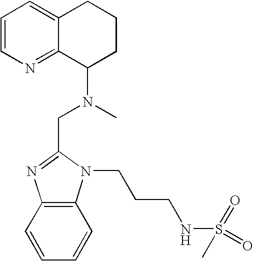 | B |
| 64 | 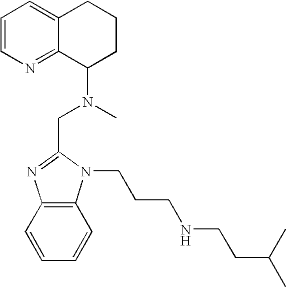 | A |
| 65 | 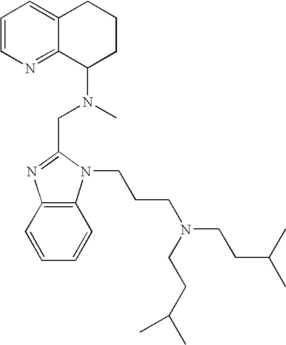 | C |
| 66 | 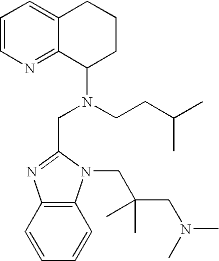 | C |
| 67 | 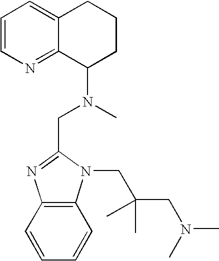 | A |
| 68 | 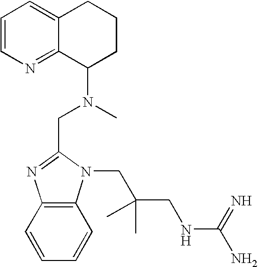 | A |
| 69 | 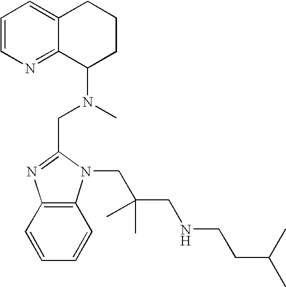 | B |
| 70 | 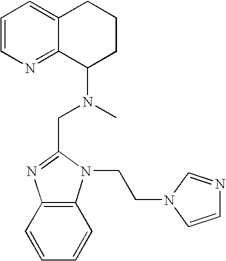 | B |
| 71 | 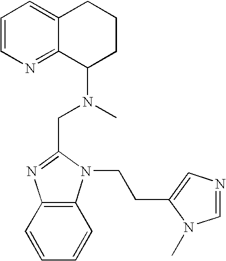 | B |
| 72 | 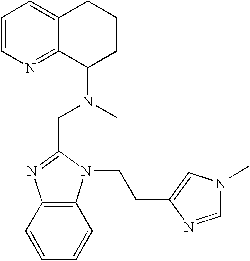 | C |
| 73 | 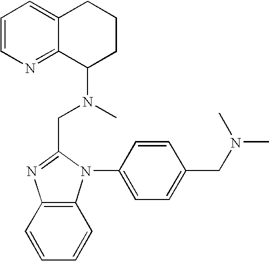 | C |
| 74 | 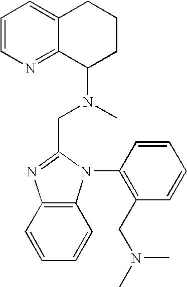 | C |
| 75e | 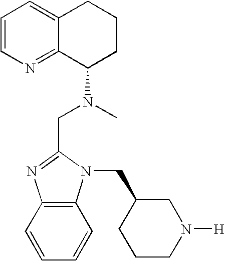 | A |
| 75f | 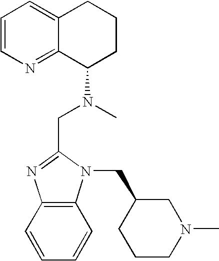 | A |
| 76 | 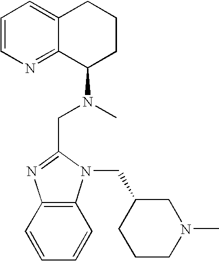 | B |
| 77 | 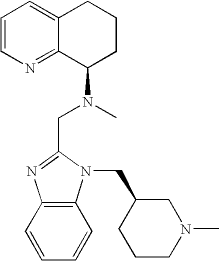 | B |
| 78 | 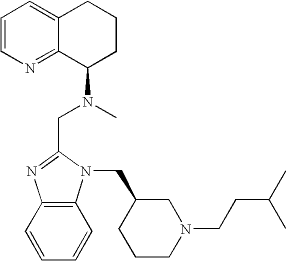 | B |
| 79 | 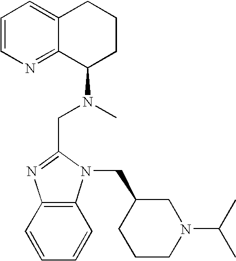 | A |
| 80 | 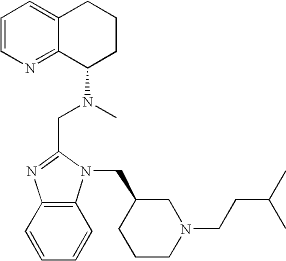 | A |
| 81 | 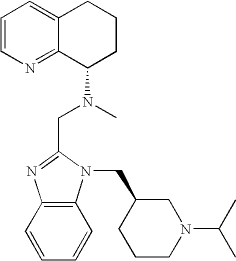 | A |
| 82 | 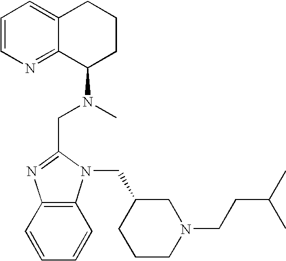 | A |
| 83 | 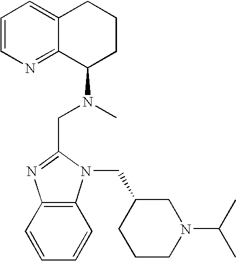 | A |
| 84 | 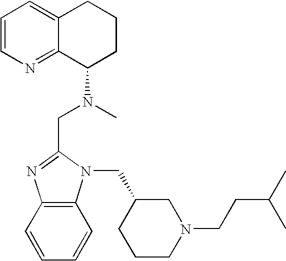 | A |
| 85 | 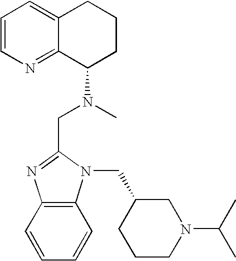 | A |
| 86 | 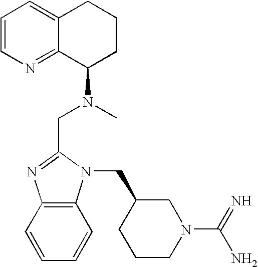 | B |
| 87 | 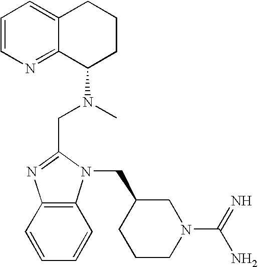 | A |
| 88 | 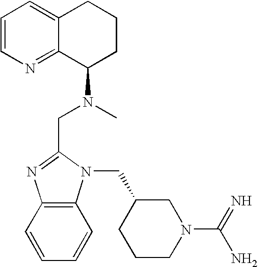 | B |
| 89 | 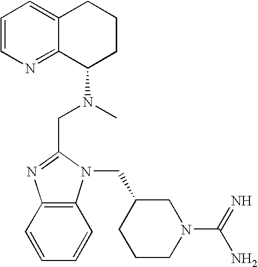 | A |
| 90 | 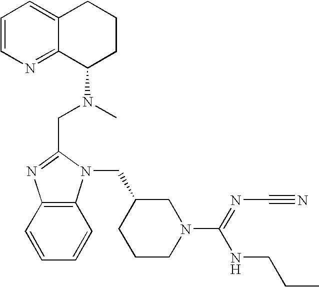 | B |
| 91 | 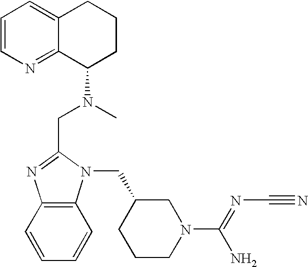 | A |
| 92 | 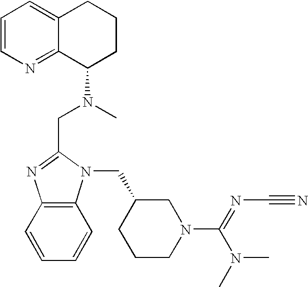 | B |
| 93 | 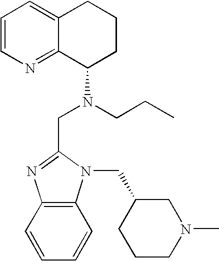 | A |
| 94 | 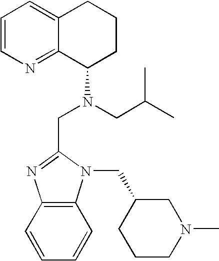 | B |
| 95 | 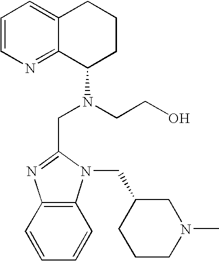 | A |
| 96 | 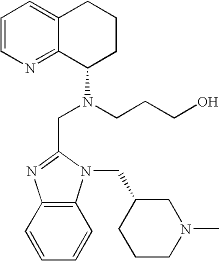 | A |
| 97 | 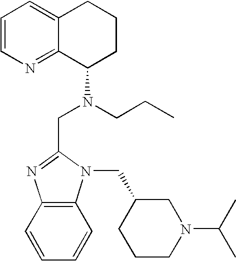 | A |
| 98 | 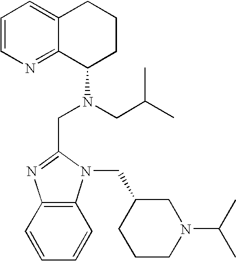 | B |
| 99 | 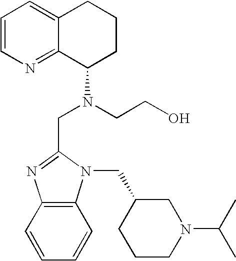 | A |
| 100 | 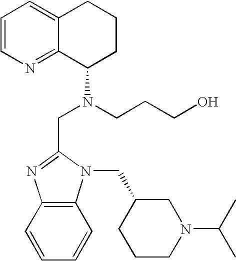 | A |
| 101 | 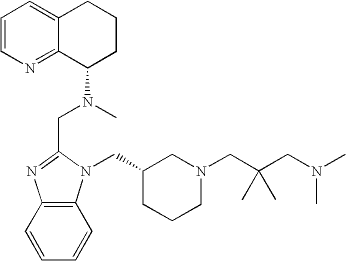 | A |
| 102 | 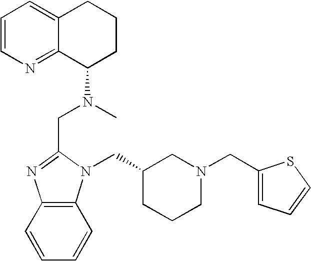 | A |
| 103 | 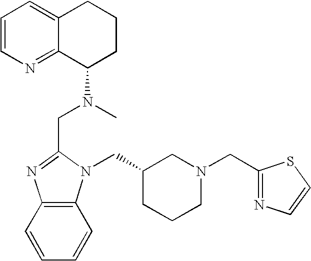 | B |
| 104 | 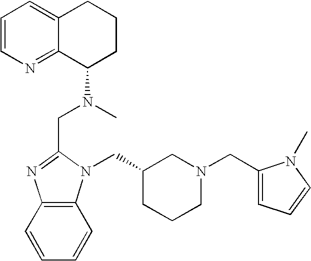 | A |
| 105 | 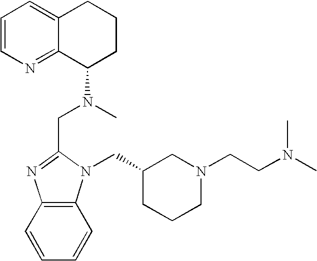 | A |
| 106 | 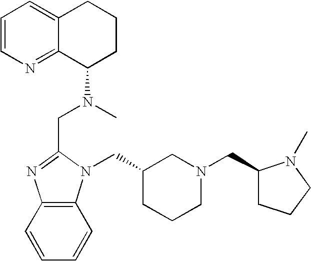 | A |
| 107 | 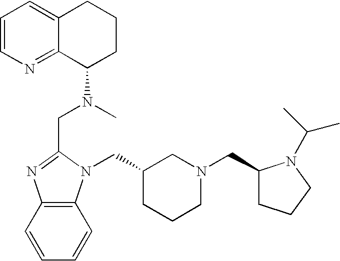 | A |
| 108 | 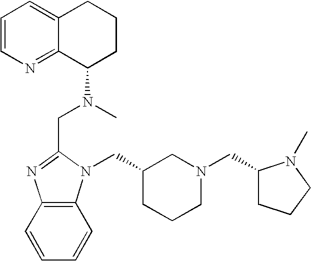 | A |
| 109 | 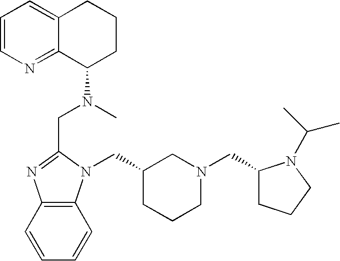 | B |
| 110 | 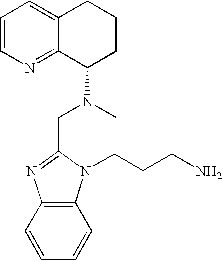 | A |

*“A” indicates compounds with activity in the range of less than 100 nM as determined by the infectivity assay.
“B” indicates compounds with activity in the range of from 100 nM to 500 nM as determined by the infectivity assay.
“C” indicates compounds with activity in the range of from 500 nm to 10 μM as determined by the infectivity assay.

### Test Compounds were Employed in Free or Salt Form.

All research complied with the principles of laboratory animal care (NIH publication No. 85-23, revised 1985) and GlaxoSmithKline policy on animal use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of formula (I) 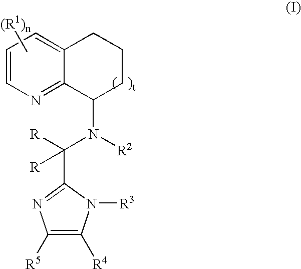
wherein:
t is 0, 1, or 2;
each R independently is H, alkyl, alkenyl, alkynyl, haloalkyl, cycloalkyl, —R^{a}Ay, —R^{a}OR¹⁰, or R^{a}S(O)ₘR¹⁰;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)ₘR¹⁰, —S(O)ₘAy, cyano, nitro, or azido;
n is 0, 1, or 2;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₙR¹⁰ wherein R² is not amine or alkylamine, or substituted with amine or alklyamine;
R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}NR⁶R⁷]ₚ, —R^{a}Ay[R^{a}NR⁶R⁷]ₚ, -Het[NR⁶R⁷]ₚ, —R^{a}Het[NR⁶R⁷]ₚ-Het[R^{a}NR⁶R⁷]ₚ, or —R^{a}Het[R^{a}NR⁶R⁷]ₚ;
each p independently is 1 or 2;
each of R⁴ and R⁵ independently are selected from H, alkyl, alkenyl, alkynyl, cycloalkyl, -Ay, -Het, —R^{a}Ay, —R^{a}Het, —OR¹⁰, —NR⁶R⁷, —R^{a}NR⁶R⁷, —C(O)R¹⁰, —CO₂R¹⁰—C(O)NR⁶R⁷, —S(O)₂NR⁶R⁷, —S(O)ₘR¹⁰, cyano, nitro, or azido; or
R⁴ and R⁵ may combine to form a ring containing one or more degrees of unsaturation that is fused with the depicted imidazole ring optionally substituted with (R¹)ₙ;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het, —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene; and
each Ay independently represents an optionally substituted aryl group; and
each Het independently represents an optionally substituted 4-, 5-, or 6-membered heterocyclyl or heteroaryl group; or a pharmaceutically acceptable salt or solvate thereof.

2. The compound of claim 1 wherein R⁴ and R⁵ combine to form a benzene ring so as to form a benzimidazole ring.

3. The compound of claim 1 wherein R⁴ and R⁵ are independently H, alkyl, Ay, Het, —NR⁶R⁷, —R^{a}NR⁶R⁷, —C(O)R¹⁰, —C(O)NR⁶R⁷, —C(O)Ay, —C(O)Het, or —SO₂NR⁶R⁷.

4. The compound of claim 3 wherein R⁴ and R⁵ are independently H, alkyl, Ay, or —R^{a}NR⁶R⁷.

5. The compound of claim 4 wherein alkyl is C₁-C₆ alkyl and Ay is phenyl.

6. The compound of claim 1 wherein n is 1 or 2 and each R¹ independently is selected from halogen, C₁-C₆ alkyl, —OR¹⁰, —NR⁶R⁷, —C(O)R¹⁰, —CO₂R¹⁰, —C(O)NR⁶R⁷, or —S(O)₂NR⁶R⁷.

7. The compound of claim 1 wherein n is 0.

8. The compound of claim 1 wherein R is H, C₁-C₆ alkyl, C₁-C₆ haloalkyl, or C₃-C₆ cycloalkyl.

9. The compound of claim 8 wherein R² is C₁-C₆ alkyl, C₁-C₆ haloalkyl, or C₃-C₆ cycloalkyl.

10. The compound of claim 1 wherein each of R⁶ and R⁷ independently are selected from H, C₁-C₆ alkyl, C₃-C₆ cycloalkyl, —R^{a}OH, and —R^{a}OR¹⁰.

11. The compound of claim 1 wherein R¹⁰ is H, C₁-C₆ alkyl, or C₃-C₆ cycloalkyl.

12. The compound of claim 1 wherein R^{a} is C₁-C₆ alkylene or C₃-C₆ cycloalkylene.

13. The compound of claim 1 wherein R is H, alkyl, or cycloalkyl.

14. The compound of claim 13 wherein R is H.

15. The compound of claim 1 wherein -Het is a nitrogen-containing heterocyclyl or heteroaryl ring.

16. The compound of claim 1 wherein -Het is an optionally substituted pyridinyl, piperidinyl, piperizinyl, morpholinyl, pyrrolidinyl, imidazolyl, or azetedinyl.

17. The compound of claim 1 wherein -Het is optionally substituted with one or more C₁-C₆ alkyl, C₃-C₆ cycloalkyl, amino, C₁-C₆ alkylamino, hydroxyl, C₁-C₆ alkoxy, C₁-C₆ cycloalkoxy, and halogen.

18. (canceled)

19. The compound of claim 1 wherein -Ay is optionally substituted with one or more C₁-C₆ alkyl, C₃-C₆ cycloalkyl, amino, C₁-C₆ alkylamino, hydroxyl, C₁-C₆ alkoxy, C₁-C₆ cycloalkoxy, and halogen.

20. The compound of claim 1 wherein t is 1.

21. The compound of claim 1 wherein R³ is -Het, —R^{a}NR⁶R⁷, —Het[NR⁶R⁷]ₚ, —R^{a}Het[NR⁶R⁷]ₚ, -Het[R^{a}NR⁶R⁷]ₚ, or —R^{a}Het[R^{a}NR⁶R⁷]ₚ; —R^{a}Het, and -Het is a nitrogen-containing heterocyclyl or heteroaryl ring optionally substituted with one or more C₁-C₆ alkyl, C₃-C₆ cycloalkyl, amino, C₁-C₆ alkylamino, hydroxyl, C₁-C₆ alkoxy, C₁-C₆ cycloalkoxy, and halogen.

22. The compound of claim 1 wherein R³ is -Het, -Het[NR⁶R⁷]ₚ, —R^{a}Het[NR⁶R⁷]ₚ; or —R^{a}Het, and -Het is a nitrogen-containing heterocyclyl or heteroaryl ring optionally substituted with one or more C₁-C₆ alkyl, C₃-C₆ cycloalkyl, amino, C₁-C₆ alkylamino, hydroxyl, C₁-C₆ alkoxy, C₁-C₆ cycloalkoxy, and halogen.

23. The compound of claim 1 wherein R³ is —R^{a}Het, and -Het is a nitrogen-containing heterocyclyl or heteroaryl ring optionally substituted with one or more C₁-C₆ alkyl, C₃-C₆ cycloalkyl, amino, C₁-C₆alkylamino, hydroxyl, C₁-C₆ alkoxy, C₁-C₆ cycloalkoxy, and halogen.

24. A compound selected from the group consisting of:
N-Methyl-N-{[1-(3-pyridinylmethyl)-1H-benzimidazol-2-yl]methyl}-5,6,7,8-tetrahydro-8-quinolinamine;
N-Methyl-N-({1-[2-(1-piperidinyl)ethyl]-1H-benzimidazol-2-yl}methyl)-5,6,7,8-tetrahydro-8-quinolinamine;
N-Methyl-N-({1-[2-(4-morpholinyl)ethyl]-1H-benzimidazol-2-yl}methyl)-5,6,7,8-tetrahydro-8-quinolinamine;
N-Methyl-N-{[1-(4-piperidinylmethyl)-1H-benzimidazol-2-yl]methyl}-5,6,7,8-tetrahydro-8-quinolinamine;
N-Methyl-N-({1-[(1-methyl-3-piperidinyl)methyl]-1H-benzimidazol-2-yl}methyl)-5,6,7,8-tetrahydro-8-quinolinamine,
N-Methyl-N-({1-[(1-methyl-3-pyrrolidinyl)methyl]-1H-benzimidazol-2-yl}methyl)-5,6,7,8-tetrahydro-8-quinolinamine;
N-Methyl-N-({1-[3-(4-methyl-1-piperazinyl)propyl]-1H-benzimidazol-2-yl}methyl)-5,6,7,8-tetrahydro-8-quinolinamine;
N-Methyl-N-({1-[(1-methyl-3-azetidinyl)methyl]-1H-benzimidazol-2-yl}methyl)-5,6,7,8-tetrahydro-8-quinolinamine;
N-Methyl-N-{[1-(1-methyl-4-piperidinyl)-1H-benzimidazol-2-yl]methyl}-5,6,7,8-tetrahydro-8-quinolinamine;
N-Methyl-N-({1-[(4-{[(2-pyridinylmethyl)amino]methyl}phenyl)methyl]-1H-benzimidazol-2-yl}methyl)-5,6,7,8-tetrahydro-8-quinolinamine;
N-{[1-(4-Aminobutyl)-1H-benzimidazol-2-yl]methyl}-N-methyl-5,6,7,8-tetrahydro-8-quinolinamine;
N-[(1-{[4-(Aminomethyl)phenyl]methyl}-1H-benzimidazol-2-yl)methyl]-N-methyl-5,6,7,8-tetrahydro-8-quinolinamine;
N-({1-[3-(Dimethylamino)propyl]-1H-benzimidazol-2-yl}methyl)-N-methyl-5,6,7,8-tetrahydro-8-quinolinamine;
N-Methyl-N-[(1-{3-[(2-pyridinylmethyl)amino]propyl}-1H-benzimidazol-2-yl)methyl]-5,6,7,8-tetrahydro-8-quinolinamine;
N-({1-[5-(Dimethylamino)pentyl]-1H-benzimidazol-2-yl}methyl)-N-methyl-5,6,7,8-tetrahydro-8-quinolinamine;
N-{[1-(2-Aminoethyl)-1H-benzimidazol-2-yl]methyl}-N-methyl-5,6,7,8-tetrahydro-8-quinolinamine;
N-{[1-(3-Aminopropyl)-1H-benzimidazol-2-yl]methyl}-5,6,7,8-tetrahydro-8-quinolinamine;
N-{[1-(3-Aminopropyl)-1H-benzimidazol-2-yl]methyl}-N-ethyl-5,6,7,8-tetrahydro-8-quinolinamine;
N-{[1-(3-Aminopropyl)-1H-benzimidazol-2-yl]methyl}-N-(phenylmethyl)-5,6,7,8-tetrahydro-8-quinolinamine;
N-{[1-(3-Aminopropyl)-1H-benzimidazol-2-yl]methyl}-N-(3-methylbutyl)-5,6,7,8-tetrahydro-8-quinolinamine;
N-Methyl-N-({1-[2-(1-methyl-2-piperidinyl)ethyl]-1H-benzimidazol-2-yl}methyl)-5,6,7,8-tetrahydro-8-quinolinamine;
N-({1-[(2Z)-4-(Dimethylamino)-2-buten-1-yl]-1H-benzimidazol-2-yl}methyl)-N-methyl-5,6,7,8-tetrahydro-8-quinolinamine; and
N-Methyl-N-({1-[(4-methyl-2-morpholinyl)methyl]-1H-benzimidazol-2-yl}methyl)-5,6,7,8-tetrahydro-8-quinolinamine;
N-Methyl-N-{[1-(2-pyridinylmethyl)-1H-benzimidazol-2-yl]methyl}-5,6,7,8-tetrahydro-8-quinolinamine;
N-Methyl-N-{[1-(4-pyridinylmethyl)-1H-benzimidazol-2-yl]methyl}-5,6,7,8-tetrahydro-8-quinolinamine;
2-(2-{[Methyl(5,6,7,8-tetrahydro-8-quinolinyl)amino]methyl}-1H-benzimidazol-1-yl)-N-(4-pyridinylmethyl)acetamide;
2-(2-{[Methyl(5,6,7,8-tetrahydro-8-quinolinyl)amino]methyl}-1H-benzimidazol-1-yl)-N-(3-pyridinylmethyl)acetamide;
N-(3-Aminopropyl)-2-(2-{[methyl(5,6,7,8-tetrahydro-8-quinolinyl)amino]methyl}-1H-benzimidazol-1-yl)acetamide;
N-(2-Aminoethyl)-2-(2-{[methyl(5,6,7,8-tetrahydro-8-quinolinyl)amino]methyl}-1H-benzimidazol-1-yl)acetamide;
N-Methyl-N-({1-[2-oxo-2-(1-piperazinyl)ethyl]-1H-benzimidazol-2-yl}methyl)-5,6,7,8-tetrahydro-8-quinolinamine;
2-(2-{[Methyl(5,6,7,8-tetrahydro-8-quinolinyl)amino]methyl}-1H-benzimidazol-1-yl)-N-(2-pyridinylmethyl)acetamide;
N-Methyl-N-({1-[(1-methyl-3-pyrrolidinyl)methyl]-1H-benzimidazol-2-yl}methyl)-5,6,7,8-tetrahydro-8-quinolinamine HCl salt;
N-({1-[trans-4-(Dimethylamino)cyclohexyl]-1H-benzimidazol-2-yl}methyl)-N-methyl-5,6,7,8-tetrahydro-8-quinolinamine;
N-Methyl-N-({1-[2-(3-pyridinyl)ethyl]-1H-benzimidazol-2-yl}methyl)-5,6,7,8-tetrahydro-8-quinolinamine;
N-methyl-N-({1-[2-(2-pyridinyl)ethyl]-1H-benzimidazol-2-yl}methyl)-5,6,7,8-tetrahydro-8-quinolinamine;
N-({1-[3-(Dimethylamino)propyl]-1H-imidazol-2-yl}methyl)-N-methyl-5,6,7,8-tetrahydro-8-quinolinamine;
(8S)—N-Methyl-N-[(1-{[(3S)-1-methyl-3-piperidinyl]methyl}-1H-benzimidazol-2-yl)methyl]-5,6,7,8-tetrahydro-8-quinolinamine;
(8S)—N-Methyl-N-[(1-{[(3S)-1-(2-pyridinylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(3-pyridinylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(4-pyridinylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(phenylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(2-methylpropyl)-3-piperidinyl]methyl}-1H-benzimidazol-2-yl)methyl]-5,6,7,8-tetrahydro-8-quinolinamine;
(8S)—N-[(1-{[(3S)-1-(1H-Imidazol-2-ylmethyl)-3-piperidinyl]methyl}-1H-benzimidazol-2-yl)methyl]-N-methyl-5,6,7,8-tetrahydro-8-quinolinamine;
2-((3S)-3-{[2-({Methyl[(8S)-5,6,7,8-tetrahydro-8-quinolinyl]amino}methyl)-1H-benzimidazol-1-yl]methyl}-1-piperidinyl)ethanol;
3-((3S)-3-{[2-({Methyl[(8S)-5,6,7,8-tetrahydro-8-quinolinyl]amino}methyl)-1H-benzimidazol-1-yl]methyl}-1-piperidinyl)-1-propanol;
N-{[1-({3-[(Dimethylamino)methyl]phenyl}methyl)-1H-benzimidazol-2-yl]methyl}-N-methyl-5,6,7,8-tetrahydro-8-quinolinamine;
N-({1-[6-(Dimethylamino)hexyl]-1H-benzimidazol-2-yl}methyl)-N-methyl-5,6,7,8-tetrahydro-8-quinolinamine;
N-{[1-({2-[(Dimethylamino)methyl]phenyl}methyl)-1H-benzimidazol-2-yl]methyl}-N-methyl-5,6,7,8-tetrahydro-8-quinolinamine;
N-[4-(2-{[Methyl(5,6,7,8-tetrahydro-8-quinolinyl)amino]methyl}-1H-benzimidazol-1-yl)butyl]methanesulfonamide;
N-{[1-(3-Aminopropyl)-1H-benzimidazol-2-yl]methyl}-N-(2-phenylethyl)-5,6,7,8-tetrahydro-8-quinolinamine;
N-{[1-(4-Aminobutyl)-1H-benzimidazol-2-yl]methyl}-N-(2-phenylethyl)-5,6,7,8-tetrahydro-8-quinolinamine;
N-{[1-(4-Aminobutyl)-1H-benzimidazol-2-yl]methyl}-N-(3-methylbutyl)-5,6,7,8-tetrahydro-8-quinolinamine;
N-{[1-(4-Aminobutyl)-1H-benzimidazol-2-yl]methyl}-N-(phenylmethyl)-5,6,7,8-tetrahydro-8-quinolinamine;
N-({1-[4-(Dimethylamino)-2-butyn-1-yl]-1H-benzimidazol-2-yl}methyl)-N-methyl-5,6,7,8-tetrahydro-8-quinolinamine;
N-Methyl-N-({1-[3-(4-morpholinyl)propyl]-1H-benzimidazol-2-yl}methyl)-5,6,7,8-tetrahydro-8-quinolinamine;
N-({1-[(2E)-4-Amino-2-buten-1-yl]-1H-benzimidazol-2-yl}methyl)-N-methyl-5,6,7,8-tetrahydro-8-quinolinamine;
N-Methyl-N-({1-[3-(1-methyl-2-piperidinyl)propyl]-1H-benzimidazol-2-yl}methyl)-5,6,7,8-tetrahydro-8-quinolinamine;
N-({1-[3-(Dimethylamino)propyl]-1H-benzimidazol-2-yl}methyl)-N-(1-methylethyl)-5,6,7,8-tetrahydro-8-quinolinamine;
N-[3-(2-{[Methyl(5,6,7,8-tetrahydro-8-quinolinyl)amino]methyl}-1H-benzimidazol-1-yl)propyl]guanidine;
N-[3-(2-{[Methyl(5,6,7,8-tetrahydro-8-quinolinyl)amino]methyl}-1H-benzimidazol-1-yl)propyl]benzenesulfonamide;
N-[3-(2-{[Methyl(5,6,7,8-tetrahydro-8-quinolinyl)amino]methyl}-1H-benzimidazol-1-yl)propyl]methanesulfonamide;
N-Methyl-N-[(1-{3-[(3-methylbutyl)amino]propyl}-1H-benzimidazol-2-yl)methyl]-5,6,7,8-tetrahydro-8-quinolinamine;
N-[(1-{3-[Bis(3-methylbutyl)amino]propyl}-1H-benzimidazol-2-yl)methyl]-N-methyl-5,6,7,8-tetrahydro-8-quinolinamine;
N-({1-[3-(Dimethylamino)-2,2-dimethylpropyl]-1H-benzimidazol-2-yl}methyl)-N-(3-methylbutyl)-5,6,7,8-tetrahydro-8-quinolinamine;
N-({1-[3-(Dimethylamino)-2,2-dimethylpropyl]-1H-benzimidazol-2-yl}methyl)-N-methyl-5,6,7,8-tetrahydro-8-quinolinamine;
N-[2,2-Dimethyl-3-(2-{[methyl(5,6,7,8-tetrahydro-8-quinolinyl)amino]methyl}-1H-benzimidazol-1-yl)propyl]guanidine;
N-[(1-{2,2-Dimethyl-3-[(3-methylbutyl)amino]propyl}-1H-benzimidazol-2-yl)methyl]-N-methyl-5,6,7,8-tetrahydro-8-quinolinamine;
N-({1-[2-(1H-Imidazol-1-yl)ethyl]-1H-benzimidazol-2-yl}methyl)-N-methyl-5,6,7,8-tetrahydro-8-quinolinamine;
N-Methyl-N-({1-[2-(1-methyl-1H-imidazol-5-yl)ethyl]-1H-benzimidazol-2-yl}methyl)-5,6,7,8-tetrahydro-8-quinolinamine;
N-Methyl-N-({1-[2-(1-methyl-1H-imidazol-4-yl)ethyl]-1H-benzimidazol-2-yl}methyl)-5,6,7,8-tetrahydro-8-quinolinamine;
N-[(1-{4-[(Dimethylamino)methyl]phenyl}-1H-benzimidazol-2-yl)methyl]-N-methyl-5,6,7,8-tetrahydro-8-quinolinamine;
N-[(1-{2-[(Dimethylamino)methyl]phenyl}-1H-benzimidazol-2-yl)methyl]-N-methyl-5,6,7,8-tetrahydro-8-quinolinamine;
(8S)—N-Methyl-N-[(1-{[(3R)-1-methyl-3-piperidinyl]methyl}-1H-benzimidazol-2-yl)methyl]-5,6,7,8-tetrahydro-8-quinolinamine;
(8R)—N-Methyl-N-[(1-{[(3S)-1-methyl-3-piperidinyl]methyl}-1H-benzimidazol-2-yl)methyl]-5,6,7,8-tetrahydro-8-quinolinamine,
(8R)—N-Methyl-N-[(1-{[(3R)-1-methyl-3-piperidinyl]methyl}-1H-benzimidazol-2-yl)methyl]-5,6,7,8-tetrahydro-8-quinolinamine,
(8R)—N-Methyl-N-[(1-{[(3R)-1-(3-methylbutyl)-3-piperidinyl]methyl}-1H-benzimidazol-2-yl)methyl]-5,6,7,8-tetrahydro-8-quinolinamine;
(8R)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine;
(8S)—N-Methyl-N-[(1-{[(3R)-1-(3-methylbutyl)-3-piperidinyl]methyl}-1H-benzimidazol-2-yl)methyl]-5,6,7,8-tetrahydro-8-quinolinamine;
(8S)—N-Methyl-N-[(1-{[(3R)-1-(1-methylethyl)-3-piperidinyl]methyl}-1H-benzimidazol-2-yl)methyl]-5,6,7,8-tetrahydro-8-quinolinamine;
(8R)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine;
(8R)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(3-methylbutyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(1-methylethyl)-3-piperidinyl]methyl}-1H-benzimidazol-2-yl)methyl]-5,6,7,8-tetrahydro-8-quinolinamine;
(3S)-3-{[2-({Methyl[(8R)-5,6,7,8-tetrahydro-8-quinolinyl]amino}methyl)-1H-benzimidazol-1-yl]methyl}-1-piperidinecarboximidamide;
(3S)-3-{[2-({Methyl[(8S)-5,6,7,8-tetrahydro-8-quinolinyl]amino}methyl)-1H-benzimidazol-1-yl]methyl}-1-piperidinecarboximidamide;
(3R)-3-{[2-({Methyl[(8R)-5,6,7,8-tetrahydro-8-quinolinyl]amino}methyl)-1H-benzimidazol-1-yl]methyl}-1-piperidinecarboximidamide;
(3R)-3-{[2-({Methyl[(8S)-5,6,7,8-tetrahydro-8-quinolinyl]amino}methyl)-1H-benzimidazol-1-yl]methyl}-1-piperidinecarboximidamide;
(3R)—N-Cyano-3-{[2-({methyl[(8S)-5,6,7,8-tetrahydro-8-quinolinyl]amino}methyl)-1H-benzimidazol-1-yl]methyl}-N′-propyl-1-piperidinecarboximidamide;
(3R)—N-Cyano-3-{[2-({methyl[(8S)-5,6,7,8-tetrahydro-8-quinolinyl]amino}methyl)-1H-benzimidazol-1-yl]methyl}-1-piperidinecarboximidamide;
(8R)—N′-Cyano-N,N-dimethyl-3-{[2-({methyl[(8S)-5,6,7,8-tetrahydro-8-quinolinyl]amino}methyl)-1H-benzimidazol-1-yl]methyl}-1-piperidinecarboximidamide;
(8S)—N-[(1-{[(3S)-1-Methyl-3-piperidinyl]methyl}-1H-benzimidazol-2-yl)methyl]-N-propyl-5,6,7,8-tetrahydro-8-quinolinamine;
(8S)—N-[(1-{[(3S)-1-Methyl-3-piperidinyl]methyl}-1H-benzimidazol-2-yl)methyl]-N-(2-methylpropyl)-5,6,7,8-tetrahydro-8-quinolinamine;
2-{[(1-{[(3S)-1-Methyl-3-piperidinyl]methyl}-1H-benzimidazol-2-yl)methyl][(8S)-5,6,7,8-tetrahydro-8-quinolinyl]amino}ethanol;
3-{[(1-{[(3S)-1-Methyl-3-piperidinyl]methyl}-1H-benzimidazol-2-yl)methyl][(8S)-5,6,7,8-tetrahydro-8-quinolinyl]amino}-1-propanol;
(8S)—N-[(1-{[(3S)-1-(1-Methylethyl)-3-piperidinyl]methyl}-1H-benzimidazol-2-yl)methyl]-N-propyl-5,6,7,8-tetrahydro-8-quinolinamine;
(8S)—N-[(1-{[(3S)-1-(1-Methylethyl)-3-piperidinyl]methyl}-1H-benzimidazol-2-yl)methyl]-N-(2-methylpropyl)-5,6,7,8-tetrahydro-8-quinolinamine-2-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}ethanol;
3-{[(1-{[(3S)-1-(1-Methylethyl)-3-piperidinyl]methyl}-1H-benzimidazol-2-yl)methyl][(8S)-5,6,7,8-tetrahydro-8-quinolinyl]amino}-1-propanol;
(8S)—N-{[1-({(3S)-1-[3-(Dimethylamino)-2,2-dimethylpropyl]-3-piperidinyl}methyl)-1H-benzimidazol-2-yl]methyl}-N-methyl-5,6,7,8-tetrahydro-8-quinolinamine;
(8S)—N-Methyl-N-[(1-{[(3S)-1-(2-thienylmethyl)-3-piperidinyl]methyl}-1H-benzimidazol-2-yl)methyl]-5,6,7,8-tetrahydro-8-quinolinamine;
(8S)—N-Methyl-N-[(1-{[(3S)-1-(1,3-thiazol-2-ylmethyl)-3-piperidinyl]methyl}-1H-benzimidazol-2-yl)methyl]-5,6,7,8-tetrahydro-8-quinolinamine;
(8S)—N-Methyl-N-{[1-({(3S)-1-[(1-methyl-1H-pyrrol-2-yl)methyl]-3-piperidinyl}methyl)-1H-benzimidazol-2-yl]methyl}-5,6,7,8-tetrahydro-8-quinolinamine;
(8S)—N-{[1-({(3S)-1-[2-(Dimethylamino)ethyl]-3-piperidinyl}methyl)-1H-benzimidazol-2-yl]methyl}-N-methyl-5,6,7,8-tetrahydro-8-quinolinamine;
(8S)—N-Methyl-N-({1-[((3S)-1-{[(2S)-1-methyl-2-pyrrolidinyl]methyl}-3-piperidinyl)methyl]-1H-benzimidazol-2-yl}methyl)-5,6,7,8-tetrahydro-8-quinolinamine;
(8S)—N-Methyl-N-({1-[((3S)-1-{[(2S)-1-(1-methylethyl)-2-pyrrolidinyl]methyl}-3-piperidinyl)methyl]-1H-benzimidazol-2-yl}methyl)-5,6,7,8-tetrahydro-8-quinolinamine
(8S)—N-Methyl-N-({1-[((3S)-1-{[(2R)-1-methyl-2-pyrrolidinyl]methyl}-3-piperidinyl)methyl]-1H-benzimidazol-2-yl}methyl)-5,6,7,8-tetrahydro-8-quinolinamine;
(8S)—N-Methyl-N-({1-[((3S)-1-{[(2R)-1-(1-methylethyl)-2-pyrrolidinyl]methyl}-3-piperidinyl)methyl]-1H-benzimidazol-2-yl}methyl)-5,6,7,8-tetrahydro-8-quinolinamine;
(8S)—N-{[1-(3-Aminopropyl)-1H-benzimidazol-2-yl]methyl}-N-methyl-5,6,78-tetrahydro-8-quinolinamine;
(8S)—N-({1-[3-(Dimethylamino)propyl]-1H-benzimidazol-2-yl}methyl)-N-methyl-5,6,7,8-tetrahydro-8-quinolinamine;
(8S)—N-Methyl-N-[(1-{3-[(2-methylpropyl)amino]propyl}-1H-benzimidazol-2-yl)methyl]-5,6,7,8-tetrahydro-8-quinolinamine;
(8S)—N-Methyl-N-[(1-{3-[(1-methylethyl)amino]propyl}-1H-benzimidazol-2-yl)methyl]-5,6,7,8-tetrahydro-8-quinolinamine; and
(8S)—N-[(1-{3-[(1H-Imidazol-2-ylmethyl)amino]propyl}-1H-benzimidazol-2-yl)methyl]-N-methyl-5,6,7,8-tetrahydro-8-quinolinamine;
or pharmaceutically acceptable salts or solvates thereof.

25. (canceled)

26. (canceled)

27. (canceled)

28. (canceled)

29. (canceled)

30. (canceled)

31. A pharmaceutical composition comprising a compound according to claim 1, and a pharmaceutically acceptable carrier.

32. A compound according to claim 1 for use as an active therapeutic substance.

33. A compound according to claim 1 for use in the treatment or prophylaxis of diseases and conditions caused by inappropriate activity of CXCR4.

34. A compound according to claim 1 for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers.

35. The compound of claim 34 wherein the condition or disease is HIV infection, rheumatoid arthritis, inflammation, or cancer.

36. The compound of claim 35 wherein the condition or disease is HIV.

37. Use of a compound according to claim 1 in the manufacture of a medicament for use in the treatment or prophylaxis of a condition or disease modulated by a chemokine receptor.

38. Use of a compound according to claim 37 wherein the chemokine receptor is CXCR4.

39. Use of a compound according to claim 1 in the manufacture of a medicament for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers.

40. Use of a compound as in claim 39 wherein the condition or disorder is HIV infection, rheumatoid arthritis, inflammation, or cancer.

41. Use of a compound as in claim 40 wherein the condition is HIV infection.

42. A method for the treatment or prophylaxis of a condition or disease modulated by a chemokine receptor comprising the administration of a compound according to claim 1.

43. The method of claim 42 wherein the chemokine receptor is CXCR4.

44. A method for the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of a compound according to claim 1.

45. A method for the treatment or prophylaxis of HIV infection, rheumatoid arthritis, inflammation, or cancer comprising the administration of a compound according to claim 1.

46. A method for the treatment or prophylaxis of HIV infection comprising the administration of a compound according to claim 1.

47. A method of treatment or prevention of a viral infection in a human comprising administering to said human a composition comprising a compound according to claim 1 and another therapeutic agent.

48. A composition according to claim 31, wherein said composition comprises at least one additional therapeutic agent selected from the group consisting of nucleotide reverse transcriptase inhibitors, non-nucleotide reverse transcriptase inhibitors, protease inhibitors, entry inhibitors, Integrase inhibitors, budding inhibitors, CCR5 inhibitors, or other CXCR4 inhibitors.

49. (canceled)

50. A process for the preparation of a compound of formula (I) 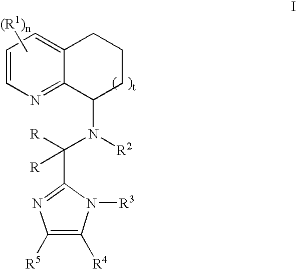
wherein
t is 1
each R is H
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)ₘR¹⁰, —S(O)ₘAy, cyano, nitro, or azido;
n is 0, 1, or 2;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₘR¹⁰, wherein R² is not amine or alkylamine, or substituted with amine or aklyamine;
R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}R^{a}NR⁶R⁷]ₚ, —R^{a}Ay[R^{a}NR⁶R⁷]ₚ-Het[NR⁶R⁷]ₚ, —R^{a}Het[NR⁶R⁷]ₚ, -Het[R^{a}NR⁶R⁷]ₚ or —R^{a}Het[R^{a}NR⁶R⁷]ₚ;
each p independently is 1 or 2;
each of R⁴ and R⁵ independently are selected from H, alkyl, alkenyl, alkynyl, cycloalkyl, -Ay, -Het, —R^{a}Ay, —R^{a}Het, —OR¹⁰, —NR⁶R⁷, —R^{a}NR⁶R⁷, C(O)R¹⁰, —CO₂R¹⁰, —C(O)NR⁶R⁷, —S(O)₂NR⁶R⁷, —S(O)ₘR¹⁰, cyano, nitro, or azido; or
R⁴ and R⁵ may combine to form a ring containing one or more degrees of unsaturation that is fused with the depicted imidazole ring;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay -Het, —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group;
comprising the step of reacting a compound of formula (VII) 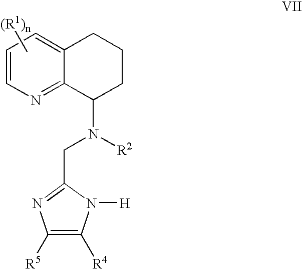
wherein R¹, R², R⁴ and R⁵ are as defined herein;
with a compound of formula Lg-R³ wherein Lg is a leaving group and R³ is as defined herein to form a compound of formula (I).

51. A process for the preparation of a compound of formula (I) 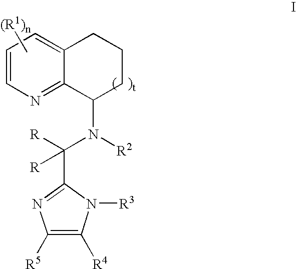
wherein
t is 1
each R is H
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, C(O)Het, —S(O)₂NR⁶R⁷, —S(O)ₘR¹⁰, —S(O)ₘAy, cyano, nitro, or azido;
n is 0, 1, or 2;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₘR¹⁰, wherein R² is not amine or alkylamine, or substituted with amine or aklyamine;
R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}NR⁶R⁷]ₚ R^{a}Ay[R^{a}NR⁶R⁷]ₚ, -Het[NR⁶R⁷]ₚ, —R^{a}Het[NR⁶R⁷]ₚ, -Het[R^{a}NR⁶R⁷]ₚ, or —R^{a}Het[R^{a}NR⁶R⁷]ₚ;
each p independently is 1 or 2;
each of R⁴ and R⁵ independently are selected from H, alkyl, alkenyl, alkynyl, cycloalkyl, -Ay, -Het, —R^{a}Ay, —R^{a}Het, —OR¹⁰, —NR⁶R⁷, —R^{a}NR⁶R⁷, —C(O)R¹⁰, —CO₂R¹⁰, —C(O)NR⁶R⁷, —S(O)₂NR⁶R⁷, —S(O)ₘR¹⁰), cyano, nitro, or azido; or
R⁴ and R⁵ may combine to form a ring containing one or more degrees of unsaturation that is fused with the depicted imidazole ring;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het, —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group;
comprising the step of reacting a compound of formula (II) 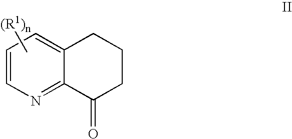
wherein R¹ and n are as defined herein;
with a compound of formula (VIII) 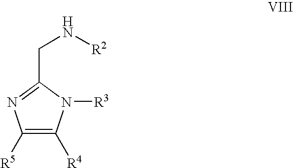
wherein R², R³, R⁴ and R⁵ are as defined herein;
under reductive amination conditions to form a compound of formula (I).

52. A process for the preparation of a compound of formula (I) 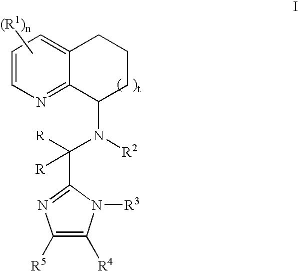
wherein
t is 1
each R is H
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, C(O)Het, —S(O)₂NR⁶R⁷, —S(O)ₘR¹⁰, —S(O)ₘAy, cyano, nitro, or azido;
n is 0, 1, or 2;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₘR¹⁰, wherein R is not amine or alkylamine, or substituted with amine or aklyamine;
R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}NR⁶R⁷]ₚ, —R^{a}Ay[R^{a}NR⁶R⁷]ₚ-Het[NR⁶R⁷]ₚ, —R^{a}Het[NR⁶R⁷]ₚ, -Het[R^{a}NR⁶R⁷]ₚ, or —R^{a}Het[R^{a}NR⁶R⁷]ₚ;
each p independently is 1 or 2;
each of R⁴ and R⁵ independently are selected from H, alkyl, alkenyl, alkynyl, cycloalkyl, -Ay, -Het, —R^{a}Ay, —R^{a}Het, —OR¹⁰, —NR⁶R⁷, —R^{a}NR⁶R⁷, —C(O)R¹⁰, —CO₂R¹⁰, —C(O)NR⁶R⁷, —S(O)₂NR⁶R⁷, —S(O)ₘR¹⁰, cyano, nitro, or azido; or
R⁴ and R⁵ may combine to form a ring containing one or more degrees of unsaturation that is fused with the depicted imidazole ring;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰), —R^{a}NR⁸R⁹, -Ay, -Het, —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group;
comprising the step of reacting a compound of formula (IV) 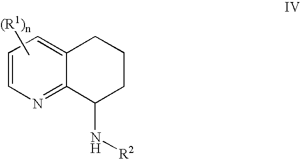
wherein R¹, R² and n are as defined herein;
with a compound of formula (IX) 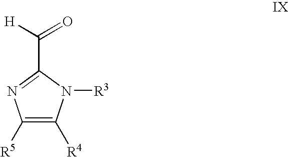
wherein R³, R⁴ and R⁵ are as defined herein;
under reductive amination conditions to form a compound of formula (I).

53. A process for the preparation of a compound of formula (I) 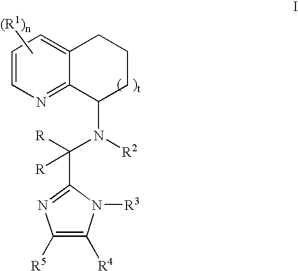
wherein
t is 1
each R is H
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)ₘR¹⁰, —S(O)ₘAy, cyano, nitro, or azido-n
n is 0, 1, or 2;
each m independently is 0, 1, or 2;
each R independently is H, alkyl, alkenyl, alkynyl, haloalkyl, cycloalkyl, —R^{a}Ay, —R^{a}OR¹⁰, or —R^{a}S(O)ₘR¹⁰, wherein R is not amine or alkylamine, or substituted with amine or aklyamine,
R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}NR⁶R⁷]ₚ R^{a}Ay[R^{a}NR⁶R⁷]ₚ-Het[NR⁶R⁷]ₚ, —R⁸Het[NR⁶R⁷]ₚ, -Het[R^{a}NR⁶R⁷]ₚ or —R^{a}Het[R^{a}NR⁶R⁷]ₚ;
each p independently is 1 or 2;
each of R⁴ and R⁵ combine to form a ring containing one or more degrees of unsaturation that is fused with the depicted imidazole ring and substituted with (R¹)ₙ;
each of R⁶ and R⁷ independently are selected from H, alkyl, alkenyl, alkynyl, cycloalkyl, cycloalkenyl, —R^{a}cycloalkyl, —R¹⁰H, —R^{a}OR¹⁰, —R^{a}NR⁸R⁹, -Ay, -Het, —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group;
comprising the steps of reacting a compound of formula (XV) 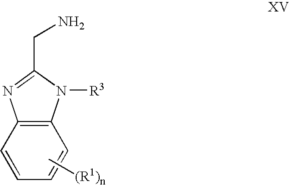
wherein R¹, R³ and n are as defined herein;
with a compound of formula (II) 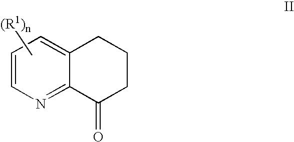
wherein R¹ and n are as defined herein;
to form a compound of formula (I-A); 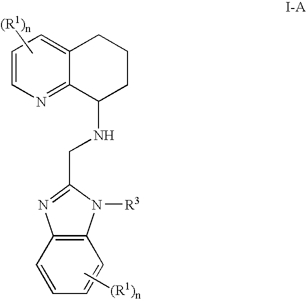
wherein R¹, R³ and n are as defined herein;
and subsequent reductive amination of formula (I-A) with an aldehyde to form a compound of formula (I).

54. A process for the preparation of a compound of formula (I) 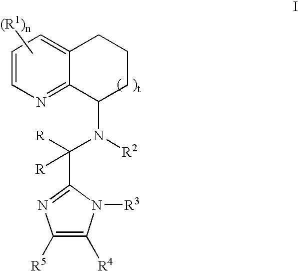
wherein
t is 1
each R is H each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, S(O)ₘR¹l₇-S(O)ₘAy, cyano, nitro, or azido;
n is 0, 1, or 2;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₘR¹⁰, wherein R² is not amine or alkylamine, or substituted with amine or aklyamine;
R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}NR⁶R⁷]ₚ, —R^{a}Ay[R^{a}NR⁶R⁷]ₚ, -Het[NR⁶R⁷]ₚ, —R^{a}Het[NR⁶R⁷]ₚ, -Het[R^{a}NR⁶R⁷]ₚ, or —R^{a}Het[R^{a}NR⁶R⁷]ₚ;
each p independently is 1 or 2;
each of R⁴ and R⁵ combine to form a ring containing one or more degrees of unsaturation that is fused with the depicted imidazole ring and substituted with (R¹)ₙ;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het, —R^{a}Ay —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group;
comprising the step of treating a compound of formula (XVII) 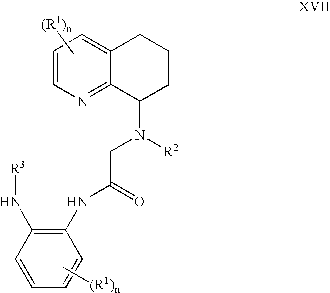
wherein R¹, R², R³ and n are as defined herein;
with an acid to form a compound of formula (I).

55. A process for the preparation of a compound of formula (I) 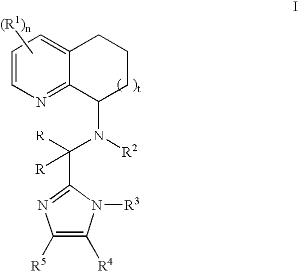
wherein
t is 1
each R is H
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)ₘR¹⁰, —S(O)ₘAy, cyano, nitro, or azido;
n is 0, 1, or 2;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₘR¹⁰, wherein R² is not amine or alkylamine, or substituted with amine or aklyamine;
R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}NR⁶R⁷]ₚ, —R^{a}Ay[R^{a}NR⁶R⁷]ₚ, -Het[NR⁶R⁷]ₚ, —R^{a}Het[NR⁶R⁷]ₚ, -Het[R^{a}NR⁶R⁷]ₚ or —R^{a}Het[R^{a}NR⁶R⁷]ₚ;
each p independently is 1 or 2;
each of R⁴ and R⁵ combine to form a ring containing one or more degrees of unsaturation that is fused with the depicted imidazole ring and substituted with (R¹)ₙ;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het, —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group;
comprising reacting a compound of formula (XVIII) 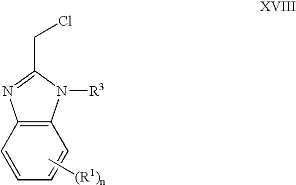
wherein R¹, R³ and n are as defined herein;
with an amine of formula (IV) 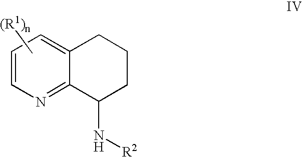
wherein R¹, R² and n are as defined herein;
to form a compound of formula (I).

56. A process for the preparation of a compound of formula (I) 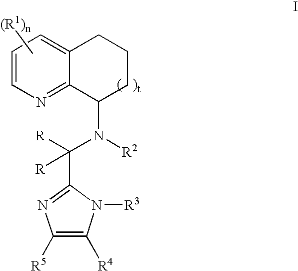
wherein
t is 1
each R is H
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)ₘR¹⁰, —S(O)ₘAy, cyano, nitro, or azido;
n is 0, 1, or 2;
each m independently is 0, 1, or 2;
each R² independently is H, alkyl, alkenyl, alkynyl, haloalkyl, cycloalkyl, —R^{a}Ay, —R^{a}OR¹⁰, or —R^{a}S(O)ₘR¹⁰, wherein R² is not amine or alkylamine, or substituted with amine or aklyamine;
R³ is -Het where Het is optionally substituted, —R^{a}Het where Het is optionally substituted, —R^{a}NR⁶R⁷, -Ay[NR⁶R⁷]ₚ, —R^{a}Ay[NR⁶R⁷]ₚ, -Ay[R^{a}NR⁶R⁷]ₚ, —R^{a}Ay[R^{a}NR⁶R⁷]ₚ, -Het[NR⁶R⁷]ₚ, —R^{a}Het[NR⁶R⁷]ₚ, -Het[R^{a}NR⁶R⁷]ₚ, or —R^{a}Het[R^{a}NR⁶R⁷]ₚ;
each p independently is 1 or 2;
each of R⁴ and R⁵ combine to form a ring containing one or more degrees of unsaturation that is fused with the depicted imidazole ring and substituted with (R¹)ₙ;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het —R^{a}Ay, —R^{a}Het, or —S(O)ₘR¹⁰;
each of R⁸ and R⁹ independently are selected from H or alkyl;
each R¹⁰ independently is H, alkyl, alkenyl, alkynyl, cycloalkyl, or -Ay;
each R^{a} independently is alkylene, cycloalkylene, alkenylene, cycloalkenylene, or alkynylene;
each Ay independently represents an optionally substituted aryl group; and
each Het independently represents an optionally substituted 4-, 5-, or 6-membered heterocyclyl or heteroaryl group;
comprising reacting a compound of formula (XX) 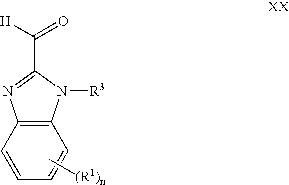
wherein R¹, R³ and n are as defined herein;
with a compound of formula (IV) 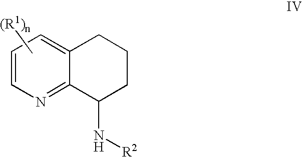
wherein R¹, R² and n are as defined herein;
to form a compound of formula (I).
